(19) United States
(12) Patent Application Publication
(10) Pub. No.: US 2024/0158394 A1
(43) Pub. Date: May 16, 2024

(54) NEK7 INHIBITORS

(71) Applicant: Halia Therapeutics, Inc., Lehi, UT (US)

(72) Inventors: Ramesh Subbiah, Hosur (IN); Alexis Henri Abel Mollard, Lehi, UT (US); David James Bearss, Salt Lake City, UT (US); John Sai Keong Kauwe, III, Salt Lake City, UT (US)

(21) Appl. No.: 18/467,436

(22) Filed: Sep. 14, 2023

Related U.S. Application Data
(60) Provisional application No. 63/406,610, filed on Sep. 14, 2022.

(51) Int. Cl. C07D 471/04; C07D 519/00

(52) U.S. Cl. CPC C07D 471/04; C07D 519/00

(57) ABSTRACT

Compounds having activity as inhibitors of NEK7 are provided. The compounds have Structure (I):

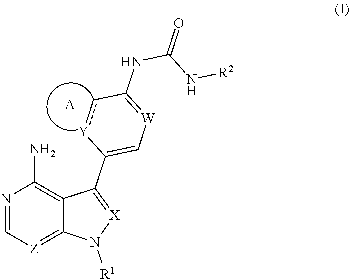

or a stereoisomer, tautomer, or salt thereof, wherein A, W, X, Y, Z, R¹, and R² are as defined herein. Methods associated with preparation and use of such compounds, pharmaceutical compositions comprising such compounds and methods to modulate the activity of the NLRP3 inflammasome are also provided.

### BACKGROUND

### Technical Field

Embodiments of the present disclosure are generally directed to compounds and methods for their preparation and use as therapeutic or prophylactic agents, for example for treatment of inflammation.

### Description of the Related Art

Inflammasomes are multi-protein complexes whose activation plays a central role in innate immunity and inflammation. To date, four inflammasomes have been described: NLRP1, NLRC4, NLRP3, and AIM2. The NLRP3 inflammasome is composed of NLRP3, ASC, and caspase-1. Its activation results in the activation of caspase-1 which promotes the secretion of IL-1β and IL-18, cytokines that mediate inflammation in animal disease models of several autoimmune diseases, myocardial infarction, metabolic syndromes, inflammatory bowel disease, and macrophage activation syndrome.

NEK7 is a member of the family of NIMA-related kinases (NEKs) and acts as an NLRP3-binding protein to regulate its oligomerization and activation. NEK7 is a serine/threonine kinase essential for mitotic entry, cell cycle progression, cell division, and mitotic progression. It is expressed in a variety of tissues such as the brain, heart, lung, liver, and spleen. Overexpression of NEK7 induces the production of abnormal cells, which has an intimate connection to tumors, such as retinoblastoma, gallbladder cancer and carcinoma of the head and neck.

A great number of inhibitors have been widely used to disturb effector signaling pathways, involving IL-1β or IL-18 without abolishing the inflammation response. Inhibitors of NLRP3 inflammasome activation that block the NLRP3-NEK7 interaction can have therapeutic or prophylactic activity in several human diseases, such as type 2 diabetes (T2D), atherosclerosis, gout, and neurodegenerative diseases. However, the exact mechanism of the NLRP3-NEK7 interaction is not well understood.

Accordingly, there is a need to develop inhibitors that will directly target NEK7 to affect the inflammatory response modulated by the NLRP3 inflammasome in several pathological diseases, such as gout, atherosclerosis, Type 2 diabetes, metabolic syndrome, macular degeneration, Alzheimer's disease, multiple sclerosis, and inflammatory bowel disease. Embodiments of the present disclosure fulfill this need and provide further related advantages.

### BRIEF SUMMARY

In brief, embodiments of the present disclosure provide compounds, including stereoisomers, tautomers, and salts thereof, which are capable of modulating the activity of the NLRP3 inflammasome.

One embodiment provides compounds of Structure (I):

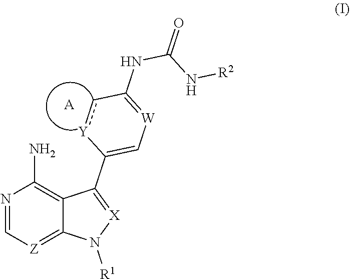

or a stereoisomer, tautomer, or salt thereof, wherein A, W, X, Y, Z, R¹, and R² are as defined herein. In another embodiment, pharmaceutical compositions comprising the disclosed compounds, and methods of use of the same for treatment of inflammation are also provided.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, ±5%, or ±1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Nitro” refers to the —NO₂ radical.

“Oxo” refers to the ═O substituent.

“Thiol” refers to the —SH substituent.

“Thioxo” refers to the ═S substituent.

“Alkyl” refers to a saturated, straight or branched hydrocarbon chain radical consisting solely of carbon and hydrogen atoms, having, for example, from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching but does not include carbon atoms belonging to any substituents. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds, having from two to twelve carbon atoms (C₂-C₁₂ alkenyl), two to eight carbon atoms (C₂-C₈ alkenyl) or two to six carbon atoms (C₂-C₆ alkenyl), or any value within these ranges, and which is attached to the rest of the molecule by a single bond, e.g., ethenyl, prop-1-enyl, but-1-enyl, pent-1-enyl, penta-1,4-dienyl, and the like. The number of carbons referred to relates to the carbon backbone and carbon branching but does not include carbon atoms belonging to any substituents. Unless stated otherwise specifically in the specification, an alkenyl group is optionally substituted.

The term “alkynyl” refers to unsaturated straight or branched hydrocarbon radical, having 2 to 12 carbon atoms (C₂-C₁₂ alkynyl), two to nine carbon atoms (C₂-C₉ alkynyl), or two to six carbon atoms (C₂-C₆ alkynyl), or any value within these ranges, and having at least one carbon-carbon triple bond. Examples of alkynyl groups may be selected from the group consisting of ethynyl, propargyl, but-1-ynyl, but-2-ynyl and the like. The number of carbons referred to relates to the carbon backbone and carbon branching but does not include carbon atoms belonging to any substituents. Unless stated otherwise specifically in the specification, an alkynyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Haloalkoxy” refers to a radical of the formula —ORₐ where Rₐ is a haloalkyl radical as defined herein containing one to twelve carbon atoms (C₁-C₁₂ haloalkoxy), one to eight carbon atoms (C₁-C₈ haloalkoxy) or one to six carbon atoms (C₁-C₆ haloalkoxy), or any value within these ranges. Unless stated otherwise specifically in the specification, a haloalkoxy group is optionally substituted.

“Aminyl” refers to a radical of the formula —NRₐR_{b}, where Rₐ and Rb are each independently H or C₁-C₆ alkyl as defined above. When both of Rₐ and R_{b} are H, an “aminyl” group is the same as an “amino” group as defined above. The C₁-C₆ alkyl portion of an aminyl group is optionally substituted unless stated otherwise.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of 7-electrons (for example, alternating double and single bonds) that is even but not a multiple of 4 (i.e., 4n+2 π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, or pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic, or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Carbocyclic” or “carbocycle” refers to a ring system, wherein each of the ring atoms are carbon.

“Cycloalkyl” refers to a non-aromatic monocyclic or polycyclic carbocyclic radical consisting solely of carbon and hydrogen atoms, which may include fused, spiro, or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, spiro[4.4]nonayl, and the like. Unless otherwise stated specifically in the specification, a cycloalkyl group is optionally substituted.

“Halo” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Hydroxylalkyl” or “hydroxyalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxyalkyl group is optionally substituted.

“Heterocyclyl” refers to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen, and sulfur. Unless stated otherwise specifically in the specification, the heterocyclyl radical is partially or fully saturated and is a monocyclic, bicyclic, tricyclic, or tetracyclic ring system, which may include fused, spirocyclic and/or bridged ring systems. Nitrogen, carbon, and sulfur atoms in a heterocyclyl radical are optionally oxidized, and nitrogen atoms may be optionally quaternized. Examples of such heterocyclyl radicals include, but are not limited to, dioxolanyl, thienyl[1,3]dithianyl, decahydroisoquinolyl, furanonyl, imidazolinyl, imidazolidinyl, isothiazolidinyl, isoxazolidinyl, morpholinyl, octahydroindolyl, octahydroisoindolyl, hexahydro-1H-pyrrolizine, 2-oxopiperazinyl, 2-oxopiperidinyl, 2-oxopyrrolidinyl, oxazolidinyl, oxiranyl, piperidinyl, piperazinyl, 4-piperidonyl, azetidinyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

“Heterocyclylalkyl” refers to a radical comprising a heterocyclyl group as defined above that is connected to the remainder of the molecule by an alkylene linker (i.e., a bivalent alkyl group). Unless stated otherwise specifically in the specification, a heterocyclylalkyl group is optionally substituted.

“Heterocyclyloxy” refers to a radical group of the formula —OR^{a} where R^{a} is a heterocyclyl group as defined above. Unless stated otherwise specifically in the specification, a heterocyclyloxy group is optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen, and sulfur, and at least one aromatic ring. Heteroaryl radicals may be a monocyclic, bicyclic, tricyclic, or tetracyclic ring system, which may include fused or bridged ring systems; and the nitrogen, carbon, or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). In some embodiments, a heteroaryl group has one of the following structures:

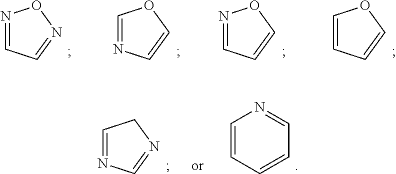 [SMILES: c1cnon1 ; c1cocn1 ; c1cnoc1 ; c1ccoc1 ; C1=NC=NC1 ; c1ccncc1]

Unless stated otherwise specifically in the specification, a heteroaryl group is optionally substituted.

The term “substituted” as used herein means any of the above groups (e.g., alkyl, alkenyl, alkylene, alkylcarbonyl, alkoxy, alkoxyalkyl, aminylalkyl, aryl, cyanoalkyl, cycloalkyl, haloalkyl, heterocyclyl, heterocyclene, heterocyclylalkyl, heteroaryl, heteroarylalkyl and/or hydroxylalkyl) wherein at least one hydrogen atom (e.g., 1, 2, 3 or all hydrogen atoms) is replaced by a bond to a non-hydrogen substituent. Examples of non-hydrogen substituents include, but are not limited to: amino, carboxyl, cyano, hydroxyl, halo, nitro, oxo, thiol, thioxo, alkyl, alkenyl, alkylcarbonyl, alkoxy, aryl, cyanoalkyl, cycloalkyl, haloalkyl, heterocyclyl, heterocyclylalkyl, heteroaryl, heteroarylalkyl, and/or hydroxylalkyl substituents, each of which may also be optionally substituted with one or more of the above substituents.

In some specific embodiments, optional substituents are independently selected from the group consisting of halo, hydroxyl, cyano, aminyl, C₁-C₆ alkyl, C₂-C₆ alkenyl, C₂-C₆ alkynyl, C₁-C₆ haloalkyl, C₃-C₈ cycloalkyl, C₃-C₈ halocycloalkyl, C₆-C₁₀ aryl, 5- or 6-membered heteroaryl, C₁-C₆ alkoxy, and 3-8 membered heterocyclyl. In some embodiments, optional substituents are independently selected from the group consisting of halo, cyano, optionally substituted C₁-C₆ alkyl, optionally substituted C₁-C₆ haloalkyl, optionally substituted C₁-C₆ alkoxy, optionally substituted C₁-C₆ haloalkoxy, and optionally substituted C₃-C₈ cycloalkyl. In some embodiments, optional substituents are independently selected from halo, cyano, optionally substituted C₁-C₆ alkyl, optionally substituted C₁-C₆ haloalkyl, optionally substituted C₁-C₆ alkoxy, optionally substituted C₁-C₆ haloalkoxy, or optionally substituted C₃-C₈ cycloalkyl, optionally substituted 5-10-membered heterocyclylalkyl, optionally substituted 5-10-membered heterocyclyloxy, and optionally substituted C₆-C₁₀ aryl.

The term “effective amount” or “therapeutically effective amount” refers to that amount of a compound described herein that is sufficient to affect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying, or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

The terms “antagonist” and “inhibitor” are used interchangeably, and they refer to a compound having the ability to inhibit a biological function of a target protein, whether by inhibiting the activity or expression of the protein, such as NLRP3 inflammasome or NEK7 or the association of NLRP3 inflammasome-NEK7. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

“Signal transduction” is a process during which stimulatory or inhibitory signals are transmitted into and within a cell to elicit an intracellular response.

The term “selective inhibition” or “selectively inhibit” refers to a biologically active agent refers to the agent's ability to preferentially reduce the target signaling activity as compared to off target signaling activity, via direct or indirect interaction with the target.

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compounds of Structure (I)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate, and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds of Structure (I), including salts, stereoisomers, tautomers, polymorphs, solvates, hydrates, and prodrugs thereof.

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some aspects, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution (“unsubstituted”). When a functional group is described as “optionally substituted,” and in turn, substituents on the functional group are also “optionally substituted” and so on, for the purposes of this disclosure, such iterations are limited to five, four, or three. In some embodiments, such iterations are limited to two. In some embodiments, such iterations are limited to one. In some embodiments, when a functional group is described as “optionally substituted” substituents on the functional group are unsubstituted.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents, or excipients therefor.

“Pharmaceutically acceptable carrier, diluent, or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (i.e., compounds of Structure (I)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I) are a mixture of enantiomers or diastereomers. In other embodiments, the compounds of Structure (I) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Professional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### Compounds

The disclosure provides compounds including stereoisomers, tautomers, and salts thereof, which are capable of modulating the activity of the NLRP3 inflammasome.

One embodiment provides a compound having the following Structure (I):

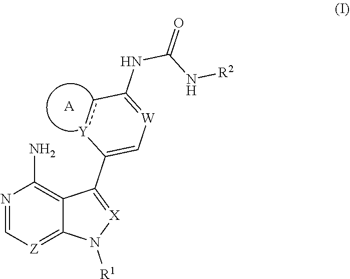

or a stereoisomer, tautomer, or salt thereof, wherein:
[symbol] represents a double or a single bond such that all valences are satisfied;
A is an optionally substituted 5-6-membered heterocyclyl, an optionally substituted 6-membered aryl, or an optionally substituted 5-6-membered heteroaryl;
X is N or CR³;
Y is C or N;
W is CH or N;
Z is CH or N;
R¹ is optionally substituted C₁-C₆ alkyl, optionally substituted C₁-C₆ alkenyl, optionally substituted C₁-C₆ alkynyl, optionally substituted C₁-C₆ hydroxyalkyl, optionally substituted C₁-C₆ alkoxyalkyl, optionally substituted C₁-C₆ carboxyalkyl, optionally substituted C₁-C₆ haloalkyl, optionally substituted C₃-C₅ cycloalkyl, optionally substituted C₆-C₁₀ aryl, optionally substituted 3-10 membered heterocyclyl, or optionally substituted 5-10 membered heteroaryl;
R² is optionally substituted aryl or optionally substituted heteroaryl; and
R³ is hydrogen, halo, cyano, optionally substituted C₁-C₆ alkyl, optionally substituted C₁-C₆ haloalkyl, optionally substituted C₁-C₆ alkoxy, optionally substituted C₁-C₆ haloalkoxy, or optionally substituted C₃-C₈ cycloalkyl.

In some embodiments, [symbol] represents a double bond. In some other embodiments, [symbol] represents a single bond.

In some embodiments, A is a 5-membered heterocyclyl. In certain embodiments, A is a 6-membered heterocyclyl. In some embodiments, A is a 6-membered heteroaryl. In certain embodiments, A is a 5-membered heteroaryl. In some embodiments, A is a 6-membered aryl.

In some embodiments, Y is N. In some embodiments, A is unsubstituted. In certain embodiments, A is substituted. In some embodiments, A is substituted with one or more substituents selected from the group consisting of halo, cyano, optionally substituted C₁-C₆ alkyl, optionally substituted C₁-C₆ haloalkyl, optionally substituted C₁-C₆ alkoxy, optionally substituted C₁-C₆ haloalkoxy, or optionally substituted C₃-C₈ cycloalkyl.

In some embodiments, A is substituted with one or more substituents selected from the group consisting of halo, optionally substituted C₁-C₆ alkyl, optionally substituted C₁-C₆ haloalkyl, optionally substituted C₁-C₆ alkoxy, and optionally substituted C₁-C₆ haloalkoxy.

In some embodiments, A is substituted with one or more substituents selected from the group consisting of C₁-C₆ alkyl, halo, and C₁-C₆ haloalkyl. In some embodiments, A is substituted with one or more substituents selected from the group consisting of methyl, fluoro, and trifluoromethyl.

In some embodiments,

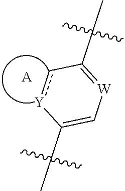

has the following structure:

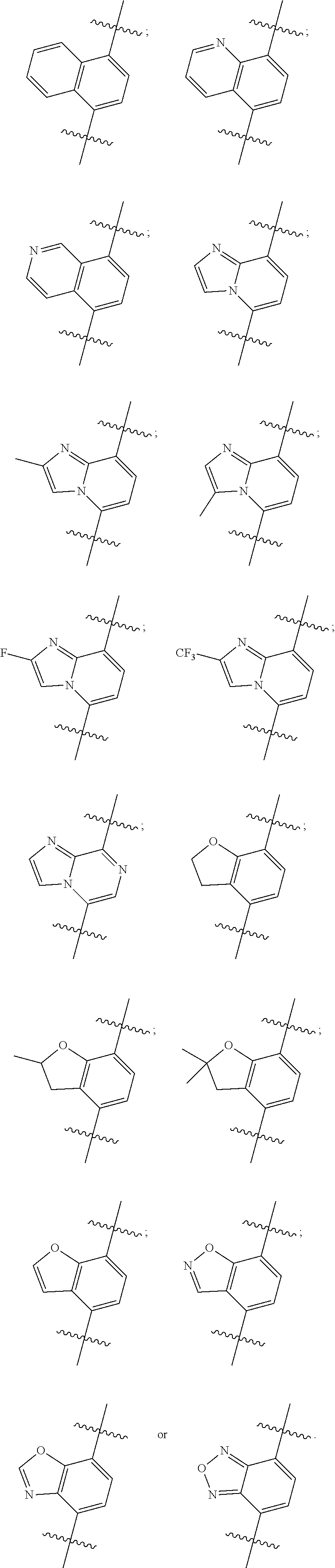

In some embodiments, Z is CH. In certain embodiments, Z is N. In certain embodiments, X is N. In some embodiments, X is CR³. In some embodiments, R³ is hydrogen or optionally substituted C₁-C₆ alkyl. In certain embodiments, X is CH.

In some embodiments, R¹ is optionally substituted C₁-C₆ alkyl, optionally substituted C₁-C₆ alkenyl, optionally substituted C₁-C₆ alkynyl, optionally substituted C₁-C₆ hydroxyalkyl, optionally substituted C₁-C₆ alkoxyalkyl, optionally substituted C₁-C₆ carboxyalkyl, optionally substituted C₃-C₈ cycloalkyl, optionally substituted 3-10 membered heterocyclyl, or optionally substituted 5-10 membered heteroaryl. In certain embodiments, R¹ is optionally substituted C₁-C₆ alkyl. In some embodiments, R¹ is methyl, ethyl, or iso-propyl. In certain embodiments, R¹ is optionally substituted C₁-C₆ alkenyl or optionally substituted C₁-C₆ alkynyl. In some more specific embodiments, R¹ has the following structure:

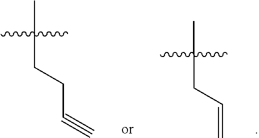

In some embodiments, R¹ is optionally substituted C₁-C₆ hydroxyalkyl. In certain embodiments, R¹ has one of the following structures:

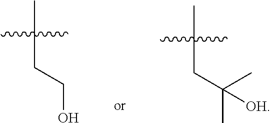

In some embodiments, R¹ is cyclopropyl, cyclobutyl, cyclopentyl, or cyclohexyl. In some more specific embodiments, R¹ has one of the following structures:

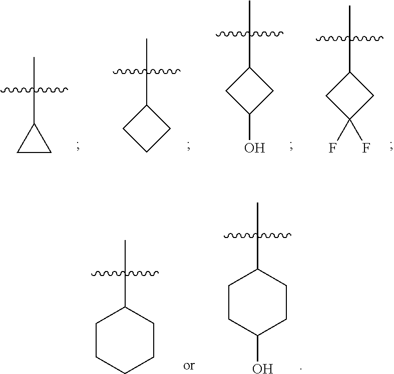

In some embodiments, R¹ is optionally substituted 3-10 membered heterocyclyl. In certain embodiments, R¹ has the following structure:

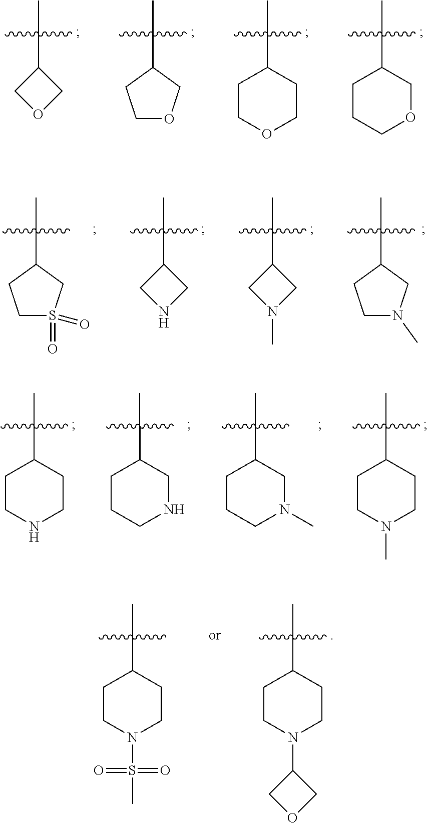

In some embodiments, R¹ is optionally substituted 5-10 membered heteroaryl. In certain embodiments, R¹ is unsubstituted pyridinyl. In some embodiments, R¹ has one of the following structures:

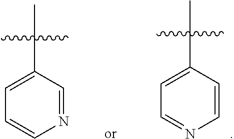 [SMILES: *c1cccnc1 ; *c1ccncc1]

In some embodiments, R¹ is optionally substituted C₁-C₆ carboxyalkyl. In certain embodiments, R¹ has the following structure:

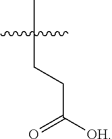 [SMILES: *CCC(=O)O]

In some embodiments, R¹ is optionally substituted C₁-C₆ alkoxyalkyl. In certain embodiments, R¹ has the following structure:

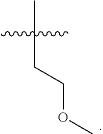 [SMILES: *CCOC]

In certain embodiments, R² is optionally substituted phenyl or optionally substituted 5-membered heteroaryl. In some embodiments, R² is optionally substituted phenyl. In certain embodiments, R² is optionally substituted 5-membered heteroaryl. In certain embodiments, R² is optionally substituted isoxazolyl or optionally substituted pyrazolyl. In some embodiments, R² is optionally substituted with one or more substituents selected from the group consisting of halo, cyano, optionally substituted C₁-C₆ alkyl, optionally substituted C₁-C₆haloalkyl, optionally substituted C₁-C₆ alkoxy, optionally substituted C₁-C₆ haloalkoxy, optionally substituted C₃-C₈ cycloalkyl, optionally substituted 5-10-membered heterocyclylalkyl, optionally substituted 5-10-membered heterocyclyloxy, and optionally substituted C₆-C₁₀ aryl.

In some embodiments, R² is optionally substituted with one or more substituents selected from the following structures:

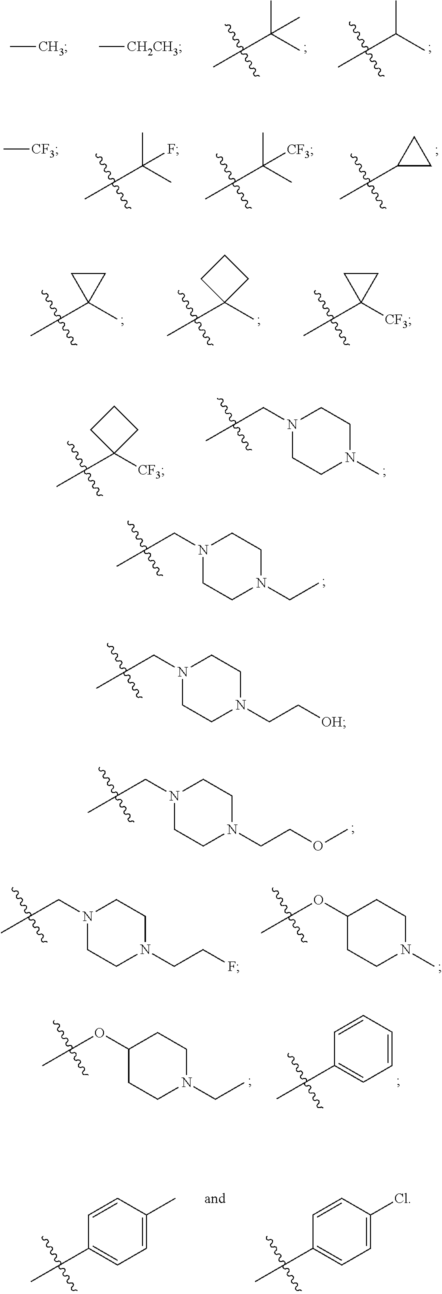

In some embodiments, R² has one of the following structures:

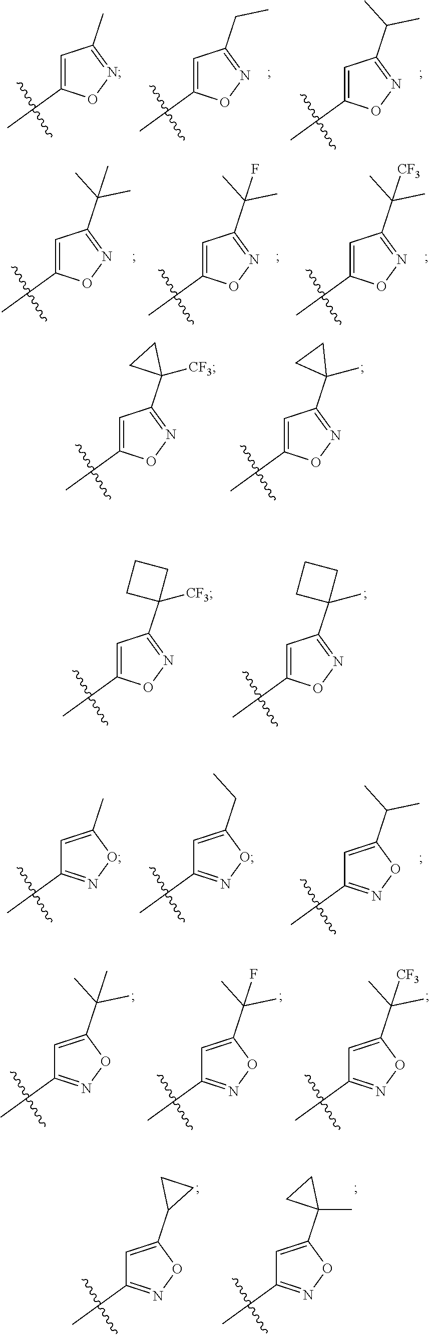

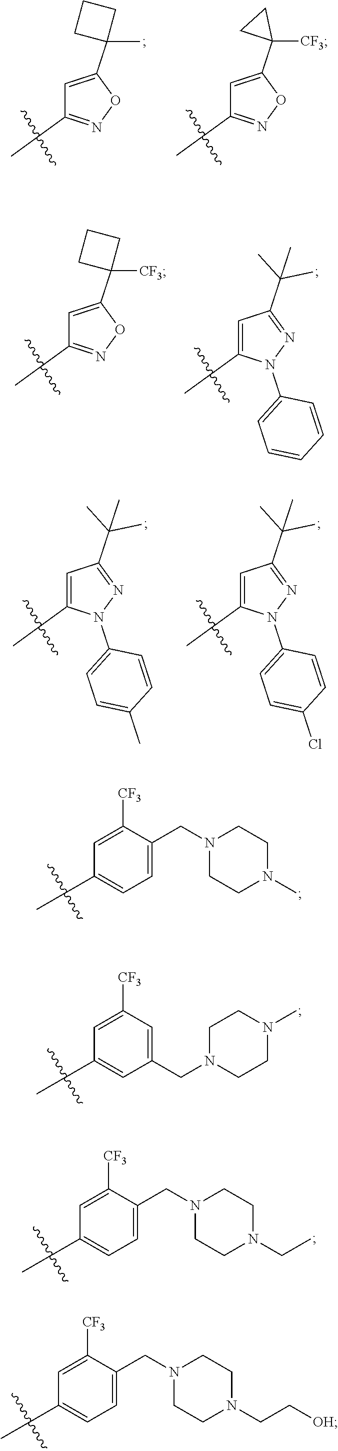

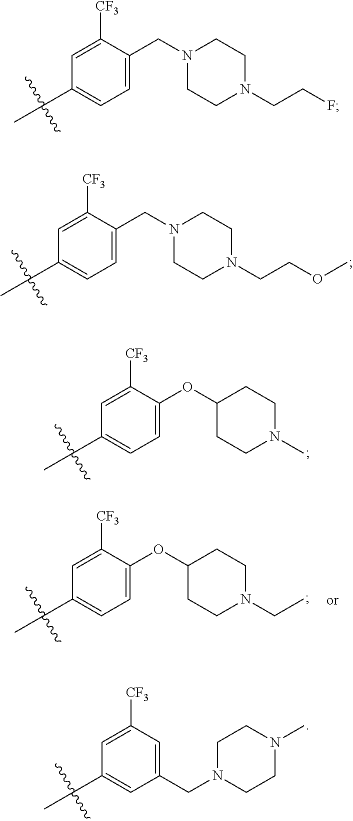 [SMILES: *c1ccc(CN2CCN(CCF)CC2)c(C(F)(F)F)c1 ; *c1ccc(CN2CCN(CCOC)CC2)c(C(F)(F)F)c1 ; *c1ccc(OC2CCN(C)CC2)c(C(F)(F)F)c1 ; *c1ccc(OC2CCN(CC)CC2)c(C(F)(F)F)c1 ; *c1cc(CN2CCN(C)CC2)cc(C(F)(F)F)c1]

In some embodiments, R² has one of the following structures:

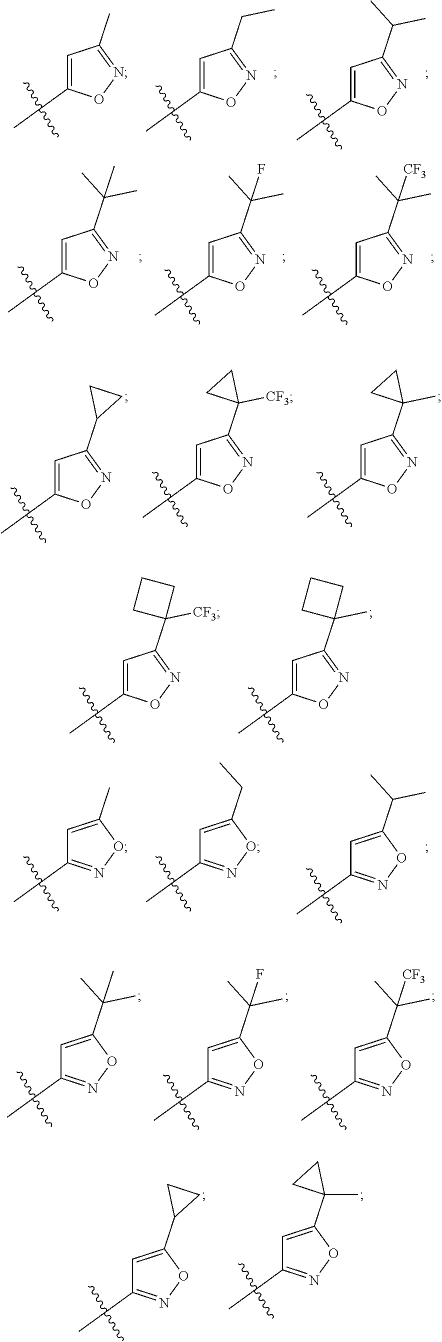

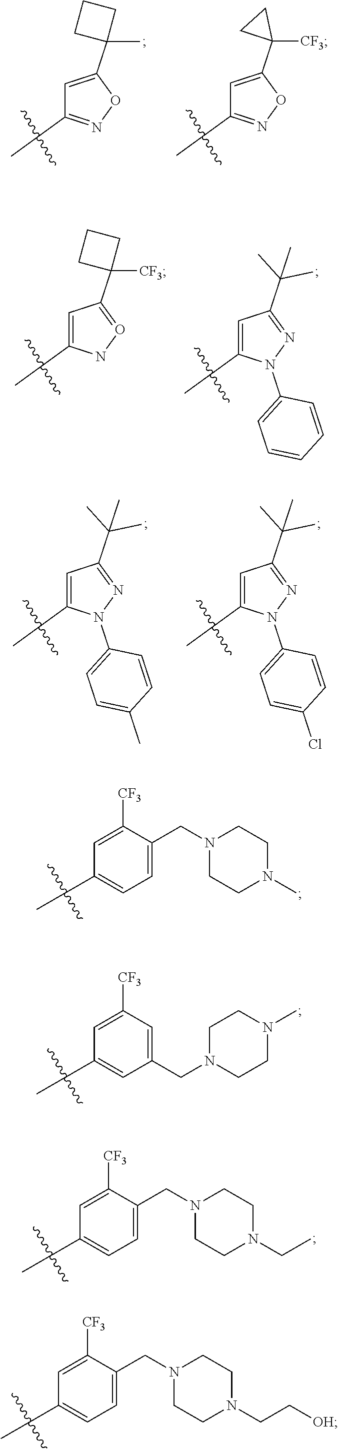

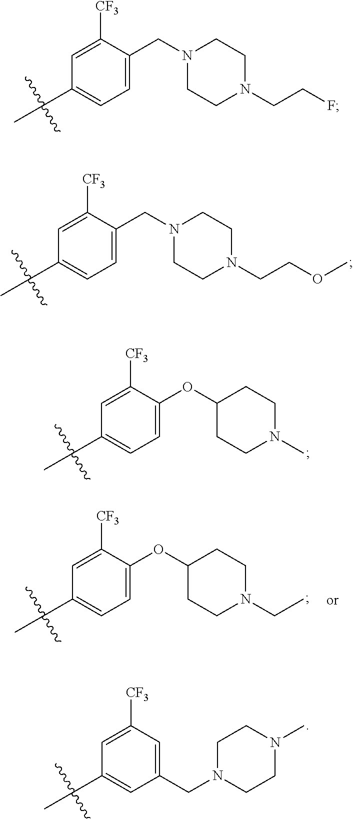 [SMILES: *c1ccc(CN2CCN(CCF)CC2)c(C(F)(F)F)c1 ; *c1ccc(CN2CCN(CCOC)CC2)c(C(F)(F)F)c1 ; *c1ccc(OC2CCN(C)CC2)c(C(F)(F)F)c1 ; *c1ccc(OC2CCN(CC)CC2)c(C(F)(F)F)c1 ; *c1cc(CN2CCN(C)CC2)cc(C(F)(F)F)c1]

In some embodiments, the compound of Structure (I) is a modulator of the NLRP3 inflammasome. In certain embodiments, the compound of Structure (I) is an inhibitor of NEK7.

In various different embodiments, the compound has one of the structures set forth in Table 1 below, or a stereoisomer, tautomer, or salt thereof. Compounds in Table 1 were prepared as described in the Examples or methods known in the art and analyzed by mass spectrometry and/or ¹H NMR.

TABLE 1
Representative compounds of Structure (I)

| No. | Structure | Name |
|---|---|---|
| I-1 | 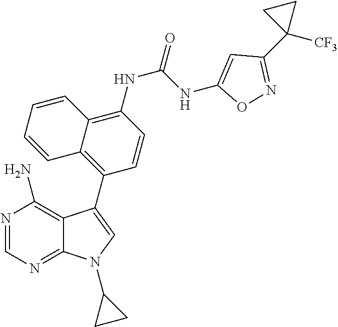 [SMILES: Nc1ncnc2c1c(-c1ccc(NC(=O)Nc3cc(C4(C(F)(F)F)CC4)no3)c3ccccc13)cn2C1CC1] | 1-(4-(4-amino-7-cyclopropyl- 7H-pyrrolo[2,3-d]pyrimidin- 5-yl)naphthalen-1-yl)-3-(3-(1- (trifluoromethyl)cyclopropyl)- isoxazol-5-yl)urea |
| I-2 | 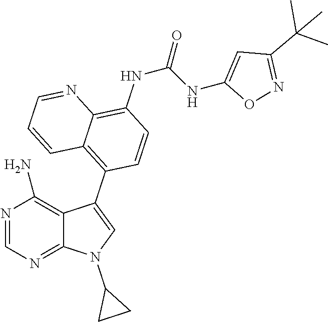 [SMILES: CC(C)(C)c1cc(NC(=O)Nc2ccc(-c3cn(C4CC4)c4ncnc(N)c34)c3cccnc23)on1] | 1-(5-(4-amino-7-cyclopropyl- 7H-pyrrolo[2,3-d]pyrimidin- 5-yl)quinolin-8-yl)-3-(3-(tert- butyl)isoxazol-5-yl)urea |
| I-3 | 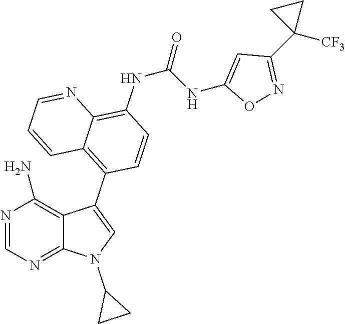 [SMILES: Nc1ncnc2c1c(-c1ccc(NC(=O)Nc3cc(C4(C(F)(F)F)CC4)no3)c3ncccc13)cn2C1CC1] | 1-(5-(4-amino-7-cyclopropyl- 7H-pyrrolo[2,3-d]pyrimidin- 5-yl)quinolin-8-yl)-3-(3-(1- (trifluoromethyl)cyclopropyl)- isoxazol-5-yl)urea |
| I-4 | 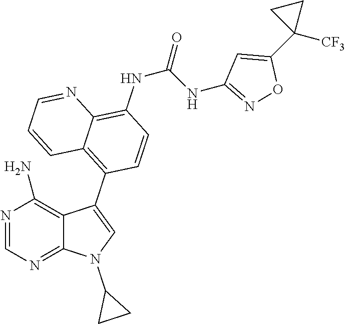 [SMILES: Nc1ncnc2c1c(-c1ccc(NC(=O)Nc3cc(C4(C(F)(F)F)CC4)on3)c3ncccc13)cn2C1CC1] | 1-(5-(4-amino-7-cyclopropyl- 7H-pyrrolo[2,3-d]pyrimidin- 5-yl)quinolin-8-yl)-3-(5-(1- (trifluoromethyl)cyclopropyl)- isoxazol-3-yl)urea |
| I-5 | 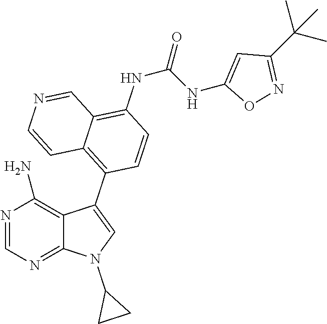 [SMILES: CC(C)(C)c1cc(NC(=O)Nc2ccc(-c3cn(C4CC4)c4ncnc(N)c34)c3ccncc23)on1] | 1-(5-(4-amino-7-cyclopropyl- 7H-pyrrolo[2,3-d]pyrimidin-5- yl)isoquinolin-8-yl)-3-(3-(tert- butyl)isoxazol-5-yl)urea |
| I-6 | 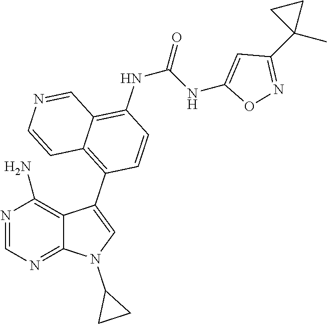 [SMILES: CC1(c2cc(NC(=O)Nc3ccc(-c4cn(C5CC5)c5ncnc(N)c45)c4ccncc34)on2)CC1] | 1-(5-(4-amino-7-cyclopropyl- 7H-pyrrolo[2,3-d]pyrimidin- 5-yl)isoquinolin-8-yl)-3-(3-(1- methylcyclopropyl)isoxazol-5- yl)urea |
| I-7 | 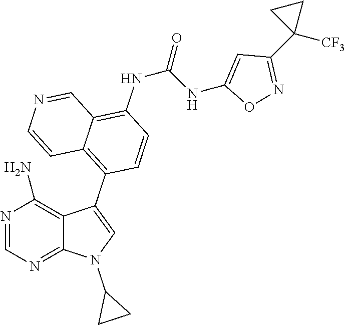 [SMILES: Nc1ncnc2c1c(-c1ccc(NC(=O)Nc3cc(C4(C(F)(F)F)CC4)no3)c3cnccc13)cn2C1CC1] | 1-(5-(4-amino-7-cyclopropyl- 7H-pyrrolo[2,3-d]pyrimidin- 5-yl)isoquinolin-8-yl)-3-(3-(1- (trifluoromethyl)cyclopropyl)- isoxazol-5-yl)urea |
| I-8 | 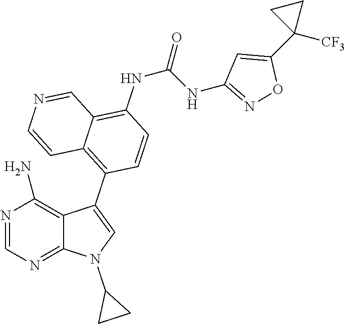 [SMILES: Nc1ncnc2c1c(-c1ccc(NC(=O)Nc3cc(C4(C(F)(F)F)CC4)on3)c3cnccc13)cn2C1CC1] | 1-(5-(4-amino-7-cyclopropyl- 7H-pyrrolo[2,3-d]pyrimidin- 5-yl)isoquinolin-8-yl)-3-(5-(1- (trifluoromethyl)cyclopropyl)- isoxazol-3-yl)urea |
| I-9 | 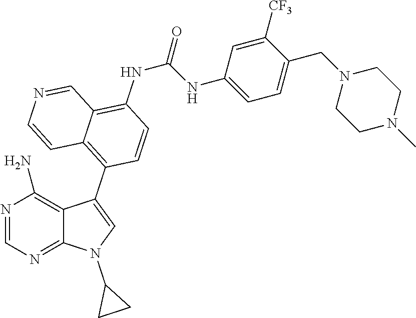 [SMILES: CN1CCN(Cc2ccc(NC(=O)Nc3ccc(-c4cn(C5CC5)c5ncnc(N)c45)c4ccncc34)cc2C(F)(F)F)CC1] | 1-(5-(4-amino-7-cyclopropyl- 7H-pyrrolo[2,3-d]pyrimidin- 5-yl)isoquinolin-8-yl)-3-(4- ((4-methylpiperazin-1-yl)- methyl)-3-(trifluoromethyl)- phenyl)urea |
| I-10 | 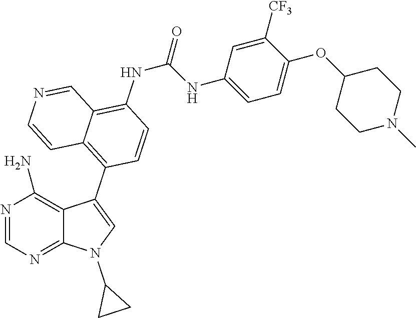 [SMILES: CN1CCC(Oc2ccc(NC(=O)Nc3ccc(-c4cn(C5CC5)c5ncnc(N)c45)c4ccncc34)cc2C(F)(F)F)CC1] | 1-(5-(4-amino-7-cyclopropyl- 7H-pyrrolo[2,3-d]pyrimidin- 5-yl)isoquinolin-8-yl)-3-(4-((1- methylpiperidin-4-yl)oxy)-3- (trifluoromethyl)phenyl)urea |
| I-11 | 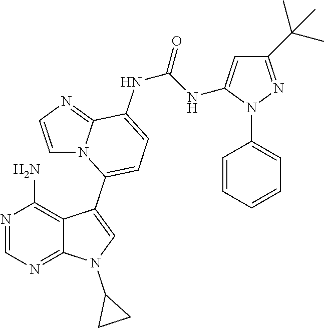 [SMILES: CC(C)(C)c1cc(NC(=O)Nc2ccc(-c3cn(C4CC4)c4ncnc(N)c34)n3ccnc23)n(-c2ccccc2)n1] | 1-(5-(4-amino-7-cyclopropyl- 7H-pyrrolo[2,3-d]pyrimidin- 5-yl)imidazo[1,2-a]pyridin-8- yl)-3-(3-(tert-butyl)-1-phenyl- 1H-pyrazol-5-yl)urea |
| I-12 | 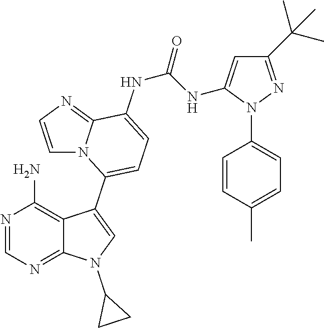 [SMILES: Cc1ccc(-n2nc(C(C)(C)C)cc2NC(=O)Nc2ccc(-c3cn(C4CC4)c4ncnc(N)c34)n3ccnc23)cc1] | 1-(5-(4-amino-7-cyclopropyl- 7H-pyrrolo[2,3-d]pyrimidin- 5-yl)imidazo[1,2-a]pyridin-8- yl)-3-(3-(tert-butyl)-1-(p- tolyl)-1H-pyrazol-5-yl)urea |
| I-13 | 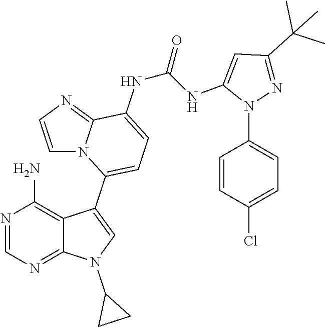 [SMILES: CC(C)(C)c1cc(NC(=O)Nc2ccc(-c3cn(C4CC4)c4ncnc(N)c34)n3ccnc23)n(-c2ccc(Cl)cc2)n1] | 1-(5-(4-amino-7-cyclopropyl- 7H-pyrrolo[2,3-d]pyrimidin- 5-yl)imidazo[1,2-a]pyridin-8- yl)-3-(3-(tert-butyl)-1-(4- chlorophenyl)-1H-pyrazol-5- yl)urea |
| I-14 | 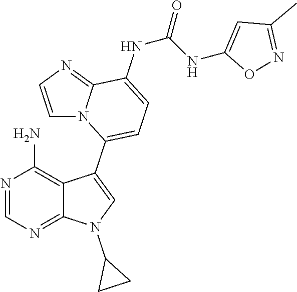 [SMILES: Cc1cc(NC(=O)Nc2ccc(-c3cn(C4CC4)c4ncnc(N)c34)n3ccnc23)on1] | 1-(5-(4-amino-7-cyclopropyl- 7H-pyrrolo[2,3-d]pyrimidin- 5-yl)imidazo[1,2-a]pyridin-8- yl)-3-(3-methylisoxazol-5-yl)- urea |
| I-15 | 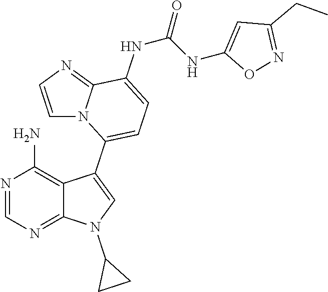 [SMILES: CCc1cc(NC(=O)Nc2ccc(-c3cn(C4CC4)c4ncnc(N)c34)n3ccnc23)on1] | 1-(5-(4-amino-7-cyclopropyl- 7H-pyrrolo[2,3-d]pyrimidin- 5-yl)imidazo[1,2-a]pyridin-8- yl)-3-(3-ethylisoxazol-5-yl)- urea |
| I-16 | 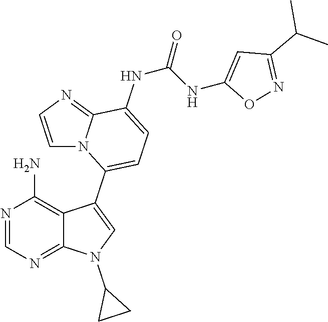 [SMILES: CC(C)c1cc(NC(=O)Nc2ccc(-c3cn(C4CC4)c4ncnc(N)c34)n3ccnc23)on1] | 1-(5-(4-amino-7-cyclopropyl- 7H-pyrrolo[2,3-d]pyrimidin- 5-yl)imidazo[1,2-a]pyridin-8- yl)-3-(3-isopropylisoxazol-5- yl)urea |
| I-17 | 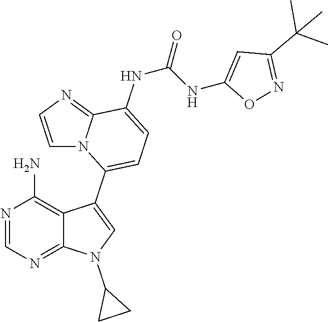 [SMILES: CC(C)(C)c1cc(NC(=O)Nc2ccc(-c3cn(C4CC4)c4ncnc(N)c34)n3ccnc23)on1] | 1-(5-(4-amino-7-cyclopropyl- 7H-pyrrolo[2,3-d]pyrimidin- 5-yl)imidazo[1,2-a]pyridin-8- yl)-3-(3-(tert-butyl)isoxazol-5- yl)urea |
| I-18 | 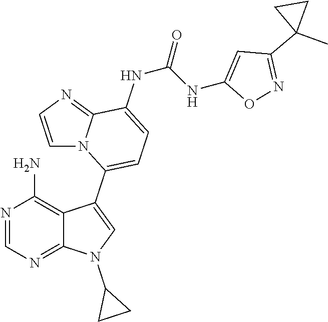 [SMILES: CC1(c2cc(NC(=O)Nc3ccc(-c4cn(C5CC5)c5ncnc(N)c45)n4ccnc34)on2)CC1] | 1-(5-(4-amino-7-cyclopropyl- 7H-pyrrolo[2,3-d]pyrimidin- 5-yl)imidazo[1,2-a]pyridin- 8-yl)-3-(3-(1-methylcyclo- propyl)isoxazol-5-yl)urea |
| I-19 | 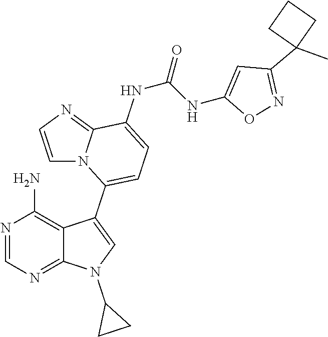 [SMILES: CC1(c2cc(NC(=O)Nc3ccc(-c4cn(C5CC5)c5ncnc(N)c45)n4ccnc34)on2)CCC1] | 1-(5-(4-amino-7-cyclopropyl- 7H-pyrrolo[2,3-d]pyrimidin- 5-yl)imidazo[1,2-a]pyridin-8- yl)-3-(3-(1-methylcyclobutyl)- isoxazol-5-yl)urea |
| I-20 | 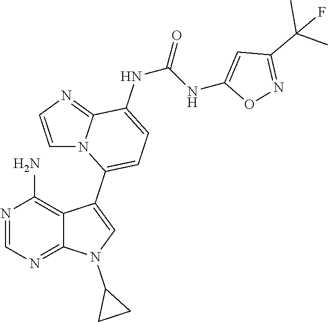 [SMILES: CC(C)(F)c1cc(NC(=O)Nc2ccc(-c3cn(C4CC4)c4ncnc(N)c34)n3ccnc23)on1] | 1-(5-(4-amino-7-cyclopropyl- 7H-pyrrolo[2,3-d]pyrimidin- 5-yl)imidazo[1,2-a]pyridin-8- yl)-3-(3-(2-fluoropropan-2- yl)isoxazol-5-yl)urea |
| I-21 | 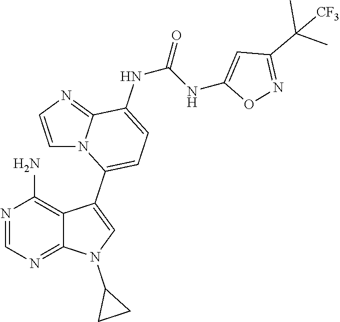 [SMILES: CC(C)(c1cc(NC(=O)Nc2ccc(-c3cn(C4CC4)c4ncnc(N)c34)n3ccnc23)on1)C(F)(F)F] | 1-(5-(4-amino-7-cyclopropyl- 7H-pyrrolo[2,3-d]pyrimidin- 5-yl)imidazo[1,2-a]pyridin-8- yl)-3-(3-(1,1,1-trifluoro-2- methylpropan-2-yl)isoxazol- 5-yl)urea |
| I-22 | 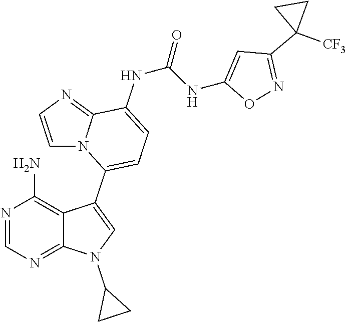 [SMILES: Nc1ncnc2c1c(-c1ccc(NC(=O)Nc3cc(C4(C(F)(F)F)CC4)no3)c3nccn13)cn2C1CC1] | 1-(5-(4-amino-7-cyclopropyl- 7H-pyrrolo[2,3-d]pyrimidin- 5-yl)imidazo[1,2-a]pyridin-8- yl)-3-(3-(1-(trifluoromethyl)- cyclopropyl)isoxazol-5-yl)- urea |
| I-23 | 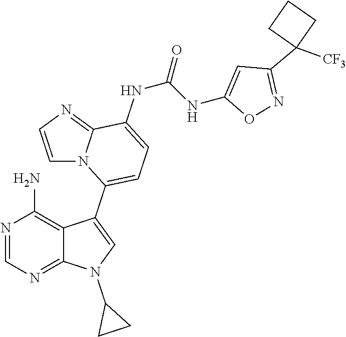 [SMILES: Nc1ncnc2c1c(-c1ccc(NC(=O)Nc3cc(C4(C(F)(F)F)CCC4)no3)c3nccn13)cn2C1CC1] | 1-(5-(4-amino-7-cyclopropyl- 7H-pyrrolo[2,3-d]pyrimidin- 5-yl)imidazo[1,2-a]pyridin-8- yl)-3-(3-(1-(trifluoromethyl)- cyclobutyl)isoxazol-5-yl)urea |
| I-24 | 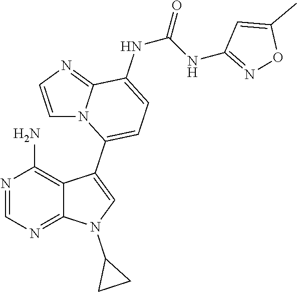 [SMILES: Cc1cc(NC(=O)Nc2ccc(-c3cn(C4CC4)c4ncnc(N)c34)n3ccnc23)no1] | 1-(5-(4-amino-7-cyclopropyl- 7H-pyrrolo[2,3-d]pyrimidin- 5-yl)imidazo[1,2-a]pyridin-8- yl)-3-(5-methylisoxazol-3- yl)urea |
| I-25 | 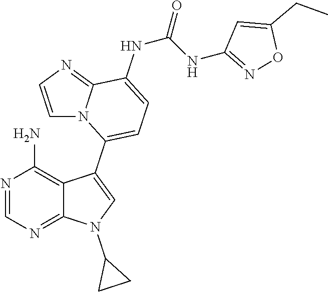 [SMILES: CCc1cc(NC(=O)Nc2ccc(-c3cn(C4CC4)c4ncnc(N)c34)n3ccnc23)no1] | 1-(5-(4-amino-7-cyclopropyl- 7H-pyrrolo[2,3-d]pyrimidin- 5-yl)imidazo[1,2-a]pyridin-8- yl)-3-(5-ethylisoxazol-3-yl)- urea |
| I-26 | 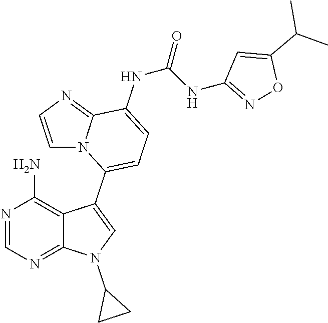 [SMILES: CC(C)c1cc(NC(=O)Nc2ccc(-c3cn(C4CC4)c4ncnc(N)c34)n3ccnc23)no1] | 1-(5-(4-amino-7-cyclopropyl- 7H-pyrrolo[2,3-d]pyrimidin- 5-yl)imidazo[1,2-a]pyridin-8- yl)-3-(5-isopropylisoxazol-3- yl)urea |
| I-27 | 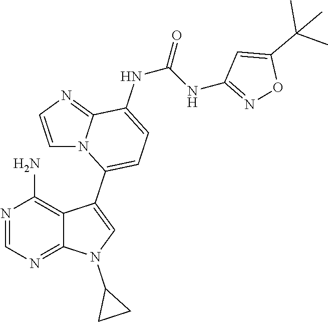 [SMILES: CC(C)(C)c1cc(NC(=O)Nc2ccc(-c3cn(C4CC4)c4ncnc(N)c34)n3ccnc23)no1] | 1-(5-(4-amino-7-cyclopropyl- 7H-pyrrolo[2,3-d]pyrimidin- 5-yl)imidazo[1,2-a]pyridin-8- yl)-3-(5-(tert-butyl)isoxazol-3- yl)urea |
| I-28 | 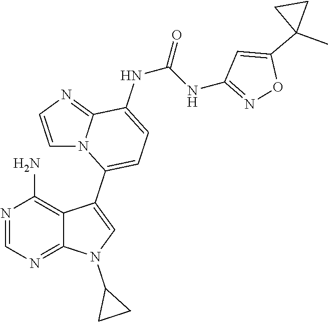 [SMILES: CC1(c2cc(NC(=O)Nc3ccc(-c4cn(C5CC5)c5ncnc(N)c45)n4ccnc34)no2)CC1] | 1-(5-(4-amino-7-cyclopropyl- 7H-pyrrolo[2,3-d]pyrimidin- 5-yl)imidazo[1,2-a]pyridin-8- yl)-3-(5-(1-methylcyclo- propyl)-isoxazol-3-yl)urea |
| I-29 | 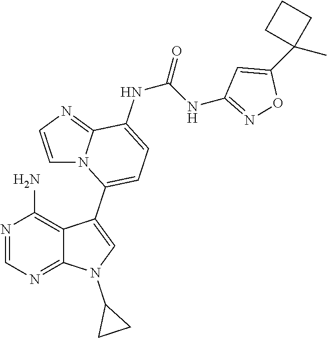 [SMILES: CC1(c2cc(NC(=O)Nc3ccc(-c4cn(C5CC5)c5ncnc(N)c45)n4ccnc34)no2)CCC1] | 1-(5-(4-amino-7-cyclopropyl- 7H-pyrrolo[2,3-d]pyrimidin- 5-yl)imidazo[1,2-a]pyridin-8- yl)-3-(5-(1-methylcyclobutyl)- isoxazol-3-yl)urea |
| I-30 | 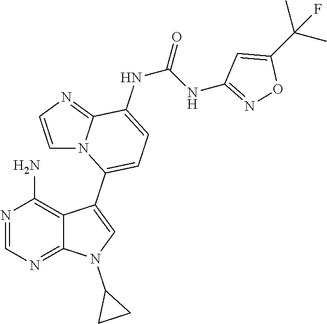 [SMILES: CC(C)(F)c1cc(NC(=O)Nc2ccc(-c3cn(C4CC4)c4ncnc(N)c34)n3ccnc23)no1] | 1-(5-(4-amino-7-cyclopropyl- 7H-pyrrolo[2,3-d]pyrimidin- 5-yl)imidazo[1,2-a]pyridin-8- yl)-3-(5-(2-fluoropropan-2-yl)- isoxazol-3-yl)urea |
| I-31 | 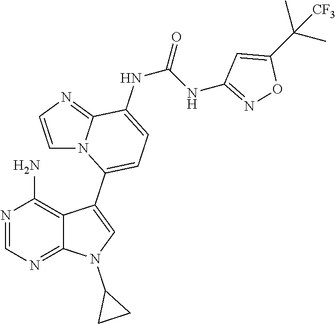 [SMILES: CC(C)(c1cc(NC(=O)Nc2ccc(-c3cn(C4CC4)c4ncnc(N)c34)n3ccnc23)no1)C(F)(F)F] | 1-(5-(4-amino-7-cyclopropyl- 7H-pyrrolo[2,3-d]pyrimidin- 5-yl)imidazo[1,2-a]pyridin-8- yl)-3-(5-(1,1,1-trifluoro-2- methylpropan-2-yl)isoxazol- 3-yl)urea |
| I-32 | 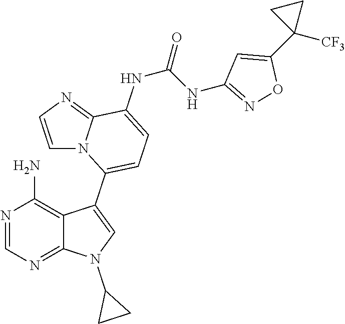 [SMILES: Nc1ncnc2c1c(-c1ccc(NC(=O)Nc3cc(C4(C(F)(F)F)CC4)on3)c3nccn13)cn2C1CC1] | 1-(5-(4-amino-7-cyclopropyl- 7H-pyrrolo[2,3-d]pyrimidin- 5-yl)imidazo[1,2-a]pyridin-8- yl)-3-(5-(1-(trifluoromethyl)- cyclopropyl)isoxazol-3-yl)- urea |
| I-33 | 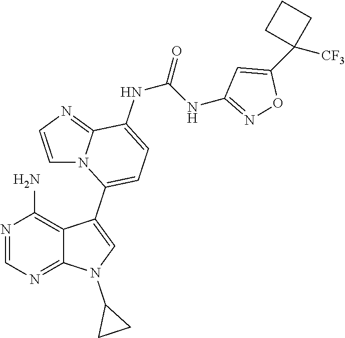 [SMILES: Nc1ncnc2c1c(-c1ccc(NC(=O)Nc3cc(C4(C(F)(F)F)CCC4)on3)c3nccn13)cn2C1CC1] | 1-(5-(4-amino-7-cyclopropyl- 7H-pyrrolo[2,3-d]pyrimidin- 5-yl)imidazo[1,2-a]pyridin-8- yl)-3-(5-(1-(trifluoromethyl)- cyclobutyl)isoxazol-3-yl)urea |
| I-34 | 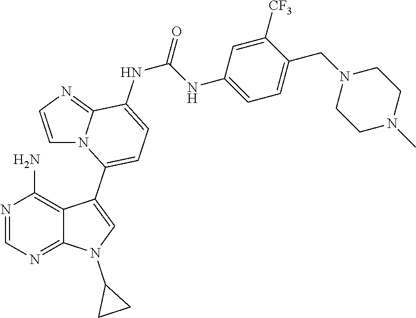 [SMILES: CN1CCN(Cc2ccc(NC(=O)Nc3ccc(-c4cn(C5CC5)c5ncnc(N)c45)n4ccnc34)cc2C(F)(F)F)CC1] | 1-(5-(4-amino-7-cyclopropyl- 7H-pyrrolo[2,3-d]pyrimidin- 5-yl)imidazo[1,2-a]pyridin-8- yl)-3-(4-((4-methylpiperazin- 1-yl)methyl)-3-(trifluorometh- yl)phenyl)urea |
| I-35 | 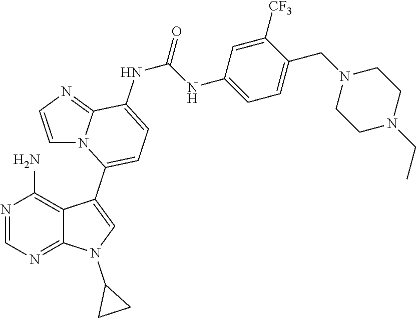 [SMILES: CCN1CCN(Cc2ccc(NC(=O)Nc3ccc(-c4cn(C5CC5)c5ncnc(N)c45)n4ccnc34)cc2C(F)(F)F)CC1] | 1-(5-(4-amino-7-cyclopropyl- 7H-pyrrolo[2,3-d]pyrimidin- 5-yl)imidazo[1,2-a]pyridin-8- yl)-3-(4-((4-ethylpiperazin-1- yl)methyl)-3-(trifluorometh- yl)phenyl)urea |
| I-36 | 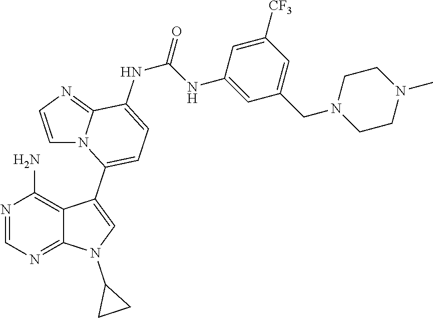 [SMILES: CN1CCN(Cc2cc(NC(=O)Nc3ccc(-c4cn(C5CC5)c5ncnc(N)c45)n4ccnc34)cc(C(F)(F)F)c2)CC1] | 1-(5-(4-amino-7-cyclopropyl- 7H-pyrrolo[2,3-d]pyrimidin- 5-yl)imidazo[1,2-a]pyridin-8- yl)-3-(3-((4-methylpiperazin- 1-yl)methyl)-5-(trifluorometh- yl)phenyl)urea |
| I-37 | 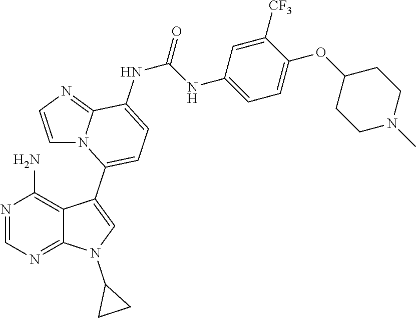 [SMILES: CN1CCC(Oc2ccc(NC(=O)Nc3ccc(-c4cn(C5CC5)c5ncnc(N)c45)n4ccnc34)cc2C(F)(F)F)CC1] | 1-(5-(4-amino-7-cyclopropyl- 7H-pyrrolo[2,3-d]pyrimidin- 5-yl)imidazo[1,2-a]pyridin-8- yl)-3-(4-((1-methylpiperidin- 4-yl)oxy)-3-(trifluorometh- yl)phenyl)urea |
| I-38 | 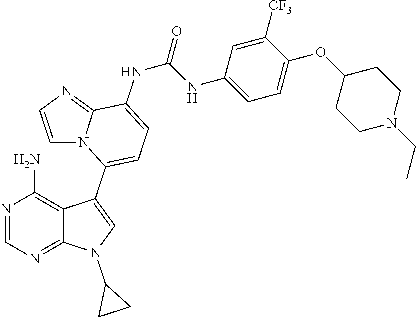 [SMILES: CCN1CCC(Oc2ccc(NC(=O)Nc3ccc(-c4cn(C5CC5)c5ncnc(N)c45)n4ccnc34)cc2C(F)(F)F)CC1] | 1-(5-(4-amino-7-cyclopropyl- 7H-pyrrolo[2,3-d]pyrimidin- 5-yl)imidazo[1,2-a]pyridin-8- yl)-3-(4-((1-ethylpiperidin-4- yl)oxy)-3-(trifluoromethyl)- phenyl)urea |
| I-39 | 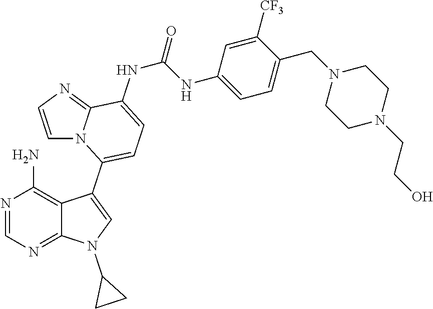 [SMILES: Nc1ncnc2c1c(-c1ccc(NC(=O)Nc3ccc(CN4CCN(CCO)CC4)c(C(F)(F)F)c3)c3nccn13)cn2C1CC1] | 1-(5-(4-amino-7-cyclopropyl- 7H-pyrrolo[2,3-d]pyrimidin- 5-yl)imidazo[1,2-a]pyridin-8- yl)-3-(4-((4-(2-hydroxyeth- yl)piperazin-1-yl)methyl)-3- (trifluoromethyl)phenyl)urea |
| I-40 | 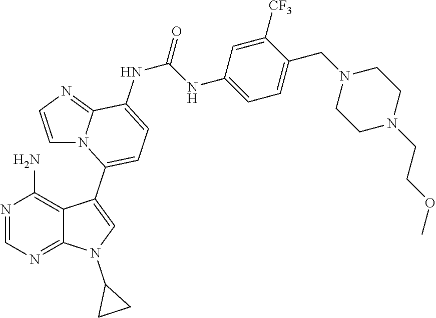 [SMILES: COCCN1CCN(Cc2ccc(NC(=O)Nc3ccc(-c4cn(C5CC5)c5ncnc(N)c45)n4ccnc34)cc2C(F)(F)F)CC1] | 1-(5-(4-amino-7-cyclopropyl- 7H-pyrrolo[2,3-d]pyrimidin- 5-yl)imidazo[1,2-a]pyridin-8- yl)-3-(4-((4-(2-methoxyeth- yl)piperazin-1-yl)methyl)-3- (trifluoromethyl)phenyl)urea |
| I-41 | 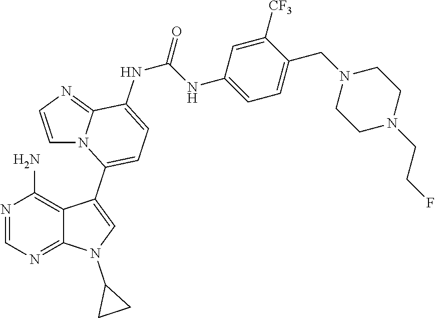 [SMILES: Nc1ncnc2c1c(-c1ccc(NC(=O)Nc3ccc(CN4CCN(CCF)CC4)c(C(F)(F)F)c3)c3nccn13)cn2C1CC1] | 1-(5-(4-amino-7-cyclopropyl- 7H-pyrrolo[2,3-d]pyrimidin- 5-yl)imidazo[1,2-a]pyridin-8- yl)-3-(4-((4-(2-fluoroethyl)- piperazin-1-yl)methyl)-3- (trifluoromethyl)phenyl)urea |
| I-42 | 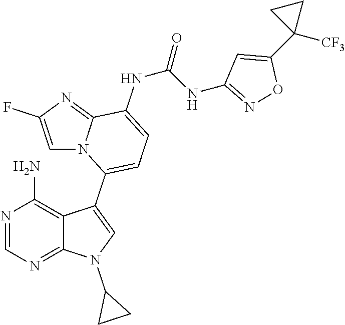 [SMILES: Nc1ncnc2c1c(-c1ccc(NC(=O)Nc3cc(C4(C(F)(F)F)CC4)on3)c3nc(F)cn13)cn2C1CC1] | 1-(5-(4-amino-7-cyclopropyl- 7H-pyrrolo[2,3-d]pyrimidin- 5-yl)-2-fluoroimidazo[1,2-a]- pyridin-8-yl)-3-(5-(1-(tri- fluoromethyl)cyclopropyl)- isoxazol-3-yl)urea |
| I-43 | 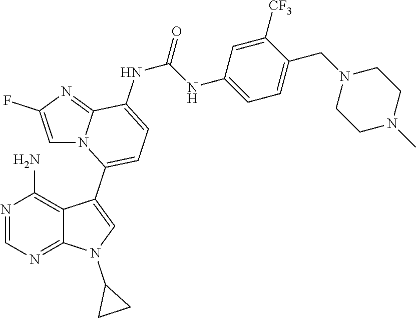 [SMILES: CN1CCN(Cc2ccc(NC(=O)Nc3ccc(-c4cn(C5CC5)c5ncnc(N)c45)n4cc(F)nc34)cc2C(F)(F)F)CC1] | 1-(5-(4-amino-7-cyclopropyl- 7H-pyrrolo[2,3-d]pyrimidin- 5-yl)-2-fluoroimidazo[1,2-a]- pyridin-8-yl)-3-(4-((4-methyl- piperazin-1-yl)methyl)-3- (trifluoromethyl)phenyl)urea |
| I-44 | 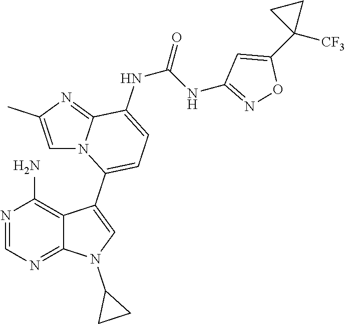 [SMILES: Cc1cn2c(-c3cn(C4CC4)c4ncnc(N)c34)ccc(NC(=O)Nc3cc(C4(C(F)(F)F)CC4)on3)c2n1] | 1-(5-(4-amino-7-cyclopropyl- 7H-pyrrolo[2,3-d]pyrimidin- 5-yl)-2-methylimidazo[1,2-a]- pyridin-8-yl)-3-(5-(1-(tri- fluoromethyl)cyclopropyl)- isoxazol-3-yl)urea |
| I-45 | 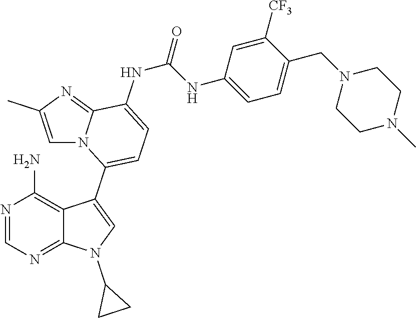 [SMILES: Cc1cn2c(-c3cn(C4CC4)c4ncnc(N)c34)ccc(NC(=O)Nc3ccc(CN4CCN(C)CC4)c(C(F)(F)F)c3)c2n1] | 1-(5-(4-amino-7-cyclopropyl- 7H-pyrrolo[2,3-d]pyrimidin- 5-yl)-2-methylimidazo[1,2-a]- pyridin-8-yl)-3-(4-((4-methyl- piperazin-1-yl)methyl)-3- (trifluoromethyl)phenyl)urea |
| I-46 | 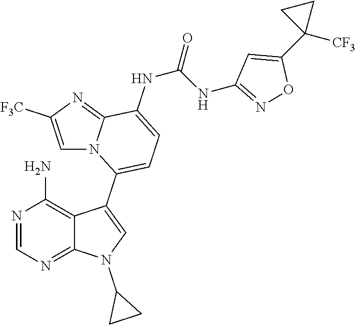 [SMILES: Nc1ncnc2c1c(-c1ccc(NC(=O)Nc3cc(C4(C(F)(F)F)CC4)on3)c3nc(C(F)(F)F)cn13)cn2C1CC1] | 1-(5-(4-amino-7-cyclopropyl- 7H-pyrrolo[2,3-d]pyrimidin- 5-yl)-2-(trifluoromethyl)- imidazo[1,2-a]pyridin-8-yl)-3- (5-(1-(trifluoromethyl)cyclo- propyl)isoxazol-3-yl)urea |
| I-47 | 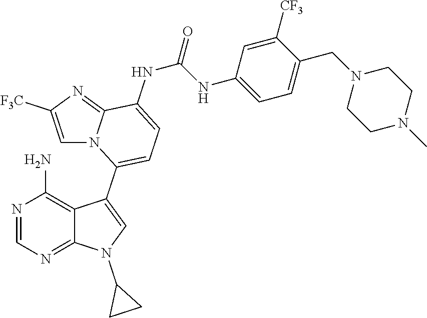 [SMILES: CN1CCN(Cc2ccc(NC(=O)Nc3ccc(-c4cn(C5CC5)c5ncnc(N)c45)n4cc(C(F)(F)F)nc34)cc2C(F)(F)F)CC1] | 1-(5-(4-amino-7-cyclopropyl- 7H-pyrrolo[2,3-d]pyrimidin- 5-yl)-2-(trifluoromethyl)- imidazo[1,2-a]pyridin-8-yl)-3- (4-((4-methylpiperazin-1-yl)- methyl)-3-(trifluoromethyl)- phenyl)urea |
| I-48 | 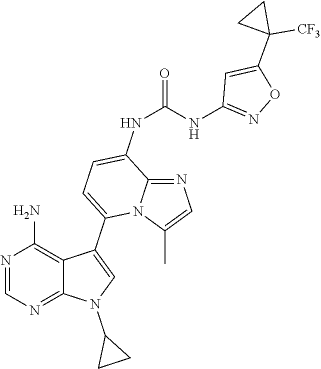 [SMILES: Cc1cnc2c(NC(=O)Nc3cc(C4(C(F)(F)F)CC4)on3)ccc(-c3cn(C4CC4)c4ncnc(N)c34)n12] | 1-(5-(4-amino-7-cyclopropyl- 7H-pyrrolo[2,3-d]pyrimidin- 5-yl)-3-methylimidazo[1,2-a]- pyridin-8-yl)-3-(5-(1-(tri- fluoromethyl)cyclopropyl)- isoxazol-3-yl)urea |
| I-49 | 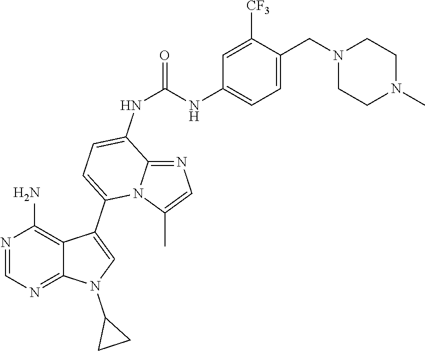 [SMILES: Cc1cnc2c(NC(=O)Nc3ccc(CN4CCN(C)CC4)c(C(F)(F)F)c3)ccc(-c3cn(C4CC4)c4ncnc(N)c34)n12] | 1-(5-(4-amino-7-cyclopropyl- 7H-pyrrolo[2,3-d]pyrimidin- 5-yl)-3-methylimidazo[1,2-a]- pyridin-8-yl)-3-(4-((4-meth- ylpiperazin-1-yl)methyl)-3- (trifluoromethyl)phenyl)urea |
| I-50 | 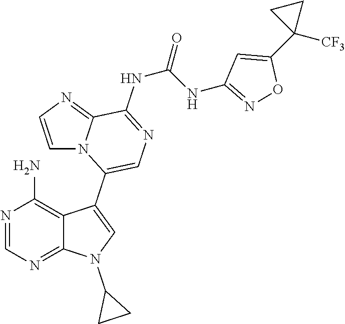 [SMILES: Nc1ncnc2c1c(-c1cnc(NC(=O)Nc3cc(C4(C(F)(F)F)CC4)on3)c3nccn13)cn2C1CC1] | 1-(5-(4-amino-7-cyclopropyl- 7H-pyrrolo[2,3-d]pyrimidin- 5-yl)imidazo[1,2-a]pyrazin-8- yl)-3-(5-(1-(trifluoromethyl)- cyclopropyl)isoxazol-3-yl)urea |
| I-51 | 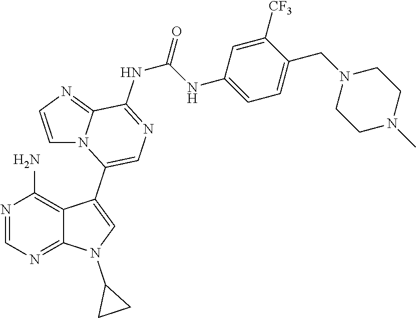 [SMILES: CN1CCN(Cc2ccc(NC(=O)Nc3ncc(-c4cn(C5CC5)c5ncnc(N)c45)n4ccnc34)cc2C(F)(F)F)CC1] | 1-(5-(4-amino-7-cyclopropyl- 7H-pyrrolo[2,3-d]pyrimidin- 5-yl)imidazo[1,2-a]pyrazin-8- yl)-3-(4-((4-methylpiperazin- 1-yl)methyl)-3-(trifluorometh- yl)phenyl)urea |
| I-52 | 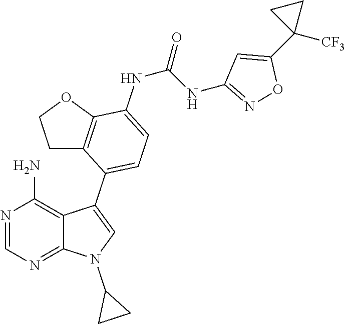 [SMILES: Nc1ncnc2c1c(-c1ccc(NC(=O)Nc3cc(C4(C(F)(F)F)CC4)on3)c3c1CCO3)cn2C1CC1] | 1-(4-(4-amino-7-cyclopropyl- 7H-pyrrolo[2,3-d]pyrimidin- 5-yl)-2,3-dihydrobenzofuran- 7-yl)-3-(5-(1-(trifluorometh- yl)cyclopropyl)isoxazol-3- yl)urea |
| I-53 | 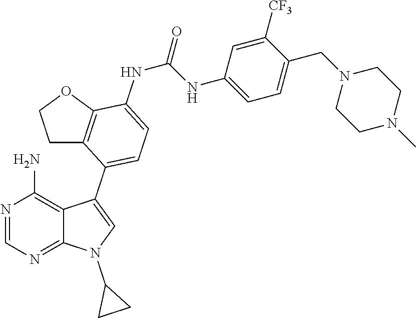 [SMILES: CN1CCN(Cc2ccc(NC(=O)Nc3ccc(-c4cn(C5CC5)c5ncnc(N)c45)c4c3OCC4)cc2C(F)(F)F)CC1] | 1-(4-(4-amino-7-cyclopropyl- 7H-pyrrolo[2,3-d]pyrimidin- 5-yl)-2,3-dihydrobenzofuran- 7-yl)-3-(4-((4-methylpiperazin- 1-yl)methyl)-3-(trifluorometh- yl)phenyl)urea |
| I-54 | 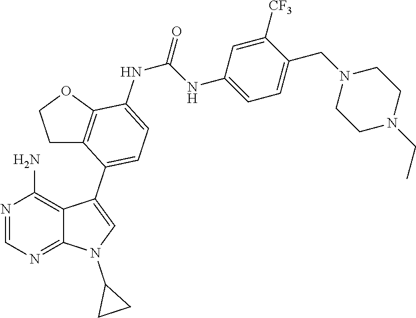 [SMILES: CCN1CCN(Cc2ccc(NC(=O)Nc3ccc(-c4cn(C5CC5)c5ncnc(N)c45)c4c3OCC4)cc2C(F)(F)F)CC1] | 1-(4-(4-amino-7-cyclopropyl- 7H-pyrrolo[2,3-d]pyrimidin- 5-yl)-2,3-dihydrobenzofuran- 7-yl)-3-(4-((4-ethylpiperazin- 1-yl)methyl)-3-(trifluorometh- yl)phenyl)urea |
| I-55 | 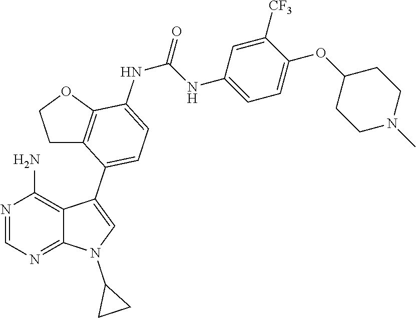 [SMILES: CN1CCC(Oc2ccc(NC(=O)Nc3ccc(-c4cn(C5CC5)c5ncnc(N)c45)c4c3OCC4)cc2C(F)(F)F)CC1] | 1-(4-(4-amino-7-cyclopropyl- 7H-pyrrolo[2,3-d]pyrimidin- 5-yl)-2,3-dihydrobenzofuran- 7-yl)-3-(4-((1-methylpiperidin- 4-yl)oxy)-3-(trifluorometh- yl)phenyl)urea |
| I-56 | 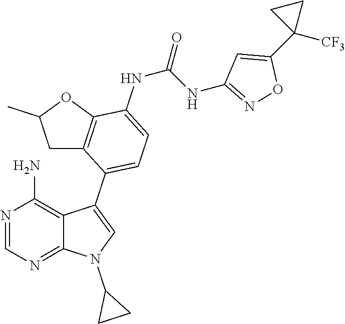 [SMILES: CC1Cc2c(-c3cn(C4CC4)c4ncnc(N)c34)ccc(NC(=O)Nc3cc(C4(C(F)(F)F)CC4)on3)c2O1] | 1-(4-(4-amino-7-cyclopropyl- 7H-pyrrolo[2,3-d]pyrimidin- 5-yl)-2-methyl-2,3-dihydro- benzofuran-7-yl)-3-(5-(1- (trifluoromethyl)cyclopropyl)- isoxazol-3-yl)urea |
| I-57 | 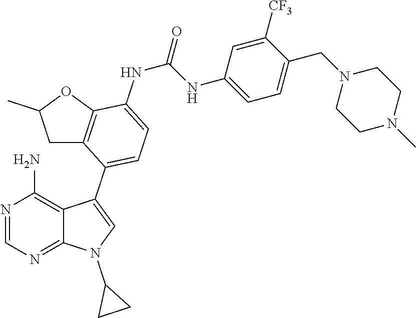 [SMILES: CC1Cc2c(-c3cn(C4CC4)c4ncnc(N)c34)ccc(NC(=O)Nc3ccc(CN4CCN(C)CC4)c(C(F)(F)F)c3)c2O1] | 1-(4-(4-amino-7-cyclopropyl- 7H-pyrrolo[2,3-d]pyrimidin- 5-yl)-2-methyl-2,3-dihydro- benzofuran-7-yl)-3-(4-((4- methylpiperazin-1-yl)methyl)- 3-(trifluoromethyl)phenyl)urea |
| I-58 | 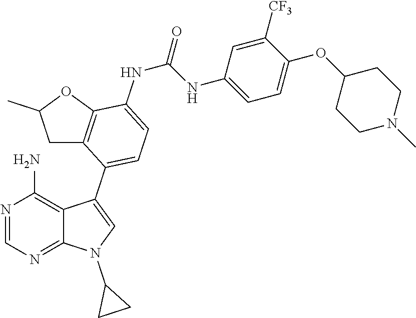 [SMILES: CC1Cc2c(-c3cn(C4CC4)c4ncnc(N)c34)ccc(NC(=O)Nc3ccc(OC4CCN(C)CC4)c(C(F)(F)F)c3)c2O1] | 1-(4-(4-amino-7-cyclopropyl- 7H-pyrrolo[2,3-d]pyrimidin- 5-yl)-2-methyl-2,3-dihydro- benzofuran-7-yl)-3-(4-((1- methylpiperidin-4-yl)oxy)-3- (trifluoromethyl)phenyl)urea |
| I-59 | 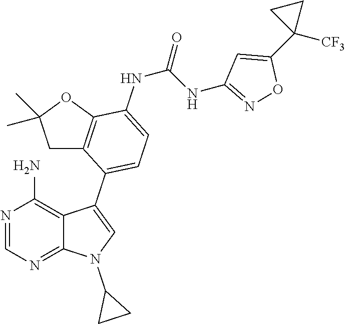 [SMILES: CC1(C)Cc2c(-c3cn(C4CC4)c4ncnc(N)c34)ccc(NC(=O)Nc3cc(C4(C(F)(F)F)CC4)on3)c2O1] | 1-(4-(4-amino-7-cyclopropyl- 7H-pyrrolo[2,3-d]pyrimidin- 5-yl)-2,2-dimethyl-2,3- dihydrobenzofuran-7-yl)-3- (5-(1-(trifluoromethyl)cyclo- propyl)isoxazol-3-yl)urea |
| I-60 | 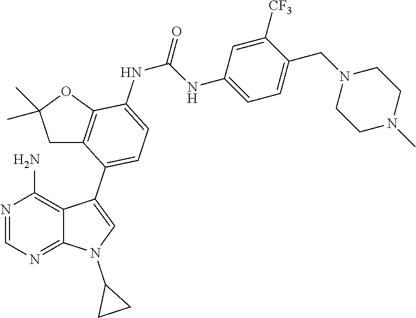 [SMILES: CN1CCN(Cc2ccc(NC(=O)Nc3ccc(-c4cn(C5CC5)c5ncnc(N)c45)c4c3OC(C)(C)C4)cc2C(F)(F)F)CC1] | 1-(4-(4-amino-7-cyclopropyl- 7H-pyrrolo[2,3-d]pyrimidin- 5-yl)-2,2-dimethyl-2,3- dihydrobenzofuran-7-yl)-3- (4-((4-methylpiperazin-1-yl)- methyl)-3-(trifluoromethyl)- phenyl)urea |
| I-61 | 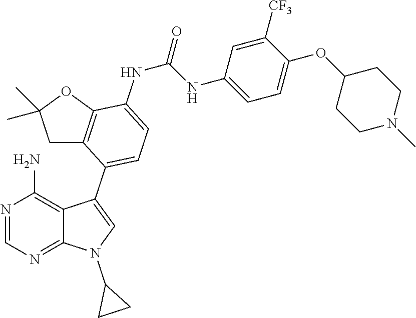 [SMILES: CN1CCC(Oc2ccc(NC(=O)Nc3ccc(-c4cn(C5CC5)c5ncnc(N)c45)c4c3OC(C)(C)C4)cc2C(F)(F)F)CC1] | 1-(4-(4-amino-7-cyclopropyl- 7H-pyrrolo[2,3-d]pyrimidin- 5-yl)-2,2-dimethyl-2,3- dihydrobenzofuran-7-yl)-3- (4-((1-methylpiperidin-4-yl)- oxy)-3-(trifluoromethyl)phen- yl)urea |
| I-62 | 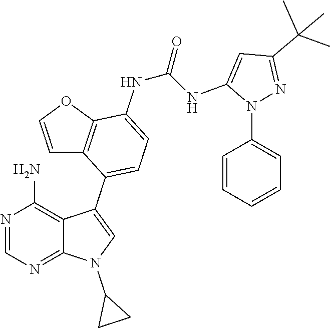 [SMILES: CC(C)(C)c1cc(NC(=O)Nc2ccc(-c3cn(C4CC4)c4ncnc(N)c34)c3ccoc23)n(-c2ccccc2)n1] | 1-(4-(4-amino-7-cyclopropyl- 7H-pyrrolo[2,3-d]pyrimidin- 5-yl)benzofuran-7-yl)-3-(3- (tert-butyl)-1-phenyl-1H- pyrazol-5-yl)urea |
| I-63 | 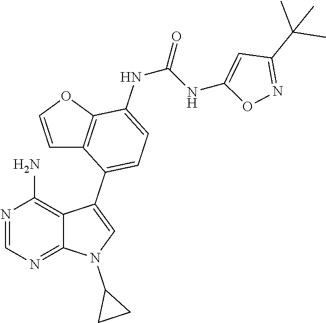 [SMILES: CC(C)(C)c1cc(NC(=O)Nc2ccc(-c3cn(C4CC4)c4ncnc(N)c34)c3ccoc23)on1] | 1-(4-(4-amino-7-cyclopropyl- 7H-pyrrolo[2,3-d]pyrimidin- 5-yl)benzofuran-7-yl)-3-(3- (tert-butyl)isoxazol-5-yl)urea |
| I-64 | 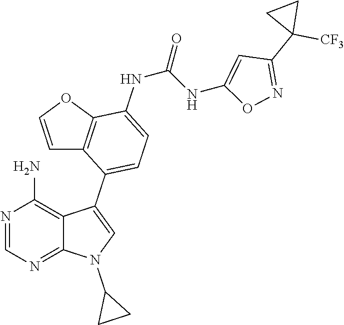 [SMILES: Nc1ncnc2c1c(-c1ccc(NC(=O)Nc3cc(C4(C(F)(F)F)CC4)no3)c3occc13)cn2C1CC1] | 1-(4-(4-amino-7-cyclopropyl- 7H-pyrrolo[2,3-d]pyrimidin- 5-yl)benzofuran-7-yl)-3-(3- (1-(trifluoromethyl)cyclo- propyl)isoxazol-5-yl)urea |
| I-65 | 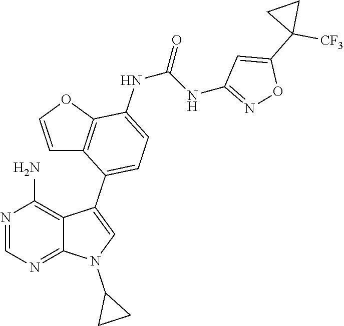 [SMILES: Nc1ncnc2c1c(-c1ccc(NC(=O)Nc3cc(C4(C(F)(F)F)CC4)on3)c3occc13)cn2C1CC1] | 1-(4-(4-amino-7-cyclopropyl- 7H-pyrrolo[2,3-d]pyrimidin- 5-yl)benzofuran-7-yl)-3-(5-(1- (trifluoromethyl)cyclopropyl)- isoxazol-3-yl)urea |
| I-66 | 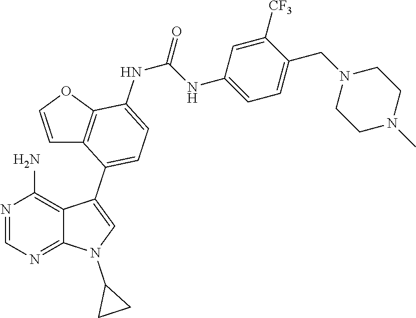 [SMILES: CN1CCN(Cc2ccc(NC(=O)Nc3ccc(-c4cn(C5CC5)c5ncnc(N)c45)c4ccoc34)cc2C(F)(F)F)CC1] | 1-(4-(4-amino-7-cyclopropyl- 7H-pyrrolo[2,3-d]pyrimidin- 5-yl)benzofuran-7-yl)-3-(4- ((4-methylpiperazin-1-yl)- methyl)-3-(trifluoromethyl)- phenyl)urea |
| I-67 | 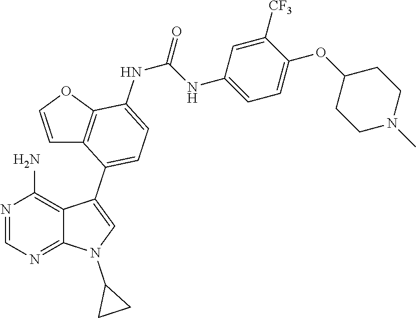 [SMILES: CN1CCC(Oc2ccc(NC(=O)Nc3ccc(-c4cn(C5CC5)c5ncnc(N)c45)c4ccoc34)cc2C(F)(F)F)CC1] | 1-(4-(4-amino-7-cyclopropyl- 7H-pyrrolo[2,3-d]pyrimidin- 5-yl)benzofuran-7-yl)-3-(4- ((1-methylpiperidin-4-yl)oxy)- 3-(trifluoromethyl)phenyl)urea |
| I-68 | 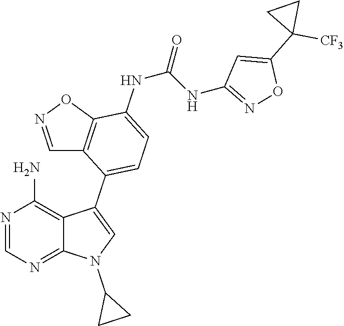 [SMILES: Nc1ncnc2c1c(-c1ccc(NC(=O)Nc3cc(C4(C(F)(F)F)CC4)on3)c3oncc13)cn2C1CC1] | 1-(4-(4-amino-7-cyclopropyl- 7H-pyrrolo[2,3-d]pyrimidin- 5-yl)benzo[d]isoxazol-7-yl)-3- (5-(1-(trifluoromethyl)cyclo- propyl)isoxazol-3-yl)urea |
| I-69 | 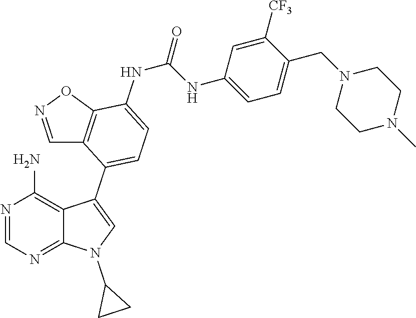 [SMILES: CN1CCN(Cc2ccc(NC(=O)Nc3ccc(-c4cn(C5CC5)c5ncnc(N)c45)c4cnoc34)cc2C(F)(F)F)CC1] | 1-(4-(4-amino-7-cyclopropyl- 7H-pyrrolo[2,3-d]pyrimidin- 5-yl)benzo[d]isoxazol-7-yl)- 3-(4-((4-methylpiperazin-1- yl)methyl)-3-(trifluorometh- yl)phenyl)urea |
| I-70 | 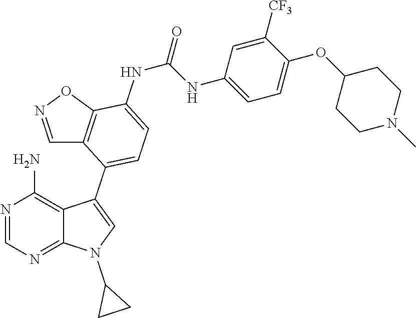 [SMILES: CN1CCC(Oc2ccc(NC(=O)Nc3ccc(-c4cn(C5CC5)c5ncnc(N)c45)c4cnoc34)cc2C(F)(F)F)CC1] | 1-(4-(4-amino-7-cyclopropyl- 7H-pyrrolo[2,3-d]pyrimidin- 5-yl)benzo[d]isoxazol-7-yl)- 3-(4-((1-methylpiperidin-4- yl)oxy)-3-(trifluoromethyl)- phenyl)urea |
| I-71 | 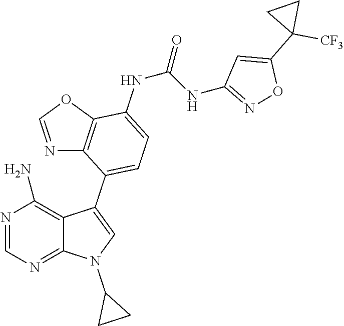 [SMILES: Nc1ncnc2c1c(-c1ccc(NC(=O)Nc3cc(C4(C(F)(F)F)CC4)on3)c3ocnc13)cn2C1CC1] | 1-(4-(4-amino-7-cyclopropyl- 7H-pyrrolo[2,3-d]pyrimidin- 5-yl)benzo[d]oxazol-7-yl)-3- (5-(1-(trifluoromethyl)cyclo- propyl)isoxazol-3-yl)urea |
| I-72 | 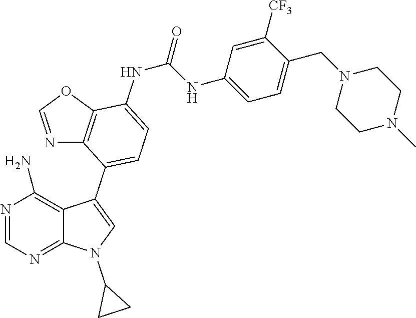 [SMILES: CN1CCN(Cc2ccc(NC(=O)Nc3ccc(-c4cn(C5CC5)c5ncnc(N)c45)c4ncoc34)cc2C(F)(F)F)CC1] | 1-(4-(4-amino-7-cyclopropyl- 7H-pyrrolo[2,3-d]pyrimidin- 5-yl)benzo[d]oxazol-7-yl)-3- (4-((4-methylpiperazin-1-yl)- methyl)-3-(trifluoromethyl)- phenyl)urea |
| I-73 | 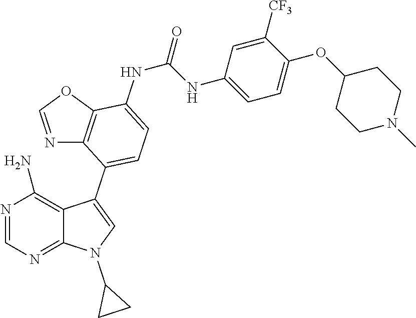 [SMILES: CN1CCC(Oc2ccc(NC(=O)Nc3ccc(-c4cn(C5CC5)c5ncnc(N)c45)c4ncoc34)cc2C(F)(F)F)CC1] | 1-(4-(4-amino-7-cyclopropyl- 7H-pyrrolo[2,3-d]pyrimidin- 5-yl)benzo[d]oxazol-7-yl)-3- (4-((1-methylpiperidin-4-yl)- oxy)-3-(trifluoromethyl)phen- yl)urea |
| I-74 | 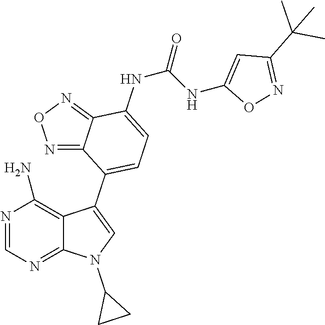 [SMILES: CC(C)(C)c1cc(NC(=O)Nc2ccc(-c3cn(C4CC4)c4ncnc(N)c34)c3nonc23)on1] | 1-(7-(4-amino-7-cyclopropyl- 7H-pyrrolo[2,3-d]pyrimidin-5- yl)benzo[c][1,2,5]oxadiazol-4- yl)-3-(3-(tert-butyl)isoxazol-5- yl)urea |
| I-75 | 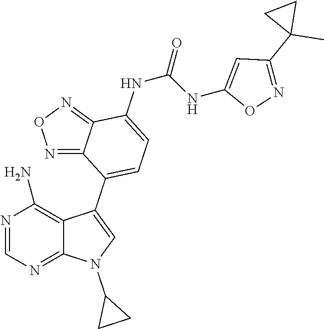 [SMILES: CC1(c2cc(NC(=O)Nc3ccc(-c4cn(C5CC5)c5ncnc(N)c45)c4nonc34)on2)CC1] | 1-(7-(4-amino-7-cyclopropyl- 7H-pyrrolo[2,3-d]pyrimidin- 5-yl)benzo[c][1,2,5]oxadiazol- 4-yl)-3-(3-(1-methylcyclo- propyl)isoxazol-5-yl)urea |
| I-76 | 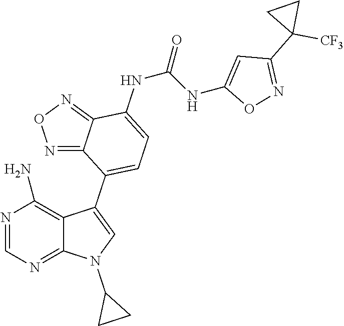 [SMILES: Nc1ncnc2c1c(-c1ccc(NC(=O)Nc3cc(C4(C(F)(F)F)CC4)no3)c3nonc13)cn2C1CC1] | 1-(7-(4-amino-7-cyclopropyl- 7H-pyrrolo[2,3-d]pyrimidin- 5-yl)benzo[c][1,2,5]oxadiazol- 4-yl)-3-(3-(1-(trifluoromethyl)- cyclopropyl)isoxazol-5-yl)urea |
| I-77 | 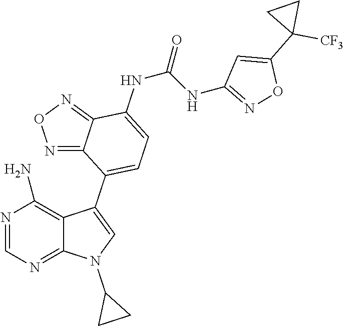 [SMILES: Nc1ncnc2c1c(-c1ccc(NC(=O)Nc3cc(C4(C(F)(F)F)CC4)on3)c3nonc13)cn2C1CC1] | 1-(7-(4-amino-7-cyclopropyl- 7H-pyrrolo[2,3-d]pyrimidin- 5-yl)benzo[c][1,2,5]oxadiazol- 4-yl)-3-(5-(1-(trifluoromethyl)- cyclopropyl)isoxazol-3-yl)urea |
| I-78 | 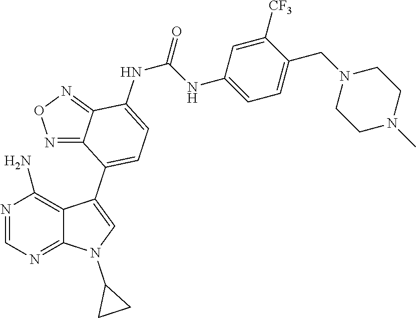 [SMILES: CN1CCN(Cc2ccc(NC(=O)Nc3ccc(-c4cn(C5CC5)c5ncnc(N)c45)c4nonc34)cc2C(F)(F)F)CC1] | 1-(7-(4-amino-7-cyclopropyl- 7H-pyrrolo[2,3-d]pyrimidin- 5-yl)benzo[c][1,2,5]oxadiazol- 4-yl)-3-(4-((4-methylpiperazin- 1-yl)methyl)-3-(trifluorometh- yl)phenyl)urea |
| I-79 | 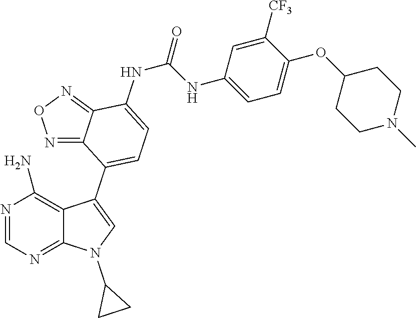 [SMILES: CN1CCC(Oc2ccc(NC(=O)Nc3ccc(-c4cn(C5CC5)c5ncnc(N)c45)c4nonc34)cc2C(F)(F)F)CC1] | 1-(7-(4-amino-7-cyclopropyl- 7H-pyrrolo[2,3-d]pyrimidin- 5-yl)benzo[c][1,2,5]oxadiazol- 4-yl)-3-(4-((1-methylpiperidin- 4-yl)oxy)-3-(trifluoromethyl)- phenyl)urea |
| I-80 | 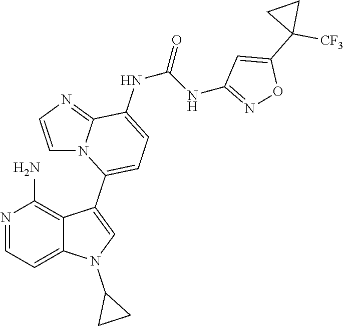 [SMILES: Nc1nccc2c1c(-c1ccc(NC(=O)Nc3cc(C4(C(F)(F)F)CC4)on3)c3nccn13)cn2C1CC1] | 1-(5-(4-amino-1-cyclopropyl- 1H-pyrrolo[3,2-c]pyridin-3- yl)imidazo[1,2-a]pyridin-8- yl)-3-(5-(1-(trifluoromethyl)- cyclopropyl)isoxazol-3-yl)urea |
| I-81 | 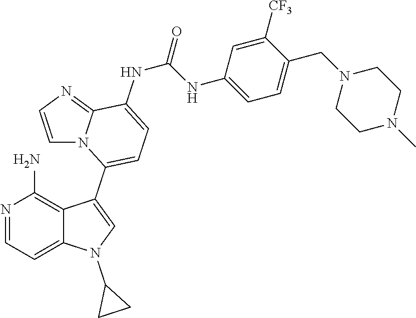 [SMILES: CN1CCN(Cc2ccc(NC(=O)Nc3ccc(-c4cn(C5CC5)c5ccnc(N)c45)n4ccnc34)cc2C(F)(F)F)CC1] | 1-(5-(4-amino-1-cyclopropyl- 1H-pyrrolo[3,2-c]pyridin-3- yl)imidazo[1,2-a]pyridin-8- yl)-3-(4-((4-methylpiperazin- 1-yl)methyl)-3-(trifluorometh- yl)phenyl)urea |
| I-82 | 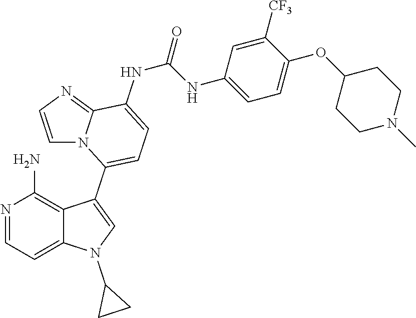 [SMILES: CN1CCC(Oc2ccc(NC(=O)Nc3ccc(-c4cn(C5CC5)c5ccnc(N)c45)n4ccnc34)cc2C(F)(F)F)CC1] | 1-(5-(4-amino-1-cyclopropyl- 1H-pyrrolo[3,2-c]pyridin-3- yl)imidazo[1,2-a]pyridin-8- yl)-3-(4-((1-methylpiperidin- 4-yl)oxy)-3-(trifluorometh- yl)phenyl)urea |
| I-83 | 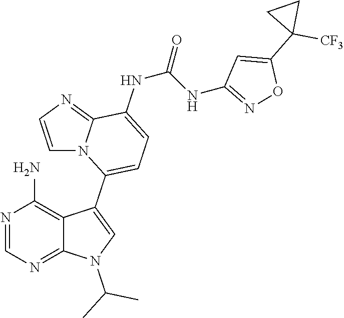 [SMILES: CC(C)n1cc(-c2ccc(NC(=O)Nc3cc(C4(C(F)(F)F)CC4)on3)c3nccn23)c2c(N)ncnc21] | 1-(5-(4-amino-7-isopropyl- 7H-pyrrolo[2,3-d]pyrimidin- 5-yl)imidazo[1,2-a]pyridin-8- yl)-3-(5-(1-(trifluorometh- yl)cyclopropyl)isoxazol-3-yl)- urea |
| I-84 | 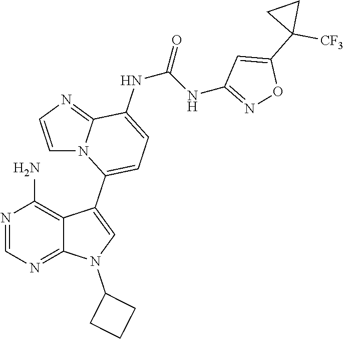 [SMILES: Nc1ncnc2c1c(-c1ccc(NC(=O)Nc3cc(C4(C(F)(F)F)CC4)on3)c3nccn13)cn2C1CCC1] | 1-(5-(4-amino-7-cyclobutyl- 7H-pyrrolo[2,3-d]pyrimidin- 5-yl)imidazo[1,2-a]pyridin-8- yl)-3-(5-(1-(trifluoromethyl)- cyclopropyl)isoxazol-3-yl)urea |
| I-85 | 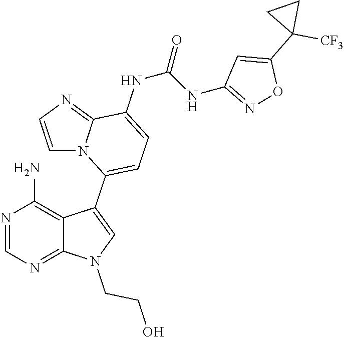 [SMILES: Nc1ncnc2c1c(-c1ccc(NC(=O)Nc3cc(C4(C(F)(F)F)CC4)on3)c3nccn13)cn2CCO] | 1-(5-(4-amino-7-(2-hydroxy- ethyl)-7H-pyrrolo[2,3-d]pyr- imidin-5-yl)imidazo[1,2-a]- pyridin-8-yl)-3-(5-(1-(tri- fluoromethyl)cyclopropyl)- isoxazol-3-yl)urea |
| I-86 | 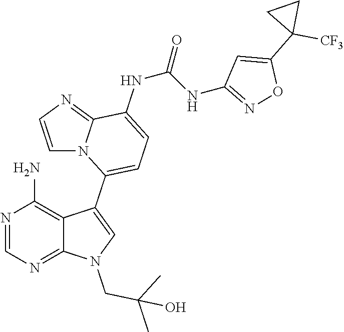 [SMILES: CC(C)(O)Cn1cc(-c2ccc(NC(=O)Nc3cc(C4(C(F)(F)F)CC4)on3)c3nccn23)c2c(N)ncnc21] | 1-(5-(4-amino-7-(2-hydroxy- 2-methylpropyl)-7H-pyrrolo- [2,3-d]pyrimidin-5-yl)- imidazo[1,2-a]pyridin-8-yl)- 3-(5-(1-(trifluoromethyl)- cyclopropyl)isoxazol-3-yl)urea |
| I-87 | 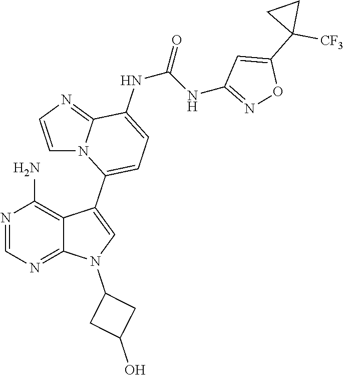 [SMILES: Nc1ncnc2c1c(-c1ccc(NC(=O)Nc3cc(C4(C(F)(F)F)CC4)on3)c3nccn13)cn2C1CC(O)C1] | 1-(5-(4-amino-7-(3-hydroxy- cyclobutyl)-7H-pyrrolo[2,3- d]pyrimidin-5-yl)imidazo[1,2- a]pyridin-8-yl)-3-(5-(1- (trifluoromethyl)cyclopropyl)- isoxazol-3-yl)urea |
| I-88 | 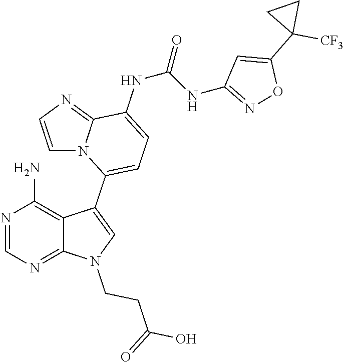 [SMILES: Nc1ncnc2c1c(-c1ccc(NC(=O)Nc3cc(C4(C(F)(F)F)CC4)on3)c3nccn13)cn2CCC(=O)O] | 3-(4-amino-5-(8-(3-(5-(1- (trifluoromethyl)cyclopro- pyl)isoxazol-3-yl)ureido)- imidazo[1,2-a]pyridin-5-yl)- 7H-pyrrolo[2,3-d]pyrimidin- 7-yl)propanoic acid |
| I-89 | 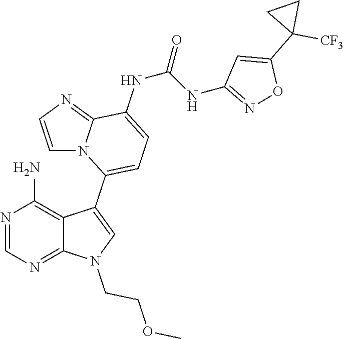 [SMILES: COCCn1cc(-c2ccc(NC(=O)Nc3cc(C4(C(F)(F)F)CC4)on3)c3nccn23)c2c(N)ncnc21] | 1-(5-(4-amino-7-(2-methoxy- ethyl)-7H-pyrrolo[2,3-d]- pyrimidin-5-yl)imidazo[1,2- a]pyridin-8-yl)-3-(5-(1- (trifluoromethyl)cyclopropyl)- isoxazol-3-yl)urea |
| I-90 | 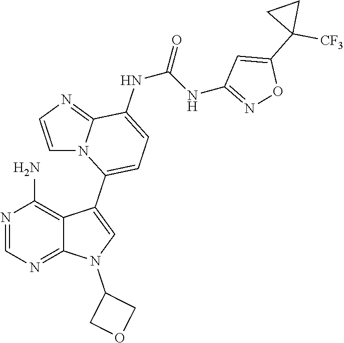 [SMILES: Nc1ncnc2c1c(-c1ccc(NC(=O)Nc3cc(C4(C(F)(F)F)CC4)on3)c3nccn13)cn2C1COC1] | 1-(5-(4-amino-7-(oxetan-3-yl)- 7H-pyrrolo[2,3-d]pyrimidin-5- yl)imidazo[1,2-a]pyridin-8- yl)-3-(5-(1-(trifluoromethyl)- cyclopropyl)isoxazol-3-yl)urea |
| I-91 | 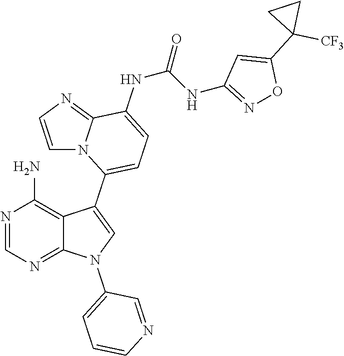 [SMILES: Nc1ncnc2c1c(-c1ccc(NC(=O)Nc3cc(C4(C(F)(F)F)CC4)on3)c3nccn13)cn2-c1cccnc1] | 1-(5-(4-amino-7-(pyridin-3-yl)- 7H-pyrrolo[2,3-d]pyrimidin- 5-yl)imidazo[1,2-a]pyridin-8- yl)-3-(5-(1-(trifluoromethyl)- cyclopropyl)isoxazol-3-yl)urea |
| I-92 | 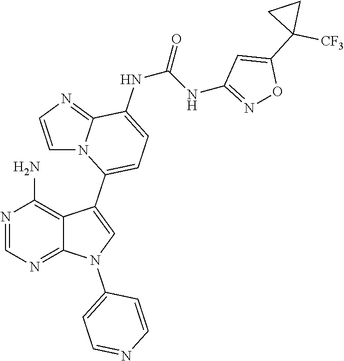 [SMILES: Nc1ncnc2c1c(-c1ccc(NC(=O)Nc3cc(C4(C(F)(F)F)CC4)on3)c3nccn13)cn2-c1ccncc1] | 1-(5-(4-amino-7-(pyridin-4-yl)- 7H-pyrrolo[2,3-d]pyrimidin- 5-yl)imidazo[1,2-a]pyridin-8- yl)-3-(5-(1-(trifluoromethyl)- cyclopropyl)isoxazol-3-yl)urea |
| I-93 | 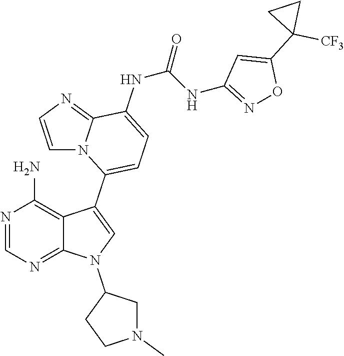 [SMILES: CN1CCC(n2cc(-c3ccc(NC(=O)Nc4cc(C5(C(F)(F)F)CC5)on4)c4nccn34)c3c(N)ncnc32)C1] | 1-(5-(4-amino-7-(1-methyl- pyrrolidin-3-yl)-7H-pyrrolo- [2,3-d]pyrimidin-5-yl)imidazo- [1,2-a]pyridin-8-yl)-3-(5-(1- (trifluoromethyl)cyclopropyl)- isoxazol-3-yl)urea |
| I-94 | 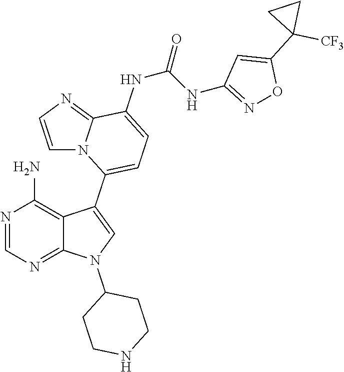 [SMILES: Nc1ncnc2c1c(-c1ccc(NC(=O)Nc3cc(C4(C(F)(F)F)CC4)on3)c3nccn13)cn2C1CCNCC1] | 1-(5-(4-amino-7-(piperidin-4- yl)-7H-pyrrolo[2,3-d]pyr- imidin-5-yl)imidazo[1,2-a]- pyridin-8-yl)-3-(5-(1-(trifluoro- methyl)cyclopropyl)isoxazol- 3-yl)urea |
| I-95 | 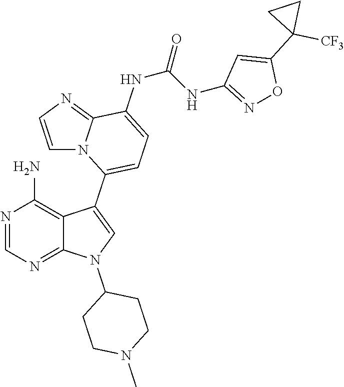 [SMILES: CN1CCC(n2cc(-c3ccc(NC(=O)Nc4cc(C5(C(F)(F)F)CC5)on4)c4nccn34)c3c(N)ncnc32)CC1] | 1-(5-(4-amino-7-(1-methyl- piperidin-4-yl)-7H-pyrrolo- [2,3-d]pyrimidin-5-yl)imid- azo[1,2-a]pyridin-8-yl)-3-(5- (1-(trifluoromethyl)cyclo- propyl)isoxazol-3-yl)urea |
| I-96 | 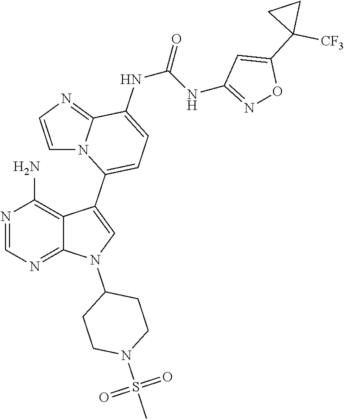 [SMILES: CS(=O)(=O)N1CCC(n2cc(-c3ccc(NC(=O)Nc4cc(C5(C(F)(F)F)CC5)on4)c4nccn34)c3c(N)ncnc32)CC1] | 1-(5-(4-amino-7-(1-(methyl- sulfonyl)piperidin-4-yl)-7H- pyrrolo[2,3-d]pyrimidin-5- yl)imidazo[1,2-a]pyridin-8-yl)- 3-(5-(1-(trifluoromethyl)cyclo- propyl)isoxazol-3-yl)urea |
| I-97 | 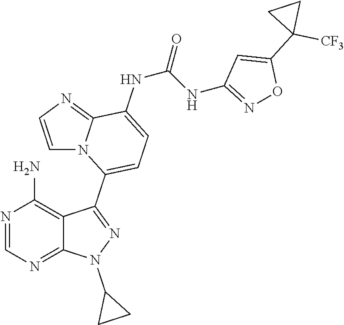 [SMILES: Nc1ncnc2c1c(-c1ccc(NC(=O)Nc3cc(C4(C(F)(F)F)CC4)on3)c3nccn13)nn2C1CC1] | 1-(5-(4-amino-1-cyclopropyl- 1H-pyrazolo[3,4-d]pyrimidin- 3-yl)imidazo[1,2-a]pyridin-8- yl)-3-(5-(1-(trifluoromethyl)- cyclopropyl)isoxazol-3-yl)urea |
| I-98 | 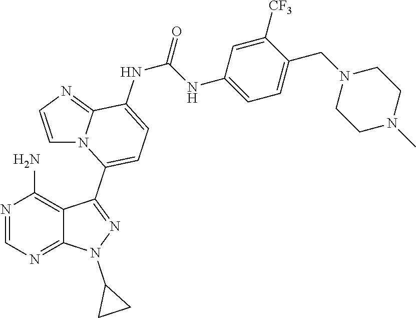 [SMILES: CN1CCN(Cc2ccc(NC(=O)Nc3ccc(-c4nn(C5CC5)c5ncnc(N)c45)n4ccnc34)cc2C(F)(F)F)CC1] | 1-(5-(4-amino-1-cyclopropyl- 1H-pyrazolo[3,4-d]pyrimidin- 3-yl)imidazo[1,2-a]pyridin-8- yl)-3-(4-((4-methylpiperazin- 1-yl)methyl)-3-(trifluorometh- yl)phenyl)urea |
| I-99 | 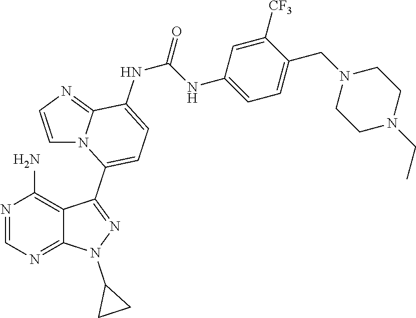 [SMILES: CCN1CCN(Cc2ccc(NC(=O)Nc3ccc(-c4nn(C5CC5)c5ncnc(N)c45)n4ccnc34)cc2C(F)(F)F)CC1] | 1-(5-(4-amino-1-cyclopropyl- 1H-pyrazolo[3,4-d]pyrimidin- 3-yl)imidazo[1,2-a]pyridin-8- yl)-3-(4-((4-ethylpiperazin-1- yl)methyl)-3-(trifluoromethyl)- phenyl)urea |
| I-100 | 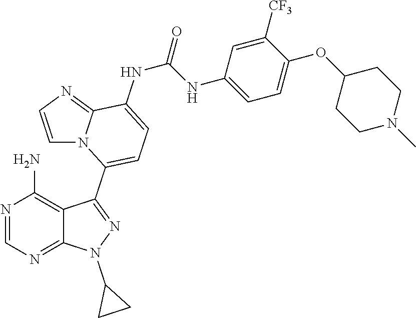 [SMILES: CN1CCC(Oc2ccc(NC(=O)Nc3ccc(-c4nn(C5CC5)c5ncnc(N)c45)n4ccnc34)cc2C(F)(F)F)CC1] | 1-(5-(4-amino-1-cyclopropyl- 1H-pyrazolo[3,4-d]pyrimidin- 3-yl)imidazo[1,2-a]pyridin-8- yl)-3-(4-((1-methylpiperidin- 4-yl)oxy)-3-(trifluoromethyl)- phenyl)urea |
| I-101 | 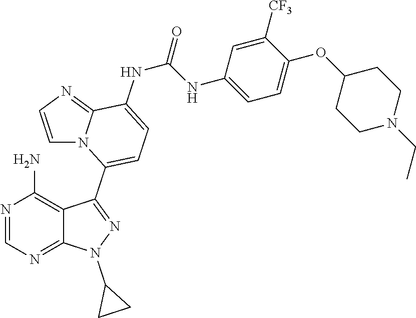 [SMILES: CCN1CCC(Oc2ccc(NC(=O)Nc3ccc(-c4nn(C5CC5)c5ncnc(N)c45)n4ccnc34)cc2C(F)(F)F)CC1] | 1-(5-(4-amino-1-cyclopropyl- 1H-pyrazolo[3,4-d]pyrimidin- 3-yl)imidazo[1,2-a]pyridin-8- yl)-3-(4-((1-ethylpiperidin-4- yl)oxy)-3-(trifluoromethyl)- phenyl)urea |
| I-102 | 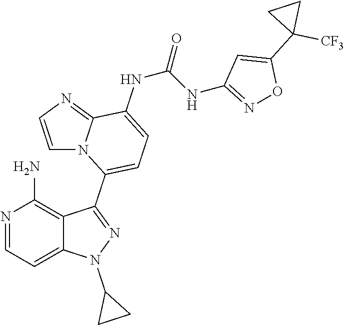 [SMILES: Nc1nccc2c1c(-c1ccc(NC(=O)Nc3cc(C4(C(F)(F)F)CC4)on3)c3nccn13)nn2C1CC1] | 1-(5-(4-amino-1-cyclopropyl- 1H-pyrazolo[4,3-c]pyridin-3- yl)imidazo[1,2-a]pyridin-8- yl)-3-(5-(1-(trifluoromethyl)- cyclopropyl)isoxazol-3-yl)urea |
| I-103 | 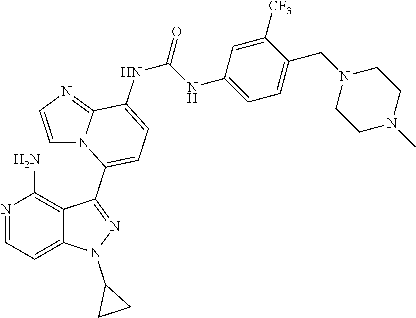 [SMILES: CN1CCN(Cc2ccc(NC(=O)Nc3ccc(-c4nn(C5CC5)c5ccnc(N)c45)n4ccnc34)cc2C(F)(F)F)CC1] | 1-(5-(4-amino-1-cyclopropyl- 1H-pyrazolo[4,3-c]pyridin-3- yl)imidazo[1,2-a]pyridin-8- yl)-3-(4-((4-methylpiperazin- 1-yl)methyl)-3-(trifluorometh- yl)phenyl)urea |
| I-104 | 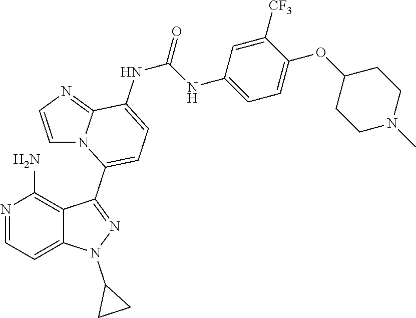 [SMILES: CN1CCC(Oc2ccc(NC(=O)Nc3ccc(-c4nn(C5CC5)c5ccnc(N)c45)n4ccnc34)cc2C(F)(F)F)CC1] | 1-(5-(4-amino-1-cyclopropyl- 1H-pyrazolo[4,3-c]pyridin-3- yl)imidazo[1,2-a]pyridin-8- yl)-3-(4-((1-methylpiperidin- 4-yl)oxy)-3-(trifluorometh- yl)phenyl)urea |
| I-105 | 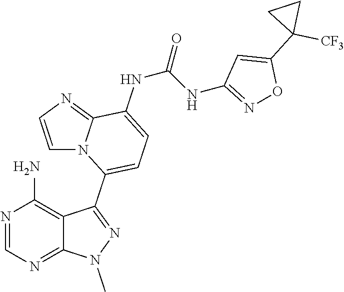 [SMILES: Cn1nc(-c2ccc(NC(=O)Nc3cc(C4(C(F)(F)F)CC4)on3)c3nccn23)c2c(N)ncnc21] | 1-(5-(4-amino-1-methyl-1H- pyrazolo[3,4-d]pyrimidin-3- yl)imidazo[1,2-a]pyridin-8- yl)-3-(5-(1-(trifluoromethyl)- cyclopropyl)isoxazol-3-yl)urea |
| I-106 | 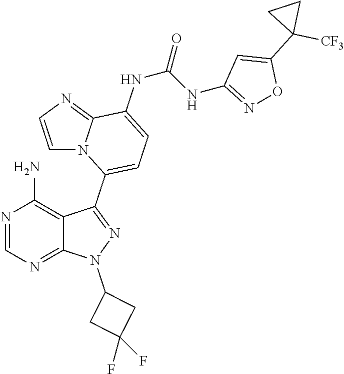 [SMILES: Nc1ncnc2c1c(-c1ccc(NC(=O)Nc3cc(C4(C(F)(F)F)CC4)on3)c3nccn13)nn2C1CC(F)(F)C1] | 1-(5-(4-amino-1-(3,3-difluoro- cyclobutyl)-1H-pyrazolo[3,4- d]pyrimidin-3-yl)imidazo[1,2- a]pyridin-8-yl)-3-(5-(1-(tri- fluoromethyl)cyclopropyl)- isoxazol-3-yl)urea |
| I-107 | 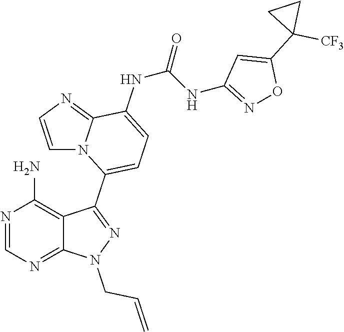 [SMILES: C=CCn1nc(-c2ccc(NC(=O)Nc3cc(C4(C(F)(F)F)CC4)on3)c3nccn23)c2c(N)ncnc21] | 1-(5-(1-allyl-4-amino-1H- pyrazolo[3,4-d]pyrimidin-3- yl)imidazo[1,2-a]pyridin-8- yl)-3-(5-(1-(trifluoromethyl)- cyclopropyl)isoxazol-3-yl)urea |
| I-108 | 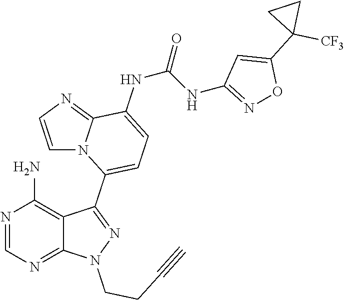 [SMILES: C#CCCn1nc(-c2ccc(NC(=O)Nc3cc(C4(C(F)(F)F)CC4)on3)c3nccn23)c2c(N)ncnc21] | 1-(5-(4-amino-1-(but-3-yn-1- yl)-1H-pyrazolo[3,4-d]- pyrimidin-3-yl)imidazo[1,2- a]pyridin-8-yl)-3-(5-(1-(tri- fluoromethyl)cyclopropyl)- isoxazol-3-yl)urea |
| I-109 | 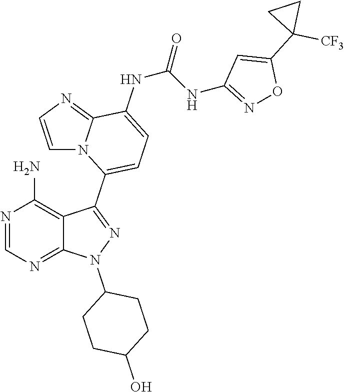 [SMILES: Nc1ncnc2c1c(-c1ccc(NC(=O)Nc3cc(C4(C(F)(F)F)CC4)on3)c3nccn13)nn2C1CCC(O)CC1] | 1-(5-(4-amino-1-(4-hydroxy- cyclohexyl)-1H-pyrazolo[3,4- d]pyrimidin-3-yl)imidazo[1,2- a]pyridin-8-yl)-3-(5-(1-(tri- fluoromethyl)cyclopropyl)- isoxazol-3-yl)urea |
| I-110 | 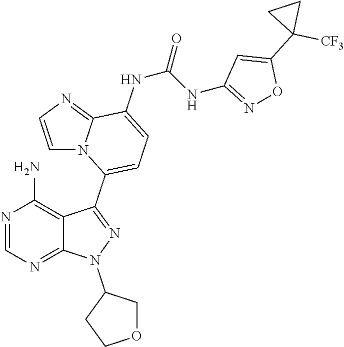 [SMILES: Nc1ncnc2c1c(-c1ccc(NC(=O)Nc3cc(C4(C(F)(F)F)CC4)on3)c3nccn13)nn2C1CCOC1] | 1-(5-(4-amino-1-(tetrahydro- furan-3-yl)-1H-pyrazolo[3,4- d]pyrimidin-3-yl)imidazo[1,2- a]pyridin-8-yl)-3-(5-(1-(tri- fluoromethyl)cyclopropyl)- isoxazol-3-yl)urea |
| I-111 | 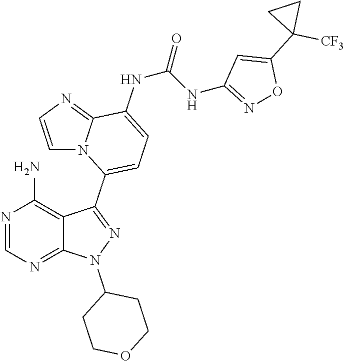 [SMILES: Nc1ncnc2c1c(-c1ccc(NC(=O)Nc3cc(C4(C(F)(F)F)CC4)on3)c3nccn13)nn2C1CCOCC1] | 1-(5-(4-amino-1-(tetrahydro- 2H-pyran-4-yl)-1H-pyrazolo- [3,4-d]pyrimidin-3-yl)imidazo- [1,2-a]pyridin-8-yl)-3-(5-(1- (trifluoromethyl)cyclopropyl)- isoxazol-3-yl)urea |
| I-112 | 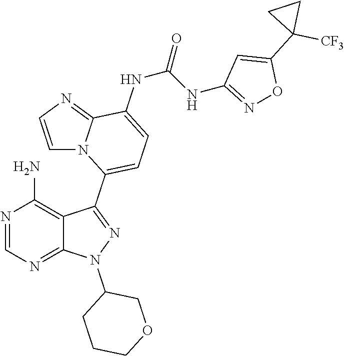 [SMILES: Nc1ncnc2c1c(-c1ccc(NC(=O)Nc3cc(C4(C(F)(F)F)CC4)on3)c3nccn13)nn2C1CCCOC1] | 1-(5-(4-amino-1-(tetrahydro- 2H-pyran-3-yl)-1H-pyrazolo- [3,4-d]pyrimidin-3-yl)imidazo- [1,2-a]pyridin-8-yl)-3-(5-(1- (trifluoromethyl)cyclopropyl)- isoxazol-3-yl)urea |
| I-113 | 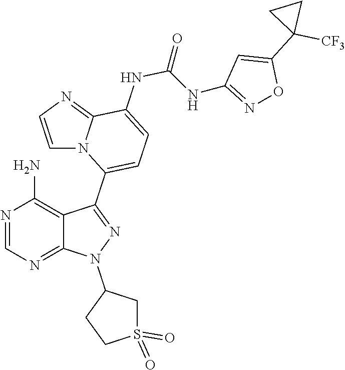 [SMILES: Nc1ncnc2c1c(-c1ccc(NC(=O)Nc3cc(C4(C(F)(F)F)CC4)on3)c3nccn13)nn2C1CCS(=O)(=O)C1] | 1-(5-(4-amino-1-(1,1-dioxido- tetrahydrothiophen-3-yl)-1H- pyrazolo[3,4-d]pyrimidin-3- yl)imidazo[1,2-a]pyridin-8- yl)-3-(5-(1-(trifluoromethyl)- cyclopropyl)isoxazol-3-yl)urea |
| I-114 | 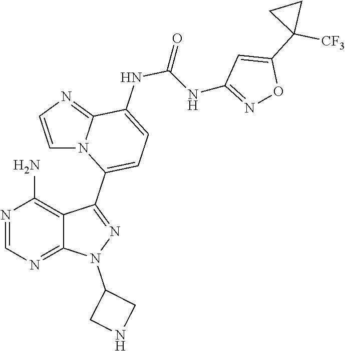 [SMILES: Nc1ncnc2c1c(-c1ccc(NC(=O)Nc3cc(C4(C(F)(F)F)CC4)on3)c3nccn13)nn2C1CNC1] | 1-(5-(4-amino-1-(azetidin-3- yl)-1H-pyrazolo[3,4-d]pyr- imidin-3-yl)imidazo[1,2-a]- pyridin-8-yl)-3-(5-(1-(tri- fluoromethyl)cyclopropyl)- isoxazol-3-yl)urea |
| I-115 | 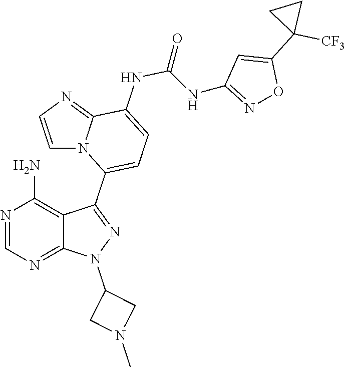 [SMILES: CN1CC(n2nc(-c3ccc(NC(=O)Nc4cc(C5(C(F)(F)F)CC5)on4)c4nccn34)c3c(N)ncnc32)C1] | 1-(5-(4-amino-1-(1-methyl- azetidin-3-yl)-1H-pyrazolo- [3,4-d]pyrimidin-3-yl)- imidazo[1,2-a]pyridin-8-yl)-3- (5-(1-(trifluoromethyl)cyclo- propyl)isoxazol-3-yl)urea |
| I-116 | 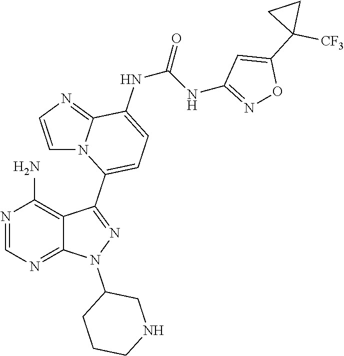 [SMILES: Nc1ncnc2c1c(-c1ccc(NC(=O)Nc3cc(C4(C(F)(F)F)CC4)on3)c3nccn13)nn2C1CCCNC1] | 1-(5-(4-amino-1-(piperidin-3- yl)-1H-pyrazolo[3,4-d]pyr- imidin-3-yl)imidazo[1,2-a]- pyridin-8-yl)-3-(5-(1-(trifluoro- methyl)cyclopropyl)isoxazol- 3-yl)urea |
| I-117 | 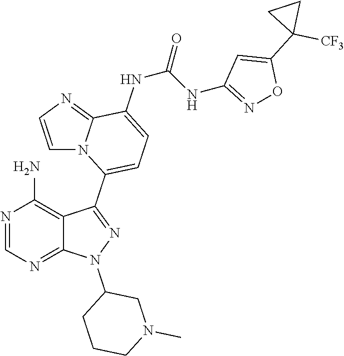 [SMILES: CN1CCCC(n2nc(-c3ccc(NC(=O)Nc4cc(C5(C(F)(F)F)CC5)on4)c4nccn34)c3c(N)ncnc32)C1] | 1-(5-(4-amino-1-(1-methyl- piperidin-3-yl)-1H-pyrazolo- [3,4-d]pyrimidin-3-yl)imidazo- [1,2-a]pyridin-8-yl)-3-(5-(1- (trifluoromethyl)cyclopropyl)- isoxazol-3-yl)urea |
| I-118 | 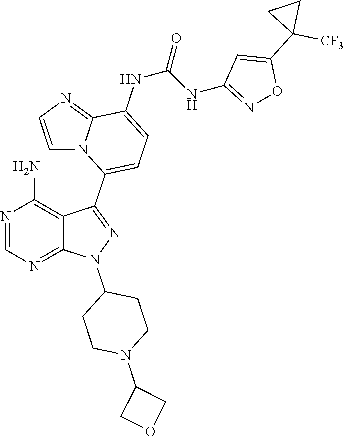 [SMILES: Nc1ncnc2c1c(-c1ccc(NC(=O)Nc3cc(C4(C(F)(F)F)CC4)on3)c3nccn13)nn2C1CCN(C2COC2)CC1] | 1-(5-(4-amino-1-(1-(oxetan-3- yl)piperidin-4-yl)-1H-pyr- azolo[3,4-d]pyrimidin-3-yl)- imidazo[1,2-a]pyridin-8-yl)- 3-(5-(1-(trifluoromethyl)cyclo- propyl)isoxazol-3-yl)urea |
| I-119 | 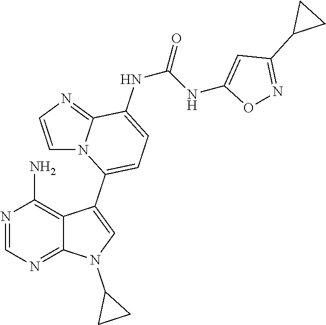 [SMILES: Nc1ncnc2c1c(-c1ccc(NC(=O)Nc3cc(C4CC4)no3)c3nccn13)cn2C1CC1] | 1-(5-(4-amino-7-cyclopropyl- 7H-pyrrolo[2,3-d]pyrimidin-5- yl)imidazo[1,2-a]pyridin-8- yl)-3-(3-cyclopropylisoxazol- 5-yl)urea |
| I-120 | 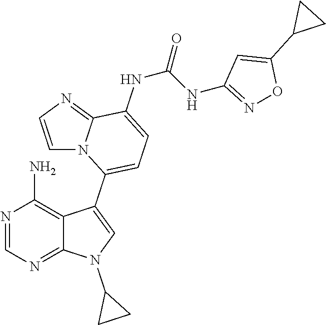 [SMILES: Nc1ncnc2c1c(-c1ccc(NC(=O)Nc3cc(C4CC4)on3)c3nccn13)cn2C1CC1] | 1-(5-(4-amino-7-cyclopropyl- 7H-pyrrolo[2,3-d]pyrimidin- 5-yl)imidazo[1,2-a]pyridin-8- yl)-3-(5-cyclopropylisoxazol- 3-yl)urea |
| I-121 | 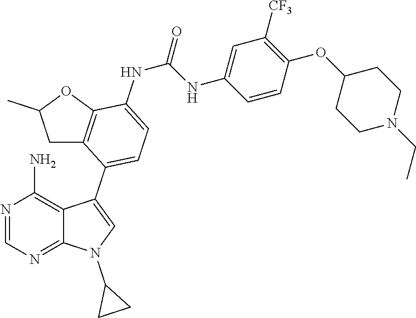 [SMILES: CCN1CCC(Oc2ccc(NC(=O)Nc3ccc(-c4cn(C5CC5)c5ncnc(N)c45)c4c3OC(C)C4)cc2C(F)(F)F)CC1] | 1-(4-(4-amino-7-cyclopropyl- 7H-pyrrolo[2,3-d]pyrimidin- 5-yl)-2-methyl-2,3-dihydro- benzofuran-7-yl)-3-(4-((1- ethylpiperidin-4-yl)oxy)-3- (trifluoromethyl)phenyl)urea |
| I-122 | 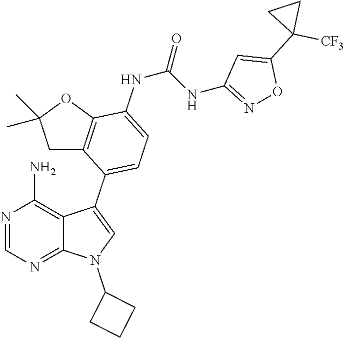 [SMILES: CC1(C)Cc2c(-c3cn(C4CCC4)c4ncnc(N)c34)ccc(NC(=O)Nc3cc(C4(C(F)(F)F)CC4)on3)c2O1] | 1-(4-(4-amino-7-cyclobutyl- 7H-pyrrolo[2,3-d]pyrimidin- 5-yl)-2,2-dimethyl-2,3- dihydrobenzofuran-7-yl)-3- (5-(1-(trifluoromethyl)cyclo- propyl)isoxazol-3-yl)urea |
| I-123 | 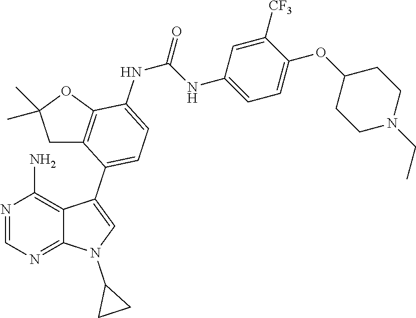 [SMILES: CCN1CCC(Oc2ccc(NC(=O)Nc3ccc(-c4cn(C5CC5)c5ncnc(N)c45)c4c3OC(C)(C)C4)cc2C(F)(F)F)CC1] | 1-(4-(4-amino-7-cyclopropyl- 7H-pyrrolo[2,3-d]pyrimidin- 5-yl)-2,2-dimethyl-2,3- dihydrobenzofuran-7-yl)-3-(4- ((1-ethylpiperidin-4-yl)oxy)- 3-(trifluoromethyl)phenyl)urea |
| I-124 | 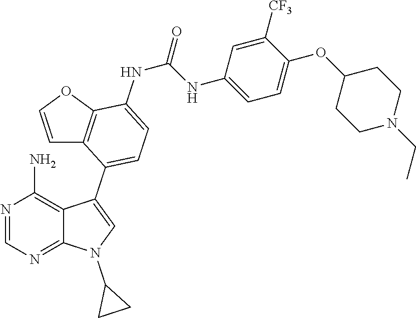 [SMILES: CCN1CCC(Oc2ccc(NC(=O)Nc3ccc(-c4cn(C5CC5)c5ncnc(N)c45)c4ccoc34)cc2C(F)(F)F)CC1] | 1-(4-(4-amino-7-cyclopropyl- 7H-pyrrolo[2,3-d]pyrimidin-5- yl)benzofuran-7-yl)-3-(4-((1- ethylpiperidin-4-yl)oxy)-3- (trifluoromethyl)phenyl)urea |
| I-125 | 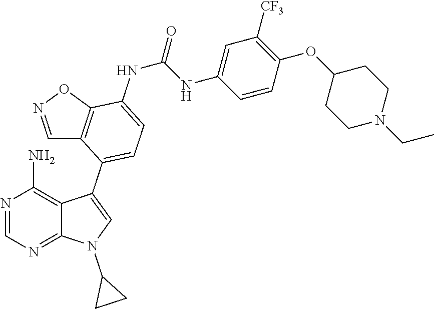 [SMILES: CCN1CCC(Oc2ccc(NC(=O)Nc3ccc(-c4cn(C5CC5)c5ncnc(N)c45)c4cnoc34)cc2C(F)(F)F)CC1] | 1-(4-(4-amino-7-cyclopropyl- 7H-pyrrolo[2,3-d]pyrimidin- 5-yl)benzo[d]isoxazol-7-yl)-3- (4-((1-ethylpiperidin-4-yl)oxy)- 3-(trifluoromethyl)phenyl)urea |
| I-126 | 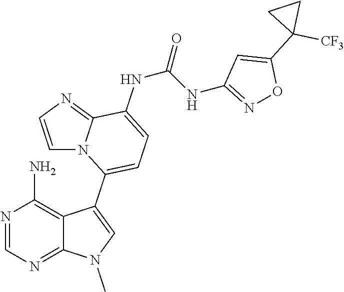 [SMILES: Cn1cc(-c2ccc(NC(=O)Nc3cc(C4(C(F)(F)F)CC4)on3)c3nccn23)c2c(N)ncnc21] | 1-(5-(4-amino-7-methyl-7H- pyrrolo[2,3-d]pyrimidin-5- yl)imidazo[1,2-a]pyridin-8- yl)-3-(5-(1-(trifluoromethyl)- cyclopropyl)isoxazol-3-yl)urea |
| I-127 | 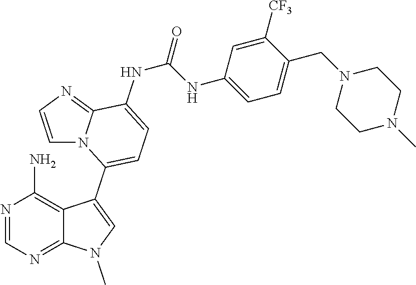 [SMILES: CN1CCN(Cc2ccc(NC(=O)Nc3ccc(-c4cn(C)c5ncnc(N)c45)n4ccnc34)cc2C(F)(F)F)CC1] | 1-(5-(4-amino-7-methyl-7H- pyrrolo[2,3-d]pyrimidin-5- yl)imidazo[1,2-a]pyridin-8- yl)-3-(4-((4-methylpiperazin- 1-yl)methyl)-3-(trifluorometh- yl)phenyl)urea |
| I-128 | 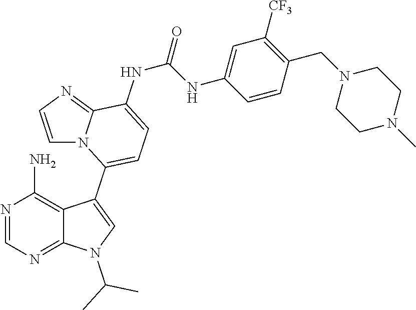 [SMILES: CC(C)n1cc(-c2ccc(NC(=O)Nc3ccc(CN4CCN(C)CC4)c(C(F)(F)F)c3)c3nccn23)c2c(N)ncnc21] | 1-(5-(4-amino-7-isopropyl- 7H-pyrrolo[2,3-d]pyrimidin- 5-yl)imidazo[1,2-a]pyridin-8- yl)-3-(4-((4-methylpiperazin- 1-yl)methyl)-3-(trifluorometh- yl)phenyl)urea |
| I-129 | 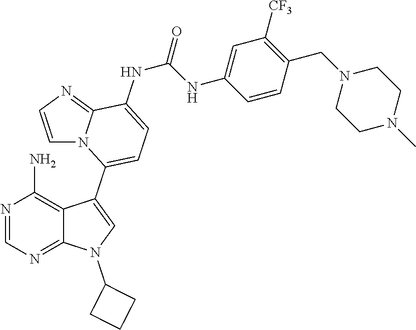 [SMILES: CN1CCN(Cc2ccc(NC(=O)Nc3ccc(-c4cn(C5CCC5)c5ncnc(N)c45)n4ccnc34)cc2C(F)(F)F)CC1] | 1-(5-(4-amino-7-cyclobutyl- 7H-pyrrolo[2,3-d]pyrimidin- 5-yl)imidazo[1,2-a]pyridin-8- yl)-3-(4-((4-methylpiperazin- 1-yl)methyl)-3-(trifluorometh- yl)phenyl)urea |
| I-130 | 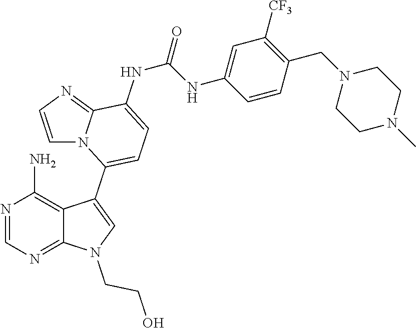 [SMILES: CN1CCN(Cc2ccc(NC(=O)Nc3ccc(-c4cn(CCO)c5ncnc(N)c45)n4ccnc34)cc2C(F)(F)F)CC1] | 1-(5-(4-amino-7-(2-hydroxy- ethyl)-7H-pyrrolo[2,3-d]pyr- imidin-5-yl)imidazo[1,2-a]- pyridin-8-yl)-3-(4-((4-meth- ylpiperazin-1-yl)methyl)-3- (trifluoromethyl)phenyl)urea |
| I-131 | 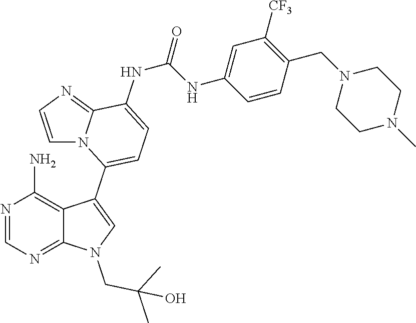 [SMILES: CN1CCN(Cc2ccc(NC(=O)Nc3ccc(-c4cn(CC(C)(C)O)c5ncnc(N)c45)n4ccnc34)cc2C(F)(F)F)CC1] | 1-(5-(4-amino-7-(2-hydroxy-2- methylpropyl)-7H-pyrrolo[2,3- d]pyrimidin-5-yl)imidazo[1,2- a]pyridin-8-yl)-3-(4-((4-meth- ylpiperazin-1-yl)methyl)-3- (trifluoromethyl)phenyl)urea |
| I-132 | 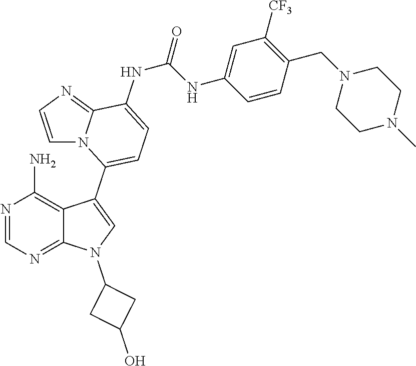 [SMILES: CN1CCN(Cc2ccc(NC(=O)Nc3ccc(-c4cn(C5CC(O)C5)c5ncnc(N)c45)n4ccnc34)cc2C(F)(F)F)CC1] | 1-(5-(4-amino-7-(3-hydroxy- cyclobutyl)-7H-pyrrolo[2,3- d]pyrimidin-5-yl)imidazo[1,2- a]pyridin-8-yl)-3-(4-((4-meth- ylpiperazin-1-yl)methyl)-3- (trifluoromethyl)phenyl)urea |
| I-133 | 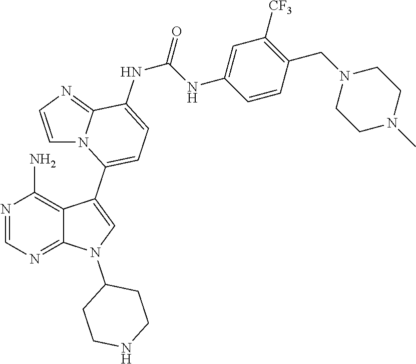 [SMILES: CN1CCN(Cc2ccc(NC(=O)Nc3ccc(-c4cn(C5CCNCC5)c5ncnc(N)c45)n4ccnc34)cc2C(F)(F)F)CC1] | 1-(5-(4-amino-7-(piperidin-4- yl)-7H-pyrrolo[2,3-d]pyr- imidin-5-yl)imidazo[1,2-a]- pyridin-8-yl)-3-(4-((4-methyl- piperazin-1-yl)methyl)-3- (trifluoromethyl)phenyl)urea |
| I-134 | 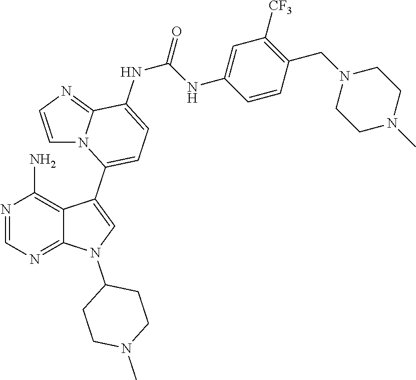 [SMILES: CN1CCC(n2cc(-c3ccc(NC(=O)Nc4ccc(CN5CCN(C)CC5)c(C(F)(F)F)c4)c4nccn34)c3c(N)ncnc32)CC1] | 1-(5-(4-amino-7-(1-methyl- piperidin-4-yl)-7H-pyrrolo- [2,3-d]pyrimidin-5-yl)imidazo- [1,2-a]pyridin-8-yl)-3-(4-((4- methylpiperazin-1-yl)methyl)- 3-(trifluoromethyl)phenyl)urea |
| I-135 | 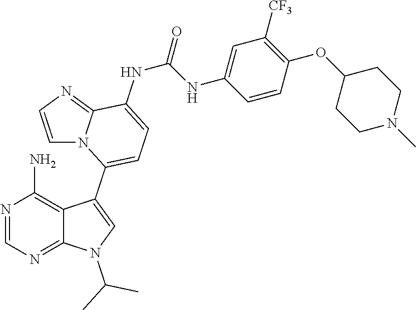 [SMILES: CC(C)n1cc(-c2ccc(NC(=O)Nc3ccc(OC4CCN(C)CC4)c(C(F)(F)F)c3)c3nccn23)c2c(N)ncnc21] | 1-(5-(4-amino-7-isopropyl- 7H-pyrrolo[2,3-d]pyrimidin- 5-yl)imidazo[1,2-a]pyridin-8- yl)-3-(4-((1-methylpiperidin- 4-yl)oxy)-3-(trifluoromethyl)- phenyl)urea |
| I-136 | 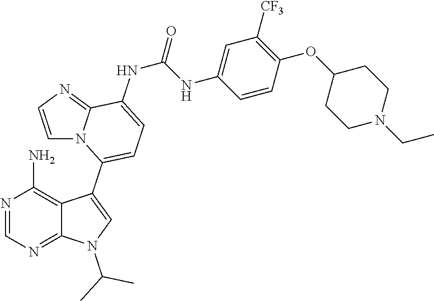 [SMILES: CCN1CCC(Oc2ccc(NC(=O)Nc3ccc(-c4cn(C(C)C)c5ncnc(N)c45)n4ccnc34)cc2C(F)(F)F)CC1] | 1-(5-(4-amino-7-isopropyl- 7H-pyrrolo[2,3-d]pyrimidin- 5-yl)imidazo[1,2-a]pyridin-8- yl)-3-(4-((1-ethylpiperidin-4- yl)oxy)-3-(trifluoromethyl)- phenyl)urea |
| I-137 | 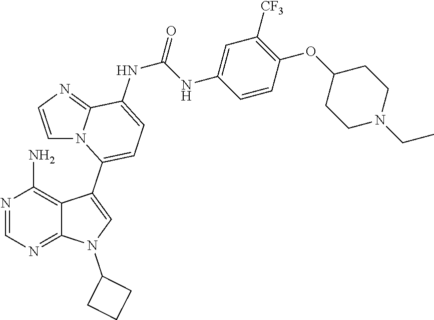 [SMILES: CCN1CCC(Oc2ccc(NC(=O)Nc3ccc(-c4cn(C5CCC5)c5ncnc(N)c45)n4ccnc34)cc2C(F)(F)F)CC1] | 1-(5-(4-amino-7-cyclobutyl- 7H-pyrrolo[2,3-d]pyrimidin- 5-yl)imidazo[1,2-a]pyridin-8- yl)-3-(4-((1-ethylpiperidin-4- yl)oxy)-3-(trifluoromethyl)- phenyl)urea |

It is understood that in the present description, combinations of substituents and/or variables of the depicted formulae are permissible only if such contributions result in stable compounds.

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

### Pharmaceutical Compositions

Other embodiments are directed to pharmaceutical compositions. The pharmaceutical composition comprises anyone (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

In treatment methods according to embodiments of the disclosure, an effective amount of at least one compound of Structure (I) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to inter-subject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structure (I), and a pharmaceutically acceptable carrier.

Provided herein are pharmaceutical compositions comprising one or more compounds selected from compounds of Structure (I) and pharmaceutically acceptable diluent(s), excipient(s), and carrier(s). In certain embodiments, the compounds described are administered as pharmaceutical compositions in which one or more compounds selected from compounds of Structure (I) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I).

In a certain embodiment, pharmaceutical compositions of the compounds of Structure (I) are modulators of the NLRP3 inflammasome.

In a specific embodiment, pharmaceutical compositions of the compounds of Structure (I) inhibit NEK7 when administered to a patient or a biological sample.

A pharmaceutical composition, as used herein, refers to a mixture of one or more compounds selected from compounds of Structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I) provided herein are administered in a pharmaceutical composition to a mammal having a disease, disorder, or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I) are formulated for trans-mucosal administration. In specific embodiments, trans-mucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions, and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions, or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent, or excipient, and one or more compounds selected from compounds of Structure (I), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass un-solvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions, and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I) illustratively takes the form of a liquid where the agents are present in solution, in suspension or both. Typically, when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium, or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-re-closable containers. Alternatively, multiple-dose re-closable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds selected from compounds of Structure (I) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v, or v/v.

In some embodiments, the amount the one or more compounds selected from compounds of Structure (I) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial, and/or tube labels listing contents and/or instructions for use, and package inserts with instructions for use. A set of instructions will also typically be included. A label is optionally on or associated with the container. For example, a label is on a container when letters, numbers or other characters forming the label are attached, molded, or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or the pack or dispenser device is accompanied by instructions for administration. Or the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### Methods

Embodiments of the present disclosure are useful as modulators of the NLRP3 inflammasome via the inhibition of NEK7 in a host species. Therefore, the compounds of Structure (I) are also useful in the treatment of conditions mediated by effector signaling molecules like IL-1β and IL-18.

The host or patient can belong to any mammalian species, for example a primate species, particularly humans; rodents, including mice, rats, and hamsters; rabbits; horses, cows, dogs, cats, etc. Animal models are of interest for experimental investigations, providing a model for treatment of human disease.

In one embodiment, the present disclosure is useful as an inhibitor of the NLRP3 inflammasome activation mechanism. Therefore, the compounds of Structure (I) are also useful in the treatment of conditions resulting from that activation in a host species.

In another embodiment, the compounds of Structure (I) are useful as inhibitors of the NLRP3 (protein)-NEK7 (protein) interaction. Therefore, the compounds are also useful in the treatment of conditions resulting from the association of NLRP3-NEK7 in a host species.

In certain embodiments, the compounds of Structure (I) are useful in treating human conditions mediated by effectors selected from the group consisting of IL-1β, IL-18, and caspase-1.

Embodiments of the disclosure also relate to the use of compounds according to Structure (I) and/or physiologically acceptable salts thereof for the prophylactic or therapeutic treatment and/or monitoring of diseases that are caused, mediated and/or modulated by the NLRP3 inflammasome activity. Furthermore, embodiments of the disclosure relate to the use of compounds according to Structure (I) and/or physiologically acceptable salts thereof to produce a medicament for the prophylactic or therapeutic treatment and/or monitoring of diseases that are caused, mediated and/or modulated by NLRP3 inflammasome activity. In certain embodiments, the disclosure provides the use of a compound according to Structure (I) or physiologically acceptable salts thereof, to produce a medicament for the prophylactic or therapeutic treatment of a NLRP3-mediated disorder.

In another embodiment, the present disclosure relates to a method of treating inflammatory diseases or conditions mediated by NLRP3 inflammasome by administering to a patient in need thereof a therapeutically effective amount of the compound of Structure (I).

In certain embodiments, the diseases which can be treated with a compound of Structure (I) include type II diabetes, atherosclerosis, Alzheimer's disease, aging, fatty liver, metabolic syndrome, asthma, psoriasis, obesity, acute and chronic tissue damage caused by infection, gout, arthritis, enteritis, hepatitis, peritonitis, silicosis, UV-induced skin sunburn, contact hypersensitivity, sepsis, cancer, neurodegenerative disease, multiple sclerosis, Muckle Wells syndrome, and combinations thereof.

In certain other embodiments, the compounds of Structure (I) are used in methods for treatment of disorders or diseases selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases, ischemic conditions, cancer, and combinations thereof.

In some embodiments, the disorders associated with NEK7 which are treatable with a compound of Structure (I) are selected from rheumatoid arthritis, psoriatic arthritis, osteoarthritis, systemic lupus erythematosus, lupus nephritis, ankylosing spondylitis, osteoporosis, systemic sclerosis, multiple sclerosis, psoriasis, type I diabetes, type II diabetes, inflammatory bowel disease (Crohn's Disease and ulcerative colitis), hyperimmunoglobulinemia D and periodic fever syndrome, cryopyrin associated periodic syndromes, Schnitzler's syndrome, systemic juvenile idiopathic arthritis, adult's onset Still's disease, gout, pseudogout, SAPHO syndrome, Castleman's disease, sepsis, stroke, atherosclerosis, celiac disease, DIRA (Deficiency of IL-1 Receptor Antagonist), Alzheimer's disease, Parkinson's disease, cancer, and combinations thereof.

Also included herein are methods of treatment in which at least one compound of Structure (I) is administered in combination with an anti-inflammatory or a therapeutic agent. Anti-inflammatory agents include but are not limited to NSAIDs, non-specific and COX-2 specific cyclooxygenase enzyme inhibitors, gold compounds, corticosteroids, methotrexate, tumor necrosis factor (TNF) antagonists, immunosuppressants, and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, hydroxychloroquine, and combinations thereof.

Examples of NSAIDs also include COX-2 specific inhibitors such as celecoxib, valdecoxib, lumiracoxib, and/or etoricoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The disclosure also includes embodiments in which the anti-inflammatory agent is a metabolic inhibitor such as a dihydrofolate reductase inhibitor, such as methotrexate or a dihydroorotate dehydrogenase inhibitor, such as leflunomide.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Other embodiments of the disclosure pertain to combinations in which at least one anti-inflammatory compound is an anti-monoclonal antibody (such as eculizumab or pexelizumab), a TNF antagonist, such as entanercept, or infliximab, which is an anti-TNF alpha monoclonal antibody.

Therapeutic agents used in combination with the compounds of Structure (I) can also include small molecule compounds that inhibit the activation of NLRP3 inflammasomes, such as MCC950, sulforaphane, iisoliquiritigenin, β-hydroxybutyrate, flufenamic acid, mefenamic acid, 3,4-methylenedioxy-β-nitrostyrene (MNS), and parthenolide.

Still other embodiments of the disclosure pertain to combinations in which at least one active agent is an immunosuppressant compound such as an immunosuppressant compound chosen from methotrexate, leflunomide, cyclosporine, tacrolimus, azathioprine, and mycophenolate mofetil.

The disclosed compounds of Structure (I) can be administered in combination with other known therapeutic agents, including anticancer agents. As used here, the term “anticancer agent” relates to any agent which is administered to a patient with cancer for the purposes of treating the cancer.

In some embodiments the anti-cancer agents belong to the following categories—
Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-3024, VAL-0834;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, laromustine;
Topoisomerase Inhibitors: such as etoposide, irinotecan, razoxane, sobuzoxane, teniposide, topotecan; amonafide, belotecan, elliptinium acetate, voreloxin;
Microtubule modifiers: such as cabazitaxel, docetaxel, eribulin, ixabepilone, paclitaxel, vinblastine, vincristine, vinorelbine, vindesine, vinflunine; fosbretabulin, tesetaxel;
Antimetabolites: such as asparaginase3, 76mbody76line, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur, trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide;
Aromatase inhibitors: such as aminoglutethimide, anastrozole, exemestane, fadrozole, letrozole, testolactone; formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, 77mbody77lin, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, cediranib.

In some embodiments, medicaments which are administered in conjunction with the compounds described herein include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, 77mbody77line, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics, e.g., ipratropium, atropine or oxitropium; hormones, e.g., cortisone, hydrocortisone or prednisolone; xanthines, e.g., aminophylline, choline theophyllinate, lysine theophyllinate or theophylline; and therapeutic proteins and peptides, e.g., insulin or glucagon. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

The agents disclosed herein, or other suitable agents are administered depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, a few hours apart, or a few days apart.

In some embodiments, the compounds of Structure (I) are administered as a monotherapy.

For identification of a signal transduction or a mechanistic pathway and for detection of interactions between various signal transduction pathways, various scientists have developed suitable models or model systems, for example cell culture models and models of transgenic animals. For the determination of certain stages in the signal transduction cascade, interacting compounds can be utilized to modulate the signal. The compounds of embodiments of the disclosure can also be used as reagents for testing NEK7-dependent signal transduction pathways in animals and/or cell culture models or in the clinical diseases mentioned in this application.

In some embodiments, the methods of the disclosure can be performed either in vitro or in vivo. The susceptibility of a particular cell to treatment with the compounds of Structure (I) can be particularly determined by in vitro tests, whether during research or clinical application. Typically, a culture of the cell is combined with a compound at various concentrations for a period which is sufficient to allow the active agents to inhibit NEK7 activity, usually between about one hour and one week. In vitro treatment can be carried out using cultivated cells from a biopsy sample or cell line.

In some embodiments, the IC₅₀ of the compounds of Structure (I) to inhibit NEK7 was determined by the concentration of the compound required to inhibit 50% of the activity of the NEK kinase. The compounds of Structure (I) exhibited potency values of IC₅₀ of less than about 5 mM, preferably less than about 1 mM and even more preferably less than about 0.100 mM as described in further detail in the Examples.

The examples and preparations provided below further illustrate and exemplify the compounds of the present disclosure and methods of preparing and testing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art.

Methods for producing the compounds described herein is provided below. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5^{th} edition (Wiley, December 2000)) or prepared as described herein.

The following General Reaction Schemes illustrate exemplary methods for preparation of compounds of Structure (I) (as well as various Intermediates A-E):

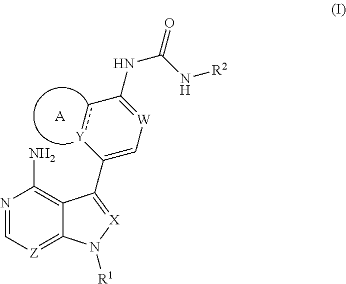

Or a stereoisomer, tautomer, or salt thereof, wherein X^{H} is a halogen, R^{P}, and R^{P′} are independently protecting groups, and A, W, X, Y, Z, R¹, and R² are as defined herein.

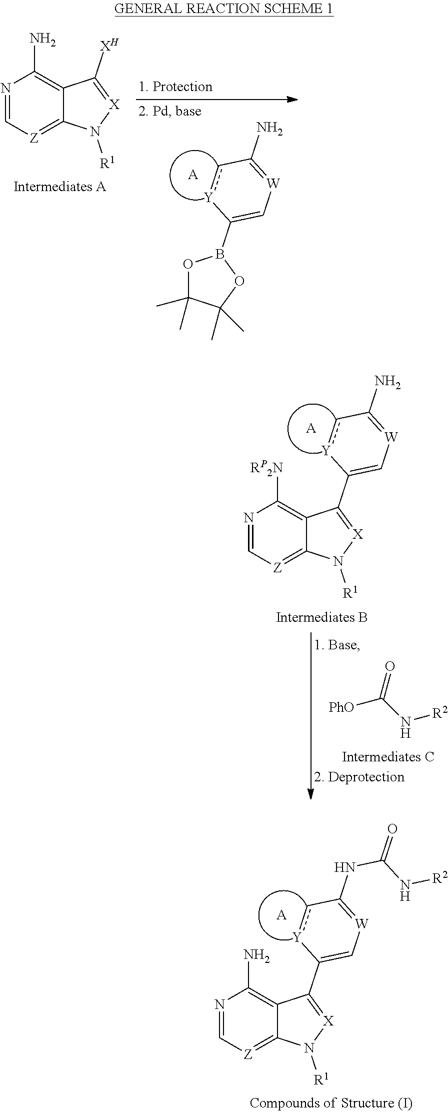

As shown in General Reaction Scheme 1, protection of the primary amine of bicyclic Intermediate A followed by palladium catalyzed arylation with a boronate ester yields amine Intermediate B. This can in turn be reacted with carbamate Intermediate C in the presence of base, affording compounds of Structure (I) following deprotection of the masked primary amine.

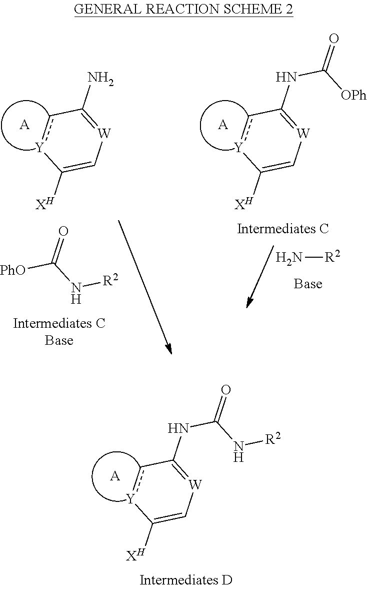

As shown in General Reaction Scheme 2, reaction of carbamate Intermediate C with an amine in the presence of base affords aryl halide Intermediate D.

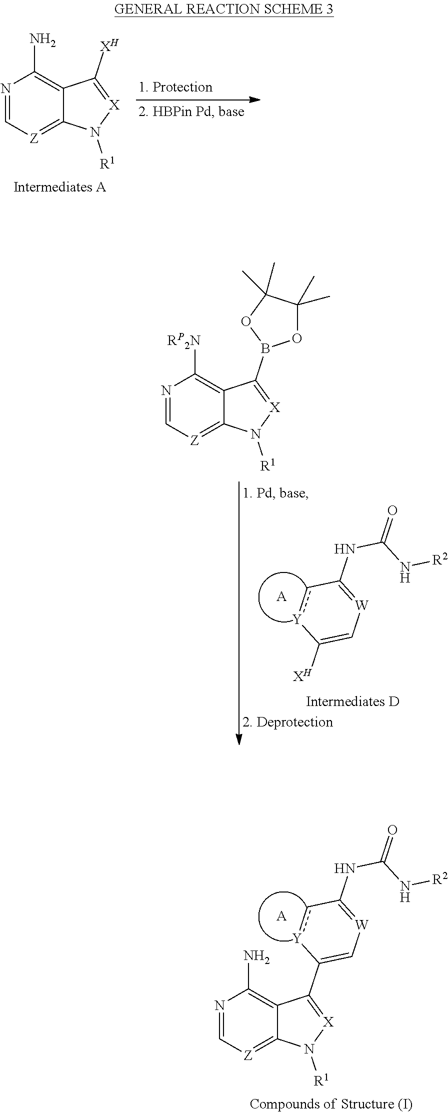

As shown in General Reaction Scheme 3, protection of the primary amine of bicyclic Intermediate A followed by palladium catalyzed borylation in the presence of a base yields a boronate ester which can be coupled with aryl halide Intermediate D in the presence of palladium and base, affording compounds of Structure (I) following deprotection of the masked primary amine.

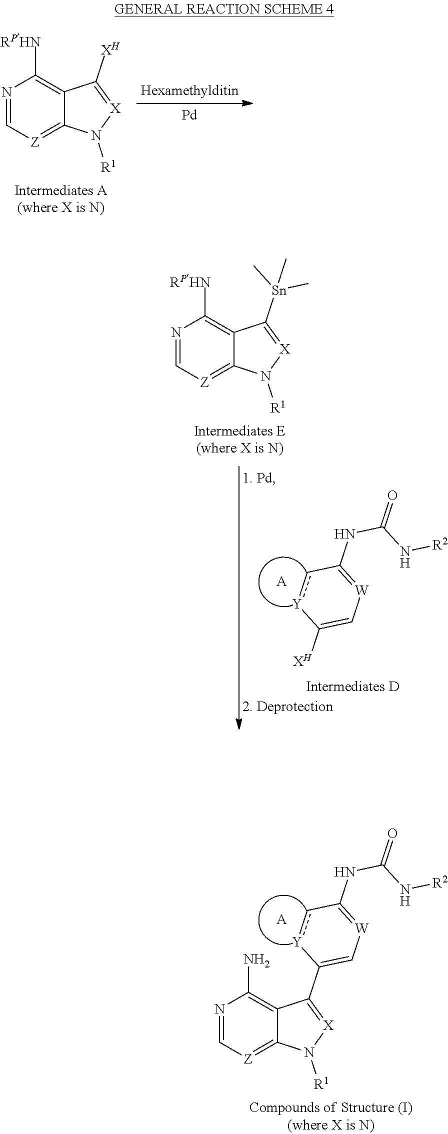

As shown in General Reaction Scheme 4, stannylation of aryl halide Intermediate A (in which X is N) to afford Intermediate E, followed by palladium catalyzed coupling with aryl halide Intermediate D in the presence of base affords compounds of Structure (I) (in which X is N) following deprotection of the masked primary amine.

### EXAMPLES

The following examples are provided for exemplary purposes.

### General Procedures

All proton NMR experiments were recorded on a Bruker NEO Spectrometer equipped with a BBFO probe at 400 MHz. Deuterated solvents contained less than 0.05% v/v tetramethylsilane which was used as the reference signal (set at 0.00 ppm). When deuterated solvents did not contain tetramethylsilane, the residual nondeuterated solvent peaks were used as a reference signal, as per published guidelines (J. Org. Chem. 1997, 62(21), 7512-7515). Chemical shifts are expressed in parts per million (ppm, δ units). Coupling constants are in hertz (Hz). Splitting patterns describe apparent multiplicities and are designated as s (singlet), d (doublet), t (triplet), q (quartet), m (multiplet), qt (quintuplet) or bs (broad singlet).

LC/MS analyses were performed on an Agilent Technologies UHPLC 1290 Infinity II with a G6125 MS detector.

Microwave reactions were conducted with a Monowave 300 by Anton Paar GmbH using standard protocols.

### NEK7 Enzymatic Assay

Casein substrate (from bovine milk, hydrolyzed and partially dephosphorylated mixture of α, β and κ caseins, obtained from Sigma Aldrich, catalogue #C4765, diluted in distilled water to a final concentration of 1 mg/mL) and full-length recombinant human NEK7 (expressed by baculovirus in Sf9 insect cells using a N-terminal GST tag, obtained from SignalChem, catalogue #N09-10G, 0.1 μg/μL) were mixed in assay buffer (20 mM HEPES pH 7.5, 10 mM MgCl₂, 1 mM EGTA, 0.02% Brij35, 0.02 mg/mL BSA, 0.1 mM Na₃VO₄, 2 mM DTT, 1% DMSO). Compounds of interest (serial 3-fold dilution in DMSO from 10 μM to 0.5 nM) or vehicle (1% DMSO) were dispensed into the kinase reaction mixture by Acoustic technology (Echo550; nanoliter range). After incubation at room temperature for 20 minutes, the kinase reaction was initiated by addition of [³³P]-ATP (specific activity 10 μCi/μL) and the mixture was incubated at room temperature for 2 hours. The reaction was then stopped by spotting the reaction mixture on strips of phosphocellulose P81 paper. Following washing, the radioactivity of the P81 paper was measured and kinase activity data were expressed as the percent remaining kinase activity in test samples compared to vehicle reactions. IC₅₀ values and curve fits were obtained using Prism (GraphPad Software).

### IL-1β Release Assay

Approximately 1.5 million THP-1 cells were plated in each well of a 6-well TC plate and incubated with 40 nM PMA in RPMI (10% FBS, 1% Penstrep) for 24 hours. The media was then removed, and cells were rested in RPMI (10% FBS, 1% Penstrep) for 24 hours after which time the media was removed and cells were pre-treated for 2 hours with various concentrations of compounds of interest (typically serial 3-fold dilution in RPMI+5% FBS, concentrations ranging from 1 μM to 0.5 nM) in RPMI (5% FBS). The media was again removed, and cells were incubated with 250 ng/mL LPS and compounds of interest (concentrations as above) in RMPI (5% FBS) for 2 hours. The media was removed for a last time and cells were incubated with 20 μM nigericin and compounds of interest (concentrations as above) in Opti-MEM for 30 minutes. Cell media was then collected and the amount of cleaved IL-1β was determined using a JESS instrument (Protein Simple) and standard protocols. Cleaved Il-1β antibody was obtained from Cell Signaling (catalogue #83186S) and was used at 1:20 dilution in antibody diluent 2. Protein Simple 1× anti-Rabbit HRP secondary antibody was used along with Protein Simple luminol and peroxide for chemiluminescent detection. Primary antibody incubation time was increased from 30 minutes to 60 minutes.

### Abbreviations

° C. (degree Celsius); ¹H NMR (proton Nuclear Magnetic Resonance); ACN (acetonitrile); bipy (2,2′-bipyridine); Boc (tert-butyloxycarbonyl); (Boc)₂O (di-tert-butyl dicarbonate); Cu(OAc)₂ (copper (II) acetate); DCM (dichloromethane); DIPEA (N,N-diisopropylethylamine); DMAP (4-dimethylaminopyridine); DMF (N,N-dimethylformamide); DMSO-d₆ (deuterated dimethylsulfoxide); eq (equivalent); EtOAc (ethyl acetate); EtOH (ethanol); g (gram); h (hour); HBpin (4,4,5,5-tetramethyl-1,3,2-dioxaborolane); HPLC (High Performance Liquid Chromatography); LCMS (Liquid Chromatography Mass Spectrometry); LDA (lithium diisopropyl amide); MeOH (methanol); mg (milligram); min (minute); mL (milliliter); μL (microliter); mmol (millimole); MsCl (methanesulfonyl chloride); NBS (N-bromosuccinimide); NCS (N-chlorosuccinimide); NIS (N-iodosuccinimide); Pd/C (palladium on carbon); Pd₂(dba)₃ (tris(dibenzylideneacetone)dipalladium); Pd(PPh₃)₄ ((tetrakis(triphenylphosphine)palladium(0));)); PdCl₂(dppf) ([1,1′-bis(diphenylphosphino)ferrocene]palladium(II) dichloride); SEM-Cl (2-(trimethylsilyl)ethoxymethyl chloride); STAB (sodium triacetoxyborohydride); T₃P (1-propanephosphonic anhydride); TBAF (tetra-n-butylammonium fluoride); TEA (triethylamine); TFA (trifluoroacetic acid); THF (tetrahydrofuran); TLC (Thin Layer Chromatography); UPLC (Ultra performance liquid chromatography); XPhos (dicyclohexyl[2′,4′,6′-tris(propan-2-yl)[1,1′-biphenyl]-2-yl]phosphane).

### Preparation of Synthetic Intermediates

### Intermediate A1

### 7-Cyclopropyl-5-Iodo-7H-Pyrrolo[2,3-D]Pyrimidin-4-Amine

### Step 1: Synthesis of 4-chloro-7-cyclopropyl-7H-pyrrolo[2,3-d]pyrimidine

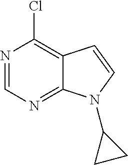 [SMILES: Clc1ncnc2c1ccn2C1CC1]

Cu(OAc)₂ (11.83 g, 65.1 mmol), bipy (10.17 g, 65.1 mmol), and NaHCO₃ (10.94 g, 130.2 mmol) were added to a solution of 4-chloro-7H-pyrrolo[2,3-d]pyrimidine (10.00 g, 65.1 mmol) and cyclopropylboronic acid (11.19 g, 130.2 mmol) in dichloroethane (150 mL) and the resulting mixture was stirred at 80° C. under oxygen atmosphere for 12 h. Following completion of the reaction (as indicated by TLC), the reaction mixture was filtered through a pad of Celite® (i.e., diatomaceous earth) which was then rinsed with DCM (2×100 mL). The combined filtrates were washed with water (100 mL) and brine (100 mL), dried over Na₂SO₄, filtered, and concentrated under reduced pressure to yield crude material which was purified by GRACE (silica gel 230-400 mesh, eluting with 6% EtOAc in petroleum ether), giving the title compound as an off-white solid (7.3 g, 58% yield). ¹H NMR (400 MHz, DMSO-d₆) δ=8.65 (s, 1H), 7.70 (d, J=3.6 Hz, 1H), 6.59 (d, J=4.0 Hz, 1H), 3.62-3.68 (m, 1H), 1.08-1.10 (m, 4H). MS: 194.0 [M+H].

### Step 2: Synthesis of 4-chloro-7-cyclopropyl-5-iodo-7H-pyrrolo[2,3-d]pyrimidine

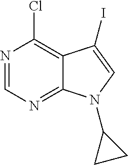 [SMILES: Clc1ncnc2c1c(I)cn2C1CC1]

NIS (8.25 g, 36.7 mmol) was added to a solution of 4-chloro-7-cyclopropyl-7H-pyrrolo[2,3-d]pyrimidine (7.10 g, 36.7 mmol) in DMF (50 mL) at 0° C. and the resulting mixture was stirred at 25° C. for 12 h. Following completion of the reaction (as indicated by TLC), the reaction mixture was poured into ice-cold water (600 mL) and stirred for 15 min. The resulting solid was filtered, washed with water (2×250 mL), and dried under reduced pressure to afford the title compound (8.3 g, 70% yield) as a pale brown solid. ¹H NMR (400 MHz, DMSO-d₆) δ=8.65 (s, 1H), 7.94 (s, 1H), 3.62-3.68 (m, 1H), 1.05-1.10 (m, 1H). MS: 319.9 [M+H].

### Step 3: Synthesis of 7-cyclopropyl-5-iodo-7H-pyrrolo[2,3-d]pyrimidin-4-amine

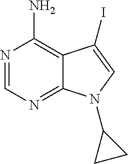 [SMILES: Nc1ncnc2c1c(I)cn2C1CC1]

A mixture of 4-chloro-7-cyclopropyl-5-iodo-7H-pyrrolo[2,3-d]pyrimidine (8.3 g, 26.0 mmol) and NH₄OH (25% in water, 25 mL) in 1,4-dioxane (25 mL) was stirred at 120° C. for 16 h in a tinyclave. Following completion of the reaction (as indicated by TLC), the reaction mixture was cooled to 25° C. and the resulting solid was filtered, washed with petroleum ether (2×50 mL x 2), and dried under reduced pressure to yield the title compound as an off-white solid (6.0 g, 76% yield). ¹H NMR (400 MHz, DMSO-d₆) δ=8.11 (s, 1H), 7.38 (s, 1H), 6.59 (bs, 2H), 3.48-3.54 (m, 1H), 0.97-0.99 (m, 4H). MS: 300.9 [M+H].

### Intermediate A2

### 1-Cyclopropyl-N-(2,4-Dimethoxybenzyl)-3-Iodo-1H-Pyrrolo[3,2-C]Pyridin-4-Amine

### Step 1: Synthesis of 4-chloro-1-cyclopropyl-1H-pyrrolo[3,2-c]pyridine

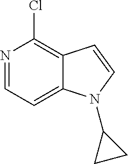 [SMILES: Clc1nccc2c1ccn2C1CC1]

TEA (0.332 g, 3.280 mmol), Cu(OAc)₂ (0.298 g, 1.638 mmol), and molecular sieves (powdered, 0.050 g) were added to a solution of 4-chloro-1H-pyrrolo[3,2-c]pyridine (0.250 g, 1.638 mmol) and cyclopropyl boronic acid (0.279 g, 3.280 mmol) in DMF (10 mL) and the resulting suspension was stirred at 60° C. for 12 h in a sealed tube. Following completion of the reaction (as indicated by TLC), the reaction mixture was filtered through a pad of Celite® (i.e., diatomaceous earth) which was then rinsed with EtOAc. The combined filtrates were concentrated under reduced pressure, giving crude material which was purified by Isolera (silica gel 230-400 mesh, eluting with 20% EtOAc in petroleum ether), affording the title compound as a yellow solid (0.19 g, 59% yield). ¹H NMR (400 MHz, DMSO-d₆) δ=8.04 (d, J=5.6 Hz, 1H), 7.56-7.60 (m, 2H), 6.52-6.53 (m, 1H), 3.55-3.58 (m, 1H), 1.00-1.13 (m, 4H). MS: 193.1 [M+H].

### Step 2: Synthesis of 4-chloro-1-cyclopropyl-3-iodo-1H-pyrrolo[3,2-c]pyridine

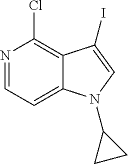 [SMILES: Clc1nccc2c1c(I)cn2C1CC1]

NIS (0.350 g, 1.557 mmol) was added to a solution of 4-chloro-1-cyclopropyl-1H-pyrrolo[3,2-c]pyridine (0.200 g, 1.038 mmol) in DMF (5 mL) and the resulting mixture was stirred at 80° C. for 1 hour. Following completion of the reaction (as indicated by LCMS), the reaction mixture was poured into crushed ice (25 g) and extracted with EtOAc (2×25 mL). The combined organic extracts were washed with brine, dried over Na₂SO₄, filtered, and concentrated under reduced pressure to yield the title product (0.2 g) which was used without further purification. LCMS: 319.0 [M+H].

### Step 3: Synthesis of 1-cyclopropyl-N-(2,4-dimethoxybenzyl)-3-iodo-1H-pyrrolo[3,2-c]pyridin-4-amine

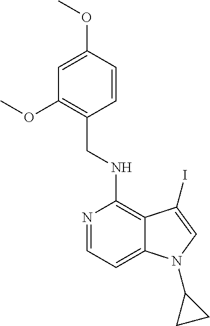 [SMILES: COc1ccc(CNc2nccc3c2c(I)cn3C2CC2)c(OC)c1]

A mixture of 4-chloro-1-cyclopropyl-3-iodo-1H-pyrrolo[3,2-c]pyridine (0.300 g, 0.942 mmol) and (2,5-dimethoxyphenyl)methanamine (0.429 mL, 2.83 mmol) in n-BuOH (10 mL) was stirred at 110° C. for 12 h. Following completion of the reaction (as indicated by LCMS), the reaction mixture was concentrated under reduced pressure to give crude material which was purified by Isolera (silica gel 230-400 mesh, eluting with 30% EtOAc in petroleum ether). Affording the title product as a yellow gum (0.10 g, 19% yield). MS: 450.0 [M+H].

### Intermediate A3

### 5-Bromo-7-Isopropyl-7H-Pyrrolo[2,3-D]Pyrimidin-4-Amine

### Step 1: Synthesis of 5-bromo-4-chloro-7H-pyrrolo[2,3-d]pyrimidine

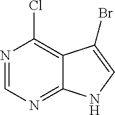 [SMILES: Clc1ncnc2[nH]cc(Br)c12]

The title compound was prepared by following a similar procedure described for step 2 of Intermediate A1, starting from 4-chloro-7H-pyrrolo[2,3-d] pyrimidine (1.00 g, 6.51 mmol) and NBS (1.16 g, 6.51 mmol), and was isolated as a yellow solid (1.2 g, 80% yield). ¹H NMR (400 MHz, DMSO-d₆) δ=12.95 (bs, 1H), 8.63 (s, 1H), 7.96 (d, J=2.4 Hz, 1H); LCMS: 232.0 [M+H].

### Step 2: Synthesis of 5-bromo-4-chloro-7-isopropyl-7H-pyrrolo[2,3-d]pyrimidine

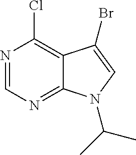

A mixture or 5-bromo-4-chloro-7H-pyrrolo[2,3-d]pyrimidine (0.50 g, 2.15 mmol), K₂CO₃ (0.59 g, 4.30 mmol), and 2-iodopropane (0.22 g, 1.30 mmol) in dry DMF (5 mL) was stirred at 90° C. for 16 h. Following completion of the reaction (as indicated by TLC), the reaction mixture was poured into crushed ice (50 g) and stirred for 15 min. The resulting precipitate was filtered, washed with water (2×5 mL), and dried to afford the title compound as a white solid (0.3 g, 59% yield). ¹H NMR (400 MHz, DMSO-d₆) δ=8.67 (s, 1H), 8.20 (s, 1H), 5.06-5.09 (m, 1H), 1.48 (d, J=6.8 Hz, 6H); LCMS: 273.7 [M+H].

### Step 3: Synthesis of 5-bromo-7-isopropyl-7H-pyrrolo[2,3-d]pyrimidin-4-amine

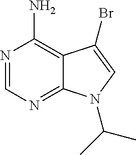

A mixture of 5-bromo-4-chloro-7-isopropyl-7H-pyrrolo[2,3-d]pyrimidine (0.3 g, 1.1 mmol) and NH₄OH (25% in water, 1.5 mL) was subjected to microwave irradiation at 150° C. for 1 h. Following completion of the reaction (as indicated by TLC), the reaction mixture was concentrated under reduced pressure to yield the title compound as an off-white solid (0.17 g, 61% yield). MS: 256.9 [M+H].

### Intermediate A4

### 7-Cyclobutyl-5-Iodo-7H-Pyrrolo[2,3-D]Pyrimidin-4-Amine

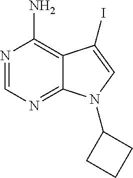 [SMILES: Nc1ncnc2c1c(I)cn2C1CCC1]

The title compound was prepared by following a similar procedure described for steps 2 and 3 of Intermediate A3, starting from 4-chloro-5-iodo-7H-pyrrolo[2,3-d]pyrimidine (1.000 g, 3.58 mmol) and bromocyclobutane (0.580 g, 4.29 mmol) in step 1 and using NH₄OH (25% in water, 4.5 mL) in step 2, and was isolated as an off-white solid (0.30 g, 20% yield over 2 steps). ¹H NMR (400 MHz, DMSO-d₆) δ=8.09 (s, 1H), 7.73 (s, 1H), 6.61 (bs, 2H), 5.09-5.16 (m, 1H), 2.50-2.50 (m, 2H), 2.31-2.38 (m, 2H), 1.75-1.83 (m, 2H). LCMS: 315.0 [M+H].

### Intermediate A5

### 2-(4-Amino-5-Iodo-7H-Pyrrolo[2,3-D]Pyrimidin-7-Yl)Ethan-1-Ol

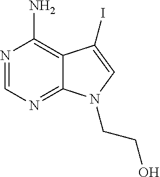 [SMILES: Nc1ncnc2c1c(I)cn2CCO]

The title compound was prepared by following a similar procedure described for steps 2 and 3 of Intermediate A3, starting from 4-chloro-5-iodo-7H-pyrrolo[2,3-d]pyrimidine (1.000 g, 3.58 mmol) and 2-bromoethan-1-ol (0.537 g, 4.29 mmol) in step 1 and using NH₄OH (25% in water, 8 mL) in step 2, and was isolated as an off-white solid (0.94 g, 44% yield over 2 steps). MS: 305.0 [M+H].

### Intermediate A6

### 1-(4-Amino-5-Iodo-7H-Pyrrolo[2,3-D]Pyrimidin-7-Yl)-2-Methylpropan-2-Ol

### Step 1: Synthesis of 1-(4-chloro-5-iodo-7H-pyrrolo[2,3-d]pyrimidin-7-yl)-2-methylpropan-2-ol

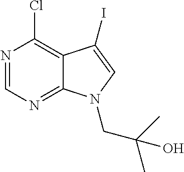 [SMILES: CC(C)(O)Cn1cc(I)c2c(Cl)ncnc21]

NaH₂PO₄ (0.105 g, 0.877 mmol) was added to a mixture of 4-chloro-5-iodo-7H-pyrrolo[2,3-d]pyrimidine (0.250 g, 0.895 mmol), 2,2-dimethyloxirane (0.157 mL, 1.762 mmol), and K₂CO₃ (0.121 g, 0.877 mmol) in ACN (3 mL) and water (1 mL). The resulting mixture was subjected to microwave irradiation at 150° C. for 1 h in a sealed tube. Following completion of the reaction (as indicated by TLC), the reaction mixture was concentrated under reduced pressure and the resulting crude material was purified by flash chromatography (silica gel 230-400 mesh, eluting with 18% EtOAc in petroleum ether), affording the title compound as a pale brown solid (0.1 g, 17% yield). MS: 351.9 [M+H].

### Step 2: Synthesis of 1-(4-amino-5-iodo-7H-pyrrolo[2,3-d]pyrimidin-7-yl)-2-methylpropan-2-ol

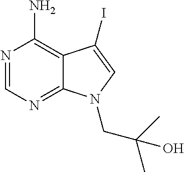 [SMILES: CC(C)(O)Cn1cc(I)c2c(N)ncnc21]

The title compound was obtained by following a similar procedure described for step 3 of Intermediate A3, starting from 1-(4-chloro-5-iodo-7H-pyrrolo[2,3-d]pyrimidin-7-yl)-2-methylpropan-2-ol (0.1 g, 0.284 mmol) and NH₄OH (25% in water, 0.5 mL), and was isolated as an off-white solid (0.08 g, 85% yield). ¹H NMR (400 MHz, DMSO-d6) δ=8.12 (s, 1H), 7.38 (s, 1H), 4.81 (s, 1H), 4.04 (s, 2H), 1.03 (s, 6H). MS: 333.0 [M+H].

### Intermediate A7

### 7-(3-(Benzyloxy)Cyclobutyl)-5-Iodo-7H-Pyrrolo[2,3-D]Pyrimidin-4-Amine

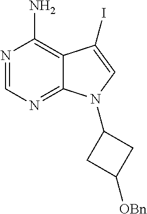 [SMILES: Nc1ncnc2c1c(I)cn2C1CC(OCc2ccccc2)C1]

The title compound was prepared by following a similar procedure described for steps 2 and 3 of Intermediate A3, starting from 4-chloro-5-iodo-7H-pyrrolo[2,3-d]pyrimidine (0.250 g, 0.895 mmol), 3-(benzyloxy)cyclobutyl methanesulfonate (prepared as reported in PCT Publication No. WO 2019/092170, 0.459 g, 1.789 mmol), and Cs₂CO₃ (0.583 g, 1.789 mmol) in step 1 and using NH₄OH (25% in water, 1.4 mL) in step 2, and was isolated as an off-white solid (0.06 g, 14% yield over 2 steps). MS: 421.1 [M+H].

### Intermediate A8

### Tert-Butyl 3-(4-Amino-5-Iodo-7H-Pyrrolo[2,3-D]Pyrimidin-7-Yl)Propanoate

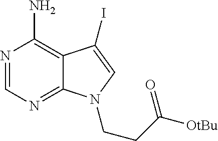 [SMILES: CC(C)(C)OC(=O)CCn1cc(I)c2c(N)ncnc21]

The title compound was prepared by following a similar procedure described for step 2 of Intermediate A3, starting from 5-iodo-7H-pyrrolo[2,3-d]pyrimidin-4-amine (1.000 g, 3.85 mmol), tert-butyl 3-bromopropanoate (0.804 g, 3.85 mmol), and Cs₂CO₃ (2.506 g, 7.69 mmol), and was isolated as a as a pale brown solid (0.34 g, 23% yield) following purification by flash chromatography (silica gel 230-400 mesh, eluting with 40% EtOAc in petroleum ether). MS: 389.0 [M+H].

### Intermediate A9

### 5-Iodo-7-(2-Methoxyethyl)-7H-Pyrrolo[2,3-D]Pyrimidin-4-Amine

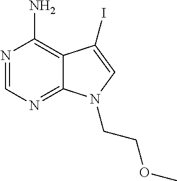

The title compound was prepared as reported in PCT Publication No. WO 2014/184069.

### Intermediate A10

### 5-Iodo-7-(Oxetan-3-Yl)-7H-Pyrrolo[2,3-D]Pyrimidin-4-Amine

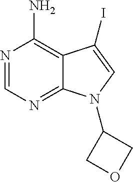 [SMILES: Nc1ncnc2c1c(I)cn2C1COC1]

The title compound was prepared by following a similar procedure described for steps 2 and 3 of Intermediate A3, starting from 4-chloro-5-iodo-7H-pyrrolo[2,3-d]pyrimidine (0.40 g, 1.43 mmol) and 3-iodooxetane (0.32 g, 1.71 mmol) in step 1 and using NH₄OH (25% in water, 2.5 mL) in step 2, and was isolated as a pale brown solid (0.27 g, 24% yield over 2 steps). MS: 316.8 [M+H].

### Intermediate A11

### 5-Iodo-7-(Pyridin-3-Yl)-7H-Pyrrolo[2,3-D]Pyrimidin-4-Amine

### Step 1: Synthesis of 4-chloro-5-iodo-7-(pyridin-3-yl)-7H-pyrrolo[2,3-d]pyrimidine

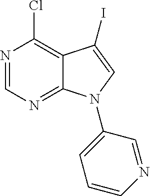 [SMILES: Clc1ncnc2c1c(I)cn2-c1cccnc1]

TEA (0.905 g, 8.95 mmol) and Cu(OAc)₂ (0.975 g, 5.37 mmol) were added to a solution of 4-chloro-5-iodo-7H-pyrrolo[2,3-d]pyrimidine (1.000 g, 3.58 mmol) and 3-pyridinylboronic acid (0.880 g, 7.16 mmol) in DCM (25 mL) and the resulting mixture was stirred at 40° C. under oxygen atmosphere for 40 h. Following completion of the reaction (as indicated by LCMS), the reaction mixture was filtered through a pad of Celite® (i.e., diatomaceous earth) which was then rinsed with DCM (2×50 mL). The combined filtrates were washed with water (10 mL) and brine (10 mL), dried over Na₂SO₄, filtered, and concentrated under reduced pressure to give crude material. This was stirred with 30% diethyl ether in petroleum ether for 30 minutes at 25° C., filtered, and dried to afford the title compound as a brown solid (0.4 g, 29% yield). ¹H NMR (400 MHz, DMSO-d₆) δ=9.05 (bs, 1H), 8.73 (s, 1H), 8.67 (bs, 1H), 8.48 (s, 1H), 8.26-8.28 (m, 1H), 7.64-7.67 (m, 1H). MS: 356.8 [M+H].

### Step 2: Synthesis of 5-iodo-7-(pyridin-3-yl)-7H-pyrrolo[2,3-d]pyrimidin-4-amine

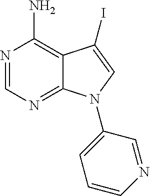 [SMILES: Nc1ncnc2c1c(I)cn2-c1cccnc1]

The title compound was obtained by following a similar procedure described for step 3 of Intermediate A3, starting from 4-chloro-5-iodo-7-(pyridin-3-yl)-7H-pyrrolo[2,3-d]pyrimidine (0.30 g, 0.841 mmol) and NH₄OH (25% in water, 1 mL), and was isolated as an off-white solid (0.21 g, 63% yield). MS: 337.8 [M+H].

### Intermediate A12

### 5-Iodo-7-(Pyridin-4-Yl)-7H-Pyrrolo[2,3-D]Pyrimidin-4-Amine

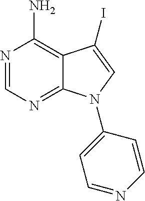 [SMILES: Nc1ncnc2c1c(I)cn2-c1ccncc1]

The title compound was prepared as described for Intermediate A11, starting from 4-chloro-5-iodo-7H-pyrrolo[2,3-d]pyrimidine (0.50 g, 1.789 mmol) and 4-pyridinylboronic acid (0.44 g, 3.58 mmol) in step 1, and was isolated as an off-white solid (0.16 g, 20% yield over 2 steps). MS: 337.9 [M+H].

### Intermediate A13

### 5-Iodo-7-(1-Methylpyrrolidin-3-Yl)-7H-Pyrrolo[2,3-D]Pyrimidin-4-Amine

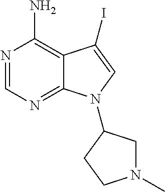 [SMILES: CN1CCC(n2cc(I)c3c(N)ncnc32)C1]

The title compound was prepared as reported in PCT Publication No. WO 2014/184069.

### Intermediate A14

### Tert-Butyl 4-(4-Amino-5-Iodo-7H-Pyrrolo[2,3-D]Pyrimidin-7-Yl)Piperidine-1-Carboxylate

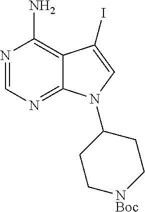 [SMILES: CC(C)(C)OC(=O)N1CCC(n2cc(I)c3c(N)ncnc32)CC1]

The title compound was prepared by following a similar procedure described for step 2 of Intermediate A3, starting from 5-iodo-7H-pyrrolo[2,3-d]pyrimidin-4-amine (1.50 g, 5.77 mmol), tert-butyl 4-(tosyloxy)piperidine-1-carboxylate (synthesized as reported in PCT Publication No. WO 2018/050771, 4.10 g, 11.54 mmol), and Cs₂CO₃ (3.76 g, 11.54 mmol), and was isolated as a as a yellow solid (1.2 g, 47% yield) following purification by flash chromatography (silica gel 230-400 mesh, eluting with 70% EtOAc in petroleum ether). MS: 444.1 [M+H].

### Intermediate A15

### 5-Iodo-7-(1-(Methylsulfonyl)Piperidin-4-Yl)-7H-Pyrrolo[2,3-D]Pyrimidin-4-Amine

### Step 1: Synthesis of 5-iodo-7-(piperidin-4-yl)-7H-pyrrolo[2,3-d]pyrimidin-4-amine

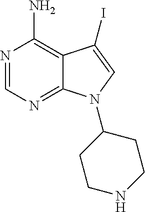 [SMILES: Nc1ncnc2c1c(I)cn2C1CCNCC1]

HCl in 1,4-dioxane (4 M, 6 mL) was added to a solution of tert-butyl 4-(4-amino-5-iodo-7H-pyrrolo[2,3-d]pyrimidin-7-yl)piperidine-1-carboxylate (A14, 1.20 g, 2.71 mmol) in DCM (15 mL) at 0° C. and the resulting mixture was stirred at 25° C. for 16 h. Following completion of the reaction (as indicated by LCMS), the reaction mixture was concentrated under reduced pressure to give the title compound (0.697 g, crude) as an off-white solid which was used without further purification. LCMS: 344.1 [M+H].

### Step 2: Synthesis of 5-iodo-7-(1-(methylsulfonyl)piperidin-4-yl)-7H-pyrrolo[2,3-d]pyrimidin-4-amine

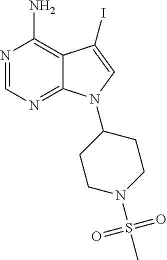 [SMILES: CS(=O)(=O)N1CCC(n2cc(I)c3c(N)ncnc32)CC1]

TEA (0.205 mL, 1.457 mmol) and MsCl (0.057 mL, 0.728 mmol) were added to a solution of 5-iodo-7-(piperidin-4-yl)-7H-pyrrolo[2,3-d]pyrimidin-4-amine (0.250 g, 0.728 mmol) in DCM (5 mL) at 0° C. and the resulting mixture was stirred at 25° C. for 16 h. Following completion of the reaction (as indicated by TLC), the reaction mixture was diluted with DCM (10 mL) and washed with brine (2 mL). The organic layer was dried over Na₂SO₄, filtered, and concentrated under reduced pressure to give crude material which was purified by flash chromatography (silica gel 230-400 mesh, eluting with 10% MeOH in DCM), affording the title compound as a pale brown solid (0.28 g, 91% yield). MS: 422.0 [M+H].

### Intermediate A16

### 1-Cyclopropyl-N-(2,4-Dimethoxybenzyl)-3-Iodo-1H-Pyrazolo[3,4-D]Pyrimidin-4-Amine

### Step 1: Synthesis of N-(2,4-dimethoxybenzyl)-1H-pyrazolo[3,4-d]pyrimidin-4-amine

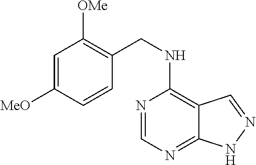 [SMILES: COc1ccc(CNc2ncnc3[nH]ncc23)c(OC)c1]

(2,4-Dimethoxyphenyl)methanamine (3.25 g, 19.41 mmol) was added to a solution of 4-chloro-1H-pyrazolo[3,4-d]pyrimidine (1.00 g, 6.47 mmol) in n-BuOH (10 mL) and the resulting mixture was stirred at 110° C. for 16 h. Following completion of the reaction (as indicated by LCMS), the reaction mixture was concentrated under reduced pressure to give crude material which was purified by preparative HPLC (eluting with 0.1% formic acid in H₂O and ACN), affording the title compounds as a yellow solid (1.0 g, 54% yield). MS: 286.1 [M+H].

### Step 2: Synthesis of N-(2,4-dimethoxybenzyl)-3-iodo-1H-pyrazolo[3,4-d]pyrimidin-4-amine

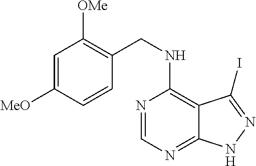 [SMILES: COc1ccc(CNc2ncnc3[nH]nc(I)c23)c(OC)c1]

Iodine (0.890 g, 3.50 mmol) and KOH (0.344 g, 6.13 mmol) were added to a solution of N-(2,4-dimethoxybenzyl)-1H-pyrazolo[3,4-d]pyrimidin-4-amine (0.500 g, 1.752 mmol) in 1,4-dioxane (10 mL) and the resulting mixture was stirred at 75° C. for 12 h. Following completion of the reaction (as indicated by LCMS), the reaction mixture was filtered through a pad of Celite® (i.e., diatomaceous earth) which was then rinsed with EtOAc (2×20 mL). The combined filtrates were concentrated under reduced pressure, giving crude material which was taken in a saturated sodium thiosulphate solution (20 mL) and stirred for 15 min. The resulted solid was filtered, washed with water (3×5 mL), and dried to afford the title compound as a pale brown solid (0.390 g, 54% yield). ¹H NMR (400 MHz, DMSO-d₆) δ=13.87 (s, 1H), 8.26 (s, 1H), 7.18 (d, J=8.3 Hz, 1H), 7.05-7.08 (m, 1H), 6.63 (d, J=2.3 Hz, 1H), 6.48 (dd, J=2.3, 8.3 Hz, 1H), 4.70 (d, J=5.9 Hz, 2H), 3.90 (s, 3H), 3.75 (s, 3H). MS: 412.0 [M+H].

### Step 3: Synthesis of 1-cyclopropyl-N-(2,4-dimethoxybenzyl)-3-iodo-1H-pyrazolo[3,4-d]pyrimidin-4-amine

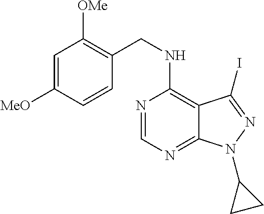 [SMILES: COc1ccc(CNc2ncnc3c2c(I)nn3C2CC2)c(OC)c1]

The title compound was prepared by following a similar procedure described for step 1 of Intermediate A1, starting from N-(2,4-dimethoxybenzyl)-3-iodo-1H-pyrazolo[3,4-d]pyrimidin-4-amine (0.390 g, 0.948 mmol) and cyclopropylboronic acid (0.161 g, 1.897 mmol), and was isolated as an off-white solid (0.150 g, 35% yield). ¹H NMR (400 MHz, DMSO-d₆) δ=8.30 (s, 1H), 7.17 (d, J=8.4 Hz, 1H), 7.08-7.09 (m, 1H), 6.62-6.62 (m, 1H), 6.47 (dd, J=2.4, 8.4 Hz, 1H), 4.70 (d, J=6.0 Hz, 2H), 3.89 (s, 3H), 3.75-3.79 (m, 4H), 1.11-1.14 (m, 2H), 1.04-1.07 (m, 2H). MS: 452.0 [M+H].

### Intermediate A17

### 1-Cyclopropyl-N-(2,4-Dimethoxybenzyl)-3-Iodo-1H-Pyrazolo[4,3-C]Pyridin-4-AMINE

### Step 1: Synthesis of 4-chloro-3-iodo-1H-pyrazolo[4,3-c]pyridine

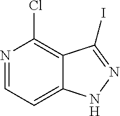 [SMILES: Clc1nccc2[nH]nc(I)c12]

The title compound was prepared by following a similar procedure described for step 2 of Intermediate A16, starting from 4-chloro-1H-pyrazolo[4,3-c]pyridine (1.00 g, 6.4 mmol), KOH (1.32 g, 23.0 mmol), and iodine (1.62 g, 12.8 mmol), and was isolated as a white solid (0.633 g, 63% yield) following purification by reverse-phase column chromatography. ¹H NMR (400 MHz, DMSO-d₆) δ=14.12 (bs, 1H), 8.14 (d, J=6.0 Hz, 1H), 7.66 (d, J=5.6 Hz, 1H). MS: 279.9 [M+H].

### Step 2: Synthesis of 4-chloro-1-cyclopropyl-3-iodo-1H-pyrazolo[4,3-c]pyridine

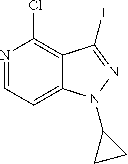 [SMILES: Clc1nccc2c1c(I)nn2C1CC1]

The title compound was prepared by following a similar procedure described for step 1 of Intermediate A1, starting from 4-chloro-3-iodo-1H-pyrazolo[4,3-c]pyridine (0.630 g, 2.20 mmol) and cyclopropylboronic acid (0.329 g, 3.83 mmol), and was isolated as a white solid (0.430 g, 60% yield). ¹H NMR (400 MHz, DMSO-d₆) δ=8.21 (d, J=6.0 Hz, 1H), 7.81 (d, J=5.6 Hz, 1H), 3.84-3.89 (m, 1H), 1.14-1.17 (m, 4H). MS: 319.7 [M+H].

### Step 3: Synthesis of 1-cyclopropyl-N-(2,4-dimethoxybenzyl)-3-iodo-1H-pyrazolo[4,3-c]pyridin-4-amine

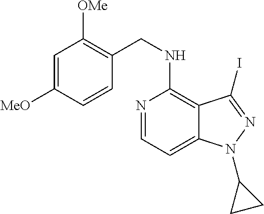 [SMILES: COc1ccc(CNc2nccc3c2c(I)nn3C2CC2)c(OC)c1]

The title compound was prepared by following a similar procedure described for step 1 of Intermediate A16, starting from 4-chloro-1-cyclopropyl-3-iodo-1H-pyrazolo[4,3-c]pyridine (0.880 g, 2.75 mmol) and (2,4-dimethoxyphenyl)methanamine (1.245 mL, 8.26 mmol), and was isolated as a yellow gum (1.0 g, 80% yield). ¹H NMR (400 MHz, DMSO-d₆) δ=7.81 (d, J=6.4 Hz, 1H), 7.19 (d, J=8.4 Hz, 1H), 6.87 (d, J=6.0 Hz, 1H), 6.60 (d, J=2.4 Hz, 1H), 6.53-6.56 (m, 1H), 6.45-6.47 (m, 1H), 4.62 (d, J=5.6 Hz, 2H), 3.88 (s, 3H), 3.73 (s, 3H), 3.66-3.70 (m, 1H), 1.01-1.10 (m, 4H). MS: 451.0 [M+H].

### Intermediate A18

### N-(2,4-Dimethoxybenzyl)-3-Iodo-1-Isopropyl-1H-Pyrazolo[3,4-D]Pyrimidin-4-Amine

### Step 1: Synthesis of 4-chloro-3-iodo-1-isopropyl-1H-pyrazolo[3,4-d]pyrimidine

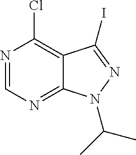

Cs₂CO₃ (2.607 g, 8.0 mmol) and 2-iodopropane (0.748 g, 4.4 mmol) are added to a solution of 4-chloro-3-iodo-1H-pyrazolo[3,4-d]pyrimidine (1.122 g, 4.0 mmol) in DMF (5 mL) and the reaction mixture is stirred at 90° C. for 16 h in a sealed tube. Following completion of the reaction (as indicated by TLC), the mixture is poured into crushed ice (10 g) and stirred for 15 min. The resulting solid is filtered, washed with water (2×5 mL), and dried to afford the title compound. MS (expected): 323.0 [M+H].

### Step 2: Synthesis of N-(2,4-dimethoxybenzyl)-3-iodo-1-isopropyl-1H-pyrazolo[3,4-d]pyrimidin-4-amine

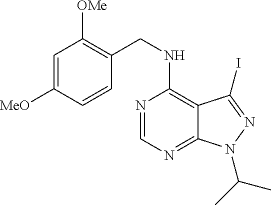 [SMILES: COc1ccc(CNc2ncnc3c2c(I)nn3C(C)C)c(OC)c1]

The title compound is prepared by following a similar procedure described for step 1 of Intermediate A16, starting from 4-chloro-3-iodo-1-isopropyl-1H-pyrazolo[3,4-d]pyrimidine (645 mg, 2.0 mmol) and (2,4-dimethoxyphenyl)methanamine (901 μL, 6.0 mmol). MS (expected): 454.1 [M+H].

### Intermediate A19

### 1-(3,3-Difluorocyclobutyl)-N-(2,4-Dimethoxybenzyl)-3-Iodo-1H-Pyrazolo[3,4-D]Pyrimidin-4-Amine

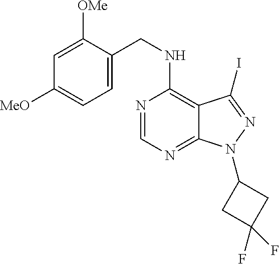 [SMILES: COc1ccc(CNc2ncnc3c2c(I)nn3C2CC(F)(F)C2)c(OC)c1]

The title compound is prepared by following a similar 2 steps procedure described for intermediate A18, starting from 4-chloro-3-iodo-1H-pyrazolo[3,4-d]pyrimidine (1.122 g, 4.0 mmol) and 3,3-difluorocyclobutyl 4-methylbenzenesulfonate (1.154 g, 4.4 mmol) in step 1. MS (expected): 502.0 [M+H].

### Intermediate A20

### 1-Allyl-N-(2,4-Dimethoxybenzyl)-3-Iodo-1H-Pyrazolo[3,4-D]Pyrimidin-4-Amine

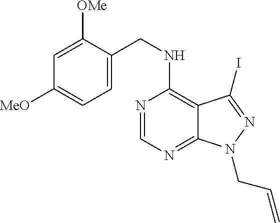 [SMILES: C=CCn1nc(I)c2c(NCc3ccc(OC)cc3OC)ncnc21]

The title compound is prepared by following a similar 2 steps procedure described for Intermediate A18, starting from 4-chloro-3-iodo-1H-pyrazolo[3,4-d]pyrimidine (1.122 g, 4.0 mmol) and allyl bromide (380 μL, 4.4 mmol) in step 1. MS (expected): 452.0 [M+H].

### Intermediate A21

### 1-(but-3-Yn-1-Yl)-N-(2,4-Dimethoxybenzyl)-3-Iodo-1H-Pyrazolo[3,4-D]Pyrimidin-4-Amine

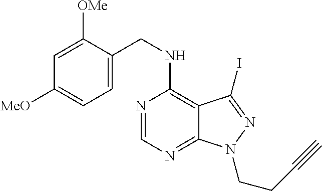 [SMILES: C#CCCn1nc(I)c2c(NCc3ccc(OC)cc3OC)ncnc21]

The title compound is prepared by following a similar 2 steps procedure described for Intermediate A18, starting from 4-chloro-3-iodo-1H-pyrazolo[3,4-d]pyrimidine (1.122 g, 4.0 mmol) and 4-bromobut-1-yne (413 μL, 4.4 mmol) in step 1. MS (expected): 464.0 [M+H].

### Intermediate A22

### 4-(4-Amino-3-Iodo-1H-Pyrazolo[3,4-D]Pyrimidin-1-Yl)Cyclohexan-1-Ol

### Step 1: Synthesis of N-(2,4-dimethoxybenzyl)-3-iodo-1-(1,4-dioxaspiro[4.5]decan-8-yl)-1H-pyrazolo[3,4-d]pyrimidin-4-amine

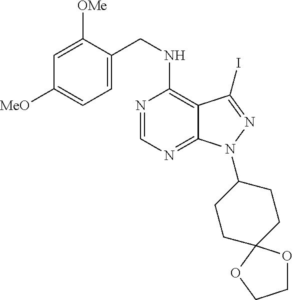 [SMILES: COc1ccc(CNc2ncnc3c2c(I)nn3C2CCC3(CC2)OCCO3)c(OC)c1]

The title compound is prepared by following a similar 2 steps procedure described for Intermediate A18, starting from 4-chloro-3-iodo-1H-pyrazolo[3,4-d]pyrimidine (1.122 g, 4.0 mmol) and 1,4-dioxaspiro[4.5]decan-8-yl 4-methylbenzenesulfonate (1.374 g, 4.4 mmol) in step 1. MS (expected): 552.1 [M+H].

### Step 2: Synthesis of 4-(4-((2,4-dimethoxybenzyl)amino)-3-iodo-1H-pyrazolo[3,4-d]pyrimidin-1-yl)cyclohexan-1-ol

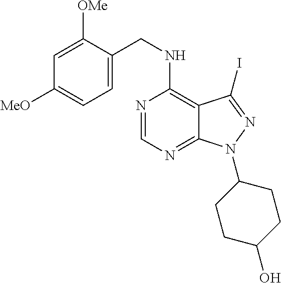 [SMILES: COc1ccc(CNc2ncnc3c2c(I)nn3C2CCC(O)CC2)c(OC)c1]

A solution of aqueous HCl (1.5 N, 6.5 mL) and N-(2,4-dimethoxybenzyl)-3-iodo-1-(1,4-dioxaspiro[4.5]decan-8-yl)-1H-pyrazolo[3,4-d]pyrimidin-4-amine (551 mg, 1.0 mmol) is stirred at room temperature for 12 h. Following completion of the reaction (as indicated by UPLC), the reaction mixture is concentrated under reduced pressure to give a residue which is dissolved in THF (5 mL). The resulting solution is cooled to 0° C., NaBH₄ (76 mg, 2.0 mmol) is added, and the resulting mixture is stirred at room temperature for 1 h. Following completion of the reaction (as indicated by UPLC), aqueous HCl (1.5 N, 6.5 mL) is added, and the mixture is extracted with EtOAc (2×10 mL). The combined organic layers are dried over Na₂SO₄, filtered, and concentrated under reduced pressure to give the title product. MS (expected): 510.1 [M+H].

### Intermediate A23

### N-(2,4-Dimethoxybenzyl)-3-Iodo-1-(Tetrahydrofuran-3-Yl)-1H-Pyrazolo[3,4-D]Pyrimidin-4-Amine

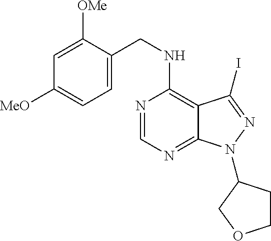 [SMILES: COc1ccc(CNc2ncnc3c2c(I)nn3C2CCOC2)c(OC)c1]

The title compound is prepared by following a similar 2 steps procedure described for Intermediate A18, starting from 4-chloro-3-iodo-1H-pyrazolo[3,4-d]pyrimidine (1.122 g, 4.0 mmol) and 3-iodotetrahydrofuran (451 μL, 4.4 mmol) in step 1. MS (expected): 552.1 [M+H].

### Intermediate A24

### N-(2,4-Dimethoxybenzyl)-3-Iodo-1-(Tetrahydro-2H-Pyran-4-Yl)-1H-Pyrazolo[3,4-D]Pyrimidin-4-Amine

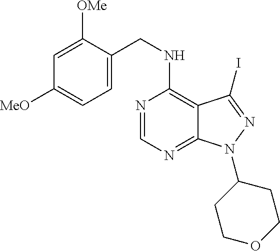 [SMILES: COc1ccc(CNc2ncnc3c2c(I)nn3C2CCOCC2)c(OC)c1]

The title compound is prepared by following a similar 2 steps procedure described for Intermediate A18, starting from 4-chloro-3-iodo-1H-pyrazolo[3,4-d]pyrimidine (1.122 g, 4.0 mmol) and tetrahydro-2H-pyran-4-yl 4-methylbenzenesulfonate (1128 mg, 4.4 mmol) in step 1. MS (expected): 496.1 [M+H].

### Intermediate A25

### N-(2,4-Dimethoxybenzyl)-3-Iodo-1-(Tetrahydro-2H-Pyran-3-Yl)-1H-Pyrazolo[3,4-D]Pyrimidin-4-Amine

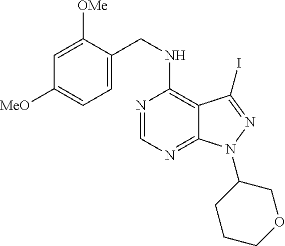 [SMILES: COc1ccc(CNc2ncnc3c2c(I)nn3C2CCCOC2)c(OC)c1]

The title compound is prepared by following a similar 2 step procedure described for Intermediate A18, starting from 4-chloro-3-iodo-1H-pyrazolo[3,4-d]pyrimidine (1.122 g, 4.0 mmol) and 3-bromotetrahydro-2H-pyran (492 μL, 4.4 mmol) in step 1. MS (expected): 496.1 [M+H].

### Intermediate A26

### 3-(4-((2,4-Dimethoxybenzyl)Amino)-3-Iodo-1H-Pyrazolo[3,4-D]Pyrimidin-1-Yl)Tetrahydrothiophene 1,1-Dioxide

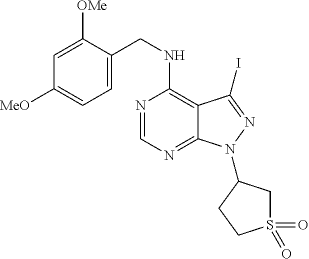 [SMILES: COc1ccc(CNc2ncnc3c2c(I)nn3C2CCS(=O)(=O)C2)c(OC)c1]

The title compound is prepared by following a similar 2 steps procedure described for Intermediate A18, starting from 4-chloro-3-iodo-1H-pyrazolo[3,4-d]pyrimidine (1.122 g, 4.0 mmol) and 3-bromotetrahydrothiophene 1,1-dioxide (876 mg, 4.4 mmol) in step 1. MS (expected): 530.0 [M+H].

### Intermediate A27

### Tert-Butyl 3-(4-((2,4-Dimethoxybenzyl)Amino)-3-Iodo-1H-Pyrazolo[3,4-D]Pyrimidin-1-Yl)Azetidine-1-Carboxylate

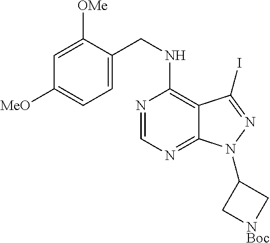 [SMILES: COc1ccc(CNc2ncnc3c2c(I)nn3C2CN(C(=O)OC(C)(C)C)C2)c(OC)c1]

The title compound is prepared by following a similar 2 steps procedure described for Intermediate A18, starting from 4-chloro-3-iodo-1H-pyrazolo[3,4-d]pyrimidine (1.122 g, 4.0 mmol) and tert-butyl 3-iodoazetidine-1-carboxylate (1246 mg, 4.4 mmol) in step 1. MS (expected): 567.1 [M+H].

### Intermediate A28

### Tert-Butyl 3-(4-((2,4-Dimethoxybenzyl)Amino)-3-Iodo-1H-Pyrazolo[3,4-D]Pyrimidin-1-Yl)Piperidine-1-Carboxylate

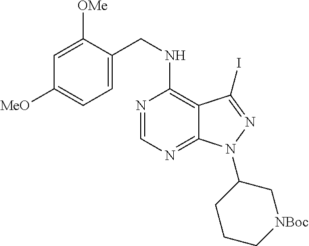 [SMILES: COc1ccc(CNc2ncnc3c2c(I)nn3C2CCCN(C(=O)OC(C)(C)C)C2)c(OC)c1]

The title compound was prepared by following a similar procedure described for step 1 of Intermediate A16, starting from tert-butyl 3-(4-chloro-3-iodo-1H-pyrazolo[3,4-d]pyrimidin-1-yl)piperidine-1-carboxylate (synthesized as reported in PCT Publication No. WO 2012/158795, 927 mg, 2.0 mmol) and (2,4-dimethoxyphenyl)methanamine (1003 mg, 6.0 mmol). MS (expected): 595.2 [M+H].

### Intermediate A29

### Tert-Butyl 4-(4-((2,4-Dimethoxybenzyl)Amino)-3-Iodo-1H-Pyrazolo[3,4-D]Pyrimidin-1-Yl)Piperidine-1-Carboxylate

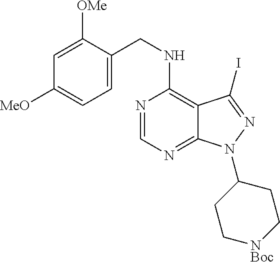 [SMILES: COc1ccc(CNc2ncnc3c2c(I)nn3C2CCN(C(=O)OC(C)(C)C)CC2)c(OC)c1]

The title compound is prepared by following a similar 2 steps procedure described for Intermediate A18, starting from 4-chloro-3-iodo-1H-pyrazolo[3,4-d]pyrimidine (1.122 g, 4.0 mmol) and tert-butyl 4-(tosyloxy)piperidine-1-carboxylate (1564 mg, 4.4 mmol) in step 1. MS (expected): 595.2 [M+H].

### Intermediate A30

### N-(2,4-Dimethoxybenzyl)-5-Iodo-7-Methyl-7H-Pyrrolo[2,3-D]Pyrimidin-4-Amine

### Step 1: Synthesis of 4-chloro-5-iodo-7-methyl-7H-pyrrolo[2,3-d]pyrimidine

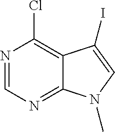

NaH (60% in mineral oil, 0.43 g, 10.73 mmol) was added portionwise to a suspension of 4-chloro-5-iodo-7H-pyrrolo[2,3-d]pyrimidine (2.00 g, 7.16 mmol) in THF (20 mL) at 0° C. followed by iodomethane (0.89 mL, 14.31 mmol) the resulting mixture was stirred at 25° C. for 2 h. Following completion of the reaction (as indicated by UPLC), the reaction mixture was quenched with ice water (10 mL) and stirred for 30 minutes. The resulting solid was filtered, washed with water (2×5 mL), and dried to afford the title compound as an off-white solid (2.1 g, quantitative yield). ¹H NMR (400 MHz, DMSO-d₆) δ=8.65 (s, 1H), 7.99 (s, 1H), 3.83 (s, 3H). MS: 294.0 [M+H].

### Step 2: Synthesis of N-(2,4-dimethoxybenzyl)-5-iodo-7-methyl-7H-pyrrolo[2,3-d]pyrimidin-4-amine

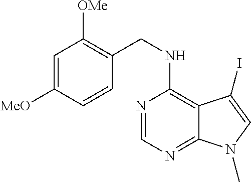 [SMILES: COc1ccc(CNc2ncnc3c2c(I)cn3C)c(OC)c1]

The title compound was prepared by following a similar procedure described for step 1 of Intermediate A16, starting from 4-chloro-5-iodo-7-methyl-7H-pyrrolo[2,3-d]pyrimidine (2.00 g, 6.81 mmol) and (2,4-dimethoxyphenyl)methanamine (1.14 g, 6.81 mmol), and was isolated as a pale yellow solid (1.9 g, 65% yield). MS: 425.0 [M+H].

### Intermediate A31

### N-(2,4-Dimethoxybenzyl)-5-Iodo-7-Isopropyl-7H-Pyrrolo[2,3-D]Pyrimidin-4-Amine

### Step 1: Synthesis of 4-chloro-5-iodo-7-isopropyl-7H-pyrrolo[2,3-d]pyrimidine

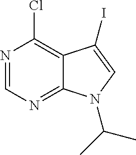

A mixture of 4-chloro-5-iodo-7H-pyrrolo[2,3-d]pyrimidine (1.00 g, 3.58 mmol), K₂CO₃ (1.48 g, 10.73 mmol), and 2-iodopropane (1.22 g, 7.16 mmol) in dry DMF (5 mL) was stirred at 80° C. for 16 h. Following completion of the reaction (as indicated by TLC), the reaction mixture was poured into crushed ice (50 g) and stirred for 15 min. The resulting precipitate was filtered, washed with water (2×5 mL), and dried to afford the title compound as a white solid (0.8 g, 69% yield). ¹H NMR (400 MHz, DMSO-d₆) δ=8.64 (s, 1H), 8.18 (s, 1H), 5.02-5.08 (m, 1H), 1.48 (d, J=6.4 Hz, 6H). MS: 322.0 [M+H]

### Step 2: Synthesis of N-(2,4-dimethoxybenzyl)-5-iodo-7-isopropyl-7H-pyrrolo[2,3-d]pyrimidin-4-amine

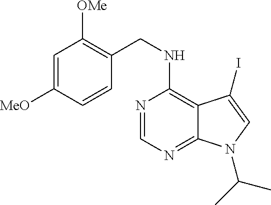 [SMILES: COc1ccc(CNc2ncnc3c2c(I)cn3C(C)C)c(OC)c1]

The title compound was prepared by following a similar procedure described for step 1 of Intermediate A16, starting from 4-chloro-5-iodo-7-isopropyl-7H-pyrrolo[2,3-d]pyrimidine (0.80 g, 2.49 mmol) and (2,4-dimethoxyphenyl)methanamine (1.25 g, 7.46 mmol), and was isolated as a pale yellow solid (0.7 g, 62% yield). ¹H NMR (400 MHz, DMSO-d₆) δ=8.18 (s, 1H), 7.57 (s, 1H), 7.19 (s, 1H), 6.70-6.76 (m, 1H), 6.61 (d, J=2.8 Hz, 1H), 6.45-6.48 (m, 1H), 4.85-4.94 (m, 1H), 4.64-4.66 (m, 2H), 3.92 (s, 3H), 3.74 (s, 3H), 1.39-1.41 (m, 6H). MS: 453.1 [M+H].

### Intermediate A32

### Tert-Butyl 4-(4-((2,4-Dimethoxybenzyl)Amino)-5-Iodo-7H-Pyrrolo[2,3-D]Pyrimidin-7-Yl)Piperidine-1-Carboxylate

### Step 1: Synthesis of tert-butyl 4-(4-chloro-5-iodo-7H-pyrrolo[2,3-d]pyrimidin-7-yl)piperidine-1-carboxylate

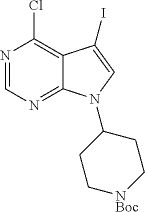 [SMILES: CC(C)(C)OC(=O)N1CCC(n2cc(I)c3c(Cl)ncnc32)CC1]

The title compound was prepared by following a similar procedure described for step 2 of Intermediate A3, starting from 4-chloro-5-iodo-7H-pyrrolo[2,3-d]pyrimidine (2.50 g, 8.95 mmol), tert-butyl 4-(tosyloxy)piperidine-1-carboxylate (synthesized as reported in PCT Publication No. WO 2018/050771, 3.20 g, 9.00 mmol), and K₂CO₃ (3.11 g, 22.51 mmol), and was isolated as a yellow solid (1.2 g, 29% yield). MS: 463.2 [M+H].

### Step 2: Synthesis of tert-butyl 4-(4-((2,4-dimethoxybenzyl)amino)-5-iodo-7H-pyrrolo[2,3-d]pyrimidin-7-yl)piperidine-1-carboxylate

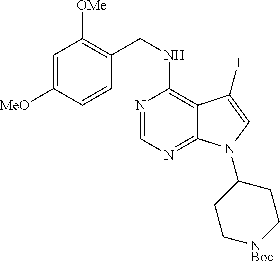 [SMILES: COc1ccc(CNc2ncnc3c2c(I)cn3C2CCN(C(=O)OC(C)(C)C)CC2)c(OC)c1]

The title compound was prepared by following a similar procedure described for step 1 of Intermediate A16, starting from tert-butyl 4-(4-chloro-5-iodo-7H-pyrrolo[2,3-d]pyrimidin-7-yl)piperidine-1-carboxylate (0.800 g, 1.729 mmol) and (2,4-dimethoxyphenyl)methanamine (0.269 g, 1.729 mmol), and was isolated as pale yellow solid (0.7 g, 68% yield). MS: 594.3 [M+H].

### Intermediate B1

### 5-(4-Aminonaphthalen-1-Yl)-7-Cyclopropyl-7H-Pyrrolo[2,3-D]Pyrimidin-4-Amine

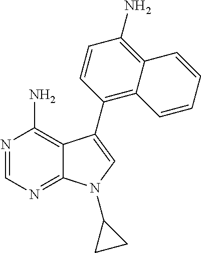 [SMILES: Nc1ccc(-c2cn(C3CC3)c3ncnc(N)c23)c2ccccc12]

A mixture of 7-cyclopropyl-5-iodo-7H-pyrrolo[2,3-d]pyrimidin-4-amine (A1, 250 mg, 0.833 mmol), 4-(4,4,5,5-tetramethyl-1,3,2-dioxaborolan-2-yl)naphthalen-1-amine (224 mg, 0.833 mmol), and K₂CO₃ (230 mg, 1.666 mmol) in 1,4-dioxane (5 mL), EtOH (2 mL), and water (1 mL) was purged with N₂ for 10 min. PdCl₂(dppf) (30 mg, 0.042 mmol) was then added and the resulting mixture was stirred at 80° C. for 16 h. Following completion of the reaction (as indicated by TLC), the reaction mixture was filtered through a pad of Celite® (i.e., diatomaceous earth) which was then rinsed with EtOAc (2×5 mL). The combined filtrates were concentrated under reduced pressure to give crude material which was purified by GRACE (silica gel 230-400 mesh, eluting with 2% MeOH in DCM), affording the title compound as a yellow solid (80 mg, 30% yield). MS: 316.1 [M+H].

### Intermediate B2

### 5-(4-Amino-7-Cyclopropyl-7H-Pyrrolo[2,3-D]Pyrimidin-5-Yl)Quinolin-8-Amine

### Step 1: Synthesis of tert-butyl (tert-butoxycarbonyl)(7-cyclopropyl-5-iodo-7H-pyrrolo[2,3-d]pyrimidin-4-yl)carbamate

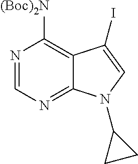

A mixture of DMAP (0.081 g, 0.663 mmol), (Boc)₂O (1.745 g, 8.000 mmol), and 7-cyclopropyl-5-iodo-7H-pyrrolo[2,3-d]pyrimidin-4-amine (A1, 1.000 g, 3.330 mmol) in THF (10 mL) was stirred at 25° C. for 12 h. Following completion of the reaction (as indicated by LCMS), the reaction mixture was concentrated under reduced pressure to give crude material which was purified by flash chromatography (silica gel 230-400 mesh, eluting with 30% EtOAc in petroleum ether), affording the title compound as an off-white solid (1.6 g, 99% yield). ¹H NMR (400 MHz, DMSO-d₆) δ=8.78 (s, 1H), 7.88 (s, 1H), 3.68-3.69 (m, 1H), 1.36 (s, 18H), 1.07-1.10 (m, 4H). MS: 501.1 [M+H].

### Step 2: Synthesis of tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate

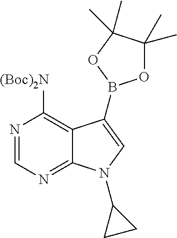

A mixture of TEA (1.573 mL, 11.19 mmol), XPhos (0.133 g, 0.28 mmol), tert-butyl (tert-butoxycarbonyl)(7-cyclopropyl-5-iodo-7H-pyrrolo[2,3-d]pyrimidin-4-yl)carbamate (1.400 g, 2.80 mmol), and HBpin (1.421 g, 11.19 mmol) in 1,4-dioxane (20 mL) was purged with N₂ for 10 minutes. Pd₂(dba)₃ (0.256 g, 0.280 mmol) was then added, and the reaction mixture was stirred at 100° C. for 6 h. Following the completion of the reaction (as indicated by TLC), the reaction mixture was filtered through a pad of Celite® (i.e., diatomaceous earth) which was then washed with DCM (2×20 mL). The combined filtrates were concentrated under reduced pressure, giving crude material which was purified by flash chromatography (silica gel 230-400 mesh, eluting with 25% EtOAc in petroleum ether) to afford the title compound as a pale-yellow gum (1.2 g, 86% yield). ¹H NMR (400 MHz, DMSO-d₆) δ=8.78 (s, 1H), 7.88 (s, 1H), 3.68-3.72 (m, 1H), 1.24-1.26 (m, 30H), 1.08-1.13 (m, 4H). MS: 401.3 [M+H-Boc].

### Step 3: Synthesis of 5-(4-amino-7-cyclopropyl-7H-pyrrolo[2,3-d]pyrimidin-5-yl)quinolin-8-amine

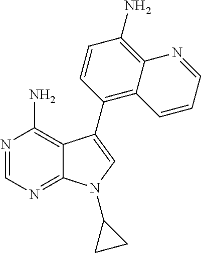 [SMILES: Nc1ccc(-c2cn(C3CC3)c3ncnc(N)c23)c2cccnc12]

The title compound was obtained by following a similar procedure described for Intermediate B1, starting from 5-bromoquinolin-8-amine (89 mg, 0.40 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (200 mg, 0.40 mmol), and was obtained as a pale-yellow gum (40 mg, 32% yield). Cleavage of the di-Boc protecting group was observed during the reaction. LCMS: 317.2 [M+H].

### Intermediate B3

### Tert-Butyl (5-(8-Aminoisoquinolin-5-Yl)-7-Cyclopropyl-7H-Pyrrolo[2,3-D]Pyrimidin-4-Yl)(Tert-Butoxycarbonyl)Carbamate

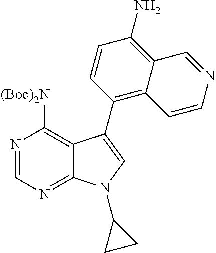

The title compound was obtained by following a similar procedure described for Intermediate B1, starting from 5-bromoisoquinolin-8-amine (89 mg, 0.40 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 200 mg, 0.40 mmol), and was isolated as a yellow solid (190 mg, 92% yield). MS: 517.2 [M+H].

### Intermediates B4 and B5

### 5-(8-Aminoimidazo[1,2-A]Pyridin-5-Yl)-7-Cyclopropyl-7H-Pyrrolo[2,3-D]Pyrimidin-4-Amine

### Tert-Butyl (5-(8-Aminoimidazo[1,2-A]Pyridin-5-Yl)-7-Cyclopropyl-7H-Pyrrolo[2,3-D]Pyrimidin-4-Yl)(Tert-Butoxycarbonyl)Carbamate

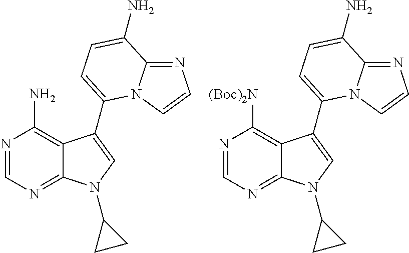

The title compounds were obtained as a mixture by following a similar procedure described for Intermediate B1, starting from 5-bromoimidazo[1,2-a]pyridin-8-amine (synthesized as reported in PCT Publication No. WO 2020/081508, 191 mg, 0.899 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 450 mg, 0.899 mmol). 5-(8-Aminoimidazo[1,2-a]pyridin-5-yl)-7-cyclopropyl-7H-pyrrolo[2,3-d]pyrimidin-4-amine (B4, major) was obtained as pale brown solid (150 mg, 54% yield). MS: 306.1 [M+H]. Tert-butyl (5-(8-aminoimidazo[1,2-a]pyridin-5-yl)-7-cyclopropyl-7H-pyrrolo[2,3-d]pyrimidin-4-yl)(tert-butoxycarbonyl)carbamate (B5, minor) was obtained as a pale brown solid (20 mg, 4% yield). MS: 506.1 [M+H].

### Intermediate B6

### 5-(8-Amino-2-Fluoroimidazo[1,2-A]Pyridin-5-Yl)-7-Cyclopropyl-7H-Pyrrolo[2,3-D]Pyrimidin-4-Amine

### Step 1: Synthesis of 5-bromo-2-fluoroimidazo[1,2-a]pyridin-8-amine

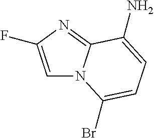 [SMILES: Nc1ccc(Br)n2cc(F)nc12]

Selectfluor® (195 mg, 0.55 mmol) is added to a solution of 5-bromoimidazo[1,2-a]pyridin-8-amine (synthesized as reported in PCT Publication No. WO 2020/081508, 106 mg, 0.50 mmol) in ACN (5 mL) and the resulting mixture is stirred at room temperature for 12 h. Following completion of the reaction (as indicated by TLC), the reaction mixture is diluted with water (15 mL) and extracted with DCM (2×10 mL). The combined organic phases are dried over Na2SO4, filtered, and concentrated under reduced pressure to give crude material which is purified by flash chromatography (silica gel 230-400 mesh, eluting with EtOAc in petroleum ether), affording the title compound. MS (expected): 230.0 [M+H].

### Step 2: Synthesis of tert-butyl (5-(8-amino-2-fluoroimidazo[1,2-a]pyridin-5-yl)-7-cyclopropyl-7H-pyrrolo[2,3-d]pyrimidin-4-yl)(tert-butoxycarbonyl)carbamate

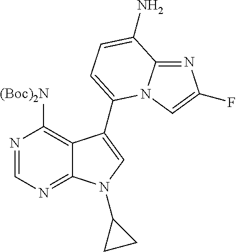

The title compound is prepared by following a similar procedure described for Intermediate B1, starting from 5-bromo-2-fluoroimidazo[1,2-a]pyridin-8-amine (100 mg, 0.435 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 218 mg, 0.435 mmol). MS (expected: 524.2 [M+H].

### Intermediate B7

### Tert-Butyl (5-(8-Amino-2-Methylimidazo[1,2-A]Pyridin-5-Yl)-7-Cyclopropyl-7H-Pyrrolo[2,3-D]Pyrimidin-4-Yl)(Tert-Butoxycarbonyl)Carbamate

### Step 1: Synthesis of 5-bromo-2-methylimidazo[1,2-a]pyridin-8-amine

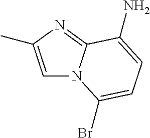

A mixture of 6-bromopyridine-2,3-diamine (188 mg, 1.0 mmol) and 1-chloropropan-2-one (96 μL, 1.2 mmol) in EtOH (15 mL) is heated to reflux for 12 h. Following completion of the reaction (as indicated by TLC), the reaction mixture is concentrated under reduced pressure to give a residue which is dissolved in EtOAc (20 mL) and washed with a saturated Na₂CO₃ solution (15 mL) then brine (15 mL). The organic phase is dried over Na₂SO₄, filtered, and concentrated under reduced pressure to give crude material which is purified by flash chromatography (silica gel 230-400 mesh, eluting with EtOAc in petroleum ether), affording the title compound. MS (expected): 226.0 [M+H].

### Step 2: Synthesis of tert-butyl (5-(8-amino-2-methylimidazo[1,2-a]pyridin-5-yl)-7-cyclopropyl-7H-pyrrolo[2,3-d]pyrimidin-4-yl)(tert-butoxycarbonyl)carbamate

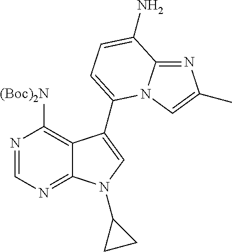

The title compound is prepared by following a similar procedure described for Intermediate B1, starting from 5-bromo-2-methylimidazo[1,2-a]pyridin-8-amine (100 mg, 0.442 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 221 mg, 0.435 mmol). MS (expected): 520.3 [M+H].

### Intermediate B8

### Tert-Butyl (5-(8-Amino-3-Methylimidazo[1,2-A]Pyridin-5-Yl)-7-Cyclopropyl-7H-Pyrrolo[2,3-D]Pyrimidin-4-Yl)(Tert-Butoxycarbonyl)Carbamate

### Step 1: Synthesis of 5-bromo-3-methylimidazo[1,2-a]pyridin-8-amine

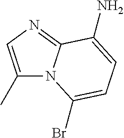

Concentrated HCl (40 μL) is added to a solution of 2-bromo-1,1-diethoxypropane (500 μL, 2.837 mmol) in water (0.7 mL) and dioxane (1.2 mL) and the resulting mixture is heated to reflux for 30 min. After cooling, NaHCO₃ (477 mg, 5.674 mmol) is added followed by a solution of 6-bromopyridine-2,3-diamine (267 mg, 1.419 mmol) in water (0.7 mL) and dioxane (1.2 mL) and the resulting mixture is heated to reflux for 24 h. Following cooling, the reaction mixture is poured in water (10 mL) and acidified with HCl (1 M solution). The aqueous phase is washed with EtOAc (15 mL), basified with NaOH (1 M solution), and extracted with EtOAc (15 mL). The combined organic extracts are washed with a saturated Na₂CO₃ solution (15 mL) then brine (10 mL), dried over Na₂SO₄, filtered, and concentrated under reduced pressure to give crude material which is purified by flash chromatography (silica gel 230-400 mesh, eluting with EtOAc in petroleum ether), affording the title compound. MS (expected): 226.0 [M+H].

### Step 2: Synthesis of tert-butyl (5-(8-amino-3-methylimidazo[1,2-a]pyridin-5-yl)-7-cyclopropyl-7H-pyrrolo[2,3-d]pyrimidin-4-yl)(tert-butoxycarbonyl)carbamate

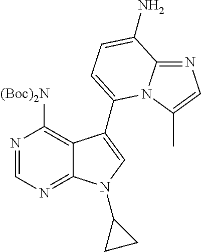

The title compound is prepared by following a similar procedure described for Intermediate B1, starting from 5-bromo-3-methylimidazo[1,2-a]pyridin-8-amine (100 mg, 0.442 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 221 mg, 0.435 mmol). MS (expected: 520.3 [M+H].

### Intermediate B9

### 5-(8-Aminoimidazo[1,2-A]Pyrazin-5-Yl)-7-Cyclopropyl-7H-Pyrrolo[2,3-D]Pyrimidin-4-Amine

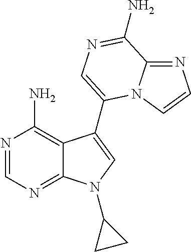 [SMILES: Nc1ncnc2c1c(-c1cnc(N)c3nccn13)cn2C1CC1]

The title compound was obtained by following a similar procedure described for Intermediate B1, starting from 5-bromoimidazo[1,2-a]pyrazin-8-amine (synthesized as reported in PCT Publication no. WO 2009/017701, 247 mg, 1.159 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 580 mg, 1.159 mmol), and was isolated as an off-white solid (100 mg, 28% yield). Cleavage of the di-Boc protecting group was observed during the reaction. LCMS: 307.1 [M+H].

### Intermediate B10

### Tert-Butyl (5-(7-Amino-2,3-Dihydrobenzofuran-4-Yl)-7-Cyclopropyl-7H-Pyrrolo[2,3-D]Pyrimidin-4-Yl)(Tert-Butoxycarbonyl)Carbamate

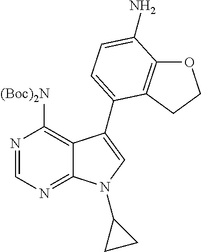

The title compound was obtained by following a similar procedure described for Intermediate B1, starting from 4-bromo-2,3-dihydrobenzofuran-7-amine (synthesized as reported in PCT Publication No. WO 2021/244582, 193 mg, 0.899 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 450 mg, 0.899 mmol), and was isolated as a pale brown gum (25 mg, 5% yield). MS: 508.2 [M+H].

### Intermediate B11

### Tert-Butyl (5-(7-Amino-2-Methyl-2,3-Dihydrobenzofuran-4-Yl)-7-Cyclopropyl-7H-Pyrrolo[2,3-D]Pyrimidin-4-Yl)(Tert-Butoxycarbonyl)Carbamate

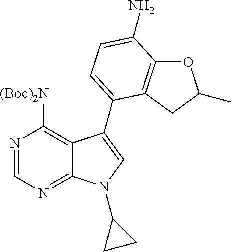

The title compound is prepared by following a similar procedure described for Intermediate B1, starting from 4-bromo-2-methyl-2,3-dihydrobenzofuran-7-amine (synthesized as reported in PCT Publication No. WO 2016/169504, 228 mg, 1.0 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 500 mg, 1.0 mmol). MS (expected): 522.3 [M+H].

### Intermediate B12

### Tert-Butyl (5-(7-Amino-2,2-Dimethyl-2,3-Dihydrobenzofuran-4-Yl)-7-Cyclopropyl-7H-Pyrrolo[2,3-D]Pyrimidin-4-Yl)(Tert-Butoxycarbonyl)Carbamate

### Step 1: Synthesis of 4-bromo-2,2-dimethyl-2,3-dihydrobenzofuran-7-amine

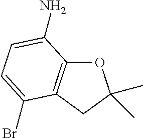

Iron powder (1117 mg, 20.0 mmol) and NH₄Cl (1070 mg, 20.0 mmol) are added to a solution of 4-bromo-2,2-dimethyl-7-nitro-2,3-dihydrobenzofuran (synthesized as reported in PCT Publication No. WO 2016/169504, 544 mg, 2.0 mmol) in EtOH (20 mL) and water (5 mL) and the resulting mixtures is stirred at 80° C. for 3 h. Following completion of the reaction (as indicated by TLC), the reaction mixture is filtered through a pad of Celite® (i.e., diatomaceous earth) which is then rinsed with EtOAc (2×20 mL). The combined filtrates are concentrated under reduced pressure, the residue is dissolved in EtOAc (25 mL), washed with brine (10 mL), dried over Na₂SO₄, filtered, and evaporated under reduced pressure to give the title compound. MS (expected): 242.0 [M+H].

Iron powder (144 mg, 2.57 mmol) and NH₄Cl (177 mg, 3.31 mmol) were added to a solution of 4-bromo-2,2-dimethyl-7-nitro-2,3-dihydrobenzofuran (synthesized as reported in PCT Publication No. WO 2016/169504, 200 mg, 0.735 mmol) in EtOH (5 mL) and water (1 mL) and the resulting mixture was stirred at 80° C. for 16 h. Following completion of the reaction (as indicated by TLC), the reaction mixture is filtered through a pad of Celite® (i.e., diatomaceous earth) which is then rinsed with EtOAc (2×10 mL). The combined filtrates are concentrated under reduced pressure, the residue is dissolved in EtOAc (25 mL), washed with brine (5 mL), dried over Na₂SO₄, filtered, and evaporated under reduced pressure to give the title compound (150 mg, 84% yield) as a brown solid. ¹H NMR (400 MHz, DMSO-d₆) δ=6.70 (d, J=8.4 Hz, 1H), 6.42 (d, J=8.4 Hz, 1H), 4.67 (bs, 2H), 2.94 (s, 2H), 1.43 (s, 6H). MS: 241.9 [M+H].

### Step 2: Synthesis of tert-butyl (5-(7-amino-2,2-dimethyl-2,3-dihydrobenzofuran-4-yl)-7-cyclopropyl-7H-pyrrolo[2,3-d]pyrimidin-4-yl)(tert-butoxycarbonyl)carbamate

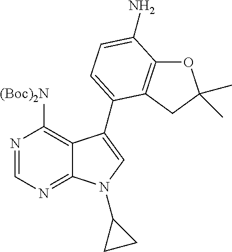

The title compound is prepared by following a similar procedure described for Intermediate B1, starting from 4-bromo-2,2-dimethyl-2,3-dihydrobenzofuran-7-amine (242 mg, 1.0 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 500 mg, 1.0 mmol). MS (expected): 536.3 [M+H].

### Intermediate B13

### 4-(4-Amino-7-Cyclopropyl-7H-Pyrrolo[2,3-D]Pyrimidin-5-Yl)Benzo[D]Isoxazol-7-Amine

### Step 1: Synthesis of 4-bromobenzo[d]isoxazol-7-amine

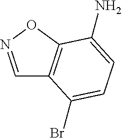 [SMILES: Nc1ccc(Br)c2cnoc12]

A solution of NBS (0.133 g, 0.746 mmol) in DMF (2 mL) was added to a solution of benzo[d]isoxazol-7-amine (0.100 g, 0.746 mmol) in DMF (0.5 mL) at 0° C. and the resulting mixture was stirred at 25° C. for 16 h. Following completion of the reaction (as indicated by LCMS), the reaction mixture was poured into crushed ice (5 g). The resulting solid was filtered, washed with water (2 mL), and dried to afford the title compound as a pale yellow solid (135 mg, 85% yield). ¹H NMR (400 MHz, DMSO-d₆) δ=9.13 (s, 1H), 7.25 (d, J=8.0 Hz, 1H), 6.72 (d, J=8.4 Hz, 1H), 5.95 (s, 2H). MS: 212.9 [M+H].

### Step 2: Synthesis of 4-(4-amino-7-cyclopropyl-7H-pyrrolo[2,3-d]pyrimidin-5-yl)benzo[d]isoxazol-7-amine

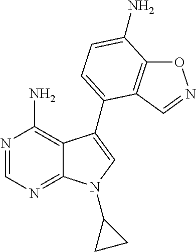 [SMILES: Nc1ccc(-c2cn(C3CC3)c3ncnc(N)c23)c2cnoc12]

The title compound was obtained by following a similar procedure described for Intermediate B1, starting from 4-bromobenzo[d]isoxazol-7-amine (128 mg, 0.600 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 300 mg, 0.600 mmol), and was isolated as a pale yellow solid (60 mg, 33% yield). MS: 307.0 [M+H].

### Intermediate B14

### Tert-Butyl (5-(7-Aminobenzo[C][1,2,5]Oxadiazol-4-Yl)-7-Cyclopropyl-7H-Pyrrolo[2,3-D]Pyrimidin-4-Yl)(Tert-Butoxycarbonyl)Carbamate

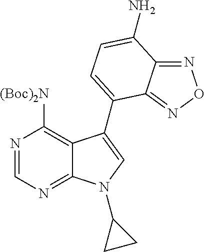

The title compound was obtained by following a similar procedure described for Intermediate B1, starting from 7-bromobenzo[c][1,2,5]oxadiazol-4-amine (synthesized as reported in PCT Publication No. WO 2003/062241, 141 mg, 0.659 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 330 mg, 0.659 mmol), and was isolated as an orange solid (110 mg, 33% yield). MS: 508.2 [M+H].

### Intermediate B15

### 5-(1-Cyclopropyl-4-((2,4-Dimethoxybenzyl)Amino)-1H-Pyrrolo[3,2-C]Pyridin-3-Yl)Imidazo[1,2-A]Pyridin-8-Amine

### Step 1: Synthesis of 1-cyclopropyl-N-(2,4-dimethoxybenzyl)-3-(4,4,5,5-tetramethyl-1,3,2-dioxaborolan-2-yl)-1H-pyrrolo[3,2-c]pyridin-4-amine

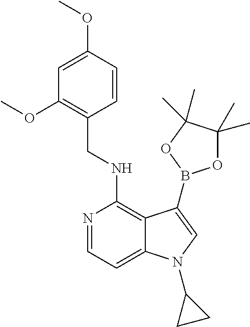

The title compound is prepared by following a similar procedure described for step 2 of Intermediate B2, starting from 1-cyclopropyl-N-(2,4-dimethoxybenzyl)-3-iodo-1H-pyrrolo[3,2-c]pyridin-4-amine (A2, 449 mg, 1.0 mmol) and HBpin (512 mg, 4.0 mmol). MS (expected): 450.3 [M+H].

The title compound was prepared by following a similar procedure described for step 2 of Intermediate B2, starting from 1-cyclopropyl-N-(2,4-dimethoxybenzyl)-3-iodo-1H-pyrrolo[3,2-c]pyridin-4-amine (A2, 200 mg, 0.445 mmol) and HBpin (228 mg, 1.781 mmol), and was obtained as a yellow gum (0.2 g, 37% yield). LCMS: 450.2 [M+H].

### Step 2: Synthesis of 5-(1-cyclopropyl-4-((2,4-dimethoxybenzyl)amino)-1H-pyrrolo[3,2-c]pyridin-3-yl)imidazo[1,2-a]pyridin-8-amine

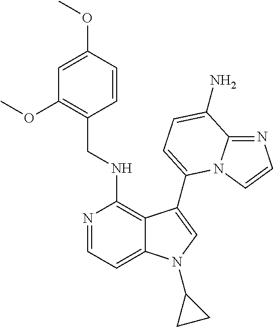 [SMILES: COc1ccc(CNc2nccc3c2c(-c2ccc(N)c4nccn24)cn3C2CC2)c(OC)c1]

The title compound is prepared by following a similar procedure described for intermediate B1, starting from 5-bromoimidazo[1,2-a]pyridin-8-amine (synthesized as reported in PCT Publication No. WO 2020/081508, 106 mg, 0.50 mmol) and 1-cyclopropyl-N-(2,4-dimethoxybenzyl)-3-(4,4,5,5-tetramethyl-1,3,2-dioxaborolan-2-yl)-1H-pyrrolo[3,2-c]pyridin-4-amine (225 mg, 0.50 mmol). MS (expected): 455.2 [M+H].

The title compound was prepared by following a similar procedure described for intermediate B1, starting from 5-bromoimidazo[1,2-a]pyridin-8-amine (synthesized as reported in PCT Publication No. WO 2020/081508, 35 mg, 0.165 mmol) and 1-cyclopropyl-N-(2,4-dimethoxybenzyl)-3-(4,4,5,5-tetramethyl-1,3,2-dioxaborolan-2-yl)-1H-pyrrolo[3,2-c]pyridin-4-amine (200 mg, 0.165 mmol), and was obtained as a brown gum (50 mg, 25% yield). LCMS: 455.2 [M+H].

### Intermediate B16

### Tert-Butyl (5-(8-Aminoimidazo[1,2-A]Pyridin-5-Yl)-7-Isopropyl-7H-Pyrrolo[2,3-D]Pyrimidin-4-Yl)(Tert-Butoxycarbonyl)Carbamate

### Step 1: Synthesis of tert-butyl (5-bromo-7-isopropyl-7H-pyrrolo[2,3-d]pyrimidin-4-yl)(tert-butoxycarbonyl)carbamate

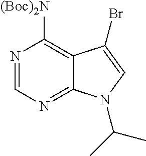

The title compound is prepared by following a similar procedure described for step 1 of Intermediate B2, starting from 5-bromo-7-isopropyl-7H-pyrrolo[2,3-d]pyrimidin-4-amine (A3, 255 mg, 1.0 mmol), (Boc)₂O (546 mg, 2.5 mmol), and DMAP (24 mg, 0.2 mmol). MS (expected): 455.1 [M+H].

### Step 2: Synthesis of tert-butyl (tert-butoxycarbonyl)(7-isopropyl-5-(4,4,5,5-tetramethyl-1,3,2-dioxaborolan-2-yl)-7H-pyrrolo[2,3-d]pyrimidin-4-yl)carbamate

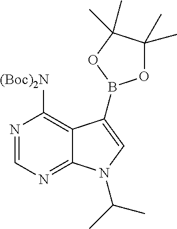

The title compound is prepared by following a similar procedure described for step 2 of Intermediate B2, starting from tert-butyl (5-bromo-7-isopropyl-7H-pyrrolo[2,3-d]pyrimidin-4-yl)(tert-butoxycarbonyl)carbamate (100 mg, 0.220 mmol), HBpin (112 mg, 0.878 mmol), TEA (122 μL, 0.878 mmol), XPhos (10.5 mg, 0.022 mmol), and Pd₂dba₃ (20.1 mg, 0.022 mmol). MS (expected): 503.3 [M+H].

### Step 3: Synthesis of tert-butyl (5-(8-aminoimidazo[1,2-a]pyridin-5-yl)-7-isopropyl-7H-pyrrolo[2,3-d]pyrimidin-4-yl)(tert-butoxycarbonyl)carbamate

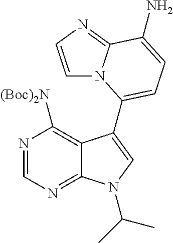

The title compound is prepared by following a similar procedure described for step 3 of Intermediate B2, starting from tert-butyl (tert-butoxycarbonyl)(7-isopropyl-5-(4,4,5,5-tetramethyl-1,3,2-dioxaborolan-2-yl)-7H-pyrrolo[2,3-d]pyrimidin-4-yl)carbamate (100 mg, 0.199 mmol), 5-bromoimidazo[1,2-a]pyridin-8-amine (synthesized as reported in PCT Publication No. WO 2020/081508, 42 mg, 0.199 mmol), K₂CO₃ (55 mg, 0.398 mmol), and PdCl₂(dppf) (7.3 mg, 0.010 mmol). MS (expected): 508.3 [M+H].

### Intermediate B17

### Tert-Butyl (5-(8-Aminoimidazo[1,2-A]Pyridin-5-Yl)-7-Cyclobutyl-7H-Pyrrolo[2,3-D]Pyrimidin-4-Yl)(Tert-Butoxycarbonyl)Carbamate and 5-(8-Aminoimidazo[1,2-A]Pyridin-5-Yl)-7-Cyclobutyl-7H-Pyrrolo[2,3-D]Pyrimidin-4-Amine

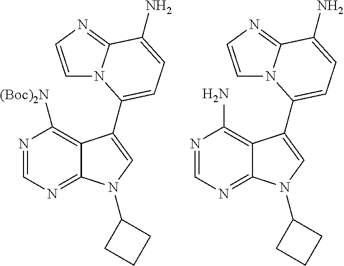

The title compound is prepared by following a similar 3 steps procedure described for Intermediate B16, starting from 7-cyclobutyl-5-iodo-7H-pyrrolo[2,3-d]pyrimidin-4-amine (A4), (Boc)₂O, and DMAP in step 1, from tert-butyl (tert-butoxycarbonyl)(7-cyclobutyl-5-iodo-7H-pyrrolo[2,3-d]pyrimidin-4-yl)carbamate, HBpin, TEA, XPhos, and Pd₂dba₃ in step 2, and from tert-butyl (tert-butoxycarbonyl)(7-cyclobutyl-5-(4,4,5,5-tetramethyl-1,3,2-dioxaborolan-2-yl)-7H-pyrrolo[2,3-d]pyrimidin-4-yl)carbamate, 5-bromoimidazo[1,2-a]pyridin-8-amine, K₂CO₃, and PdCl₂(dppf) in step 3. MS (expected): 520.3 [M+H].

The title compound was prepared by following a similar 3 steps procedure described for Intermediate B16, starting from 7-cyclobutyl-5-iodo-7H-pyrrolo[2,3-d]pyrimidin-4-amine (A4, 0.20 g, 0.637 mmol) and (Boc)₂O (0.347 g, 1.592 mmol) in step 1, from tert-butyl (tert-butoxycarbonyl)(7-cyclobutyl-5-iodo-7H-pyrrolo[2,3-d]pyrimidin-4-yl)carbamate (0.25 g, 0.486 mmol) and HBpin (0.311 g, 2.43 mmol) in step 2, and from 5-bromoimidazo[1,2-a]pyridin-8-amine (synthesized as reported in PCT Publication No. WO 2020/081508, 41 mg, 0.194 mmol) and tert-butyl (tert-butoxycarbonyl)(7-cyclobutyl-5-(4,4,5,5-tetramethyl-1,3,2-dioxaborolan-2-yl)-7H-pyrrolo[2,3-d]pyrimidin-4-yl)carbamate (100 mg, 0.194 mmol) in step 3, and was isolated as an off-white solid (40 mg, 20% yield over 3 steps). Cleavage of the di-Boc protecting group was observed during step 3. MS: 320.2 [M+H].

### Intermediate B18

### Tert-Butyl (5-(8-Aminoimidazo[1,2-A]Pyridin-5-Yl)-7-(2-Hydroxyethyl)-7H-Pyrrolo[2,3-D]Pyrimidin-4-Yl)(Tert-Butoxycarbonyl)Carbamate

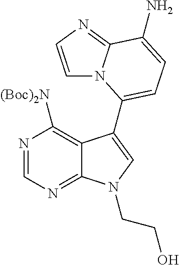

The title compound is prepared by following a similar 3 steps procedure described for Intermediate B16, starting from 2-(4-amino-5-iodo-7H-pyrrolo[2,3-d]pyrimidin-7-yl)ethan-1-ol (A5), (Boc)₂O, and DMAP in step 1, from tert-butyl (5-bromo-7-(2-hydroxyethyl)-7H-pyrrolo[2,3-d]pyrimidin-4-yl)(tert-butoxycarbonyl)carbamate, HBpin, TEA, XPhos, and Pd₂dba₃ in step 2, and from tert-butyl (tert-butoxycarbonyl)(7-(2-hydroxyethyl)-5-(4,4,5,5-tetramethyl-1,3,2-dioxaborolan-2-yl)-7H-pyrrolo[2,3-d]pyrimidin-4-yl)carbamate, 5-bromoimidazo[1,2-a]pyridin-8-amine, K₂CO₃, and PdCl₂(dppf) in step 3. MS (expected): 510.2 [M+H].

The title compound was prepared by following a similar 3 steps procedure described for Intermediate B16, starting from 2-(4-amino-5-iodo-7H-pyrrolo[2,3-d]pyrimidin-7-yl)ethan-1-ol (A5, 0.25 g, 0.822 mmol) and (Boc)₂O (0.45 g, 2.055 mmol) in step 1, from tert-butyl (tert-butoxycarbonyl)(7-(2-((tert-butoxycarbonyl)oxy)ethyl)-5-iodo-7H-pyrrolo[2,3-d]pyrimidin-4-yl)carbamate (0.35 g, 0.405 mmol) and HBpin (0.207 g, 1.62 mmol) in step 2, and from tert-butyl (tert-butoxycarbonyl)(7-(2-hydroxyethyl)-5-(4,4,5,5-tetramethyl-1,3,2-dioxaborolan-2-yl)-7H-pyrrolo[2,3-d]pyrimidin-4-yl)carbamate (200 mg, 0.397 mmol) and 5-bromoimidazo[1,2-a]pyridin-8-amine (synthesized as reported in PCT Publication No. WO 2020/081508, 105 mg, 0.397 mmol) in step 3, and was isolated as an off-white solid (35 mg, 8.3% yield over 3 steps). Cleavage of the mono-Boc protecting group was observed during step 2. MS: 410.2 [M-Boc].

### Intermediate B19

### Tert-Butyl (5-(8-Aminoimidazo[1,2-A]Pyridin-5-Yl)-7-(2-Hydroxy-2-Methylpropyl)-7H-Pyrrolo[2,3-D]Pyrimidin-4-Yl)(Tert-Butoxycarbonyl)Carbamate

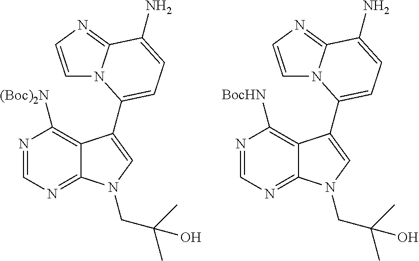

The title compound is prepared by following a similar 3 steps procedure described for Intermediate B16, starting from 1-(4-amino-5-iodo-7H-pyrrolo[2,3-d]pyrimidin-7-yl)-2-methylpropan-2-ol (A6), (Boc)₂O, and DMAP in step 1, from tert-butyl (tert-butoxycarbonyl)(7-(2-hydroxy-2-methylpropyl)-5-iodo-7H-pyrrolo[2,3-d]pyrimidin-4-yl)carbamate, HBpin, TEA, XPhos, and Pd₂dba₃ in step 2, and from tert-butyl (tert-butoxycarbonyl)(7-(2-hydroxy-2-methylpropyl)-5-(4,4,5,5-tetramethyl-1,3,2-dioxaborolan-2-yl)-7H-pyrrolo[2,3-d]pyrimidin-4-yl)carbamate, 5-bromoimidazo[1,2-a]pyridin-8-amine, K₂CO₃, and PdCl₂(dppf) in step 3. MS (expected): 538.3 [M+H].

The title compound was prepared by following a similar 3 steps procedure described for Intermediate B16, starting from 1-(4-amino-5-iodo-7H-pyrrolo[2,3-d]pyrimidin-7-yl)-2-methylpropan-2-ol (A6, 0.10 g, 0.301 mmol) and (Boc)₂O (0.197 g, 0.903 mmol) in step 1, from tert-butyl (tert-butoxycarbonyl)(7-(2-((tert-butoxycarbonyl)oxy)-2-methylpropyl)-5-iodo-7H-pyrrolo[2,3-d]pyrimidin-4-yl)carbamate (0.150 g, 0.237 mmol) and HBpin (0.121 g, 0.950 mmol) in step 2, and from tert-butyl (7-(2-((tert-butoxycarbonyl)oxy)-2-methylpropyl)-5-(4,4,5,5-tetramethyl-1,3,2-dioxaborolan-2-yl)-7H-pyrrolo[2,3-d]pyrimidin-4-yl)carbamate (100 mg, 0.118 mmol) and 5-bromoimidazo[1,2-a]pyridin-8-amine (synthesized as reported in PCT Publication No. WO 2020/081508, 31 mg, 0.118 mmol) in step 3, and was isolated as an off-white solid (25 mg, 15% yield over 3 steps). Cleavage of the mono-Boc protecting group was observed during step 3. MS: 438.2 [M+H].

### Intermediate B20

### Tert-Butyl (5-(8-Aminoimidazo[1,2-A]Pyridin-5-Yl)-7-(3-(Benzyloxy)Cyclobutyl)-7H-Pyrrolo[2,3-D]Pyrimidin-4-Yl)(Tert-Butoxycarbonyl)Carbamate and 5-(8-Aminoimidazo[1,2-A]Pyridin-5-Yl)-7-(3-(Benzyloxy)Cyclobutyl)-7H-Pyrrolo[2,3-D]Pyrimidin-4-Amine

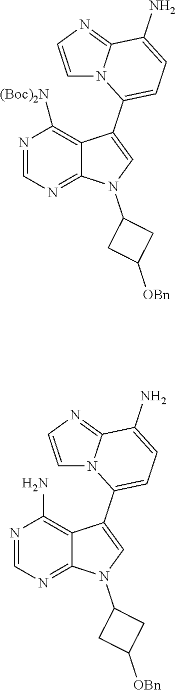

The title compound is prepared by following a similar 3 steps procedure described for Intermediate B16, starting from 7-(3-(benzyloxy)cyclobutyl)-5-iodo-7H-pyrrolo[2,3-d]pyrimidin-4-amine (A7), (Boc)₂O, and DMAP in step 1, from tert-butyl (7-(3-(benzyloxy)cyclobutyl)-5-iodo-7H-pyrrolo[2,3-d]pyrimidin-4-yl)(tert-butoxycarbonyl)carbamate, HBpin, TEA, XPhos, and Pd₂dba₃ in step 2, and from tert-butyl (7-(3-(benzyloxy)cyclobutyl)-5-(4,4,5,5-tetramethyl-1,3,2-dioxaborolan-2-yl)-7H-pyrrolo[2,3-d]pyrimidin-4-yl)(tert-butoxycarbonyl)carbamate, 5-bromoimidazo[1,2-a]pyridin-8-amine, K₂CO₃, and PdCl₂(dppf) in step 3. MS (expected): 538.3 [M+H].

The title compound was prepared by following a similar 3 steps procedure described for Intermediate B16, starting from 7-(3-(benzyloxy)cyclobutyl)-5-iodo-7H-pyrrolo[2,3-d]pyrimidin-4-amine (A7, 0.120 g, 0.286 mmol) and (Boc)₂O (0.125 g, 0.571 mmol) in step 1, from tert-butyl (7-(3-(benzyloxy)cyclobutyl)-5-iodo-7H-pyrrolo[2,3-d]pyrimidin-4-yl)(tert-butoxycarbonyl)carbamate (0.150 g, 0.242 mmol) and HBpin (0.124 g, 0.970 mmol) in step 2, and from tert-butyl (7-(3-(benzyloxy)cyclobutyl)-5-(4,4,5,5-tetramethyl-1,3,2-dioxaborolan-2-yl)-7H-pyrrolo[2,3-d]pyrimidin-4-yl)(tert-butoxycarbonyl)carbamate (150 mg, 0.288 mmol) and 5-bromoimidazo[1,2-a]pyridin-8-amine (synthesized as reported in PCT Publication No. WO 2020/081508, 61 mg, 0.288 mmol) in step 3, and was isolated as an off-white solid (20 mg, 13% yield over 3 steps). Cleavage of the di-Boc protecting group was observed during step 3. LCMS: 426.2 [M+H].

### Intermediate B21

### Tert-Butyl 3-(5-(8-Aminoimidazo[1,2-A]Pyridin-5-Yl)-4-(Bis(Tert-Butoxycarbonyl)Amino)-7H-Pyrrolo[2,3-D]Pyrimidin-7-Yl)Propanoate

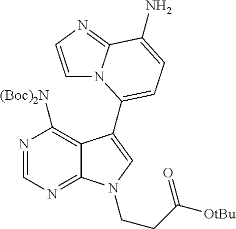

The title compound is prepared by following a similar 3 steps procedure described for Intermediate B16, starting from tert-butyl 3-(4-amino-5-iodo-7H-pyrrolo[2,3-d]pyrimidin-7-yl)propanoate (A8), (Boc)₂O, and DMAP in step 1, from tert-butyl 3-(4-(bis(tert-butoxycarbonyl)amino)-5-iodo-7H-pyrrolo[2,3-d]pyrimidin-7-yl)propanoate, HBpin, TEA, XPhos, and Pd₂dba₃ in step 2, and from tert-butyl 3-(4-(bis(tert-butoxycarbonyl)amino)-5-(4,4,5,5-tetramethyl-1,3,2-dioxaborolan-2-yl)-7H-pyrrolo[2,3-d]pyrimidin-7-yl)propanoate, 5-bromoimidazo[1,2-a]pyridin-8-amine, K₂CO₃, and PdCl₂(dppf) in step 3. MS (expected): 594.3 [M+H].

### Intermediate B22

### Tert-Butyl (5-(8-Aminoimidazo[1,2-A]Pyridin-5-Yl)-7-(2-Methoxyethyl)-7H-Pyrrolo[2,3-D]Pyrimidin-4-Yl)(Tert-Butoxycarbonyl)Carbamate

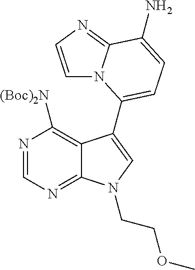

The title compound is prepared by following a similar 3 steps procedure described for Intermediate B16, starting from 5-iodo-7-(2-methoxyethyl)-7H-pyrrolo[2,3-d]pyrimidin-4-amine (A9), (Boc)₂O, and DMAP in step 1, from tert-butyl (tert-butoxycarbonyl)(5-iodo-7-(2-methoxyethyl)-7H-pyrrolo[2,3-d]pyrimidin-4-yl)carbamate, HBpin, TEA, XPhos, and Pd₂dba₃ in step 2, and from tert-butyl (5-(8-aminoimidazo[1,2-a]pyridin-5-yl)-7-(2-methoxyethyl)-7H-pyrrolo[2,3-d]pyrimidin-4-yl)(tert-butoxycarbonyl)carbamate, 5-bromoimidazo[1,2-a]pyridin-8-amine, K₂CO₃, and PdCl₂(dppf) in step 3. MS (expected): 524.3 [M+H].

The title compound was prepared by following a similar 3 steps procedure described for Intermediate B16, starting from 5-iodo-7-(2-methoxyethyl)-7H-pyrrolo[2,3-d]pyrimidin-4-amine (A9, 0.200 g, 0.629 mmol) and (Boc)₂O (0.274 g, 1.258 mmol) in step 1, from tert-butyl (tert-butoxycarbonyl)(5-iodo-7-(2-methoxyethyl)-7H-pyrrolo[2,3-d]pyrimidin-4-yl)carbamate (0.250 g, 0.482 mmol) and HBpin (0.247 g, 1.930 mmol) in step 2, and from tert-butyl (5-(8-aminoimidazo[1,2-a]pyridin-5-yl)-7-(2-methoxyethyl)-7H-pyrrolo[2,3-d]pyrimidin-4-yl)(tert-butoxycarbonyl)carbamate (200 mg, 0.355 mmol) and 5-bromoimidazo[1,2-a]pyridin-8-amine (synthesized as reported in PCT Publication No. WO 2020/081508, 86 mg, 0.355 mmol) in step 3, and was isolated as an off-white solid (35 mg, 11% yield over 3 steps). MS: 524.3 [M+H].

### Intermediate B23

### Tert-Butyl (5-(8-Aminoimidazo[1,2-A]Pyridin-5-Yl)-7-(Oxetan-3-Yl)-7H-Pyrrolo[2,3-D]Pyrimidin-4-Yl)(Tert-Butoxycarbonyl)Carbamate

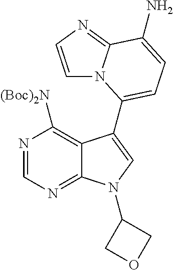

The title compound is prepared by following a similar 3 steps procedure described for Intermediate B16, starting from 5-iodo-7-(oxetan-3-yl)-7H-pyrrolo[2,3-d]pyrimidin-4-amine (A10), (Boc)₂O, and DMAP in step 1, from tert-butyl (tert-butoxycarbonyl)(5-iodo-7-(oxetan-3-yl)-7H-pyrrolo[2,3-d]pyrimidin-4-yl)carbamate, HBpin, TEA, XPhos, and Pd₂dba₃ in step 2, and from tert-butyl (tert-butoxycarbonyl)(7-(oxetan-3-yl)-5-(4,4,5,5-tetramethyl-1,3,2-dioxaborolan-2-yl)-7H-pyrrolo[2,3-d]pyrimidin-4-yl)carbamate, 5-bromoimidazo[1,2-a]pyridin-8-amine, K₂CO₃, and PdCl₂(dppf) in step 3. MS (expected): 522.2 [M+H].

### Intermediate B24

### Tert-Butyl (5-(8-Aminoimidazo[1,2-A]Pyridin-5-Yl)-7-(Pyridin-3-Yl)-7H-Pyrrolo[2,3-D]Pyrimidin-4-Yl)(Tert-Butoxycarbonyl)Carbamate and 5-(8-Aminoimidazo[1,2-A]Pyridin-5-Yl)-7-(Pyridin-3-Yl)-7H-Pyrrolo[2,3-D]Pyrimidin-4-Amine

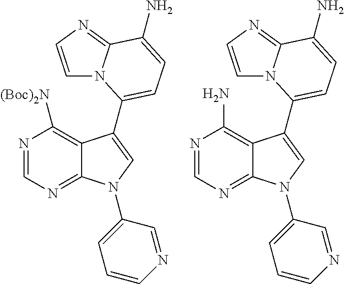

The title compound is prepared by following a similar 3 steps procedure described for Intermediate B16, starting from 5-iodo-7-(pyridin-3-yl)-7H-pyrrolo[2,3-d]pyrimidin-4-amine (A11), (Boc)₂O, and DMAP in step 1, from tert-butyl (tert-butoxycarbonyl)(5-iodo-7-(pyridin-3-yl)-7H-pyrrolo[2,3-d]pyrimidin-4-yl)carbamate, HBpin, TEA, XPhos, and Pd₂dba₃ in step 2, and from tert-butyl (tert-butoxycarbonyl)(7-(pyridin-3-yl)-5-(4,4,5,5-tetramethyl-1,3,2-dioxaborolan-2-yl)-7H-pyrrolo[2,3-d]pyrimidin-4-yl)carbamate, 5-bromoimidazo[1,2-a]pyridin-8-amine, K₂CO₃, and PdCl₂(dppf) in step 3. MS (expected): 543.2 [M+H].

The title compound was prepared by following a similar 3 steps procedure described for Intermediate B16, starting from 5-iodo-7-(pyridin-3-yl)-7H-pyrrolo[2,3-d]pyrimidin-4-amine (A11, 0.300 g, 0.890 mmol) and (Boc)₂O (0.388 g, 1.780 mmol) in step 1, from tert-butyl (tert-butoxycarbonyl)(5-iodo-7-(pyridin-3-yl)-7H-pyrrolo[2,3-d]pyrimidin-4-yl)carbamate (0.350 g, 0.651 mmol) and HBpin (0.333 g, 2.610 mmol) in step 2, and from tert-butyl (tert-butoxycarbonyl)(7-(pyridin-3-yl)-5-(4,4,5,5-tetramethyl-1,3,2-dioxaborolan-2-yl)-7H-pyrrolo[2,3-d]pyrimidin-4-yl)carbamate (150 mg, 0.279 mmol) and 5-bromoimidazo[1,2-a]pyridin-8-amine (synthesized as reported in PCT Publication No. WO 2020/081508, 59 mg, 0.279 mmol) in step 3, and was isolated as an off-white solid (15 mg, 4.9% yield over 3 steps). Cleavage of the di-Boc protecting group was observed during step 3. LCMS: 343.2 [M+H].

### Intermediate B25

### Tert-Butyl (5-(8-Aminoimidazo[1,2-A]Pyridin-5-Yl)-7-(Pyridin-4-Yl)-7H-Pyrrolo[2,3-D]Pyrimidin-4-Yl)(Tert-Butoxycarbonyl)Carbamate

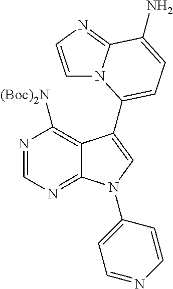

The title compound is prepared by following a similar 3 steps procedure described for Intermediate B16, starting from 5-iodo-7-(pyridin-4-yl)-7H-pyrrolo[2,3-d]pyrimidin-4-amine (A12), (Boc)₂O, and DMAP in step 1, from tert-butyl (tert-butoxycarbonyl)(5-iodo-7-(pyridin-4-yl)-7H-pyrrolo[2,3-d]pyrimidin-4-yl)carbamate, HBpin, TEA, XPhos, and Pd₂dba₃ in step 2, and from tert-butyl (tert-butoxycarbonyl)(7-(pyridin-4-yl)-5-(4,4,5,5-tetramethyl-1,3,2-dioxaborolan-2-yl)-7H-pyrrolo[2,3-d]pyrimidin-4-yl)carbamate, 5-bromoimidazo[1,2-a]pyridin-8-amine, K₂CO₃, and PdCl₂(dppf) in step 3. MS (expected): 543.2 [M+H].

### Intermediate B26

### Tert-Butyl (5-(8-Aminoimidazo[1,2-A]Pyridin-5-Yl)-7-(1-Methylpyrrolidin-3-Yl)-7H-Pyrrolo[2,3-D]Pyrimidin-4-Yl)(Tert-Butoxycarbonyl)Carbamate

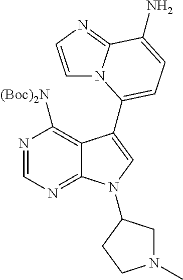

The title compound is prepared by following a similar 3 steps procedure described for Intermediate B16, starting from 5-iodo-7-(1-methylpyrrolidin-3-yl)-7H-pyrrolo[2,3-d]pyrimidin-4-amine (A13), (Boc)₂O, and DMAP in step 1, from tert-butyl (tert-butoxycarbonyl)(5-iodo-7-(1-methylpyrrolidin-3-yl)-7H-pyrrolo[2,3-d]pyrimidin-4-yl)carbamate, HBpin, TEA, XPhos, and Pd₂dba₃ in step 2, and from tert-butyl (tert-butoxycarbonyl)(7-(1-methylpyrrolidin-3-yl)-5-(4,4,5,5-tetramethyl-1,3,2-dioxaborolan-2-yl)-7H-pyrrolo[2,3-d]pyrimidin-4-yl)carbamate, 5-bromoimidazo[1,2-a]pyridin-8-amine, K₂CO₃, and PdCl₂(dppf) in step 3. MS (expected): 549.3 [M+H].

### Intermediate B27

### Tert-Butyl 4-(5-(8-Aminoimidazo[1,2-A]Pyridin-5-Yl)-4-(Bis(Tert-Butoxycarbonyl)Amino)-7H-Pyrrolo[2,3-D]Pyrimidin-7-Yl)Piperidine-1-Carboxylate

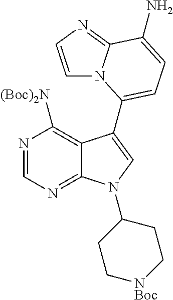

The title compound is prepared by following a similar 3 steps procedure described for Intermediate B16, starting from tert-butyl 4-(4-amino-5-iodo-7H-pyrrolo[2,3-d]pyrimidin-7-yl)piperidine-1-carboxylate (A14), (Boc)₂O, and DMAP in step 1, from tert-butyl 4-(4-(bis(tert-butoxycarbonyl)amino)-5-iodo-7H-pyrrolo[2,3-d]pyrimidin-7-yl)piperidine-1-carboxylate, HBpin, TEA, XPhos, and Pd₂dba₃ in step 2, and from tert-butyl 4-(4-(bis(tert-butoxycarbonyl)amino)-5-(4,4,5,5-tetramethyl-1,3,2-dioxaborolan-2-yl)-7H-pyrrolo[2,3-d]pyrimidin-7-yl)piperidine-1-carboxylate, 5-bromoimidazo[1,2-a]pyridin-8-amine, K₂CO₃, and PdCl₂(dppf) in step 3. MS (expected): 649.3 [M+H].

### Intermediate B28

### Tert-Butyl (5-(8-Aminoimidazo[1,2-A]Pyridin-5-Yl)-7-(1-(Methylsulfonyl)Piperidin-4-Yl)-7H-Pyrrolo[2,3-D]Pyrimidin-4-Yl)(Tert-Butoxycarbonyl)Carbamate

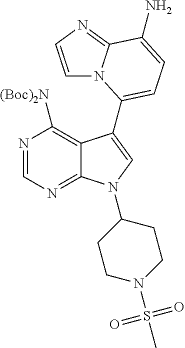

The title compound is prepared by following a similar 3 steps procedure described for Intermediate B16, starting from 5-iodo-7-(1-(methylsulfonyl)piperidin-4-yl)-7H-pyrrolo[2,3-d]pyrimidin-4-amine (A15), (Boc)₂O, and DMAP in step 1, from tert-butyl (tert-butoxycarbonyl)(5-iodo-7-(1-(methylsulfonyl)piperidin-4-yl)-7H-pyrrolo[2,3-d]pyrimidin-4-yl)carbamate, HBpin, TEA, XPhos, and Pd₂dba₃ in step 2, and from tert-butyl (tert-butoxycarbonyl)(7-(1-(methylsulfonyl)piperidin-4-yl)-5-(4,4,5,5-tetramethyl-1,3,2-dioxaborolan-2-yl)-7H-pyrrolo[2,3-d]pyrimidin-4-yl)carbamate, 5-bromoimidazo[1,2-a]pyridin-8-amine, K₂CO₃, and PdCl₂(dppf) in step 3. MS (expected): 627.3 [M+H].

### Intermediate B29

### 5-(8-Aminoimidazo[1,2-A]Pyridin-5-Yl)-N-(2,4-Dimethoxybenzyl)-7-Methyl-7H-Pyrrolo[2,3-D]Pyrimidin-4-Amine

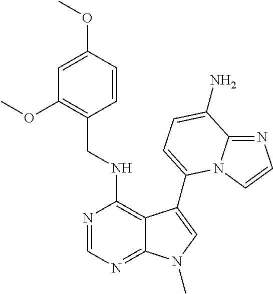 [SMILES: COc1ccc(CNc2ncnc3c2c(-c2ccc(N)c4nccn24)cn3C)c(OC)c1]

The title compound was prepared by following a similar 2 steps procedure described for Intermediate B15, starting from N-(2,4-dimethoxybenzyl)-5-iodo-7-methyl-7H-pyrrolo[2,3-d]pyrimidin-4-amine (A30, 1.00 g, 2.36 mmol) and HBpin (3.02 g, 23.57 mmol) in step 1 and from N-(2,4-dimethoxybenzyl)-7-methyl-5-(4,4,5,5-tetramethyl-1,3,2-dioxaborolan-2-yl)-7H-pyrrolo[2,3-d]pyrimidin-4-amine (200 mg, 0.471 mmol) and 5-bromoimidazo[1,2-a]pyridin-8-amine (synthesized as reported in PCT Publication No. WO 2020/081508, 100 mg, 0.471 mmol) in step 2, and was isolated as an off-white solid (180 mg, 18% yield over 2 steps). MS: 430.2 [M+H].

### Intermediate B30

### 5-(8-Aminoimidazo[1,2-A]Pyridin-5-Yl)-N-(2,4-Dimethoxybenzyl)-7-Isopropyl-7H-Pyrrolo[2,3-D]Pyrimidin-4-Amine

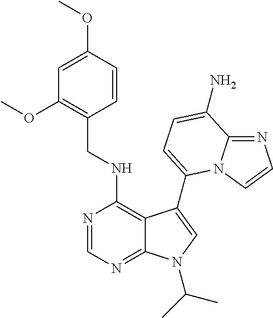 [SMILES: COc1ccc(CNc2ncnc3c2c(-c2ccc(N)c4nccn24)cn3C(C)C)c(OC)c1]

The title compound was prepared by following a similar 2 steps procedure described for Intermediate B15, starting from N-(2,4-dimethoxybenzyl)-5-iodo-7-isopropyl-7H-pyrrolo[2,3-d]pyrimidin-4-amine (A31, 0.70 g, 1.55 mmol) and HBpin (0.792 g, 6.19 mmol) in step 1 and from N-(2,4-dimethoxybenzyl)-7-isopropyl-5-(4,4,5,5-tetramethyl-1,3,2-dioxaborolan-2-yl)-7H-pyrrolo[2,3-d]pyrimidin-4-amine (200 mg, 0.442 mmol) and 5-bromoimidazo[1,2-a]pyridin-8-amine (synthesized as reported in PCT Publication No. WO 2020/081508, 94 mg, 0.442 mmol) in step 2, and was isolated as an off-white solid (60 mg, 8.5% yield over 2 steps). MS: 458.2 [M+H].

### Intermediate B31

### Tert-Butyl 4-(5-(8-Aminoimidazo[1,2-A]Pyridin-5-Yl)-4-((2,4-Dimethoxybenzyl)Amino)-7H-Pyrrolo[2,3-D]Pyrimidin-7-Yl)Piperidine-1-Carboxylate

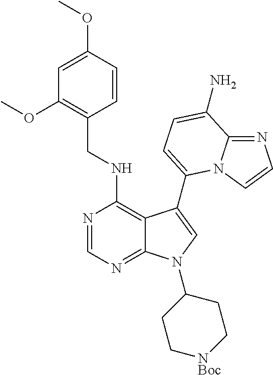 [SMILES: COc1ccc(CNc2ncnc3c2c(-c2ccc(N)c4nccn24)cn3C2CCN(C(=O)OC(C)(C)C)CC2)c(OC)c1]

The title compound was prepared by following a similar 2 steps procedure described for Intermediate B15, starting from tert-butyl 4-(4-((2,4-dimethoxybenzyl)amino)-5-iodo-7H-pyrrolo[2,3-d]pyrimidin-7-yl)piperidine-1-carboxylate (A32, 0.60 g, 1.01 mmol) and HBpin (1.54 g, 12.13 mmol) in step 1 and from tert-butyl 4-(4-((2,4-dimethoxybenzyl)amino)-5-(4,4,5,5-tetramethyl-1,3,2-dioxaborolan-2-yl)-7H-pyrrolo[2,3-d]pyrimidin-7-yl)piperidine-1-carboxylate (400 mg, 0.674 mmol) and 5-bromoimidazo[1,2-a]pyridin-8-amine (synthesized as reported in PCT Publication No. WO 2020/081508, 143 mg, 0.674 mmol) in step 2, and was isolated as an off-white solid (318 mg, 53% yield over 2 steps). MS: 599.7 [M+H].

### Intermediates C1-C38

### General Procedure for the Synthesis of Carbamate Intermediates C:

Pyridine (1.2 eq) and phenyl chloroformate (1.5 eq) were added to a solution of amine (1.0 eq) in THF (10 vol) at 0° C. The reaction mixture was allowed to warm to 25° C. and was stirred for 12 h. Following completion of the reaction (as indicated by TLC), the mixture was diluted with EtOAc (10 mL) and washed with brine (5 mL). The organic layer was dried over Na₂SO₄, filtered, and concentrated under reduced pressure to yield crude material which was purified by flash chromatography (silica gel 230-400 mesh, eluting with 10 to 20% EtOAc in petroleum ether), giving the desired carbamate.

The following carbamates were prepared using the above general procedure:

| Carbamate structure (product) | Amine structure (starting material) | LCMS |
|---|---|---|
| 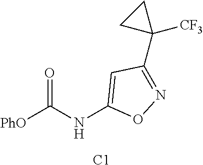 | 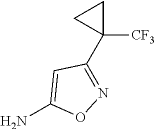 [SMILES: Nc1cc(C2(C(F)(F)F)CC2)no1] | 312.9 [M + H] |
| 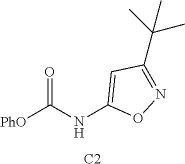 | 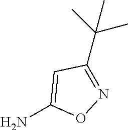 | 261.0 [M + H] |
| 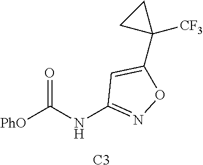 | 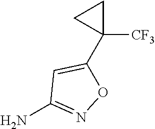 [SMILES: Nc1cc(C2(C(F)(F)F)CC2)on1] | 312.9 [M + H] |
| 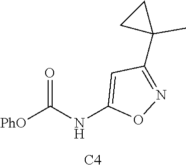 | 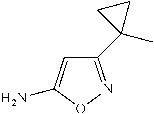 | 259.1 [M + H] |
| 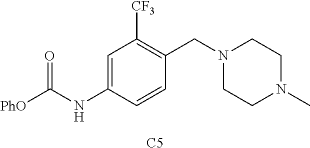 | 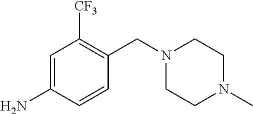 | 394.3 [M + H] |
| 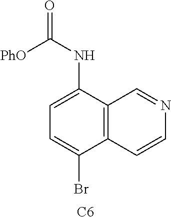 [SMILES: O=C(Nc1ccc(Br)c2ccncc12)Oc1ccccc1 ; "C6"] | 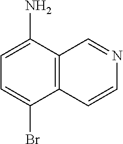 [SMILES: Nc1ccc(Br)c2ccncc12] | 343.0 [M + H] |
| 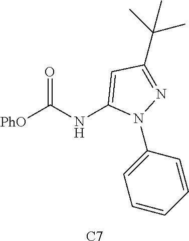 [SMILES: CC(C)(C)c1cc(NC(=O)Oc2ccccc2)n(-c2ccccc2)n1 ; "C7"] | 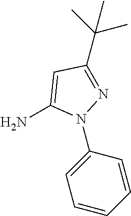 | 336.2 [M + H] |
| 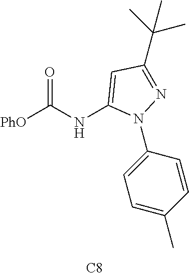 [SMILES: Cc1ccc(-n2nc(C(C)(C)C)cc2NC(=O)Oc2ccccc2)cc1 ; "C8"] | 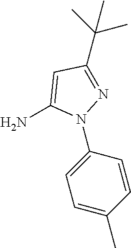 | 350.4 [M + H] |
| 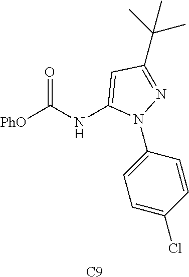 [SMILES: CC(C)(C)c1cc(NC(=O)Oc2ccccc2)n(-c2ccc(Cl)cc2)n1 ; "C9"] | 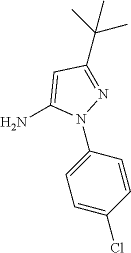 | 370.3 [M + H] |
| 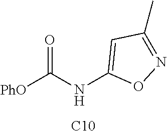 [SMILES: Cc1cc(NC(=O)Oc2ccccc2)on1 ; "C10"] | 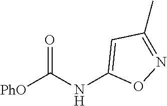 [SMILES: Cc1cc(NC(=O)Oc2ccccc2)on1] | 219.1 [M + H] |
| 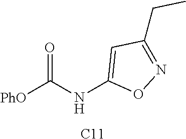 [SMILES: CCc1cc(NC(=O)Oc2ccccc2)on1 ; "C11"] | 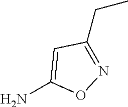 | 233.0 [M + H] |
| 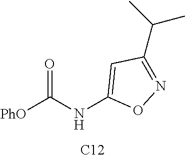 [SMILES: CC(C)c1cc(NC(=O)Oc2ccccc2)on1 ; "C12"] | 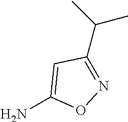 | 247.1 [M + H] |
| 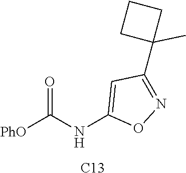 [SMILES: CC1(c2cc(NC(=O)Oc3ccccc3)on2)CCC1 ; "C13"] | 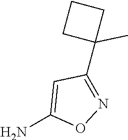 | 273.2 [M + H] |
| 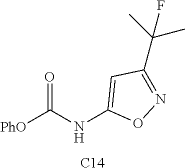 [SMILES: CC(C)(F)c1cc(NC(=O)Oc2ccccc2)on1 ; "C14"] | 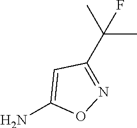 | 265.1 [M + H] |
| 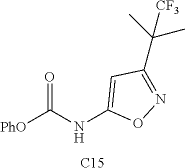 [SMILES: CC(C)(c1cc(NC(=O)Oc2ccccc2)on1)C(F)(F)F ; "C15"] | 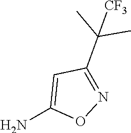 | 315.1 [M + H] |
| 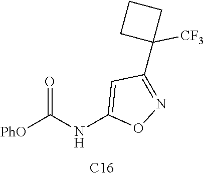 [SMILES: O=C(Nc1cc(C2(C(F)(F)F)CCC2)no1)Oc1ccccc1 ; "C16"] | 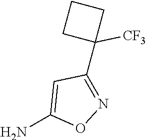 [SMILES: Nc1cc(C2(C(F)(F)F)CCC2)no1] | 327.2 [M + H] |
| 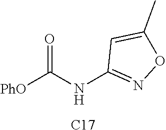 [SMILES: Cc1cc(NC(=O)Oc2ccccc2)no1 ; "C17"] | 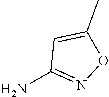 | 219.1 [M + H] |
| 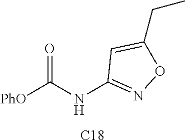 [SMILES: CCc1cc(NC(=O)Oc2ccccc2)no1 ; "C18"] | 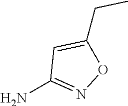 | 233.1^{†} [M + H] |
| 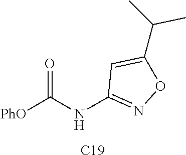 [SMILES: CC(C)c1cc(NC(=O)Oc2ccccc2)no1 ; "C19"] | 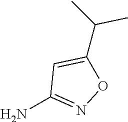 | 247.2 [M + H] |
| 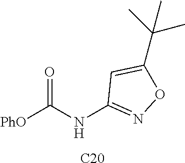 [SMILES: CC(C)(C)c1cc(NC(=O)Oc2ccccc2)no1 ; "C20"] | 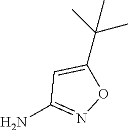 | 261.0 [M + H] |
| 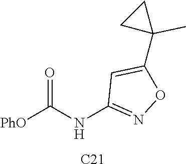 [SMILES: CC1(c2cc(NC(=O)Oc3ccccc3)no2)CC1 ; "C21"] | 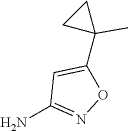 | 259.2 [M + H] |
| 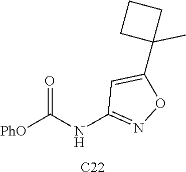 [SMILES: CC1(c2cc(NC(=O)Oc3ccccc3)no2)CCC1 ; "C22"] | 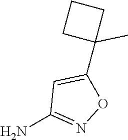 | 273.2 [M + H] |
| 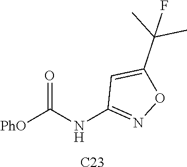 [SMILES: CC(C)(F)c1cc(NC(=O)Oc2ccccc2)no1 ; "C23"] | 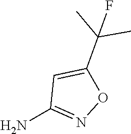 | 265.1^{†} [M + H] |
| 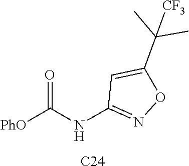 [SMILES: CC(C)(c1cc(NC(=O)Oc2ccccc2)no1)C(F)(F)F ; "C24"] | 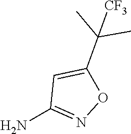 | 315.4 [M + H] |
| 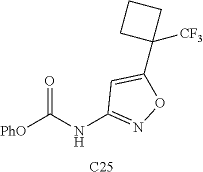 [SMILES: O=C(Nc1cc(C2(C(F)(F)F)CCC2)on1)Oc1ccccc1 ; "C25"] | 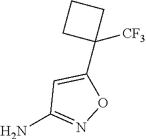 [SMILES: Nc1cc(C2(C(F)(F)F)CCC2)on1] | 327.0 [M + H] |
| 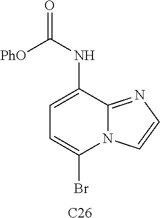 [SMILES: O=C(Nc1ccc(Br)n2ccnc12)Oc1ccccc1 ; "C26"] | 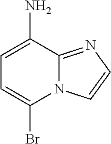 [SMILES: Nc1ccc(Br)n2ccnc12] | 332.2 [M + H] |
| 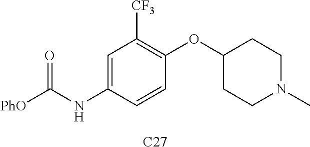 [SMILES: CN1CCC(Oc2ccc(NC(=O)Oc3ccccc3)cc2C(F)(F)F)CC1 ; "C27"] | 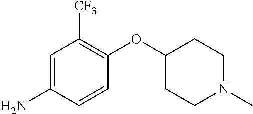 | 395.2 [M + H] |
| 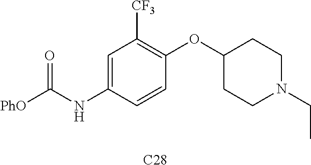 [SMILES: CCN1CCC(Oc2ccc(NC(=O)Oc3ccccc3)cc2C(F)(F)F)CC1 ; "C28"] | 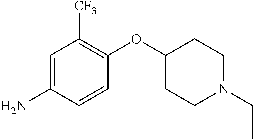 | 409.2 [M + H] |
| 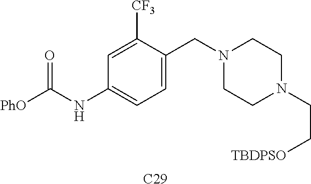 | 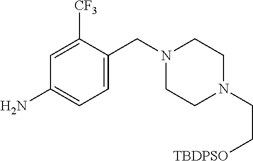 | 662.3^{†} [M + H] |
| 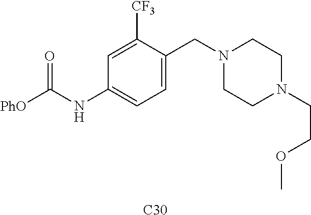 [SMILES: COCCN1CCN(Cc2ccc(NC(=O)Oc3ccccc3)cc2C(F)(F)F)CC1 ; "C30"] | 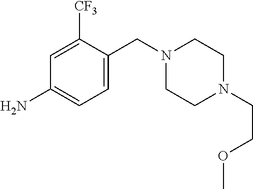 | 438.2^{†} [M + H] |
| 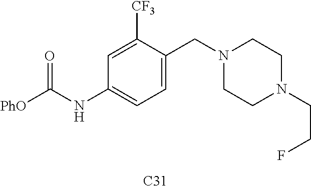 [SMILES: O=C(Nc1ccc(CN2CCN(CCF)CC2)c(C(F)(F)F)c1)Oc1ccccc1 ; "C31"] | 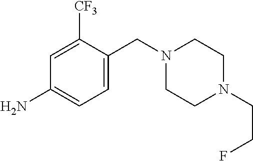 [SMILES: Nc1ccc(CN2CCN(CCF)CC2)c(C(F)(F)F)c1] | 426.2^{†} [M + H] |
| 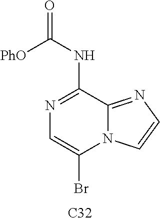 [SMILES: O=C(Nc1ncc(Br)n2ccnc12)Oc1ccccc1 ; "C32"] | 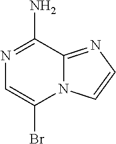 [SMILES: Nc1ncc(Br)n2ccnc12] | 333.0 [M + H] |
| 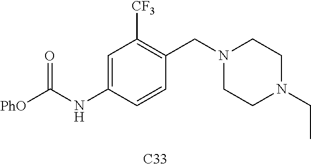 [SMILES: CCN1CCN(Cc2ccc(NC(=O)Oc3ccccc3)cc2C(F)(F)F)CC1 ; "C33"] | 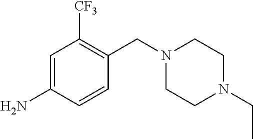 | 408.2 [M + H] |
| 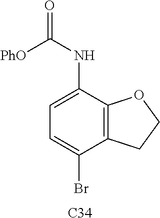 [SMILES: O=C(Nc1ccc(Br)c2c1OCC2)Oc1ccccc1 ; "C34"] | 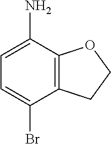 [SMILES: Nc1ccc(Br)c2c1OCC2] | 334.1 [M + H] |
| 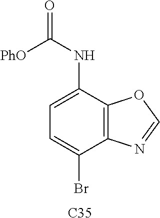 [SMILES: O=C(Nc1ccc(Br)c2ncoc12)Oc1ccccc1 ; "C35"] | 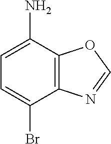 [SMILES: Nc1ccc(Br)c2ncoc12] | 333.1 [M + H] |
| 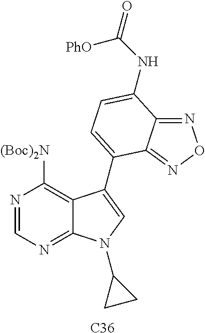 | 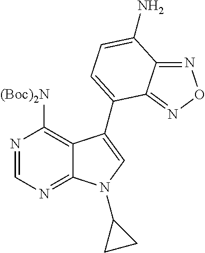 | 628.2 [M + H] |
| 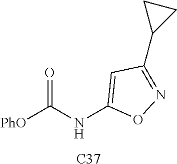 [SMILES: O=C(Nc1cc(C2CC2)no1)Oc1ccccc1 ; "C37"] | 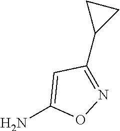 | 245.0 [M + H] |
| 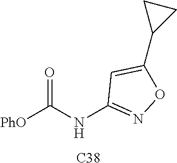 [SMILES: O=C(Nc1cc(C2CC2)on1)Oc1ccccc1 ; "C38"] | 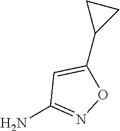 | 245.2 [M + H] |

^{†}Expected value

All amines used for the synthesis of carbamate Intermediates E are commercially available except for the following:
3-(1-(Trifluoromethyl)cyclopropyl)isoxazol-5-amine (precursor to C1), 5-(1-(trifluoromethyl)cyclopropyl)isoxazol-3-amine (precursor to C3), and 5-(1-methylcyclopropyl)isoxazol-3-amine (precursor to C21) were synthesized from 1-(trifluoromethyl)cyclopropane-1-carboxylic acid methyl ester as reported in Synthesis 2013, 45, 171-173.
3-(1,1,1-Trifluoro-2-methylpropan-2-yl)isoxazol-5-amine (precursor to C15), 3-(1-(trifluoromethyl)cyclobutyl)isoxazol-5-amine (precursor to C16), and 5-(1,1,1-trifluoro-2-methylpropan-2-yl)isoxazol-3-amine (precursor to C24) were synthesized as reported in J. Med. Chem. 2012, 55(3), 1082-1105.
5-(1-Methylcyclobutyl)isoxazol-3-amine (precursor to C22) is prepared as reported in PCT Publication No. WO 2011/022473.
5-Bromoimidazo[1,2-a]pyridin-8-amine (precursor to C26) was synthesized as reported in PCT Publication No. WO 2020/081508.
4-((1-Methylpiperidin-4-yl)oxy)-3-(trifluoromethyl)aniline (precursor to C27) was synthesized as reported in PCT Publication No. WO 2017/125530.
4-((1-Ethylpiperidin-4-yl)oxy)-3-(trifluoromethyl)aniline (precursor to C28) is prepared as reported in PCT Publication No. WO 2018/215668.
4-((4-(2-Methoxyethyl)piperazin-1-yl)methyl)-3-(trifluoromethyl)aniline (precursor to C30) and 4-((4-(2-fluoroethyl)piperazin-1-yl)methyl)-3-(trifluoromethyl)aniline (precursor to C31) were synthesized as reported in PCT Publication No. WO 2022/008383.
5-Bromoimidazo[1,2-a]pyrazin-8-amine (precursor to C32) was synthesized as reported in PCT Publication No. WO 2009/017701.
4-Bromo-2,3-dihydrobenzofuran-7-amine (precursor to C34) was synthesized as reported in PCT Publication No. WO 2021/244582.

### Synthesis of 3-(1-methylcyclobutyl)isoxazol-5-amine (Precursor to C13)

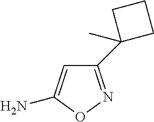

NH₂OH·H₂SO₄ (699 mg, 4.25 mmol) was added to a solution of 3-(1-methylcyclobutyl)-3-oxopropanenitrile (prepared as reported in PCT Publication No. WO 2017/060874, 500 mg, 3.86 mmol) and sodium hydroxide (170 mg, 4.25 mmol) in EtOH (10 mL) and water (10 mL). The pH of the resulting mixture was adjusted to 7.5 using aqueous NaOH (1M) and the reaction mixture was stirred at 80° C. for 15 h. Following completion of the reaction (as indicated by TLC), the reaction mixture was concentrated under reduced pressure, giving a residue which was taken in DCM (25 mL), washed with water (10 mL), dried over Na₂SO₄, filtered, and concentrated under reduced pressure. The resulting crude material was purified by flash chromatography (silica gel 230-400 mesh, eluting with 40% EtOAc in petroleum ether to afford the title product as an off-white solid (110 mg, 19% yield). ¹H NMR (400 MHz, CDCl₃) δ=5.04 (s, 1H), 2.43-2.49 (m, 2H), 1.96-2.02 (m, 4H), 1.50 (s, 3H). MS: 153.2 [M+H].

### Synthesis of 5-(1-methylcyclobutyl)isoxazol-3-amine (Precursor to C22)

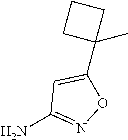

NaHCO₃ (612 mg, 7.29 mmol) and (NH₂OH)₂·H₂SO₄ (573 mg, 4.37 mmol) were added to a solution of 3-(1-methylcyclobutyl)-3-oxopropanenitrile (prepared as reported in PCT Publication No. WO 2017/060874, 400 mg, 2.92 mmol) in MeOH (1.1 mL) and water (10.0 mL) and the resulting mixture was stirred at 65° C. for 16 h. Following completion of the reaction (as indicated by TLC), 10% MeOH in water (2 mL) was added, the pH of the solution was adjusted to 1 using concentrated HCl, and the reaction mixture was stirred at 120° C. for 16 h. Following completion of the reaction (as indicated by LCMS), the reaction mixture was neutralized with saturated aqueous NaHCO₃ and extracted with DCM (2×100 mL). The combined organic layers were washed with brine (20 mL), dried over Na₂SO₄, filtered, and concentrated under reduced pressure to give crude material which was purified by GRACE (silica gel 60-120 mesh, eluting 15% EtOAc in petroleum ether), affording the title compound (191 mg, 43% yield) as an off-white solid. ¹H NMR (400 MHz, DMSO-d₆) δ=5.43 (s, 1H), 5.33 (bs, 2H), 2.26-2.35 (m, 2H), 1.82-2.05 (m, 4H), 1.42 (s, 3H); LCMS: 153.2 [M+H].

### Synthesis of 4-((4-(2-((tert-butyldiphenylsilyl)oxy)ethyl)piperazin-1-yl)methyl)-3-(trifluoromethyl)aniline (Precursor to C29)

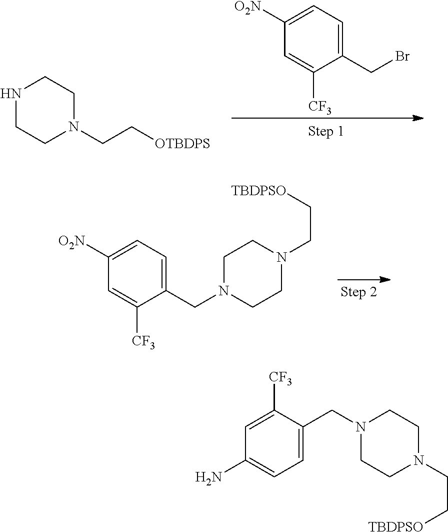

### Step 1: Synthesis of 1-(2-((tert-butyldiphenylsilyl)oxy)ethyl)-4-(4-nitro-2-(trifluoromethyl)benzyl)piperazine

Cs₂CO₃ (0.688 g, 2.112 mmol) and 1-(2-((tert-butyldiphenylsilyl)oxy)ethyl) piperazine (synthesized as reported in PCT Publication No. WO 2012/017251, 0.519 g, 1.408 mmol) were added to a solution of 1-(bromomethyl)-4-nitro-2-(trifluoromethyl)benzene (synthesized as reported in PCT Publication No. WO 2014/206343, 0.400 g, 1.408 mmol) in THF (6 mL) and the resulting mixture was stirred 25° C. for 12 h. Following completion of the reaction (as indicated by TLC), the reaction mixture was filtered through a pad of Celite® (i.e., diatomaceous earth) and the filtrate was concentrated under reduced pressure to give crude material which was purified by flash chromatography (silica gel 230-400 mesh, eluting with 10% EtOAc in petroleum ether), giving the title compound as a white solid (0.560 g, 69% yield). MS: 572.2 [M+H].

### Step 2: Synthesis of 4-((4-(2-((tert-butyldiphenylsilyl)oxy)ethyl)piperazin-1-yl)methyl)-3-(trifluoromethyl)aniline

Iron powder (547 mg, 9.80 mmol) and NH₄Cl (524 mg, 9.8 mmol) were added to a suspension of 1-(2-((tert-butyldiphenylsilyl)oxy)ethyl)-4-(4-nitro-2-(trifluoromethyl)benzyl)piperazine (560 mg, 0.98 mmol) in ethanol (6 mL) and water (1.5 mL) and the resulting mixture was stirred at 80° C. for 2 h. Following completion of the reaction (as indicated by UPLC), the reaction mixture was filtered through a pad of Celite® (i.e., diatomaceous earth) which was then rinsed with DCM (5 mL). The combined filtrates were concentrated under reduced pressure, giving crude material which was taken in DCM (10 mL), washed with brine (2 mL), dried over Na₂SO4, filtered, and concentrated under reduced pressure to afford the title compound as a yellow gum (0.530 g, quantitative yield) which was used without further purification. LCMS: 542.3 [M+H].

### Intermediates D1-D52

### General Urea Formation Procedure for the Synthesis of Intermediates D (Method B):

DMAP (0.05 eq.) and DIPEA (1.5 eq.) were added to a solution of amine starting material (1.0 eq.) and carbamate starting material (1.0 eq.) in THF (10 Vol.) and the resulting mixture was stirred at 60° C. for 12 h in a sealed tube. Following completion of the reaction (as indicated by LCMS), the reaction mixture was concentrated under reduced pressure to yield the crude material which was purified by reverse phase preparative HPLC to afford the desired product.

The following intermediates were prepared using the above general procedure:

| Product MS | Amine starting material | Carbamate starting material |
|---|---|---|
| 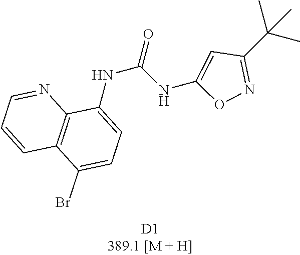 [SMILES: CC(C)(C)c1cc(NC(=O)Nc2ccc(Br)c3cccnc23)on1] | 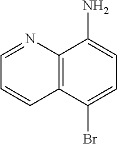 [SMILES: Nc1ccc(Br)c2cccnc12] | 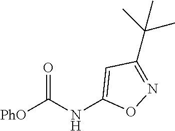 [SMILES: CC(C)(C)c1cc(NC(=O)Oc2ccccc2)on1] |
| 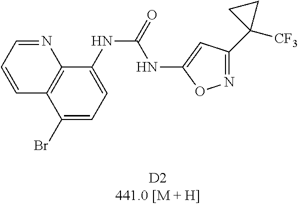 [SMILES: O=C(Nc1cc(C2(C(F)(F)F)CC2)no1)Nc1ccc(Br)c2cccnc12] | 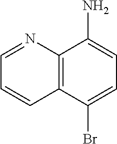 [SMILES: Nc1ccc(Br)c2cccnc12] | 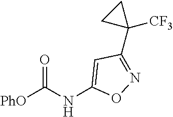 [SMILES: O=C(Nc1cc(C2(C(F)(F)F)CC2)no1)Oc1ccccc1] |
| 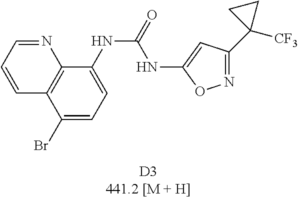 [SMILES: O=C(Nc1cc(C2(C(F)(F)F)CC2)no1)Nc1ccc(Br)c2cccnc12] | 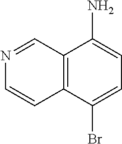 [SMILES: Nc1ccc(Br)c2ccncc12] | 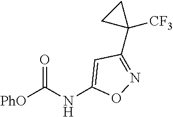 [SMILES: O=C(Nc1cc(C2(C(F)(F)F)CC2)no1)Oc1ccccc1] |
| 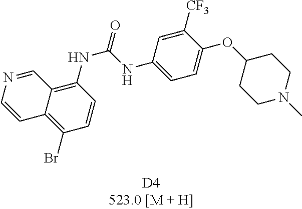 [SMILES: CN1CCC(Oc2ccc(NC(=O)Nc3ccc(Br)c4ccncc34)cc2C(F)(F)F)CC1] | 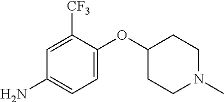 [SMILES: CN1CCC(Oc2ccc(N)cc2C(F)(F)F)CC1] | 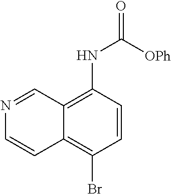 [SMILES: O=C(Nc1ccc(Br)c2ccncc12)Oc1ccccc1] |
| 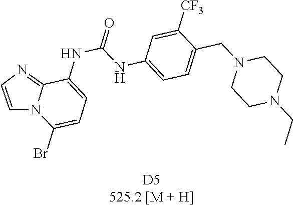 [SMILES: CCN1CCN(Cc2ccc(NC(=O)Nc3ccc(Br)n4ccnc34)cc2C(F)(F)F)CC1] | 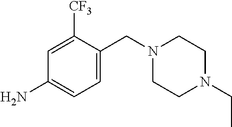 [SMILES: CCN1CCN(Cc2ccc(N)cc2C(F)(F)F)CC1] | 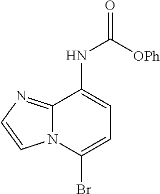 [SMILES: O=C(Nc1ccc(Br)n2ccnc12)Oc1ccccc1] |
| 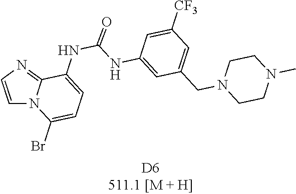 [SMILES: CN1CCN(Cc2cc(NC(=O)Nc3ccc(Br)n4ccnc34)cc(C(F)(F)F)c2)CC1] | 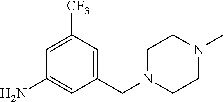 [SMILES: CN1CCN(Cc2cc(N)cc(C(F)(F)F)c2)CC1] | 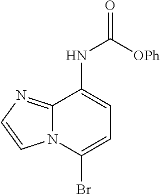 [SMILES: O=C(Nc1ccc(Br)n2ccnc12)Oc1ccccc1] |
| 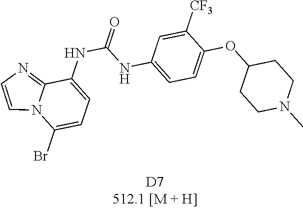 [SMILES: CN1CCC(Oc2ccc(NC(=O)Nc3ccc(Br)n4ccnc34)cc2C(F)(F)F)CC1] | 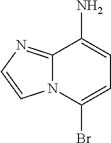 [SMILES: Nc1ccc(Br)n2ccnc12] | 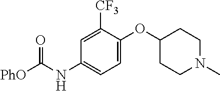 [SMILES: CN1CCC(Oc2ccc(NC(=O)Oc3ccccc3)cc2C(F)(F)F)CC1] |
| 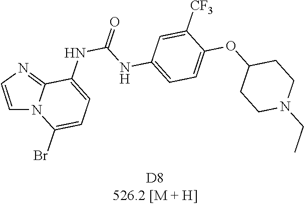 [SMILES: CCN1CCC(Oc2ccc(NC(=O)Nc3ccc(Br)n4ccnc34)cc2C(F)(F)F)CC1] | 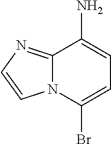 [SMILES: Nc1ccc(Br)n2ccnc12] | 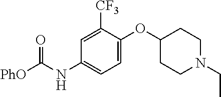 [SMILES: CCN1CCC(Oc2ccc(NC(=O)Oc3ccccc3)cc2C(F)(F)F)CC1] |
| 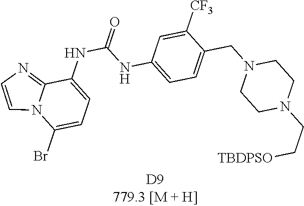 | 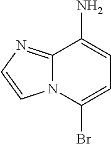 [SMILES: Nc1ccc(Br)n2ccnc12] | 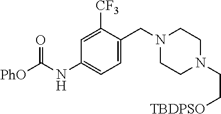 |
| 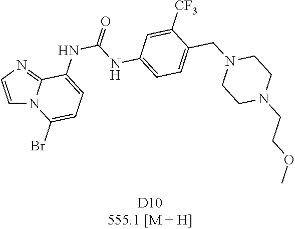 [SMILES: COCCN1CCN(Cc2ccc(NC(=O)Nc3ccc(Br)n4ccnc34)cc2C(F)(F)F)CC1] | 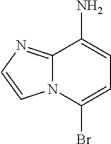 [SMILES: Nc1ccc(Br)n2ccnc12] | 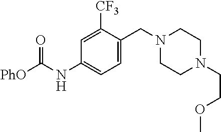 [SMILES: COCCN1CCN(Cc2ccc(NC(=O)Oc3ccccc3)cc2C(F)(F)F)CC1] |
| 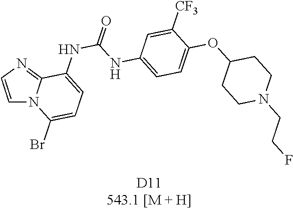 [SMILES: O=C(Nc1ccc(OC2CCN(CCF)CC2)c(C(F)(F)F)c1)Nc1ccc(Br)n2ccnc12] | 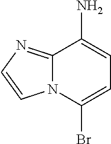 [SMILES: Nc1ccc(Br)n2ccnc12] | 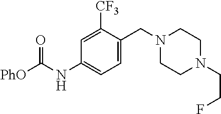 [SMILES: O=C(Nc1ccc(CN2CCN(CCF)CC2)c(C(F)(F)F)c1)Oc1ccccc1] |
| 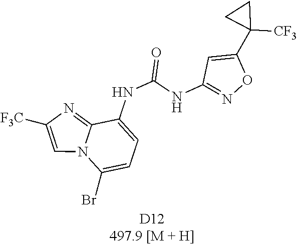 [SMILES: O=C(Nc1cc(C2(C(F)(F)F)CC2)on1)Nc1ccc(Br)n2cc(C(F)(F)F)nc12] | 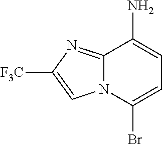 [SMILES: Nc1ccc(Br)n2cc(C(F)(F)F)nc12] | 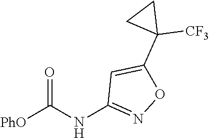 [SMILES: O=C(Nc1cc(C2(C(F)(F)F)CC2)on1)Oc1ccccc1] |
| 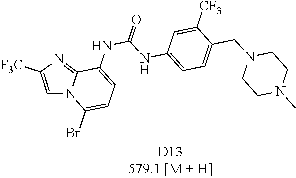 [SMILES: CN1CCN(Cc2ccc(NC(=O)Nc3ccc(Br)n4cc(C(F)(F)F)nc34)cc2C(F)(F)F)CC1] | 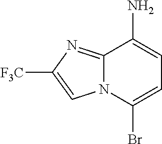 [SMILES: Nc1ccc(Br)n2cc(C(F)(F)F)nc12] | 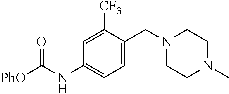 [SMILES: CN1CCN(Cc2ccc(NC(=O)Oc3ccccc3)cc2C(F)(F)F)CC1] |
| 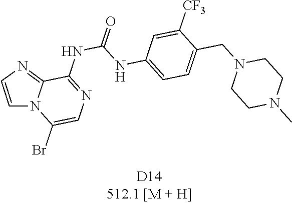 [SMILES: CN1CCN(Cc2ccc(NC(=O)Nc3ncc(Br)n4ccnc34)cc2C(F)(F)F)CC1] | 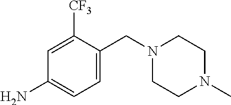 [SMILES: CN1CCN(Cc2ccc(N)cc2C(F)(F)F)CC1] | 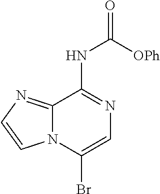 [SMILES: O=C(Nc1ncc(Br)n2ccnc12)Oc1ccccc1] |
| 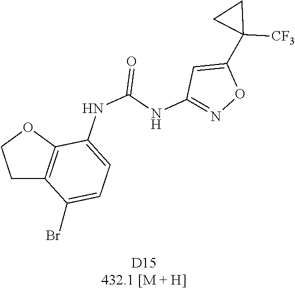 [SMILES: O=C(Nc1cc(C2(C(F)(F)F)CC2)on1)Nc1ccc(Br)c2c1OCC2] | 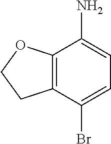 [SMILES: Nc1ccc(Br)c2c1OCC2] | 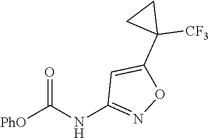 [SMILES: O=C(Nc1cc(C2(C(F)(F)F)CC2)on1)Oc1ccccc1] |
| 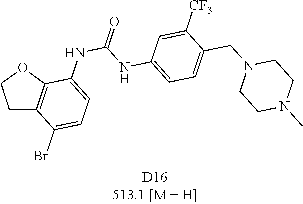 [SMILES: CN1CCN(Cc2ccc(NC(=O)Nc3ccc(Br)c4c3OCC4)cc2C(F)(F)F)CC1] | 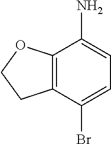 [SMILES: Nc1ccc(Br)c2c1OCC2] | 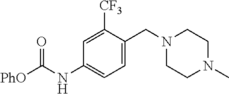 [SMILES: CN1CCN(Cc2ccc(NC(=O)Oc3ccccc3)cc2C(F)(F)F)CC1] |
| 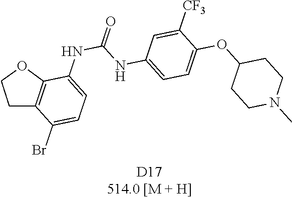 [SMILES: CN1CCC(Oc2ccc(NC(=O)Nc3ccc(Br)c4c3OCC4)cc2C(F)(F)F)CC1] | 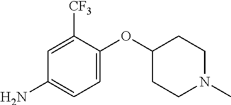 [SMILES: CN1CCC(Oc2ccc(N)cc2C(F)(F)F)CC1] | 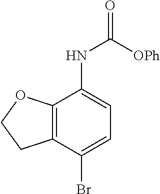 [SMILES: O=C(Nc1ccc(Br)c2c1OCC2)Oc1ccccc1] |
| 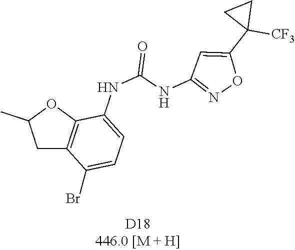 [SMILES: CC1Cc2c(Br)ccc(NC(=O)Nc3cc(C4(C(F)(F)F)CC4)on3)c2O1] | 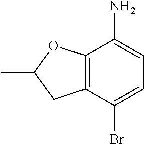 | 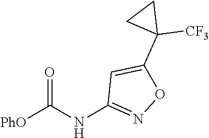 [SMILES: O=C(Nc1cc(C2(C(F)(F)F)CC2)on1)Oc1ccccc1] |
| 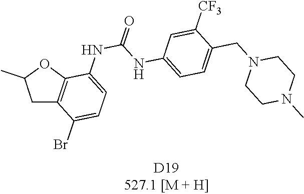 | 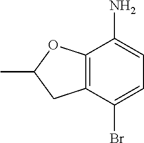 | 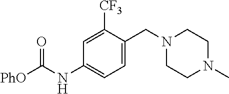 [SMILES: CN1CCN(Cc2ccc(NC(=O)Oc3ccccc3)cc2C(F)(F)F)CC1] |
| 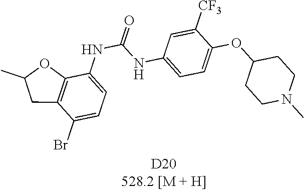 | 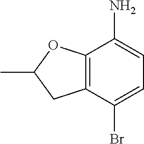 | 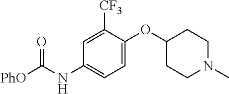 [SMILES: CN1CCC(Oc2ccc(NC(=O)Oc3ccccc3)cc2C(F)(F)F)CC1] |
| 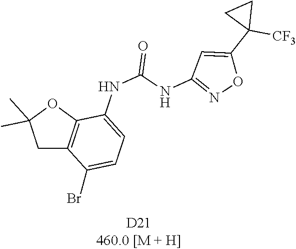 [SMILES: CC1(C)Cc2c(Br)ccc(NC(=O)Nc3cc(C4(C(F)(F)F)CC4)on3)c2O1] | 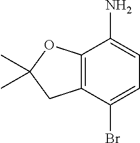 | 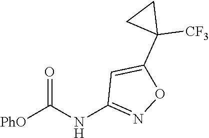 [SMILES: O=C(Nc1cc(C2(C(F)(F)F)CC2)on1)Oc1ccccc1] |
| 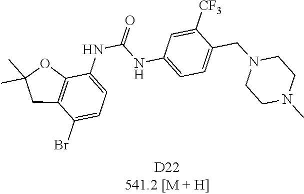 | 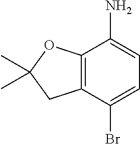 | 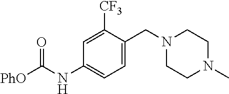 [SMILES: CN1CCN(Cc2ccc(NC(=O)Oc3ccccc3)cc2C(F)(F)F)CC1] |
| 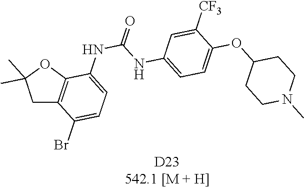 | 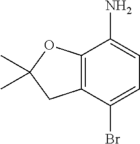 | 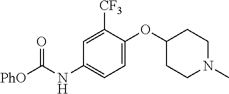 [SMILES: CN1CCC(Oc2ccc(NC(=O)Oc3ccccc3)cc2C(F)(F)F)CC1] |
| 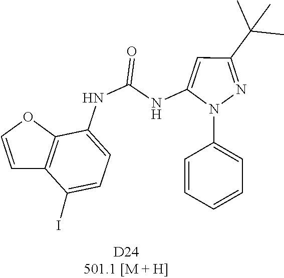 [SMILES: CC(C)(C)c1cc(NC(=O)Nc2ccc(I)c3ccoc23)n(-c2ccccc2)n1] | 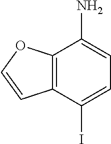 [SMILES: Nc1ccc(I)c2ccoc12] | 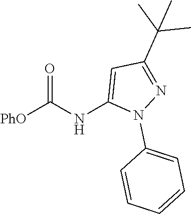 [SMILES: CC(C)(C)c1cc(NC(=O)Oc2ccccc2)n(-c2ccccc2)n1] |
| 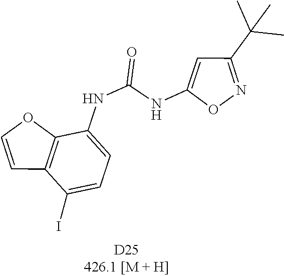 [SMILES: CC(C)(C)c1cc(NC(=O)Nc2ccc(I)c3ccoc23)on1] | 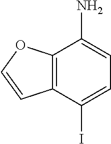 [SMILES: Nc1ccc(I)c2ccoc12] | 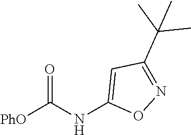 [SMILES: CC(C)(C)c1cc(NC(=O)Oc2ccccc2)on1] |
| 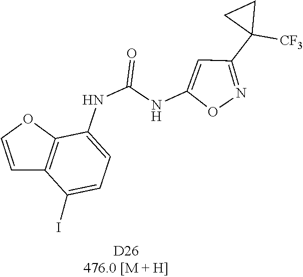 [SMILES: O=C(Nc1cc(C2(C(F)(F)F)CC2)no1)Nc1ccc(I)c2ccoc12] | 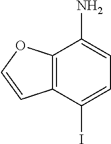 [SMILES: Nc1ccc(I)c2ccoc12] | 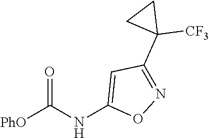 [SMILES: O=C(Nc1cc(C2(C(F)(F)F)CC2)no1)Oc1ccccc1] |
| 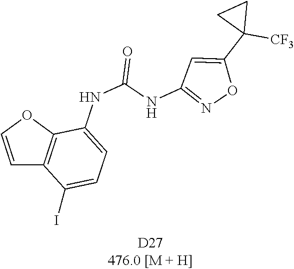 [SMILES: O=C(Nc1cc(C2(C(F)(F)F)CC2)on1)Nc1ccc(I)c2ccoc12] | 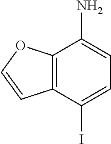 [SMILES: Nc1ccc(I)c2ccoc12] | 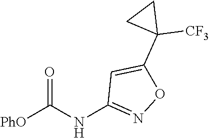 [SMILES: O=C(Nc1cc(C2(C(F)(F)F)CC2)on1)Oc1ccccc1] |
| 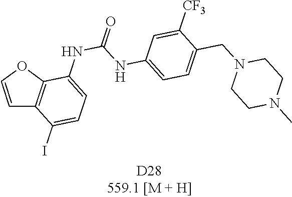 [SMILES: CN1CCN(Cc2ccc(NC(=O)Nc3ccc(I)c4ccoc34)cc2C(F)(F)F)CC1] | 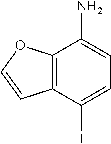 [SMILES: Nc1ccc(I)c2ccoc12] | 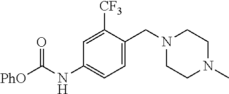 [SMILES: CN1CCN(Cc2ccc(NC(=O)Oc3ccccc3)cc2C(F)(F)F)CC1] |
| 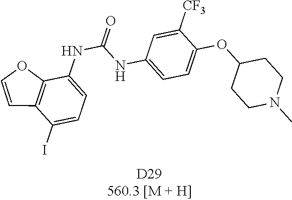 [SMILES: CN1CCC(Oc2ccc(NC(=O)Nc3ccc(I)c4ccoc34)cc2C(F)(F)F)CC1] | 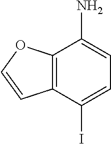 [SMILES: Nc1ccc(I)c2ccoc12] | 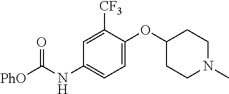 [SMILES: CN1CCC(Oc2ccc(NC(=O)Oc3ccccc3)cc2C(F)(F)F)CC1] |
| 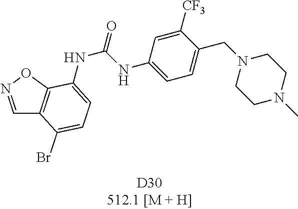 [SMILES: CN1CCN(Cc2ccc(NC(=O)Nc3ccc(Br)c4cnoc34)cc2C(F)(F)F)CC1] | 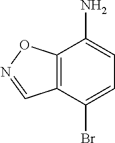 [SMILES: Nc1ccc(Br)c2cnoc12] | 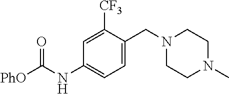 [SMILES: CN1CCN(Cc2ccc(NC(=O)Oc3ccccc3)cc2C(F)(F)F)CC1] |
| 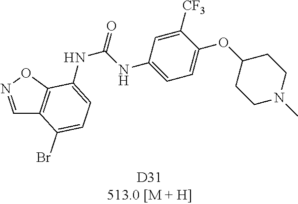 [SMILES: CN1CCC(Oc2ccc(NC(=O)Nc3ccc(Br)c4cnoc34)cc2C(F)(F)F)CC1] | 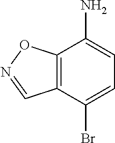 [SMILES: Nc1ccc(Br)c2cnoc12] | 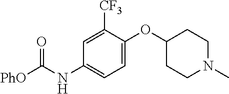 [SMILES: CN1CCC(Oc2ccc(NC(=O)Oc3ccccc3)cc2C(F)(F)F)CC1] |
| 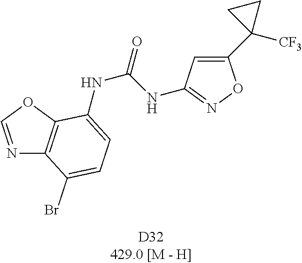 [SMILES: O=C(Nc1cc(C2(C(F)(F)F)CC2)on1)Nc1ccc(Br)c2ncoc12] | 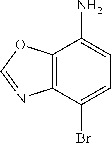 [SMILES: Nc1ccc(Br)c2ncoc12] | 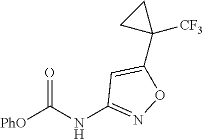 [SMILES: O=C(Nc1cc(C2(C(F)(F)F)CC2)on1)Oc1ccccc1] |
| 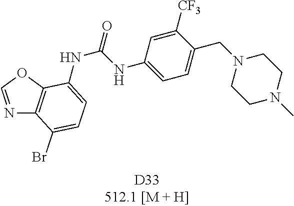 [SMILES: CN1CCN(Cc2ccc(NC(=O)Nc3ccc(Br)c4ncoc34)cc2C(F)(F)F)CC1] | 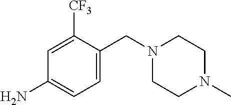 [SMILES: CN1CCN(Cc2ccc(N)cc2C(F)(F)F)CC1] | 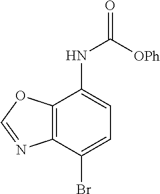 [SMILES: O=C(Nc1ccc(Br)c2ncoc12)Oc1ccccc1] |
| 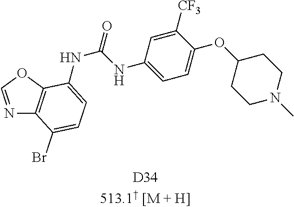 [SMILES: CN1CCC(Oc2ccc(NC(=O)Nc3ccc(Br)c4ncoc34)cc2C(F)(F)F)CC1] | 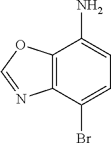 [SMILES: Nc1ccc(Br)c2ncoc12] | 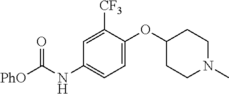 [SMILES: CN1CCC(Oc2ccc(NC(=O)Oc3ccccc3)cc2C(F)(F)F)CC1] |
| 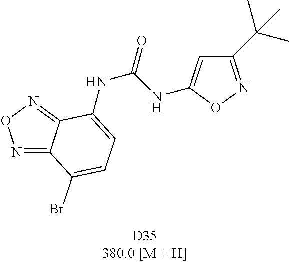 [SMILES: CC(C)(C)c1cc(NC(=O)Nc2ccc(Br)c3nonc23)on1] | 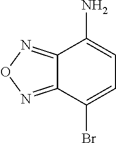 [SMILES: Nc1ccc(Br)c2nonc12] | 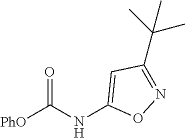 [SMILES: CC(C)(C)c1cc(NC(=O)Oc2ccccc2)on1] |
| 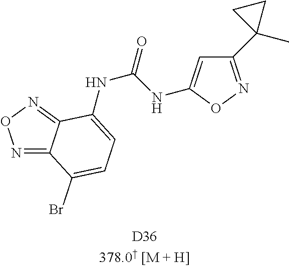 [SMILES: CC1(c2cc(NC(=O)Nc3ccc(Br)c4nonc34)on2)CC1] | 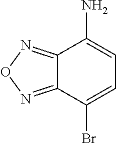 [SMILES: Nc1ccc(Br)c2nonc12] | 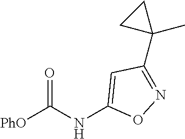 [SMILES: CC1(c2cc(NC(=O)Oc3ccccc3)on2)CC1] |
| 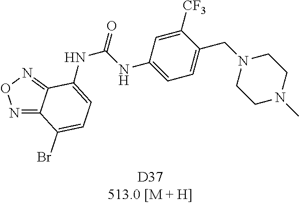 [SMILES: CN1CCN(Cc2ccc(NC(=O)Nc3ccc(Br)c4nonc34)cc2C(F)(F)F)CC1] | 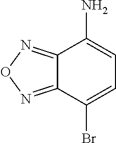 [SMILES: Nc1ccc(Br)c2nonc12] | 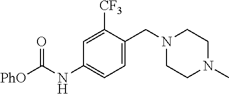 [SMILES: CN1CCN(Cc2ccc(NC(=O)Oc3ccccc3)cc2C(F)(F)F)CC1] |
| 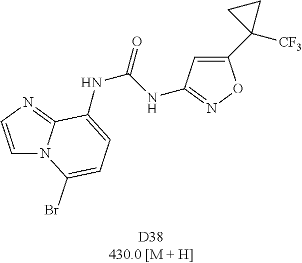 [SMILES: O=C(Nc1cc(C2(C(F)(F)F)CC2)on1)Nc1ccc(Br)n2ccnc12] | 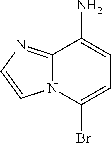 [SMILES: Nc1ccc(Br)n2ccnc12] | 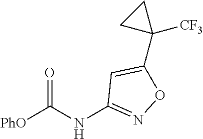 [SMILES: O=C(Nc1cc(C2(C(F)(F)F)CC2)on1)Oc1ccccc1] |
| 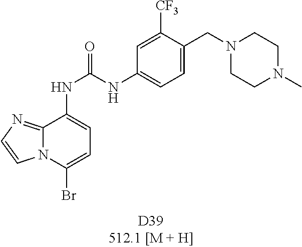 [SMILES: CN1CCN(Cc2ccc(NC(=O)Nc3ccc(Br)n4ccnc34)cc2C(F)(F)F)CC1] | 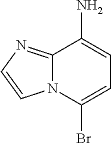 [SMILES: Nc1ccc(Br)n2ccnc12] | 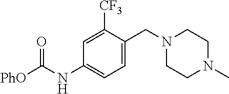 [SMILES: CN1CCN(Cc2ccc(NC(=O)Oc3ccccc3)cc2C(F)(F)F)CC1] |
| 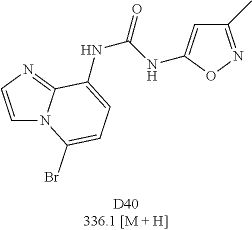 [SMILES: Cc1cc(NC(=O)Nc2ccc(Br)n3ccnc23)on1] | 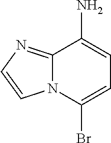 [SMILES: Nc1ccc(Br)n2ccnc12] | 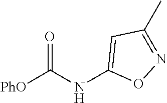 [SMILES: Cc1cc(NC(=O)Oc2ccccc2)on1] |
| 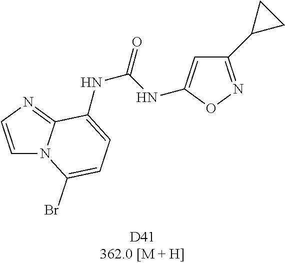 [SMILES: O=C(Nc1cc(C2CC2)no1)Nc1ccc(Br)n2ccnc12] | 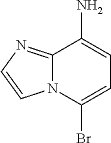 [SMILES: Nc1ccc(Br)n2ccnc12] | 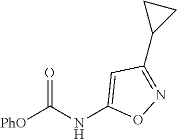 [SMILES: O=C(Nc1cc(C2CC2)no1)Oc1ccccc1] |
| 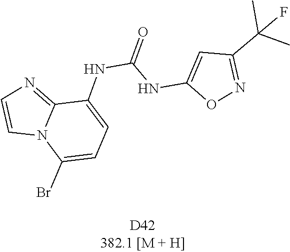 [SMILES: CC(C)(F)c1cc(NC(=O)Nc2ccc(Br)n3ccnc23)on1] | 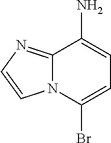 [SMILES: Nc1ccc(Br)n2ccnc12] | 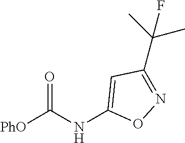 [SMILES: CC(C)(F)c1cc(NC(=O)Oc2ccccc2)on1] |
| 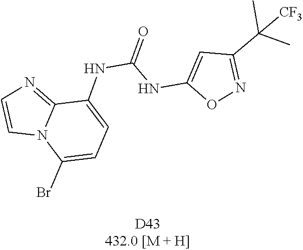 [SMILES: CC(C)(c1cc(NC(=O)Nc2ccc(Br)n3ccnc23)on1)C(F)(F)F] | 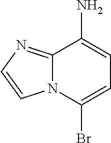 [SMILES: Nc1ccc(Br)n2ccnc12] | 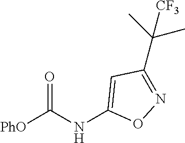 [SMILES: CC(C)(c1cc(NC(=O)Oc2ccccc2)on1)C(F)(F)F] |
| 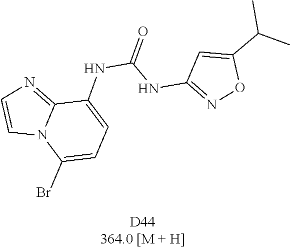 [SMILES: CC(C)c1cc(NC(=O)Nc2ccc(Br)n3ccnc23)no1] | 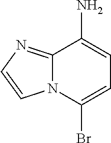 [SMILES: Nc1ccc(Br)n2ccnc12] | 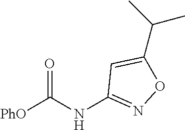 [SMILES: CC(C)c1cc(NC(=O)Oc2ccccc2)no1] |
| 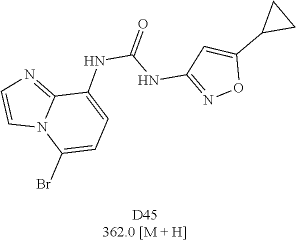 [SMILES: O=C(Nc1cc(C2CC2)on1)Nc1ccc(Br)n2ccnc12] | 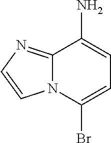 [SMILES: Nc1ccc(Br)n2ccnc12] | 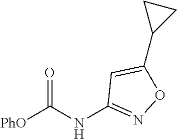 [SMILES: O=C(Nc1cc(C2CC2)on1)Oc1ccccc1] |
| 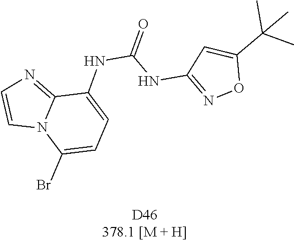 [SMILES: CC(C)(C)c1cc(NC(=O)Nc2ccc(Br)n3ccnc23)no1] | 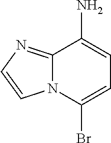 [SMILES: Nc1ccc(Br)n2ccnc12] | 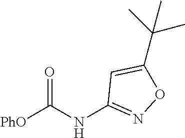 [SMILES: CC(C)(C)c1cc(NC(=O)Oc2ccccc2)no1] |
| 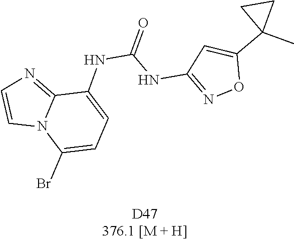 [SMILES: CC1(c2cc(NC(=O)Nc3ccc(Br)n4ccnc34)no2)CC1] | 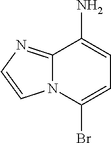 [SMILES: Nc1ccc(Br)n2ccnc12] | 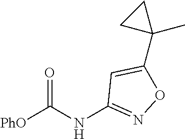 [SMILES: CC1(c2cc(NC(=O)Oc3ccccc3)no2)CC1] |
| 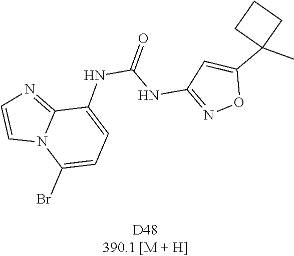 [SMILES: CC1(c2cc(NC(=O)Nc3ccc(Br)n4ccnc34)no2)CCC1] | 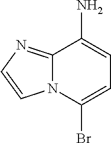 [SMILES: Nc1ccc(Br)n2ccnc12] | 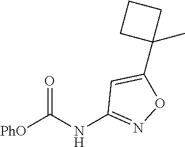 [SMILES: CC1(c2cc(NC(=O)Oc3ccccc3)no2)CCC1] |
| 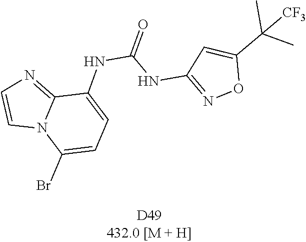 [SMILES: CC(C)(c1cc(NC(=O)Nc2ccc(Br)n3ccnc23)no1)C(F)(F)F] | 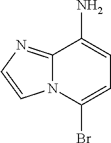 [SMILES: Nc1ccc(Br)n2ccnc12] | 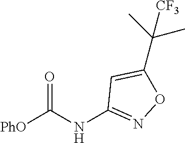 [SMILES: CC(C)(c1cc(NC(=O)Oc2ccccc2)no1)C(F)(F)F] |
| 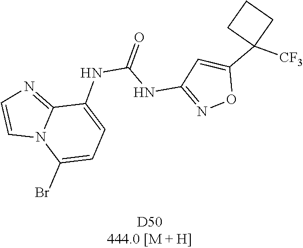 [SMILES: O=C(Nc1cc(C2(C(F)(F)F)CCC2)on1)Nc1ccc(Br)n2ccnc12] | 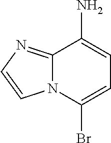 [SMILES: Nc1ccc(Br)n2ccnc12] | 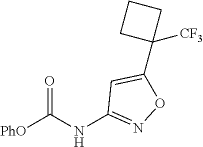 [SMILES: O=C(Nc1cc(C2(C(F)(F)F)CCC2)on1)Oc1ccccc1] |
| 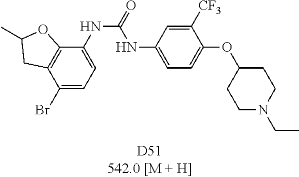 [SMILES: CCN1CCC(Oc2ccc(NC(=O)Nc3ccc(Br)c4c3OC(C)C4)cc2C(F)(F)F)CC1] | 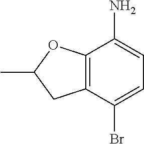 | 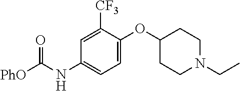 [SMILES: CCN1CCC(Oc2ccc(NC(=O)Oc3ccccc3)cc2C(F)(F)F)CC1] |
| 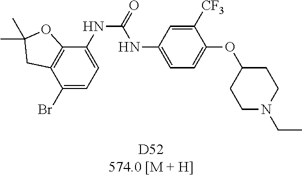 [SMILES: CCN1CCC(Oc2ccc(NC(=O)Nc3ccc(Br)c4c3OC(C)(C)C4)cc2C(F)(F)F)CC1] | 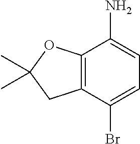 | 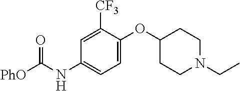 [SMILES: CCN1CCC(Oc2ccc(NC(=O)Oc3ccccc3)cc2C(F)(F)F)CC1] |
| 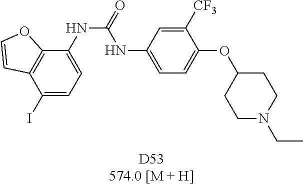 [SMILES: CCN1CCC(Oc2ccc(NC(=O)Nc3ccc(I)c4ccoc34)cc2C(F)(F)F)CC1] | 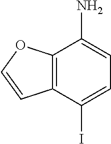 [SMILES: Nc1ccc(I)c2ccoc12] | 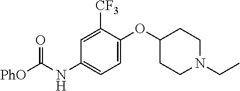 [SMILES: CCN1CCC(Oc2ccc(NC(=O)Oc3ccccc3)cc2C(F)(F)F)CC1] |
| 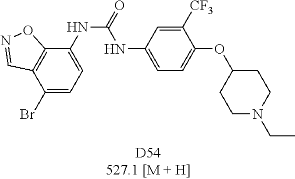 [SMILES: CCN1CCC(Oc2ccc(NC(=O)Nc3ccc(Br)c4cnoc34)cc2C(F)(F)F)CC1] | 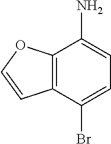 [SMILES: Nc1ccc(Br)c2ccoc12] | 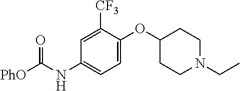 [SMILES: CCN1CCC(Oc2ccc(NC(=O)Oc3ccccc3)cc2C(F)(F)F)CC1] |

^{†}Expected value

All amine and carbamate starting materials used for the synthesis of Intermediates D are commercially available except for the following:
4-((1-Methylpiperidin-4-yl)oxy)-3-(trifluoromethyl)aniline (precursor to D4 and D17) was synthesized as reported in PCT Publication No. WO 2017/125530.
5-Bromoimidazo[1,2-a]pyridin-8-amine (precursor to D7-D11, D38, and D50) was synthesized as reported in PCT Publication No. WO 2020/081508.
5-Bromo-2-(trifluoromethyl)imidazo[1,2-a]pyridin-8-amine (precursor to D12 and D13) was synthesized as reported in PCT Publication No. WO 2010/035745.
4-Bromo-2,3-dihydrobenzofuran-7-amine (precursor to D15 and D16) was synthesized as reported in PCT Publication No. WO 2021/244582.
4-Bromo-2-methyl-2,3-dihydrobenzofuran-7-amine (precursor to D18-D20 and D51) was synthesized as reported in PCT Publication No. WO 2016/169504.
4-Iodobenzofuran-7-amine (precursor to D24-D29 and D53) was synthesized as reported in PCT Publication No. WO 2004/041811.
7-Bromobenzo[c][1,2,5]oxadiazol-4-amine (precursor to D35-D37) was synthesized as reported in PCT Publication No. WO 2003/062241.

### Synthesis of 4-bromobenzo[d]isoxazol-7-amine (Precursor to D30-D31 and D54)

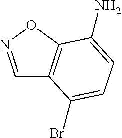 [SMILES: Nc1ccc(Br)c2cnoc12]

The title compound was prepared by following a similar procedure described for step 2 of Intermediate A1, starting from benzo[d]isoxazol-7-amine (0.100 g, 0.746 mmol) and NBS (0.133 g, 0.746 mmol), and was isolated as a pale yellow solid (135 mg, 85% yield). ¹H NMR (400 MHz, DMSO-d₆) δ=9.13 (s, 1H), 7.25 (d, J=8.0 Hz, 1H), 6.72 (d, J=8.4 Hz, 1H), 5.95 (s, 2H). MS: 212.9 [M+H].

### Synthesis of 4-bromobenzo[d]oxazol-7-amine (Precursor to D32 and D34)

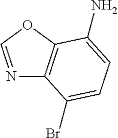 [SMILES: Nc1ccc(Br)c2ncoc12]

The title compound was prepared by following a similar procedure described for step 2 of Intermediate A1, starting from benzo[d]oxazol-7-amine (0.100 g, 0.746 mmol) and NBS (0.133 g, 0.746 mmol), and was isolated as a pale brown solid (0.150 g, 94% yield) following purification by flash chromatography (silica gel 230-400 mesh, eluting with 15% EtOAc in petroleum ether). MS: 212.9 [M+H].

### Intermediates E1-E14

### General Stannylation Procedure for the Synthesis of Intermediates E:

Hexamethylditin (1.2 eq) was added to a solution of heteroaryl halide (1.0 eq) in toluene (10 vol.) and the resulting mixture was purged with N₂ for 10 minutes. Trans-dichlorobis(triphenylphosphine)palladium(II) (0.03 eq) was then added and the resulting mixture was subjected to microwave irradiation at 140° C. for 30 min. Following completion of the reaction (as indicated by LCMS), the reaction mixture was filtered through a pad of Celite® (i.e., diatomaceous earth) which was then washed with EtOAc (2×5 vol.). The combined filtrates were concentrated under reduced pressure to afford the title product which was used without further purification.

The following intermediates were prepared using the above general procedure:

| Product | Starting material | MS |
|---|---|---|
| 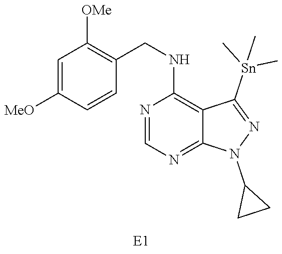 [SMILES: COc1ccc(CNc2ncnc3c2[c]([Sn]([CH3])([CH3])[CH3])nn3C2CC2)c(OC)c1] | 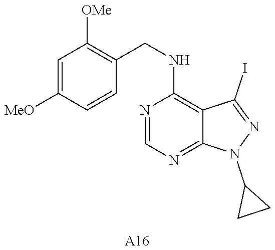 [SMILES: COc1ccc(CNc2ncnc3c2c(I)nn3C2CC2)c(OC)c1 ; "A16"] | 490.0 [M + H] |
| 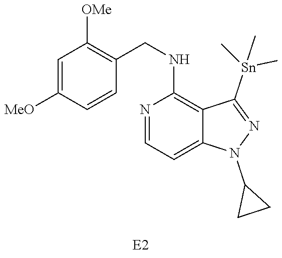 [SMILES: COc1ccc(CNc2nccc3c2[c]([Sn]([CH3])([CH3])[CH3])nn3C2CC2)c(OC)c1] | 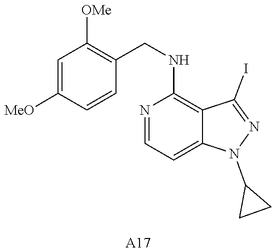 [SMILES: COc1ccc(CNc2nccc3c2c(I)nn3C2CC2)c(OC)c1 ; "A17"] | 489.1^{†} [M + H] |
| 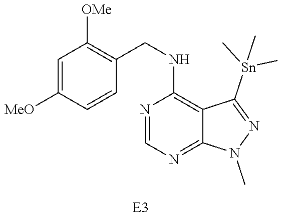 [SMILES: COc1ccc(CNc2ncnc3c2[c]([Sn]([CH3])([CH3])[CH3])nn3C)c(OC)c1] | 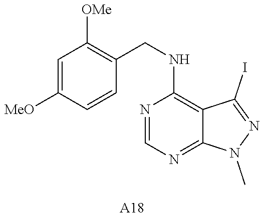 | 464.1^{†} [M + H] |
| 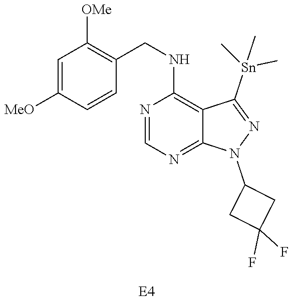 [SMILES: COc1ccc(CNc2ncnc3c2[c]([Sn]([CH3])([CH3])[CH3])nn3C2CC(F)(F)C2)c(OC)c1] | 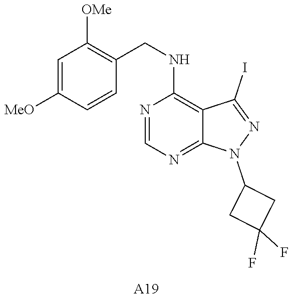 [SMILES: COc1ccc(CNc2ncnc3c2c(I)nn3C2CC(F)(F)C2)c(OC)c1 ; "A19"] | 540.1^{†} [M + H] |
| 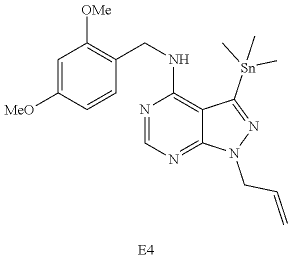 [SMILES: C=CCn1n[c]([Sn]([CH3])([CH3])[CH3])c2c(NCc3ccc(OC)cc3OC)ncnc21] | 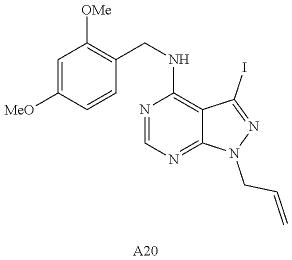 | 490.1^{†} [M + H] |
| 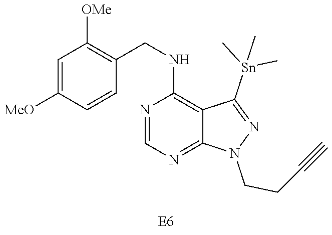 [SMILES: C#CCCn1n[c]([Sn]([CH3])([CH3])[CH3])c2c(NCc3ccc(OC)cc3OC)ncnc21] | 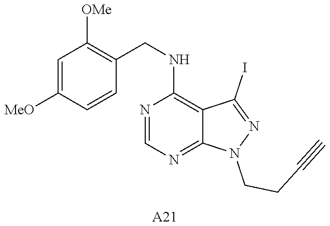 | 502.1^{†} [M + H] |
| 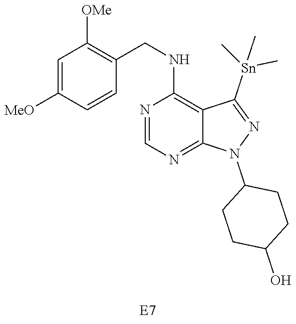 [SMILES: COc1ccc(CNc2ncnc3c2[c]([Sn]([CH3])([CH3])[CH3])nn3C2CCC(O)CC2)c(OC)c1] | 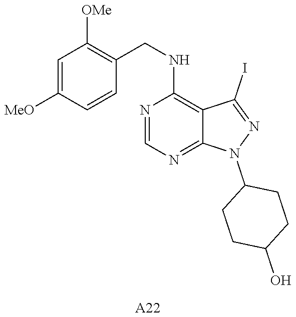 [SMILES: COc1ccc(CNc2ncnc3c2c(I)nn3C2CCC(O)CC2)c(OC)c1 ; "A22"] | 548.2^{†} [M + H] |
| 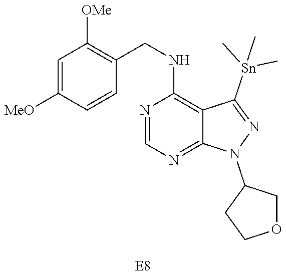 [SMILES: COc1ccc(CNc2ncnc3c2[c]([Sn]([CH3])([CH3])[CH3])nn3C2CCOC2)c(OC)c1] | 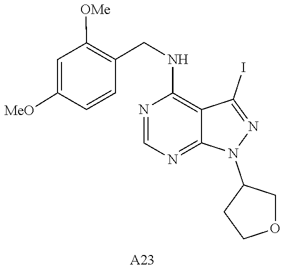 [SMILES: COc1ccc(CNc2ncnc3c2c(I)nn3C2CCOC2)c(OC)c1 ; "A23"] | 520.1^{†} [M + H] |
| 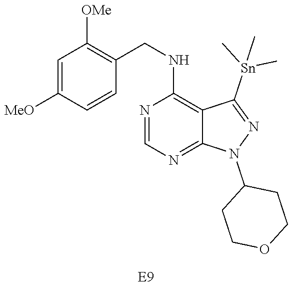 [SMILES: COc1ccc(CNc2ncnc3c2[c]([Sn]([CH3])([CH3])[CH3])nn3C2CCOCC2)c(OC)c1] | 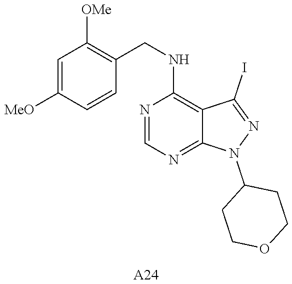 [SMILES: COc1ccc(CNc2ncnc3c2c(I)nn3C2CCOCC2)c(OC)c1 ; "A24"] | 534.1^{†} [M + H] |
| 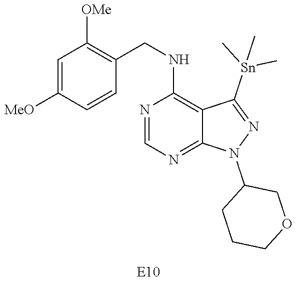 [SMILES: COc1ccc(CNc2ncnc3c2[c]([Sn]([CH3])([CH3])[CH3])nn3C2CCCOC2)c(OC)c1] | 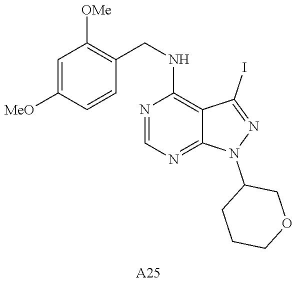 [SMILES: COc1ccc(CNc2ncnc3c2c(I)nn3C2CCCOC2)c(OC)c1 ; "A25"] | 534.1^{†} [M + H] |
| 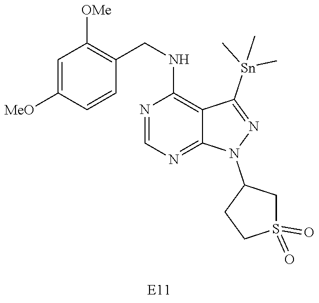 [SMILES: COc1ccc(CNc2ncnc3c2[c]([Sn]([CH3])([CH3])[CH3])nn3C2CCS(=O)(=O)C2)c(OC)c1] | 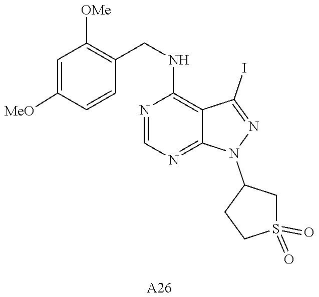 [SMILES: COc1ccc(CNc2ncnc3c2c(I)nn3C2CCS(=O)(=O)C2)c(OC)c1 ; "A26"] | 568.1^{†} [M + H] |
| 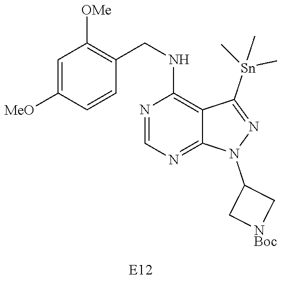 [SMILES: COc1ccc(CNc2ncnc3c2[c]([Sn]([CH3])([CH3])[CH3])nn3C2CN(C(=O)OC(C)(C)C)C2)c(OC)c1] | 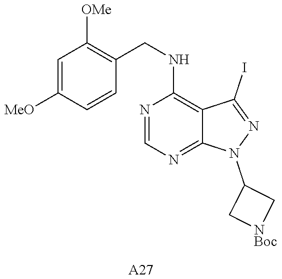 [SMILES: COc1ccc(CNc2ncnc3c2c(I)nn3C2CN(C(=O)OC(C)(C)C)C2)c(OC)c1 ; "A27"] | 605.2^{†} [M + H] |
| 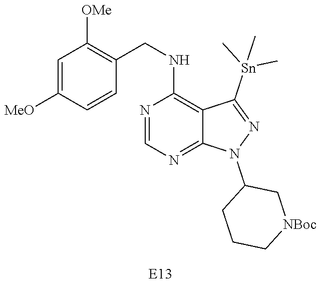 [SMILES: COc1ccc(CNc2ncnc3c2[c]([Sn]([CH3])([CH3])[CH3])nn3C2CCCN(C(=O)OC(C)(C)C)C2)c(OC)c1] | 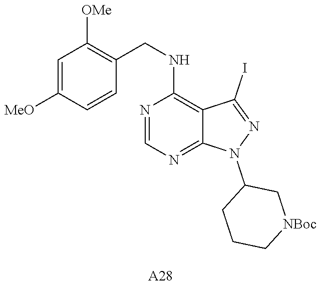 [SMILES: COc1ccc(CNc2ncnc3c2c(I)nn3C2CCCN(C(=O)OC(C)(C)C)C2)c(OC)c1 ; "A28"] | 633.2^{†} [M + H] |
| 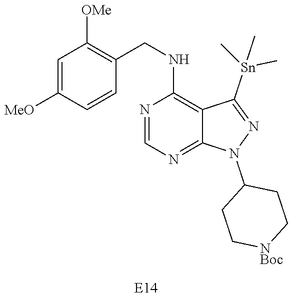 [SMILES: COc1ccc(CNc2ncnc3c2[c]([Sn]([CH3])([CH3])[CH3])nn3C2CCN(C(=O)OC(C)(C)C)CC2)c(OC)c1] | 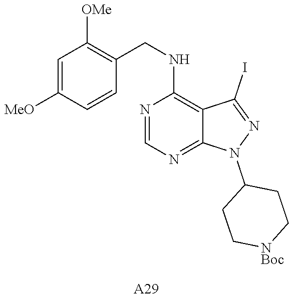 [SMILES: COc1ccc(CNc2ncnc3c2c(I)nn3C2CCN(C(=O)OC(C)(C)C)CC2)c(OC)c1 ; "A29"] | 633.2^{†} [M + H] |

^{†}Expected Value

### Preparation of Examples

### General Urea Formation Procedure for the Synthesis of Examples 1-118

Method A—Triethylamine (2.0 eq.) was added to a mixture of amine starting material (1.0 eq.) and carbamate starting material (1.0 eq.) in THF (10 Vol.) and the resulting mixture was stirred at 60° C. for 12 h in a sealed tube. Following completion of the reaction (as indicated by LCMS), the reaction mixture was concentrated under reduced pressure to give crude material which was purified by reverse phase preparative HPLC to afford the desired product.

Method B—DMAP (0.05 eq.) and DIPEA (1.5 eq.) were added to a solution of amine starting material (1.0 eq.) and carbamate starting material (1.0 eq.) in THF (10 Vol.) and the resulting mixture was stirred at 60° C. for 12 h in a sealed tube. Following completion of the reaction (as indicated by LCMS), the reaction mixture was concentrated under reduced pressure to yield the crude material which was purified by reverse phase preparative HPLC to afford the desired product.

The following compounds were prepared using the above general procedures.

### Example 1

### 1-(4-(4-Amino-7-Cyclopropyl-7H-Pyrrolo[2,3-D]Pyrimidin-5-Yl)Naphthalen-1-Yl)-3-(3-(1-(Trifluoromethyl)Cyclopropyl)Isoxazol-5-Yl)Urea

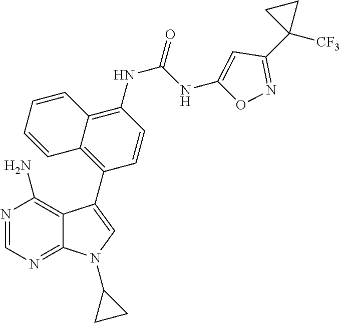 [SMILES: Nc1ncnc2c1c(-c1ccc(NC(=O)Nc3cc(C4(C(F)(F)F)CC4)no3)c3ccccc13)cn2C1CC1]

The title compound was obtained by following the general procedure for urea formation (Method A), starting from 5-(4-aminonaphthalen-1-yl)-7-cyclopropyl-7H-pyrrolo[2,3-d]pyrimidin-4-amine (B1, 80 mg, 0.254 mmol) and phenyl (3-(1-(trifluoromethyl)cyclopropyl)isoxazol-5-yl)carbamate (C1, 79 mg, 0.254 mmol), and was isolated as a grey solid (8 mg, 6% yield). ¹H NMR (400 MHz, DMSO-d₆) δ=9.94 (bs, 1H), 8.12-8.17 (m, 2H), 7.93 (d, J=7.6 Hz, 1H), 7.80 (d, J=8.0 Hz, 1H), 7.61-7.63 (m, 2H), 7.46-7.55 (m, 1H), 7.25 (s, 1H), 6.17 (s, 1H), 3.58-3.63 (m, 1H), 1.35-1.46 (m, 4H), 1.05-1.09 (m, 4H). MS: 534.2 [M+H].

### Example 2

### 1-(5-(4-Amino-7-Cyclopropyl-7H-Pyrrolo[2,3-D]Pyrimidin-5-Yl)Quinolin-8-Yl)-3-(3-(Tert-Butyl)Isoxazol-5-Yl)Urea

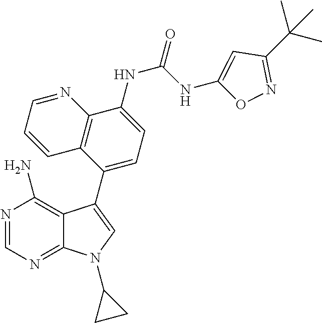 [SMILES: CC(C)(C)c1cc(NC(=O)Nc2ccc(-c3cn(C4CC4)c4ncnc(N)c34)c3cccnc23)on1]

The title compound was obtained by following a similar procedure described for Intermediate B1, starting from 1-(5-bromoquinolin-8-yl)-3-(3-(tert-butyl)isoxazol-5-yl)urea (D1, 101 mg, 0.260 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 130 mg, 0.260 mmol), and was isolated as a pale-yellow solid (2 mg, 1.6% yield). Cleavage of the di-Boc protecting group was observed during the reaction. ¹H NMR (400 MHz, CD₃OD) δ=8.95-8.96 (m, 1H), 8.67 (d, J=8.00 Hz, 1H), 8.39 (s, 1H), 8.22-8.25 (m, 1H), 7.58-7.65 (m, 2H), 7.53 (s, 1H), 6.24 (s, 1H), 3.77-3.80 (m, 1H), 1.37 (s, 9H), 1.24-1.25 (m, 4H). MS: 483.3 [M+H].

### Example 3

### 1-(5-(4-Amino-7-Cyclopropyl-7H-Pyrrolo[2,3-D]Pyrimidin-5-Yl)Quinolin-8-Yl)-3-(3-(1-(Trifluoromethyl)Cyclopropyl)Isoxazol-5-Yl)Urea

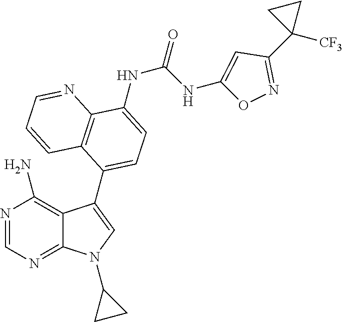 [SMILES: Nc1ncnc2c1c(-c1ccc(NC(=O)Nc3cc(C4(C(F)(F)F)CC4)no3)c3ncccc13)cn2C1CC1]

The title compound was obtained by following a similar procedure described for Intermediate B1, starting from 1-(5-bromoquinolin-8-yl)-3-(3-(1-(trifluoromethyl)cyclopropyl)isoxazol-5-yl)urea (D2, 150 mg, 0.340 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 170 mg, 0.340 mmol), and was isolated as a pale-yellow solid (25 mg, 14 yield). Cleavage of the di-Boc protecting group was observed during the reaction. ¹H NMR (400 MHz, DMSO-d₆) δ=11.50 (s, 1H), 10.16 (s, 1H), 8.97-8.98 (m, 1H), 8.61 (d, J=8.0 Hz, 1H), 8.39 (s, 1H), 8.22-8.24 (m, 1H), 7.62-7.65 (m, 1H), 7.57 (d, J=8.0 Hz, 1H), 7.51 (s, 1H), 6.28 (s, 1H), 3.72-3.75 (m, 1H), 1.40-1.50 (m, 4H), 1.10-1.16 (m, 4H). MS: 535.2 [M+H].

### Example 4

### 1-(5-(4-Amino-7-Cyclopropyl-7H-Pyrrolo[2,3-D]Pyrimidin-5-Yl)Quinolin-8-Yl)-3-(5-(1-(Trifluoromethyl)Cyclopropyl)Isoxazol-3-Yl)Urea

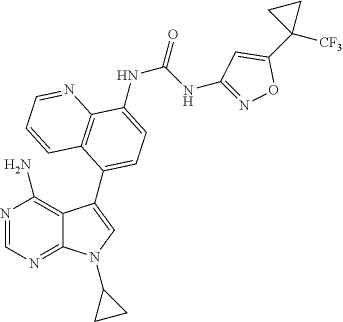 [SMILES: Nc1ncnc2c1c(-c1ccc(NC(=O)Nc3cc(C4(C(F)(F)F)CC4)on3)c3ncccc13)cn2C1CC1]

The title compound was obtained by following the general procedure for urea formation (Method A), starting from 5-(4-amino-7-cyclopropyl-7H-pyrrolo[2,3-d]pyrimidin-5-yl)quinolin-8-amine (B2, 30 mg, 0.095 mmol) and phenyl (5-(1-(trifluoromethyl)cyclopropyl)isoxazol-3-yl)carbamate (C3, 30 mg, 0.095 mmol), and was isolated as an off-white solid (3 mg, 5.9% yield). ¹H NMR (400 MHz, CD₃OD) δ=8.93-8.94 (m, 1H), 8.63-8.65 (m, 1H), 8.21-8.23 (m, 2H), 7.54-7.61 (m, 2H), 7.23 (s, 1H), 6.91 (s, 1H), 3.57-3.61 (m, 1H), 1.50-1.59 (m, 4H), 1.14-1.18 (m, 4H). MS: 535.4 [M+H].

### Example 5

### 1-(5-(4-Amino-7-Cyclopropyl-7H-Pyrrolo[2,3-D]Pyrimidin-5-Yl)Isoquinolin-8-Yl)-3-(3-(Tert-Butyl)Isoxazol-5-Yl)Urea

### Step 1: Synthesis of tert-butyl (tert-butoxycarbonyl)(5-(8-(3-(3-(tert-butyl)isoxazol-5-yl)ureido)isoquinolin-5-yl)-7-cyclopropyl-7H-pyrrolo[2,3-d]pyrimidin-4-yl)carbamate

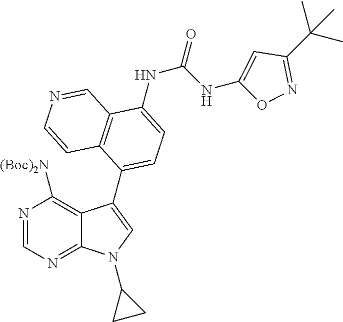

The title compound was obtained by following the general procedure for urea formation (Method B), starting from tert-butyl (5-(8-aminoisoquinolin-5-yl)-7-cyclopropyl-7H-pyrrolo[2,3-d]pyrimidin-4-yl)(tert-butoxycarbonyl)carbamate (B3, 95 mg, 0.184 mmol) and phenyl (3-(tert-butyl)isoxazol-5-yl)carbamate (C2, 48 mg, 0.184 mmol), and was isolated as an off-white solid (17 mg, 13% yield). MS: 683.5 [M+H].

### Step 2: Synthesis of 1-(5-(4-amino-7-cyclopropyl-7H-pyrrolo[2,3-d]pyrimidin-5-yl)isoquinolin-8-yl)-3-(3-(tert-butyl)isoxazol-5-yl)urea

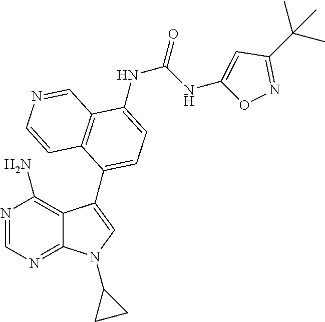 [SMILES: CC(C)(C)c1cc(NC(=O)Nc2ccc(-c3cn(C4CC4)c4ncnc(N)c34)c3ccncc23)on1]

TFA (0.075 mL) was added to a stirred of tert-butyl (tert-butoxycarbonyl)(5-(8-(3-(3-(tert-butyl)isoxazol-5-yl)ureido)isoquinolin-5-yl)-7-cyclopropyl-7H-pyrrolo[2,3-d]pyrimidin-4-yl)carbamate (15 mg, 0.022 mmol) in DCM (2 mL) at 0° C. and the resulting mixture was stirred at 25° C. for 12 h. Following completion of the reaction (as indicated by LCMS), the reaction mixture was concentrated under reduced pressure, giving crude material which was purified by preparative HPLC (eluting with 10 mM NH₄OAc in H₂O and ACN), affording the title compound as a yellow solid (7 mg, 66% yield). ¹H NMR (400 MHz, DMSO-d₆) δ=10.46 (s, 1H), 9.60 (s, 1H), 9.54 (s, 1H), 8.54 (d, J=6.0 Hz, 1H), 8.45 (s, 1H), 8.25 (d, J=8.0 Hz, 1H), 7.71-7.79 (m, 2H), 7.61 (s, 1H), 6.13 (s, 1H), 3.75-3.78 (m, 1H), 1.29 (s, 9H), 1.12-1.18 (m, 4H). MS: 483.3 [M+H].

### Example 6

### 1-(5-(4-Amino-7-Cyclopropyl-7H-Pyrrolo[2,3-D]Pyrimidin-5-Yl)Isoquinolin-8-Yl)-3-(3-(1-Methylcyclopropyl)Isoxazol-5-Yl)Urea

### Step 1: Synthesis of tert-butyl (tert-butoxycarbonyl)(7-cyclopropyl-5-(8-(3-(3-(1-methylcyclopropyl)isoxazol-5-yl)ureido)isoquinolin-5-yl)-7H-pyrrolo[2,3-d]pyrimidin-4-yl)carbamate

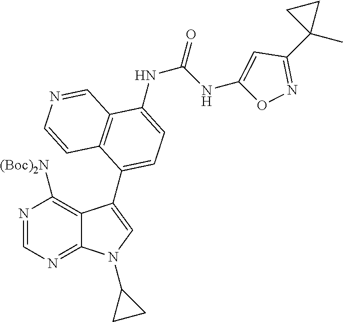

The title compound was obtained by following the general procedure for urea formation (Method B), starting from tert-butyl (5-(8-aminoisoquinolin-5-yl)-7-cyclopropyl-7H-pyrrolo[2,3-d]pyrimidin-4-yl)(tert-butoxycarbonyl)carbamate (B3, 50 mg, 0.097 mmol) and phenyl (3-(1-methylcyclopropyl)isoxazol-5-yl)carbamate (C4, 25 mg, 0.097 mmol), and was isolated as a pale-yellow solid (12 mg, 18% yield). MS: 681.3 [M+H].

### Step 2: Synthesis of 1-(5-(4-amino-7-cyclopropyl-7H-pyrrolo[2,3-d]pyrimidin-5-yl)isoquinolin-8-yl)-3-(3-(1-methylcyclopropyl)isoxazol-5-yl)urea

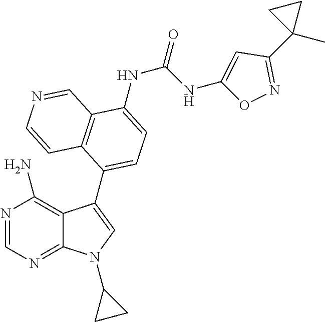 [SMILES: CC1(c2cc(NC(=O)Nc3ccc(-c4cn(C5CC5)c5ncnc(N)c45)c4ccncc34)on2)CC1]

The title compound was obtained by following a similar procedure described for step 2 of Example 5, starting from tert-butyl (tert-butoxycarbonyl)(7-cyclopropyl-5-(8-(3-(3-(1-methylcyclopropyl)isoxazol-5-yl)ureido)isoquinolin-5-yl)-7H-pyrrolo[2,3-d]pyrimidin-4-yl)carbamate (10 mg, 0.015 mmol), and was isolated as a yellow solid (2 mg, 28% yield). ¹H NMR (400 MHz, CD₃OD) δ=9.48 (s, 1H), 8.49 (d, J=6.0 Hz, 1H), 8.24 (s, 1H), 8.07 (d, J=8.0 Hz, 1H), 7.83 (d, J=7.6 Hz, 1H), 7.76-7.78 (m, 1H), 7.29 (s, 1H), 5.95 (s, 1H), 3.58-3.59 (m, 1H), 1.46 (s, 3H), 1.14-1.21 (m, 4H), 1.03-1.05 (m, 2H), 0.87-0.90 (m, 2H). MS: 481.4 [M+H].

### Example 7

### 1-(5-(4-Amino-7-Cyclopropyl-7H-Pyrrolo[2,3-D]Pyrimidin-5-Yl)Isoquinolin-8-Yl)-3-(3-(1-(Trifluoromethyl)Cyclopropyl)Isoxazol-5-Yl)Urea

### Step 1: Synthesis of tert-butyl (tert-butoxycarbonyl)(7-cyclopropyl-5-(8-(3-(3-(1-(trifluoromethyl)cyclopropyl)isoxazol-5-yl)ureido)isoquinolin-5-yl)-7H-pyrrolo[2,3-d]pyrimidin-4-yl)carbamate

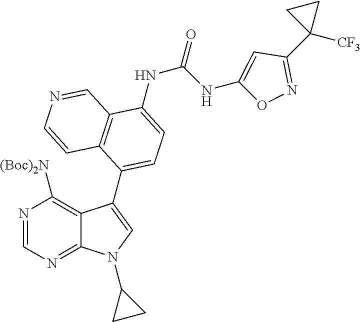

The title compound was obtained by following a similar procedure described for Intermediate B1, starting from 1-(5-bromoisoquinolin-8-yl)-3-(3-(1-(trifluoromethyl)cyclopropyl)isoxazol-5-yl)urea (D3, 51 mg, 0.116 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 58 mg, 0.116 mmol), and was isolated as a pale-yellow solid (30 mg, 35% yield). MS: 735.3 [M+H].

### Step 2: Synthesis of 1-(5-(4-amino-7-cyclopropyl-7H-pyrrolo[2,3-d]pyrimidin-5-yl)isoquinolin-8-yl)-3-(3-(1-(trifluoromethyl)cyclopropyl)isoxazol-5-yl)urea

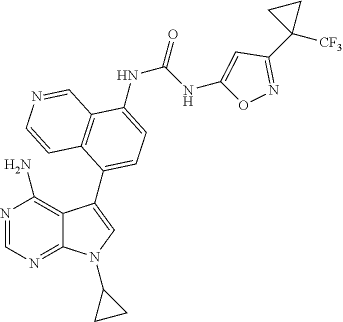 [SMILES: Nc1ncnc2c1c(-c1ccc(NC(=O)Nc3cc(C4(C(F)(F)F)CC4)no3)c3cnccc13)cn2C1CC1]

The title compound was obtained by following a similar procedure described for step 2 of Example 5, starting from tert-butyl (tert-butoxycarbonyl)(7-cyclopropyl-5-(8-(3-(3-(1-(trifluoromethyl)cyclopropyl)isoxazol-5-yl)ureido)isoquinolin-5-yl)-7H-pyrrolo[2,3-d]pyrimidin-4-yl)carbamate (30 mg, 0.041 mmol), and was isolated as a yellow solid (1.5 mg, 6.8% yield). MS: 535.2 [M+H].

### Example 8

### 1-(5-(4-Amino-7-Cyclopropyl-7H-Pyrrolo[2,3-D]Pyrimidin-5-Yl)Isoquinolin-8-Yl)-3-(5-(1-(Trifluoromethyl)Cyclopropyl)Isoxazol-3-Yl)Urea

### Step 1: Synthesis of tert-butyl (tert-butoxycarbonyl)(7-cyclopropyl-5-(8-(3-(5-(1-(trifluoromethyl)cyclopropyl)isoxazol-3-yl)ureido)isoquinolin-5-yl)-7H-pyrrolo[2,3-d]pyrimidin-4-yl)carbamate

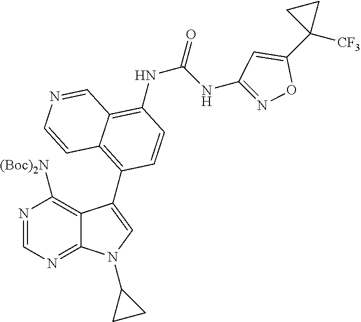

The title compound was obtained by following the general procedure for urea formation (Method B), starting from tert-butyl (5-(8-aminoisoquinolin-5-yl)-7-cyclopropyl-7H-pyrrolo[2,3-d]pyrimidin-4-yl)(tert-butoxycarbonyl)carbamate (B3, 100 mg, 0.194 mmol) and phenyl (5-(1-(trifluoromethyl)cyclopropyl)isoxazol-3-yl)carbamate (C3, 60 mg, 0.194 mmol), and was isolated as an off-white solid (20 mg, 14% yield). MS: 735.7 [M+H].

### Step 2: Synthesis of 1-(5-(4-amino-7-cyclopropyl-7H-pyrrolo[2,3-d]pyrimidin-5-yl)isoquinolin-8-yl)-3-(5-(1-(trifluoromethyl)cyclopropyl)isoxazol-3-yl)urea

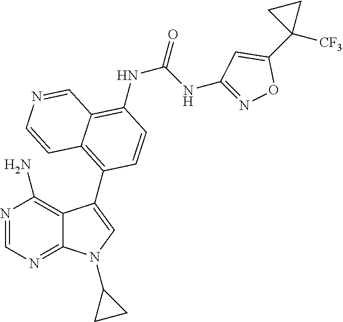 [SMILES: Nc1ncnc2c1c(-c1ccc(NC(=O)Nc3cc(C4(C(F)(F)F)CC4)on3)c3cnccc13)cn2C1CC1]

The title compound was obtained by following a similar procedure described for step 2 of Example 5, starting from tert-butyl (tert-butoxycarbonyl)(7-cyclopropyl-5-(8-(3-(5-(1-(trifluoromethyl)cyclopropyl)isoxazol-3-yl)ureido)isoquinolin-5-yl)-7H-pyrrolo[2,3-d]pyrimidin-4-yl)carbamate (20 mg, 0.027 mmol), and was isolated as a yellow solid (14 mg, 96% yield). ¹H NMR (400 MHz, DMSO-d₆) δ=10.11 (bs, 1H), 9.58 (bs, 1H), 9.49-9.51 (m, 1H), 8.52 (d, J=6.0 Hz, 1H), 8.21-8.33 (m, 2H), 7.66-7.74 (m, 2H), 7.43-7.47 (m, 1H), 6.94 (s, 1H), 6.54 (bs, 2H), 3.70 (bs, 1H), 1.52-1.59 (m, 4H), 1.09-1.13 (m, 4H). MS: 535.0 [M+H].

### Example 9

### 1-(5-(4-Amino-7-Cyclopropyl-7H-Pyrrolo[2,3-D]Pyrimidin-5-Yl)Isoquinolin-8-Yl)-3-(4-((4-Methylpiperazin-1-Yl)Methyl)-3-(Trifluoromethyl)Phenyl)Urea

### Step 1: Synthesis of tert-butyl (tert-butoxycarbonyl)(7-cyclopropyl-5-(8-(3-(4-((4-methylpiperazin-1-yl)methyl)-3-(trifluoromethyl)phenyl)ureido)isoquinolin-5-yl)-7H-pyrrolo[2,3-d]pyrimidin-4-yl)carbamate

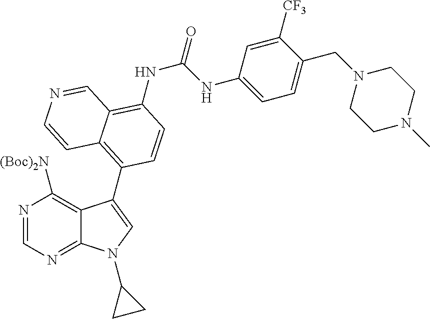

The title compound was obtained by following the general procedure for urea formation (Method B), starting from tert-butyl (5-(8-aminoisoquinolin-5-yl)-7-cyclopropyl-7H-pyrrolo[2,3-d]pyrimidin-4-yl)(tert-butoxycarbonyl)carbamate (B3, 95 mg, 0.184 mmol) and phenyl (4-((4-methylpiperazin-1-yl)methyl)-3-(trifluoromethyl)phenyl)carbamate (C5, 72 mg, 0.184 mmol), and was isolated as an off-white solid (15 mg, 10% yield). MS: 816.5 [M+H].

### Step 2: Synthesis of 1-(5-(4-amino-7-cyclopropyl-7H-pyrrolo[2,3-d]pyrimidin-5-yl)isoquinolin-8-yl)-3-(4-((4-methylpiperazin-1-yl)methyl)-3-(trifluoromethyl)phenyl)urea

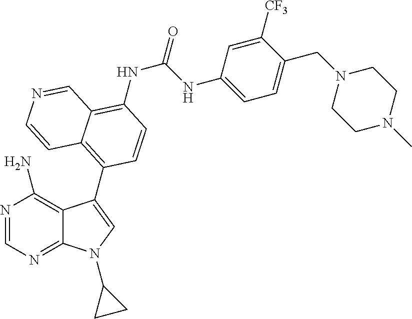 [SMILES: CN1CCN(Cc2ccc(NC(=O)Nc3ccc(-c4cn(C5CC5)c5ncnc(N)c45)c4ccncc34)cc2C(F)(F)F)CC1]

The title compound was obtained by following a similar procedure described for step 2 of Example 5, starting from tert-butyl (tert-butoxycarbonyl)(7-cyclopropyl-5-(8-(3-(4-((4-methylpiperazin-1-yl)methyl)-3-(trifluoromethyl)phenyl)ureido)isoquinolin-5-yl)-7H-pyrrolo[2,3-d]pyrimidin-4-yl)carbamate (15 mg, 0.018 mmol), and was isolated as a yellow solid (1.5 mg, 14% yield). ¹H NMR (400 MHz, CD₃OD) δ=9.63 (bs, 1H), 8.52 (d, J=6.0 Hz, 1H), 8.40 (s, 1H), 8.14 (d, J=8.0 Hz, 1H), 8.02 (s, 1H), 7.95 (d, J=8.0 Hz, 1H), 7.88 (d, J=6.0 Hz, 1H), 7.75 (bs, 2H), 7.57 (s, 1H), 3.77-3.79 (m, 3H), 3.49-3.50 (m, 2H), 3.15-3.15 (m, 2H), 3.08 (bs, 2H), 2.93 (s, 3H), 2.49 (bs, 2H), 1.25-1.26 (m, 4H). MS: 616.3 [M+H].

### Example 10

### 1-(5-(4-Amino-7-Cyclopropyl-7H-Pyrrolo[2,3-D]Pyrimidin-5-Yl)Isoquinolin-8-Yl)-3-(4-((1-Methylpiperidin-4-Yl)Oxy)-3-(Trifluoromethyl)Phenyl)Urea

### Step 1: Synthesis of tert-butyl (tert-butoxycarbonyl)(7-cyclopropyl-5-(8-(3-(4-((1-methylpiperidin-4-yl)oxy)-3-(trifluoromethyl)phenyl)ureido)isoquinolin-5-yl)-7H-pyrrolo[2,3-d]pyrimidin-4-yl)carbamate

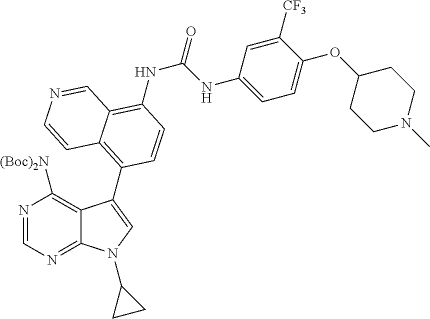

The title compound was obtained by following a similar procedure described for Intermediate B1, starting from 1-(5-bromoisoquinolin-8-yl)-3-(4-((1-methylpiperidin-4-yl)oxy)-3-(trifluoromethyl)phenyl)urea (D4, 209 mg, 0.400 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 200 mg, 0.400 mmol), and was isolated as a pale-yellow solid (60 mg, 18% yield). MS: 817.3 [M+H].

### Step 2: Synthesis of 1-(5-(4-amino-7-cyclopropyl-7H-pyrrolo[2,3-d]pyrimidin-5-yl)isoquinolin-8-yl)-3-(4-((1-methylpiperidin-4-yl)oxy)-3-(trifluoromethyl)phenyl)urea

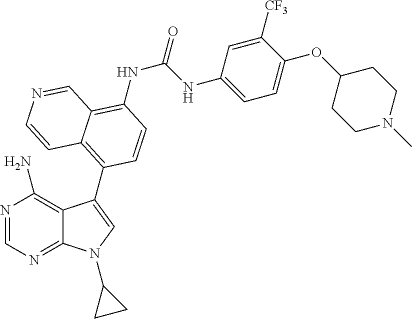 [SMILES: CN1CCC(Oc2ccc(NC(=O)Nc3ccc(-c4cn(C5CC5)c5ncnc(N)c45)c4ccncc34)cc2C(F)(F)F)CC1]

The title compound was obtained by following a similar procedure described for step 2 of Example 5, starting from tert-butyl (tert-butoxycarbonyl)(7-cyclopropyl-5-(8-(3-(4-((1-methylpiperidin-4-yl)oxy)-3-(trifluoromethyl)phenyl)ureido)isoquinolin-5-yl)-7H-pyrrolo[2,3-d]pyrimidin-4-yl)carbamate (60 mg, 0.073 mmol), and was isolated as an off-white solid (7 mg, 16% yield). ¹H NMR (400 MHz, DMSO-d₆) δ=9.59 (s, 1H), 9.24 (s, 1H), 9.16 (s, 1H), 8.51 (d, J=6.0 Hz, 1H), 8.19-8.21 (m, 2H), 7.91 (d, J=2.8 Hz, 1H), 7.69 (d, J=8.0 Hz, 1H), 7.59-7.65 (m, 2H), 7.29-7.31 (m, 2H), 5.74 (s, 2H), 4.56-4.57 (m, 1H), 3.63-3.66 (m, 1H), 2.27-2.35 (m, 4H), 2.20 (s, 3H), 1.87-1.92 (m, 2H), 1.71 (s, 2H), 1.05-1.07 (m, 4H). MS: 617.2 [M+H].

### Example 11

### 1-(5-(4-Amino-7-Cyclopropyl-7H-Pyrrolo[2,3-D]Pyrimidin-5-Yl)Imidazo[1,2-A]Pyridin-8-Yl)-3-(3-(Tert-Butyl)-1-Phenyl-1H-Pyrazol-5-Yl)Urea

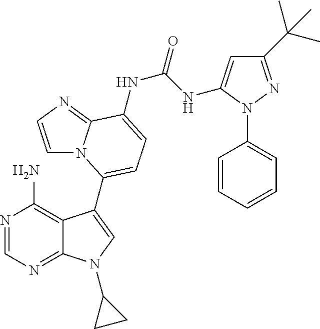 [SMILES: CC(C)(C)c1cc(NC(=O)Nc2ccc(-c3cn(C4CC4)c4ncnc(N)c34)n3ccnc23)n(-c2ccccc2)n1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (3-(tert-butyl)-1-phenyl-1H-pyrazol-5-yl)carbamate (C7, 110 mg, 0.327 mmol), and was isolated as an off-white solid (5 mg, 2.8% yield). ¹H NMR (400 MHz, DMSO-d₆) δ=9.59 (s, 1H), 9.40 (s, 1H), 8.46 (s, 1H), 7.95 (d, J=8.0 Hz, 1H), 7.83 (s, 1H), 7.77 (s, 1H), 7.53-7.62 (m, 5H), 7.42-7.46 (m, 1H), 6.92 (d, J=8.0 Hz, 1H), 6.44 (s, 1H), 3.74-3.75 (m, 1H), 1.31 (s, 9H), 1.11-1.17 (m, 4H). MS: 547.3 [M+H].

### Example 12

### 1-(5-(4-Amino-7-Cyclopropyl-7H-Pyrrolo[2,3-D]Pyrimidin-5-Yl)Imidazo[1,2-A]Pyridin-8-Yl)-3-(3-(Tert-Butyl)-1-(P-Tolyl)-1H-Pyrazol-5-Yl)Urea

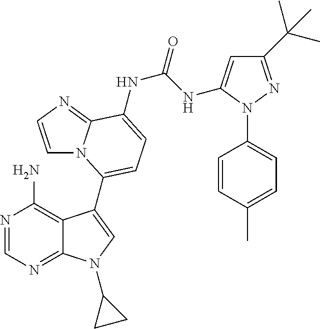 [SMILES: Cc1ccc(-n2nc(C(C)(C)C)cc2NC(=O)Nc2ccc(-c3cn(C4CC4)c4ncnc(N)c34)n3ccnc23)cc1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 50 mg, 0.163 mmol) and phenyl (3-(tert-butyl)-1-(p-tolyl)-1H-pyrazol-5-yl)carbamate (C8, 57 mg, 0.327 mmol), and was isolated as an off-white solid (2 mg, 2.2% yield). ¹H NMR (400 MHz, CD₃OD) δ=8.25 (s, 1H), 7.95 (d, J=7.6 Hz, 1H), 7.62 (s, 1H), 7.51-7.53 (m, 2H), 7.38-7.43 (m, 4H), 6.91 (d, J=8.0 Hz, 1H), 6.49 (s, 1H), 3.57-3.62 (m, 1H), 2.45 (s, 3H), 1.38 (s, 9H), 1.14-1.20 (m, 4H). MS: 561.4 [M+H].

### Example 13

### 1-(5-(4-Amino-7-Cyclopropyl-7H-Pyrrolo[2,3-D]Pyrimidin-5-Yl)Imidazo[1,2-A]Pyridin-8-Yl)-3-(3-(Tert-Butyl)-1-(4-Chlorophenyl)-1H-Pyrazol-5-Yl)Urea

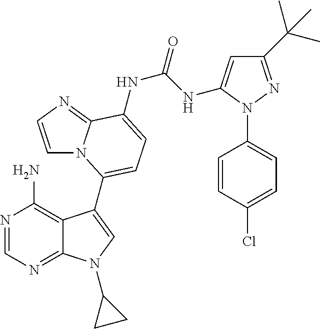 [SMILES: CC(C)(C)c1cc(NC(=O)Nc2ccc(-c3cn(C4CC4)c4ncnc(N)c34)n3ccnc23)n(-c2ccc(Cl)cc2)n1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 125 mg, 0.409 mmol) and phenyl (3-(tert-butyl)-1-(4-chlorophenyl)-1H-pyrazol-5-yl)carbamate (C9, 151 mg, 0.409 mmol), and was isolated as an off-white solid (4 mg, 1.7% yield). ¹H NMR (400 MHz, CD₃OD) δ=8.25 (s, 1H), 7.93 (d, J=7.6 Hz, 1H), 7.62 (s, 1H), 7.50-7.57 (m, 6H), 6.91 (d, J=8.0 Hz, 1H), 6.49 (s, 1H), 3.57-3.60 (m, 1H), 1.37 (s, 9H), 1.15-1.20 (m, 4H). MS: 581.2 [M+H].

### Example 14

### 1-(5-(4-Amino-7-Cyclopropyl-7H-Pyrrolo[2,3-D]Pyrimidin-5-Yl)Imidazo[1,2-A]Pyridin-8-Yl)-3-(3-Methylisoxazol-5-Yl)Urea

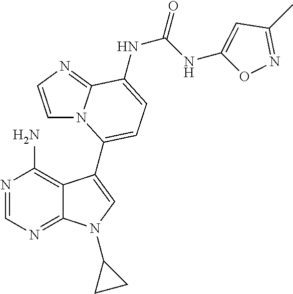 [SMILES: Cc1cc(NC(=O)Nc2ccc(-c3cn(C4CC4)c4ncnc(N)c34)n3ccnc23)on1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (3-methylisoxazol-5-yl)carbamate (C10, 71 mg, 0.327 mmol). MS (expected): 430.2 [M+H].

The title compound was obtained by following a similar procedure described for Intermediate B1, starting from 1-(5-bromoimidazo[1,2-a]pyridin-8-yl)-3-(3-methylisoxazol-5-yl)urea (D40, 50 mg, 0.149 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 112 mg, 0.223 mmol), and was isolated as an off-white solid (6 mg, 8.4% yield). Cleavage of the di-Boc protecting group was observed during the reaction. ¹H NMR (400 MHz, CD₃OD) δ=8.26 (s, 1H), 8.03-8.05 (m, 1H), 7.66 (d, J=1.2 Hz, 1H), 7.59 (s, 1H), 7.53 (s, 1H), 6.95 (d, J=8.0 Hz, 1H), 6.14 (s, 1H), 3.61-3.66 (m, 1H), 2.28 (s, 3H), 1.13-1.22 (m, 4H). MS: 430.2 [M+H].

### Example 15

### 1-(5-(4-Amino-7-Cyclopropyl-7H-Pyrrolo[2,3-D]Pyrimidin-5-Yl)Imidazo[1,2-A]Pyridin-8-Yl)-3-(3-Ethylisoxazol-5-Yl)Urea

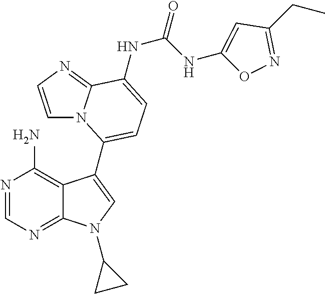 [SMILES: CCc1cc(NC(=O)Nc2ccc(-c3cn(C4CC4)c4ncnc(N)c34)n3ccnc23)on1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 30 mg, 0.098 mmol) and phenyl (3-ethylisoxazol-5-yl)carbamate (C11, 23 mg, 0.098 mmol), and was isolated as a pale brown solid (6 mg, 15% yield). ¹H NMR (400 MHz, DMSO-d₆) δ=10.88 (s, 1H), 9.47 (s, 1H), 8.33 (d, J=6.4 Hz, 1H), 7.95 (d, J=8.0 Hz, 1H), 7.71-7.73 (m, 2H), 7.61 (s, 1H), 6.88 (d, J=7.6 Hz, 1H), 6.08 (s, 1H), 3.68-3.71 (m, 1H), 2.53-2.60 (m, 2H), 1.07-1.22 (m, 7H). MS: 444.1 [M+H].

### Example 16

### 1-(5-(4-Amino-7-Cyclopropyl-7H-Pyrrolo[2,3-D]Pyrimidin-5-Yl)Imidazo[1,2-A]Pyridin-8-Yl)-3-(3-Isopropylisoxazol-5-Yl)Urea

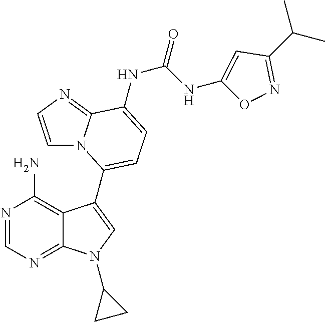 [SMILES: CC(C)c1cc(NC(=O)Nc2ccc(-c3cn(C4CC4)c4ncnc(N)c34)n3ccnc23)on1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 30 mg, 0.098 mmol) and phenyl (3-isopropylisoxazol-5-yl)carbamate (C12, 24 mg, 0.098 mmol), and was isolated as an off-white solid (2 mg, 4.4% yield). ¹H NMR (400 MHz, CD₃OD) δ=8.25 (s, 1H), 8.04 (d, J=8.0 Hz, 1H), 7.65 (d, J=1.2 Hz, 1H), 7.58 (d, J=1.2 Hz, 1H), 7.53 (s, 1H), 6.95 (d, J=7.6 Hz, 1H), 6.18 (s, 1H), 3.67-3.71 (m, 1H), 2.99-3.03 (m, 1H), 1.18-1.35 (m, 10H). MS: 458.3 [M+H].

### Example 17

### 1-(5-(4-Amino-7-Cyclopropyl-7H-Pyrrolo[2,3-D]Pyrimidin-5-Yl)Imidazo[1,2-A]Pyridin-8-Yl)-3-(3-(Tert-Butyl)Isoxazol-5-Yl)Urea

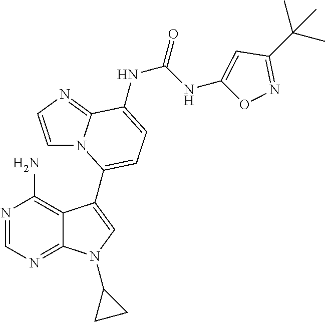 [SMILES: CC(C)(C)c1cc(NC(=O)Nc2ccc(-c3cn(C4CC4)c4ncnc(N)c34)n3ccnc23)on1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (3-(tert-butyl)isoxazol-5-yl)carbamate (C2, 85 mg, 0.327 mmol), and was isolated as an off-white solid (6 mg, 3.9% yield). ¹H NMR (400 MHz, DMSO-d₆) δ=10.87 (s, 1H), 9.41 (s, 1H), 8.20 (s, 1H), 7.95 (d, J=7.6 Hz, 1H), 7.67 (d, J=1.2 Hz, 1H), 7.57 (s, 1H), 7.57 (d, J=1.2 Hz, 1H), 6.82 (d, J=7.6 Hz, 1H), 6.14 (bs, 3H), 3.57-3.63 (m, 1H), 1.28 (s, 9H), 1.04-1.11 (m, 4H). MS: 472.2 [M+H].

### Example 18

### 1-(5-(4-Amino-7-Cyclopropyl-7H-Pyrrolo[2,3-D]Pyrimidin-5-Yl)Imidazo[1,2-A]Pyridin-8-Yl)-3-(3-(1-Methylcyclopropyl)Isoxazol-5-Yl)Urea

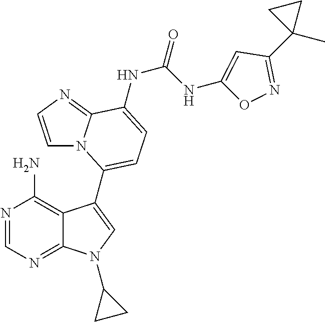 [SMILES: CC1(c2cc(NC(=O)Nc3ccc(-c4cn(C5CC5)c5ncnc(N)c45)n4ccnc34)on2)CC1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 40 mg, 0.131 mmol) and phenyl (3-(1-methylcyclopropyl)isoxazol-5-yl)carbamate (C4, 34 mg, 0.327 mmol), and was isolated as an off-white solid (2 mg, 3.2% yield). ¹H NMR (400 MHz, CD₃OD) δ=8.26 (s, 1H), 8.03 (d, J=7.6 Hz, 1H), 7.66 (d, J=1.2 Hz, 1H), 7.59 (d, J=1.6 Hz, 1H), 7.54 (s, 1H), 6.95 (d, J=7.6 Hz, 1H), 5.99 (s, 1H), 3.57-3.64 (m, 1H), 1.46 (s, 3H), 1.14-1.20 (m, 4H), 0.90-1.06 (m, 2H), 0.89-0.89 (m, 2H). MS: 470.0 [M+H].

### Example 19

### 1-(5-(4-Amino-7-Cyclopropyl-7H-Pyrrolo[2,3-D]Pyrimidin-5-Yl)Imidazo[1,2-A]Pyridin-8-Yl)-3-(3-(1-Methylcyclobutyl)Isoxazol-5-Yl)Urea

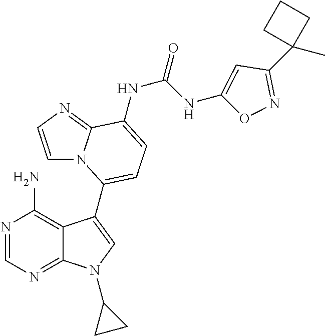 [SMILES: CC1(c2cc(NC(=O)Nc3ccc(-c4cn(C5CC5)c5ncnc(N)c45)n4ccnc34)on2)CCC1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 45 mg, 0.147 mmol) and phenyl (3-(1-methylcyclobutyl)isoxazol-5-yl)carbamate (C13, 40 mg, 0.147 mmol), and was isolated as an off-white solid (1 mg, 1.4% yield). MS: 484.2 [M+H].

### Example 20

### 1-(5-(4-Amino-7-Cyclopropyl-7H-Pyrrolo[2,3-D]Pyrimidin-5-Yl)Imidazo[1,2-A]Pyridin-8-Yl)-3-(3-(2-Fluoropropan-2-Yl)Isoxazol-5-Yl)Urea

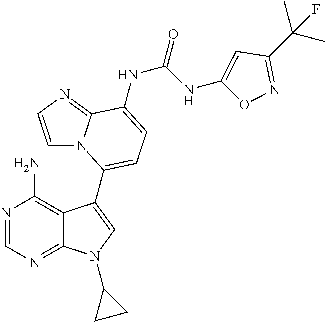 [SMILES: CC(C)(F)c1cc(NC(=O)Nc2ccc(-c3cn(C4CC4)c4ncnc(N)c34)n3ccnc23)on1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (3-(2-fluoropropan-2-yl)isoxazol-5-yl)carbamate (C14, 87 mg, 0.327 mmol). MS (expected): 476.2 [M+H].

The title compound was obtained by following a similar procedure described for Intermediate B1, starting from 1-(5-bromoimidazo[1,2-a]pyridin-8-yl)-3-(3-(2-fluoropropan-2-yl)isoxazol-5-yl)urea (D42, 90 mg, 0.235 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 177 mg, 0.343 mmol), and was isolated as an off-white solid (40 mg, 35% yield). Cleavage of the di-Boc protecting group was observed during the reaction. ¹H NMR (400 MHz, DMSO-d₆) δ=11.06 (s, 1H), 9.45 (s, 1H), 8.20 (s, 1H), 7.95 (d, J=7.6 Hz, 1H), 7.68 (d, J=1.2 Hz, 1H), 7.57-7.58 (m, 2H), 6.83 (d, J=7.6 Hz, 1H), 6.25 (s, 1H), 6.17 (bs, 2H), 3.61-3.67 (m, 1H), 1.73 (s, 3H), 1.68 (s, 3H), 1.02-1.13 (m, 4H). MS: 476.2 [M+H].

### Example 21

### 1-(5-(4-Amino-7-Cyclopropyl-7H-Pyrrolo[2,3-D]Pyrimidin-5-Yl)Imidazo[1,2-A]Pyridin-8-Yl)-3-(3-(1,1,1-Trifluoro-2-Methylpropan-2-Yl)Isoxazol-5-Yl)Urea

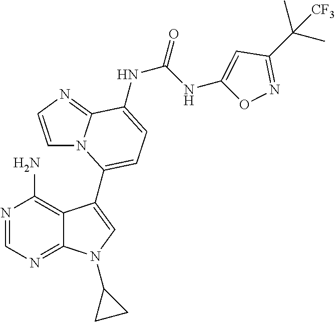 [SMILES: CC(C)(c1cc(NC(=O)Nc2ccc(-c3cn(C4CC4)c4ncnc(N)c34)n3ccnc23)on1)C(F)(F)F]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (3-(1,1,1-trifluoro-2-methylpropan-2-yl)isoxazol-5-yl)carbamate (C15, 103 mg, 0.327 mmol). MS (expected): 526.2 [M+H].

The title compound was obtained by following a similar procedure described for Intermediate B1, starting from 1-(5-bromoimidazo[1,2-a]pyridin-8-yl)-3-(3-(1,1,1-trifluoro-2-methylpropan-2-yl)isoxazol-5-yl)urea (D43, 35 mg, 0.081 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 60.8 mg, 0.121 mmol), and was isolated as an off-white solid (7 mg, 16.4% yield). Cleavage of the di-Boc protecting group was observed during the reaction. ¹H NMR (400 MHz, CD₃OD) δ=8.26 (s, 1H), 8.03 (d, J=8.0 Hz, 1H), 7.66 (d, J=1.2 Hz, 1H), 7.59 (d, J=1.2 Hz, 1H), 7.53 (s, 1H), 6.95 (d, J=7.6 Hz, 1H), 6.34 (s, 1H), 3.57-3.62 (m, 1H), 1.58 (s, 6H), 1.13-1.22 (m, 4H). MS: 526.2 [M+H].

### Example 22

### 1-(5-(4-Amino-7-Cyclopropyl-7H-Pyrrolo[2,3-D]Pyrimidin-5-Yl)Imidazo[1,2-A]Pyridin-8-Yl)-3-(3-(1-(Trifluoromethyl)Cyclopropyl)Isoxazol-5-Yl)Urea

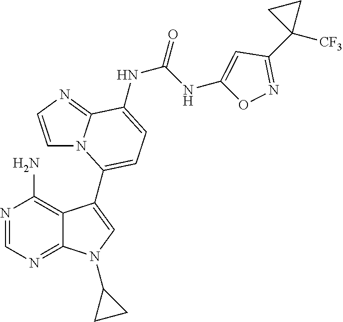 [SMILES: Nc1ncnc2c1c(-c1ccc(NC(=O)Nc3cc(C4(C(F)(F)F)CC4)no3)c3nccn13)cn2C1CC1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 110 mg, 0.360 mmol) and phenyl (3-(1-(trifluoromethyl)cyclopropyl)isoxazol-5-yl)carbamate (C1, 112 mg, 0.360 mmol), and was isolated as an off-white solid (20 mg, 11% yield). ¹H NMR (400 MHz, DMSO-d₆) δ=11.18 (s, 1H), 9.57 (s, 1H), 8.47 (s, 1H), 7.96 (d, J=8.0 Hz, 1H), 7.86 (s, 1H), 7.82 (s, 1H), 7.69 (s, 1H), 6.97 (d, J=8.0 Hz, 1H), 6.25 (s, 1H), 3.73-3.85 (m, 1H), 1.48-1.50 (m, 4H), 1.12-1.21 (m, 4H). LCMS: 524.2 [M+H].

### Example 23

### 1-(5-(4-Amino-7-Cyclopropyl-7H-Pyrrolo[2,3-D]Pyrimidin-5-Yl)Imidazo[1,2-A]Pyridin-8-Yl)-3-(3-(1-(Trifluoromethyl)Cyclobutyl)Isoxazol-5-Yl)Urea

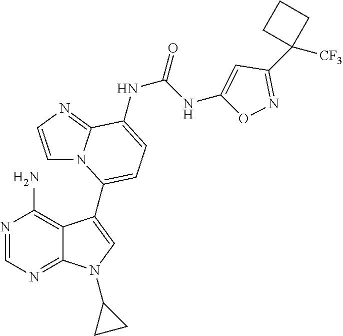 [SMILES: Nc1ncnc2c1c(-c1ccc(NC(=O)Nc3cc(C4(C(F)(F)F)CCC4)no3)c3nccn13)cn2C1CC1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 30 mg, 0.098 mmol) and phenyl (3-(1-(trifluoromethyl)cyclobutyl)isoxazol-5-yl)carbamate (C16, 44.5 mg, 0.098 mmol), and was isolated as an off-white solid (8 mg, 15% yield). ¹H NMR (400 MHz, DMSO-d₆) δ=8.26 (s, 1H), 8.04 (d, J=8.0 Hz, 1H), 7.67 (d, J=1.2 Hz, 1H), 7.60 (d, J=1.2 Hz, 1H), 7.54 (s, 1H), 6.96 (d, J=7.6 Hz, 1H), 6.26 (s, 1H), 3.58-3.63 (m, 1H), 2.63-2.71 (m, 4H), 2.11-2.17 (m, 2H), 1.13-1.22 (m, 4H); LCMS: 538.2 [M+H].

### Example 24

### 1-(5-(4-Amino-7-Cyclopropyl-7H-Pyrrolo[2,3-D]Pyrimidin-5-Yl)Imidazo[1,2-A]Pyridin-8-Yl)-3-(5-Methylisoxazol-3-Yl)Urea

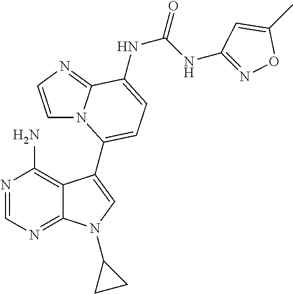 [SMILES: Cc1cc(NC(=O)Nc2ccc(-c3cn(C4CC4)c4ncnc(N)c34)n3ccnc23)no1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-methylisoxazol-3-yl)carbamate (C17, 71 mg, 0.327 mmol). MS (expected): 430.2 [M+H].

The title compound was prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 50 mg, 0.164 mmol) and phenyl (5-methylisoxazol-3-yl)carbamate (C17, 36 mg, 0.164 mmol). ¹H NMR (400 MHz, DMSO-d₆) δ=11.98 (s, 1H), 8.65 (s, 1H), 7.90-7.93 (m, 2H), 7.73 (d, J=1.2 Hz, 1H), 7.45 (s, 1H), 6.73 (d, J=7.6 Hz, 1H), 6.36-6.39 (m, 2H), 5.77 (bs, 2H), 3.72-3.75 (m, 1H), 2.37 (s, 3H), 1.10-1.24 (m, 4H). MS: 430.1 [M+H].

### Example 25

### 1-(5-(4-Amino-7-Cyclopropyl-7H-Pyrrolo[2,3-D]Pyrimidin-5-Yl)Imidazo[1,2-A]Pyridin-8-Yl)-3-(5-Ethylisoxazol-3-Yl)Urea

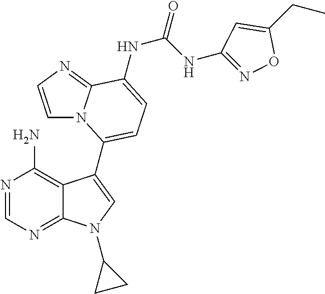 [SMILES: CCc1cc(NC(=O)Nc2ccc(-c3cn(C4CC4)c4ncnc(N)c34)n3ccnc23)no1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-ethylisoxazol-3-yl)carbamate (C18, 76 mg, 0.327 mmol). MS (expected): 444.2 [M+H].

### Example 26

### 1-(5-(4-Amino-7-Cyclopropyl-7H-Pyrrolo[2,3-D]Pyrimidin-5-Yl)Imidazo[1,2-A]Pyridin-8-Yl)-3-(5-Isopropylisoxazol-3-Yl)Urea

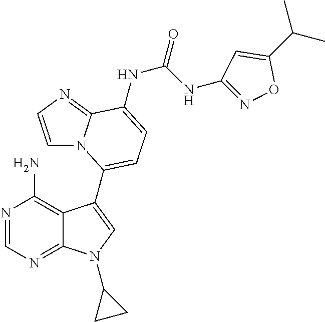 [SMILES: CC(C)c1cc(NC(=O)Nc2ccc(-c3cn(C4CC4)c4ncnc(N)c34)n3ccnc23)no1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-isopropylisoxazol-3-yl)carbamate (C19, 81 mg, 0.327 mmol). MS (expected): 458.2 [M+H].

The title compound was obtained by following a similar procedure described for Intermediate B1, starting from 1-(5-bromoimidazo[1,2-a]pyridin-8-yl)-3-(5-isopropylisoxazol-3-yl)urea (D44, 20.0 mg, 0.055 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 27.5 mg, 0.055 mmol), and was isolated as an off-white solid (3.5 mg, 14% yield). Cleavage of the di-Boc protecting group was observed during the reaction. ¹H NMR (400 MHz, DMSO-d₆) δ=10.28 (bs, 1H), 9.56 (bs, 1H), 8.20 (s, 1H), 7.96 (d, J=7.6 Hz, 1H), 7.67 (d, J=1.2 Hz, 1H), 7.56-7.57 (m, 2H), 6.81 (d, J=7.6 Hz, 1H), 6.56 (s, 1H), 6.14 (bs, 2H), 3.61-3.67 (m, 1H), 3.03-3.10 (m, 1H), 1.25-1.28 (m, 6H), 1.04-1.14 (m, 4H). MS: 458.2 [M+H].

### Example 27

### 1-(5-(4-Amino-7-Cyclopropyl-7H-Pyrrolo[2,3-D]Pyrimidin-5-Yl)Imidazo[1,2-A]Pyridin-8-Yl)-3-(5-(Tert-Butyl)Isoxazol-3-Yl)Urea

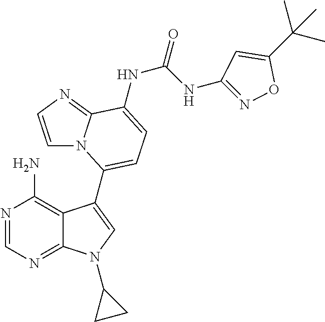 [SMILES: CC(C)(C)c1cc(NC(=O)Nc2ccc(-c3cn(C4CC4)c4ncnc(N)c34)n3ccnc23)no1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-(tert-butyl)isoxazol-3-yl)carbamate (C20, 85 mg, 0.327 mmol). MS (expected): 472.2 [M+H].

The title compound was obtained by following a similar procedure described for Intermediate B1, starting from 1-(5-bromoimidazo[1,2-a]pyridin-8-yl)-3-(5-(tert-butyl)isoxazol-3-yl)urea (D46, 50 mg, 0.132 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 99 mg, 0.198 mmol), and was isolated as an off-white solid (6 mg, 9% yield). Cleavage of the di-Boc protecting group was observed during the reaction. ¹H NMR (400 MHz, CD₃OD) δ=8.25 (s, 1H), 8.01 (d, J=8.0 Hz, 1H), 7.53-7.65 (m, 3H), 6.92 (d, J=7.6 Hz, 1H), 6.41 (s, 1H), 3.56-3.62 (m, 1H), 1.37 (s, 9H), 1.02-1.13 (m, 4H). MS: 472.2 [M+H].

### Example 28

### 1-(5-(4-Amino-7-Cyclopropyl-7H-Pyrrolo[2,3-D]Pyrimidin-5-Yl)Imidazo[1,2-A]Pyridin-8-Yl)-3-(5-(1-Methylcyclopropyl)Isoxazol-3-Yl)Urea

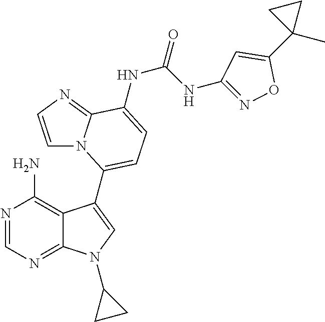 [SMILES: CC1(c2cc(NC(=O)Nc3ccc(-c4cn(C5CC5)c5ncnc(N)c45)n4ccnc34)no2)CC1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-(1-methylcyclopropyl)isoxazol-3-yl)carbamate (C21, 85 mg, 0.327 mmol). MS (expected): 470.2 [M+H].

The title compound was obtained by following a similar procedure described for Intermediate B1, starting from 1-(5-bromoimidazo[1,2-a]pyridin-8-yl)-3-(5-(1-methylcyclopropyl)isoxazol-3-yl)urea (D47, 10 mg, 0.027 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 13.3 mg, 0.027 mmol), and was isolated as an off-white solid (0.7 mg, 5.6% yield). Cleavage of the di-Boc protecting group was observed during the reaction. ¹H NMR (400 MHz, CD₃OD) δ=8.25 (s, 1H), 8.05 (d, J=8.0 Hz, 1H), 7.58-7.65 (m, 3H), 6.94 (d, J=7.6 Hz, 1H), 6.42 (s, 1H), 3.58-3.61 (m, 1H), 1.50 (s, 3H), 1.14-1.22 (m, 4H), 0.93-0.95 (m, 4H). MS: 470.1 [M+H].

### Example 29

### 1-(5-(4-Amino-7-Cyclopropyl-7H-Pyrrolo[2,3-D]Pyrimidin-5-Yl)Imidazo[1,2-A]Pyridin-8-Yl)-3-(5-(1-Methylcyclobutyl)Isoxazol-3-Yl)Urea

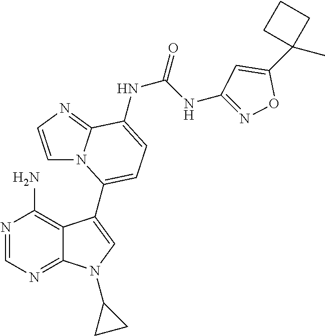 [SMILES: CC1(c2cc(NC(=O)Nc3ccc(-c4cn(C5CC5)c5ncnc(N)c45)n4ccnc34)no2)CCC1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-(1-methylcyclobutyl)isoxazol-3-yl)carbamate (C22, 89 mg, 0.327 mmol). MS (expected): 484.2 [M+H].

The title compound was obtained by following a similar procedure described for Intermediate B1, starting from 1-(5-bromoimidazo[1,2-a]pyridin-8-yl)-3-(5-(1-methylcyclobutyl)isoxazol-3-yl)urea (D48, 20.0 mg, 0.051 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 25.6 mg, 0.051 mmol), and was isolated as an off-white solid (1 mg, 4% yield). Cleavage of the di-Boc protecting group was observed during the reaction. LCMS: 484.2 [M+H].

### Example 30

### 1-(5-(4-Amino-7-Cyclopropyl-7H-Pyrrolo[2,3-D]Pyrimidin-5-Yl)Imidazo[1,2-A]Pyridin-8-Yl)-3-(5-(2-Fluoropropan-2-Yl)Isoxazol-3-Yl)Urea

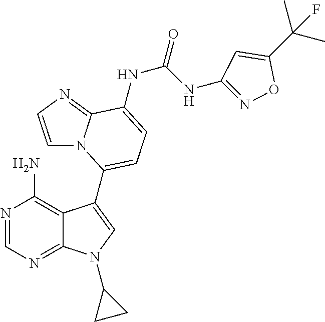 [SMILES: CC(C)(F)c1cc(NC(=O)Nc2ccc(-c3cn(C4CC4)c4ncnc(N)c34)n3ccnc23)no1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-(2-fluoropropan-2-yl)isoxazol-3-yl)carbamate (C23, 87 mg, 0.327 mmol). MS (expected): 476.2 [M+H].

### Example 31

### 1-(5-(4-Amino-7-Cyclopropyl-7H-Pyrrolo[2,3-D]Pyrimidin-5-Yl)Imidazo[1,2-A]Pyridin-8-Yl)-3-(5-(1,1,1-Trifluoro-2-Methylpropan-2-Yl)Isoxazol-3-Yl)Urea

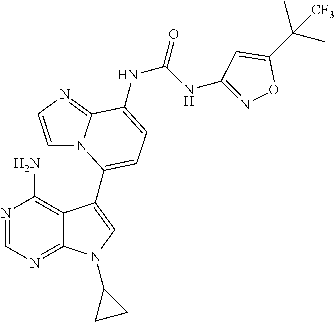 [SMILES: CC(C)(c1cc(NC(=O)Nc2ccc(-c3cn(C4CC4)c4ncnc(N)c34)n3ccnc23)no1)C(F)(F)F]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-(1,1,1-trifluoro-2-methylpropan-2-yl)isoxazol-3-yl)carbamate (C24, 103 mg, 0.327 mmol). MS (expected): 526.2 [M+H].

The title compound was obtained by following a similar procedure described for Intermediate B1, starting from 1-(5-bromoimidazo[1,2-a]pyridin-8-yl)-3-(5-(1,1,1-trifluoro-2-methylpropan-2-yl)isoxazol-3-yl)urea (D49, 20.0 mg, 0.046 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 34.7 mg, 0.069 mmol), and was isolated as an off-white solid (6 mg, 25% yield). Cleavage of the di-Boc protecting group was observed during the reaction. ¹H NMR (400 MHz, DMSO-d6) δ=10.47 (bs, 1H), 9.53 (bs, 1H), 8.21 (s, 1H), 7.96 (d, J=7.6 Hz, 1H), 7.67 (d, J=1.2 Hz, 1H), 7.57 (bs, 2H), 6.94 (s, 1H), 6.83 (d, J=7.6 Hz, 1H), 6.16 (bs, 2H), 3.61-3.66 (m, 1H), 1.58 (s, 6H), 1.04-1.13 (m, 4H). MS: 526.3. [M+H].

### Example 32

### 1-(5-(4-Amino-7-Cyclopropyl-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

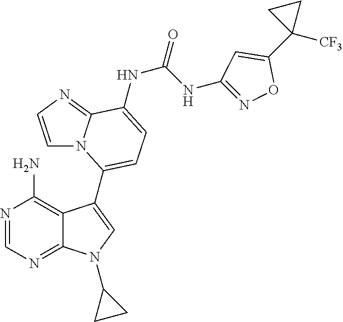 [SMILES: Nc1ncnc2c1c(-c1ccc(NC(=O)Nc3cc(C4(C(F)(F)F)CC4)on3)c3nccn13)cn2C1CC1]

The title compound was obtained by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-(1-(trifluoromethyl)cyclopropyl)isoxazol-3-yl)carbamate (C3, 102 mg, 0.327 mmol), and was isolated as an off-white solid (4 mg, 2.3% yield). ¹H NMR (400 MHz, DMSO-d₆) δ=10.45 (s, 1H), 9.52 (s, 1H), 8.20 (s, 1H), 7.96 (d, J=8.0 Hz, 1H), 7.67 (d, J=1.2 Hz, 1H), 7.57 (bs, 2H), 6.93 (s, 1H), 6.82 (d, J=7.6 Hz, 1H), 6.14 (bs, 2H), 3.61-3.65 (m, 1H), 1.51-1.58 (m, 4H), 1.04-1.13 (m, 4H). MS: 524.2 [M+H].

### Example 33

### 1-(5-(4-Amino-7-Cyclopropyl-7H-Pyrrolo[2,3-D]Pyrimidin-5-Yl)Imidazo[1,2-A]Pyridin-8-Yl)-3-(5-(1-(Trifluoromethyl)Cyclobutpyl)Isoxazol-3-Yl)Urea

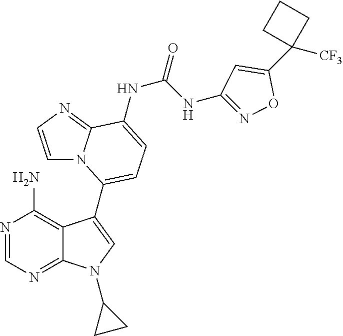 [SMILES: Nc1ncnc2c1c(-c1ccc(NC(=O)Nc3cc(C4(C(F)(F)F)CCC4)on3)c3nccn13)cn2C1CC1]

The title compound is prepared by following the general procedure for urea formation (Method B), starting from 5-(8-aminoimidazo[1,2-a]pyridin-5-yl)-7-cyclopropyl-7H-pyrrolo[2,3-d]pyrimidin-4-amine (B4, 100 mg, 0.327 mmol) and phenyl (5-(1-(trifluoromethyl)cyclobutyl)isoxazol-3-yl)carbamate (C25, 107 mg, 0.327 mmol). MS (expected): 538.2 [M+H].

The title compound was obtained by following a similar procedure described for Intermediate B1, starting from 1-(5-bromoimidazo[1,2-a]pyridin-8-yl)-3-(5-(1-(trifluoromethyl)cyclobutyl)isoxazol-3-yl)urea (D50, 26.6 mg, 0.060 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 30.0 mg, 0.060 mmol), and was isolated as an off-white solid (2.3 mg, 6.9% yield). Cleavage of the di-Boc protecting group was observed during the reaction. ¹H NMR (400 MHz, CD₃OD) δ=8.25 (s, 1H), 8.06 (d, J=7.6 Hz, 1H), 7.66 (d, J=1.2 Hz, 1H), 7.59 (d, J=1.2 Hz, 1H), 7.53 (s, 1H), 6.94 (d, J=8.0 Hz, 1H), 6.85 (s, 1H), 3.58-3.61 (m, 1H), 2.73-2.78 (m, 2H), 2.63-2.66 (m, 2H), 2.16-2.20 (m, 2H), 1.14-1.20 (m, 4H). MS: 538.2 [M+H].

### Example 34

### 1-(5-(4-Amino-7-Cyclopropyl-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of tert-butyl (tert-butoxycarbonyl)(7-cyclopropyl-5-(8-(3-(4-((4-methylpiperazin-1-yl)methyl)-3-(trifluoromethyl)phenyl)ureido)imidazo[1,2-a]pyridin-5-yl)-7H-pyrrolo[2,3-d]pyrimidin-4-yl)carbamate

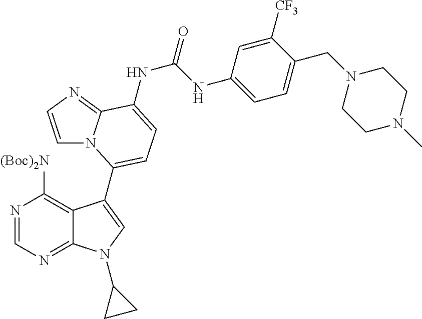

The title compound was obtained by following the general procedure for urea formation (Method B), starting from tert-butyl (5-(8-aminoimidazo[1,2-a]pyridin-5-yl)-7-cyclopropyl-7H-pyrrolo[2,3-d]pyrimidin-4-yl)(tert-butoxycarbonyl)carbamate (B5, 40 mg, 0.079 mmol) and phenyl (4-((4-methylpiperazin-1-yl)methyl)-3-(trifluoromethyl)phenyl)carbamate (C5, 31 mg, 0.079 mmol), and was isolated as an off-white solid (30 mg, 47% yield). MS: 805.3 [M+H].

### Step 2: Synthesis of 1-(5-(4-amino-7-cyclopropyl-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

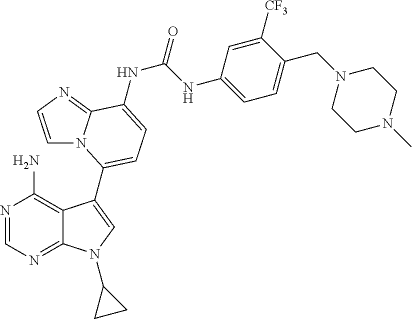 [SMILES: CN1CCN(Cc2ccc(NC(=O)Nc3ccc(-c4cn(C5CC5)c5ncnc(N)c45)n4ccnc34)cc2C(F)(F)F)CC1]

The title compound was obtained by following a similar procedure described for step 2 of Example 5, starting from tert-butyl (tert-butoxycarbonyl)(7-cyclopropyl-5-(8-(3-(4-((4-methylpiperazin-1-yl)methyl)-3-(trifluoromethyl)phenyl)ureido)imidazo[1,2-a]pyridin-5-yl)-7H-pyrrolo[2,3-d]pyrimidin-4-yl)carbamate (30 mg, 0.037 mmol), and was isolated as an off-white solid (3 mg, 13% yield). ¹H NMR (400 MHz, CD₃OD) δ=8.25 (s, 1H), 8.02 (d, J=7.6 Hz, 1H), 7.97 (s, 1H), 7.73 (bs, 2H), 7.65 (d, J=1.2 Hz, 1H), 7.58 (d, J=1.2 Hz, 1H), 7.52 (s, 1H), 6.94 (d, J=7.6 Hz, 1H), 3.70 (s, 2H), 3.58-3.61 (m, 1H), 2.84 (bs, 4H), 2.64 (bs, 4H), 2.53 (s, 3H), 1.14-1.22 (m, 4H). MS: 605.5 [M+H].

### Example 35

### 1-(5-(4-Amino-7-Cyclopropyl-7H-Pyrrolo[2,3-D]Pyrimidin-5-Yl)Imidazo[1,2-A]Pyridin-8-Yl)-3-(4-((4-Ethylpiperazin-1-Yl)Methyl)-3-(Trifluoromethyl)Phenyl)Urea

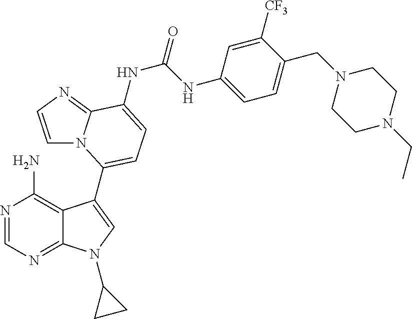 [SMILES: CCN1CCN(Cc2ccc(NC(=O)Nc3ccc(-c4cn(C5CC5)c5ncnc(N)c45)n4ccnc34)cc2C(F)(F)F)CC1]

The title compound was obtained by following a similar procedure described for Intermediate B1, starting from 1-(5-bromoimidazo[1,2-a]pyridin-8-yl)-3-(4-((4-ethylpiperazin-1-yl)methyl)-3-(trifluoromethyl)phenyl)urea (D5, 42 mg, 0.080 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 40 mg, 0.080 mmol), and was isolated as an off-white solid (2 mg, 4.1% yield). Cleavage of the di-Boc protecting group was observed during the reaction. ¹H NMR (400 MHz, CD₃OD) δ=8.26 (s, 1H), 8.02 (d, J=7.6 Hz, 1H), 7.98 (s, 1H), 7.73 (bs, 2H), 7.65 (d, J=1.6 Hz, 1H), 7.59 (d, J=1.2 Hz, 1H), 7.52 (s, 1H), 6.95 (d, J=8.0 Hz, 1H), 3.71 (s, 2H), 3.58-3.61 (m, 1H), 2.88 (bs, 4H), 2.79 (bs, 2H), 2.66 (bs, 4H), 1.14-1.25 (m, 7H). MS: 619.2 [M+H].

### Example 36

### 1-(5-(4-Amino-7-Cyclopropyl-7H-Pyrrolo[2,3-D]Pyrimidin-5-Yl)Imidazo[1,2-A]Pyridin-8-Yl)-3-(3-((4-Methylpiperazin-1-Yl)Methyl)-5-(Trifluoromethyl)Phenyl)Urea

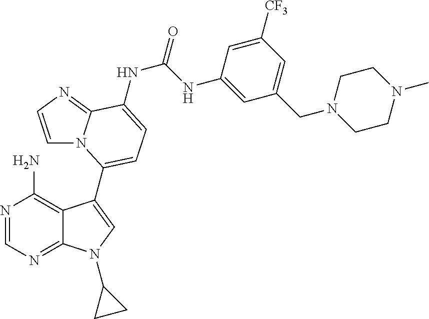 [SMILES: CN1CCN(Cc2cc(NC(=O)Nc3ccc(-c4cn(C5CC5)c5ncnc(N)c45)n4ccnc34)cc(C(F)(F)F)c2)CC1]

The title compound was obtained by following a similar procedure described for Intermediate B1, starting from 1-(5-bromoimidazo[1,2-a]pyridin-8-yl)-3-(3-((4-methylpiperazin-1-yl)methyl)-5-(trifluoromethyl)phenyl)urea (D6, 59 mg, 0.116 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 58 mg, 0.116 mmol), and was isolated as an off-white solid (8 mg, 11% yield). Cleavage of the di-Boc protecting group was observed during the reaction. ¹H NMR (400 MHz, DMSO-d₆) δ=10.04 (s, 1H), 9.18 (s, 1H), 8.20 (s, 1H), 7.97-7.99 (m, 2H), 7.97 (d, J=1.6 Hz, 1H), 7.54-7.57 (m, 3H), 7.26 (s, 1H), 6.82 (d, J=7.6 Hz, 1H), 6.13 (bs, 2H), 3.62-3.66 (m, 1H), 3.55 (s, 2H), 2.41 (bs, 8H), 2.20 (s, 3H), 1.04-1.13 (m, 4H). MS: 605.2 [M+H].

### Example 37

### 1-(5-(4-Amino-7-Cyclopropyl-7H-Pyrrolo[2,3-D]Pyrimidin-5-Yl)Imidazo[1,2-A]Pyridin-8-Yl)-3-(4-((1-Methylpiperidin-4-Yl)Oxy)-3-(Trifluoromethyl)Phenyl)Urea

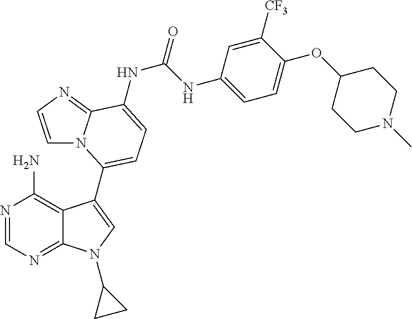 [SMILES: CN1CCC(Oc2ccc(NC(=O)Nc3ccc(-c4cn(C5CC5)c5ncnc(N)c45)n4ccnc34)cc2C(F)(F)F)CC1]

The title compound was obtained by following a similar procedure described for Intermediate B1, starting from 1-(5-bromoimidazo[1,2-a]pyridin-8-yl)-3-(4-((1-methylpiperidin-4-yl)oxy)-3-(trifluoromethyl)phenyl)urea (D7, 20 mg, 0.040 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 20 mg, 0.040 mmol), and was isolated as an off-white solid (2 mg, 8% yield). Cleavage of the di-Boc protecting group was observed during the reaction. ¹H NMR (400 MHz, CD₃O/500.2D) δ=8.40 (s, 1H), 8.05 (d, J=8.0 Hz, 1H), 7.86-7.97 (m, 3H), 7.78 (s, 1H), 7.71-7.73 (m, 1H), 7.26-7.29 (m, 2H), 4.85 (s, 1H), 3.52-3.76 (m, 1H), 3.50 (s, 2H), 2.94 (s, 3H), 1.90-2.35 (m, 6H), 1.20-1.26 (m, 4H). MS: 606.3 [M+H].

### Example 38

### 1-(5-(4-Amino-7-Cyclopropyl-7H-Pyrrolo[2,3-D]Pyrimidin-5-Yl)Imidazo[1,2-A]Pyridin-8-Yl)-3-(4-((1-Ethylpiperidin-4-Yl)Oxy)-3-(Trifluoromethyl)Phenyl)Urea

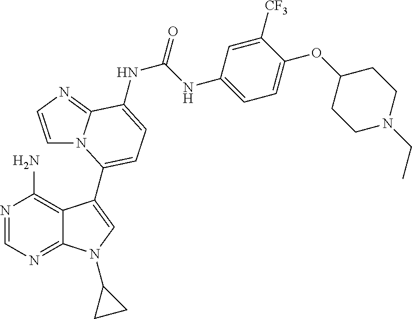 [SMILES: CCN1CCC(Oc2ccc(NC(=O)Nc3ccc(-c4cn(C5CC5)c5ncnc(N)c45)n4ccnc34)cc2C(F)(F)F)CC1]

The title compound is prepared by following a similar procedure described for Intermediate B1, starting from 1-(5-bromoimidazo[1,2-a]pyridin-8-yl)-3-(4-((1-ethylpiperidin-4-yl)oxy)-3-(trifluoromethyl)phenyl)urea (D8, 50 mg, 0.095 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 48 mg, 0.095 mmol) in the first step. MS (expected): 620.3 [M+H].

The title compound was obtained by following a similar procedure described for Intermediate B1, starting from 1-(5-bromoimidazo[1,2-a]pyridin-8-yl)-3-(4-((1-ethylpiperidin-4-yl)oxy)-3-(trifluoromethyl)phenyl)urea (D8, 150 mg, 0.285 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 214 mg, 0.427 mmol), and was isolated as an off-white solid (40 mg, 22% yield). Cleavage of the di-Boc protecting group was observed during the reaction. ¹H NMR (400 MHz, DMSO-d₆) δ=9.77 (bs, 1H), 9.08 (bs, 1H), 8.20 (s, 1H), 7.96 (d, J=7.6 Hz, 1H), 7.89 (d, J=2.4 Hz, 1H), 7.67 (d, J=1.6 Hz, 1H), 7.54-7.56 (m, 3H), 7.29 (d, J=9.2 Hz, 1H), 6.80 (d, J=8.0 Hz, 1H), 6.13 (bs, 2H), 4.55-4.56 (m, 1H), 3.62-3.65 (m, 1H), 2.56-2.61 (m, 2H), 2.24-2.34 (m, 4H), 1.90-1.94 (m, 2H), 1.67-1.71 (m, 2H), 0.99-1.13 (m, 7H). MS: 620.3 [M+H].

### Example 39

### 1-(5-(4-Amino-7-Cyclopropyl-7H-Pyrrolo[2,3-D]Pyrimidin-5-Yl)Imidazo[1,2-A]Pyridin-8-Yl)-3-(4-((4-(2-Hydroxyethyl)Piperazin-1-Yl)Methyl)-3-(Trifluoromethyl)Phenyl)Urea

### Step 1: Synthesis of 1-(5-(4-amino-7-cyclopropyl-7H-pyrrolo[2,3-d]pyrimidin-5-yl)imidazo[1,2-a]pyridin-8-yl)-3-(4-((4-(2-((tert-butyldiphenylsilyl)oxy)ethyl)piperazin-1-yl)methyl)-3-(trifluoromethyl)phenyl)urea

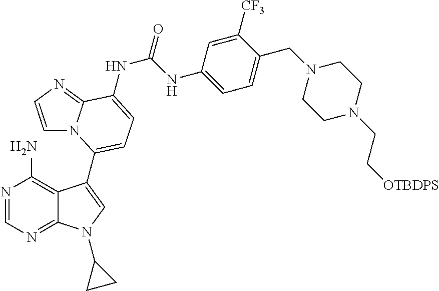

The title compound is prepared by following a similar procedure described for Intermediate B1, starting from 1-(5-bromoimidazo[1,2-a]pyridin-8-yl)-3-(4-((4-(2-((tert-butyldiphenylsilyl)oxy)ethyl)piperazin-1-yl)methyl)-3-(trifluoromethyl)phenyl)urea (D9, 100 mg, 0.128 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 64 mg, 0.128 mmol) in the first step. MS (expected): 873.4 [M+H].

The title compound was prepared by following a similar procedure described for Intermediate B1, starting from 1-(5-bromoimidazo[1,2-a]pyridin-8-yl)-3-(4-((4-(2-((tert-butyldiphenylsilyl)oxy)ethyl)piperazin-1-yl)methyl)-3-(trifluoromethyl)phenyl)urea (D9, 50.0 mg, 0.064 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 48.1 mg, 0.096 mmol), and was isolated as an off-white solid (21 mg, 34% yield). Cleavage of the di-Boc protecting group was observed during the reaction. LCMS: 873.3 [M+H].

### Step 2: Synthesis of 1-(5-(4-amino-7-cyclopropyl-7H-pyrrolo[2,3-d]pyrimidin-5-yl)imidazo[1,2-a]pyridin-8-yl)-3-(4-((4-(2-hydroxyethyl)piperazin-1-yl)methyl)-3-(trifluoromethyl)phenyl)urea

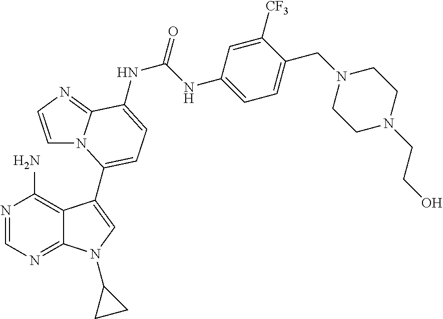 [SMILES: Nc1ncnc2c1c(-c1ccc(NC(=O)Nc3ccc(CN4CCN(CCO)CC4)c(C(F)(F)F)c3)c3nccn13)cn2C1CC1]

TBAF (1M in THF, 0.057 mL, 0.057 mmol) is added to a solution of 1-(5-(4-amino-7-cyclopropyl-7H-pyrrolo[2,3-d]pyrimidin-5-yl)imidazo[1,2-a]pyridin-8-yl)-3-(4-((4-(2-((tert-butyldiphenylsilyl)oxy)ethyl)piperazin-1-yl)methyl)-3-(trifluoromethyl)phenyl)urea (50 mg, 0.057 mmol) in THF (5 mL) at 0° C. and the resulting solution is stirred at 25° C. for 1 h. Following completion of the reaction (as indicated by LCMS), the reaction mixture is concentrated under reduced pressure to give crude material, which is purified by preparative HPLC, affording the title compound. MS (expected): 635.3 [M+H].

TBAF (1M in THF, 0.023 mL, 0.023 mmol) was added to a solution of 1-(5-(4-amino-7-cyclopropyl-7H-pyrrolo[2,3-d]pyrimidin-5-yl)imidazo[1,2-a]pyridin-8-yl)-3-(4-((4-(2-((tert-butyldiphenylsilyl)oxy)ethyl)piperazin-1-yl)methyl)-3-(trifluoromethyl)phenyl)urea (20 mg, 0.023 mmol) in THF (5 mL) at 0° C. and the resulting solution is stirred at 25° C. for 1 h. Following completion of the reaction (as indicated by LCMS), the reaction mixture is concentrated under reduced pressure to give crude material which is purified by preparative HPLC (eluting with a gradient of 0.1% formic acid in water and acetonitrile), affording the title compound (formate salt) as an off-white solid (6 mg, 41% yield). ¹H NMR (400 MHz, CD₃OD) δ=8.26 (s, 1H), 8.02 (d, J=7.6 Hz, 1H), 7.98 (s, 1H), 7.74 (bs, 2H), 7.66 (s, 1H), 7.59 (s, 1H), 7.52 (s, 1H), 6.95 (d, J=8.0 Hz, 1H), 3.81 (bs, 2H), 3.73 (bs, 2H), 3.57-3.62 (m, 1H), 3.12-3.28 (m, 2H), 2.71-3.06 (m, 8H), 1.12-1.22 (m, 4H). MS: 635.2 [M+H].

### Example 40

### 1-(5-(4-Amino-7-Cyclopropyl-7H-Pyrrolo[2,3-D]Pyrimidin-5-Yl)Imidazo[1,2-A]Pyridin-8-Yl)-3-(4-((4-(2-Methoxyethyl)Piperazin-1-Yl)Methyl)-3-(Trifluoromethyl)Phenyl)Urea

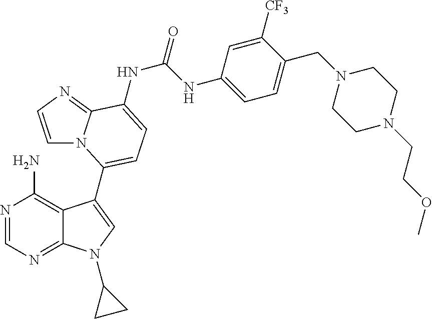 [SMILES: COCCN1CCN(Cc2ccc(NC(=O)Nc3ccc(-c4cn(C5CC5)c5ncnc(N)c45)n4ccnc34)cc2C(F)(F)F)CC1]

The title compound is prepared by following a similar procedure described for Intermediate B1, starting from 1-(5-bromoimidazo[1,2-a]pyridin-8-yl)-3-(4-((4-(2-methoxyethyl)piperazin-1-yl)methyl)-3-(trifluoromethyl)phenyl)urea (D10, 50 mg, 0.090 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 45 mg, 0.090 mmol) in the first step. MS (expected): 649.6 [M+H].

The title compound was prepared by following a similar procedure described for Intermediate B1, starting from 1-(5-bromoimidazo[1,2-a]pyridin-8-yl)-3-(4-((4-(2-methoxyethyl)piperazin-1-yl)methyl)-3-(trifluoromethyl)phenyl)urea (D10, 50.0 mg, 0.090 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 67.6 mg, 0.135 mmol), and was isolated as an off-white solid (15 mg, 25% yield). Cleavage of the di-Boc protecting group was observed during the reaction. ¹H NMR (400 MHz, DMSO-d₆) δ=9.99 (bs, 1H), 9.17 (bs, 1H), 8.20 (s, 1H), 7.97-8.03 (m, 2H), 7.66-7.68 (m, 2H), 7.56-7.60 (m, 3H), 6.81 (d, J=8.0 Hz, 1H), 6.14 (bs, 2H), 3.61-3.66 (m, 1H), 3.54 (bs, 2H), 3.37-3.47 (m, 4H), 3.23 (s, 3H), 2.33-2.48 (m, 8H), 1.02-1.16 (m, 4H). MS: 649.3 [M+H].

### Example 41

### 1-(5-(4-Amino-7-Cyclopropyl-7H-Pyrrolo[2,3-D]Pyrimidin-5-Yl)Imidazo[1,2-A]Pyridin-8-Yl)-3-(4-((4-(2-Fluoroethyl)Piperazin-1-Yl)Methyl)-3-(Trifluoromethyl)Phenyl)Urea

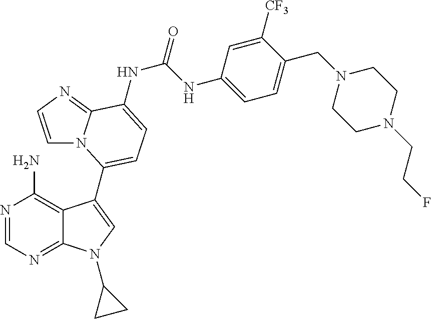 [SMILES: Nc1ncnc2c1c(-c1ccc(NC(=O)Nc3ccc(CN4CCN(CCF)CC4)c(C(F)(F)F)c3)c3nccn13)cn2C1CC1]

The title compound is prepared by following a similar procedure described for Intermediate B1, starting from 1-(5-bromoimidazo[1,2-a]pyridin-8-yl)-3-(4-((4-(2-fluoroethyl)piperazin-1-yl)methyl)-3-(trifluoromethyl)phenyl)urea (D11, 50 mg, 0.092 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 46 mg, 0.092 mmol) in the first step. MS (expected): 637.3 [M+H].

The title compound is prepared by following a similar procedure described for Intermediate B1, starting from 1-(5-bromoimidazo[1,2-a]pyridin-8-yl)-3-(4-((4-(2-fluoroethyl)piperazin-1-yl)methyl)-3-(trifluoromethyl)phenyl)urea (D11, 50.0 mg, 0.092 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 69.1 mg, 0.138 mmol), and was isolated as an off-white solid (14 mg, 24% yield). Cleavage of the di-Boc protecting group was observed during the reaction. ¹H NMR (400 MHz, DMSO-d₆) δ=9.99 (bs, 1H), 9.17 (bs, 1H), 8.20 (s, 1H), 8.03 (d, J=2.0 Hz, 1H), 7.98 (d, J=8.0 Hz, 1H), 7.66-7.68 (m, 2H), 7.56-7.60 (m, 3H), 6.81 (d, J=7.6 Hz, 1H), 6.13 (bs, 2H), 4.59 (t, J=4.8 Hz, 1H), 4.47 (t, J=4.8 Hz, 1H), 3.60-3.64 (m, 1H), 3.55 (s, 2H), 2.66 (t, J=4.8 Hz, 1H), 2.59 (t, J=4.8 Hz, 1H), 2.34-2.42 (m, 8H), 1.02-1.13 (m, 4H). MS: 637.2 [M+H].

### Example 42

### 1-(5-(4-Amino-7-Cyclopropyl-7H-Pyrrolo[2,3-D]Pyrimidin-5-Yl)-2-Fluoroimidazo[1,2-A]Pyridin-8-Yl)-3-(5-(1-(Trifluoromethyl)Cyclopropyl)Isoxazol-3-Yl)Urea

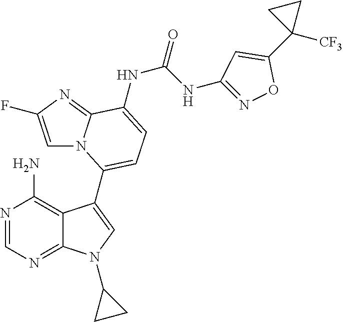 [SMILES: Nc1ncnc2c1c(-c1ccc(NC(=O)Nc3cc(C4(C(F)(F)F)CC4)on3)c3nc(F)cn13)cn2C1CC1]

The title compound is prepared by following a similar two-step procedure described for Example 5, starting from tert-butyl (5-(8-amino-2-fluoroimidazo[1,2-a]pyridin-5-yl)-7-cyclopropyl-7H-pyrrolo[2,3-d]pyrimidin-4-yl)(tert-butoxycarbonyl)carbamate (B6, 50 mg, 0.095 mmol) and phenyl (5-(1-(trifluoromethyl)cyclopropyl)isoxazol-3-yl)carbamate (C3, 30 mg, 0.095 mmol) in step 1. MS (expected): 542.3 [M+H].

### Example 43

### 1-(5-(4-Amino-7-Cyclopropyl-7H-Pyrrolo[2,3-D]Pyrimidin-5-Yl)-2-Fluoroimidazo[1,2-A]Pyridin-8-Yl)-3-(4-((4-Methylpiperazin-1-Yl)Methyl)-3-(Trifluoromethyl)Phenyl)Urea

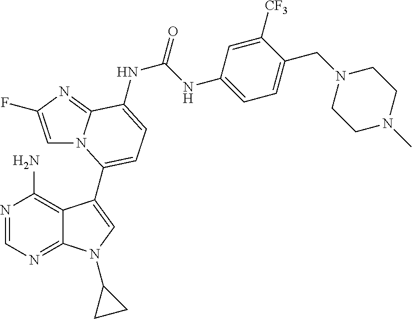 [SMILES: CN1CCN(Cc2ccc(NC(=O)Nc3ccc(-c4cn(C5CC5)c5ncnc(N)c45)n4cc(F)nc34)cc2C(F)(F)F)CC1]

The title compound is prepared by following a similar two-step procedure described for Example 5, starting from tert-butyl (5-(8-amino-2-fluoroimidazo[1,2-a]pyridin-5-yl)-7-cyclopropyl-7H-pyrrolo[2,3-d]pyrimidin-4-yl)(tert-butoxycarbonyl)carbamate (B6, 50 mg, 0.095 mmol) and phenyl (4-((4-methylpiperazin-1-yl)methyl)-3-(trifluoromethyl)phenyl)carbamate (C5, 38 mg, 0.095 mmol) in step 1. MS (expected): 605.3 [M+H].

### Example 44

### 1-(5-(4-Amino-7-Cyclopropyl-7H-Pyrrolo[2,3-D]Pyrimidin-5-Yl)-2-Methylimidazo[1,2-A]Pyridin-8-Yl)-3-(5-(1-(Trifluoromethyl)Cyclopropyl)Isoxazol-3-Yl)Urea

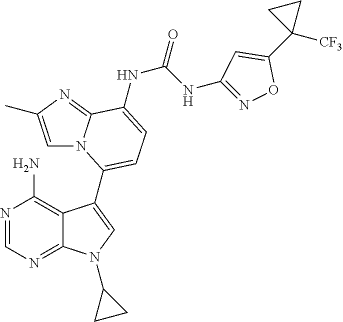 [SMILES: Cc1cn2c(-c3cn(C4CC4)c4ncnc(N)c34)ccc(NC(=O)Nc3cc(C4(C(F)(F)F)CC4)on3)c2n1]

The title compound is prepared by following a similar two-step procedure described for Example 5, starting from tert-butyl (5-(8-amino-2-methylimidazo[1,2-a]pyridin-5-yl)-7-cyclopropyl-7H-pyrrolo[2,3-d]pyrimidin-4-yl)(tert-butoxycarbonyl)carbamate (B7, 50 mg, 0.096 mmol) and phenyl (5-(1-(trifluoromethyl)cyclopropyl)isoxazol-3-yl)carbamate (C3, 30 mg, 0.096 mmol) in step 1. MS (expected): 538.2 [M+H].

### Example 45

### 1-(5-(4-Amino-7-Cyclopropyl-7H-Pyrrolo[2,3-D]Pyrimidin-5-Yl)-2-Methylimidazo[1,2-A]Pyridin-8-Yl)-3-(4-((4-Methylpiperazin-1-Yl)Methyl)-3-(Trifluoromethyl)Phenyl)Urea

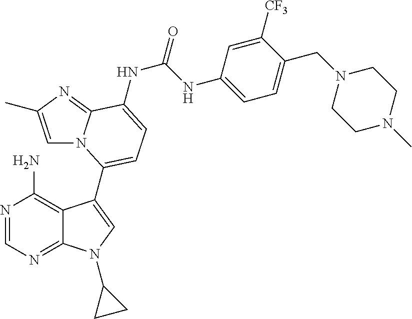

The title compound is prepared by following a similar two-step procedure described for Example 5, starting from tert-butyl (5-(8-amino-2-methylimidazo[1,2-a]pyridin-5-yl)-7-cyclopropyl-7H-pyrrolo[2,3-d]pyrimidin-4-yl)(tert-butoxycarbonyl)carbamate (B7, 50 mg, 0.096 mmol) and phenyl (4-((4-methylpiperazin-1-yl)methyl)-3-(trifluoromethyl)phenyl)carbamate (C3, 38 mg, 0.096 mmol) in step 1. MS (expected): 619.3 [M+H].

### Example 46

### 1-(5-(4-Amino-7-Cyclopropyl-7H-Pyrrolo[2,3-D]Pyrimidin-5-Yl)-2-(Trifluoromethyl)Imidazo[1,2-A]Pyridin-8-Yl)-3-(5-(1-(Trifluoromethyl)Cyclopropyl)Isoxazol-3-Yl)Urea

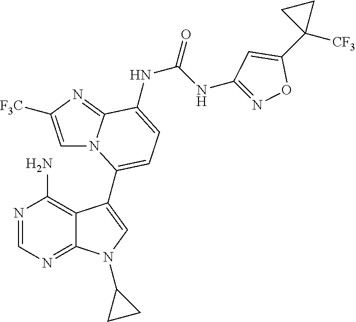 [SMILES: Nc1ncnc2c1c(-c1ccc(NC(=O)Nc3cc(C4(C(F)(F)F)CC4)on3)c3nc(C(F)(F)F)cn13)cn2C1CC1]

The title compound was obtained by following a similar procedure described for Intermediate B1, starting from, 1-(5-bromo-2-(trifluoromethyl)imidazo[1,2-a]pyridin-8-yl)-3-(5-(1-(trifluoromethyl)cyclopropyl)isoxazol-3-yl)urea (D12, 20 mg, 0.040 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 20 mg, 0.040 mmol), and was isolated as an off-white solid (0.5 mg, 2.1% yield). MS: 592.1 [M+H].

### Example 47

### 1-(5-(4-Amino-7-Cyclopropyl-7H-Pyrrolo[2,3-D]Pyrimidin-5-Yl)-2-(Trifluoromethyl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of tert-butyl (tert-butoxycarbonyl)(7-cyclopropyl-5-(8-(3-(4-((4-methylpiperazin-1-yl)methyl)-3-(trifluoromethyl)phenyl)ureido)-2-(trifluoromethyl)imidazo[1,2-a]pyridin-5-yl)-7H-pyrrolo[2,3-d]pyrimidin-4-yl)carbamate

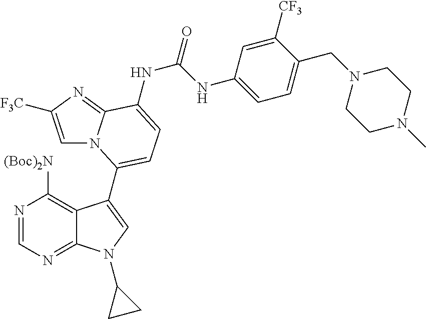

The title compound was obtained by following a similar procedure described for Intermediate C1, starting from 1-(5-bromo-2-(trifluoromethyl)imidazo[1,2-a]pyridin-8-yl)-3-(4-((4-methylpiperazin-1-yl)methyl)-3-(trifluoromethyl)phenyl)urea (D13, 25 mg, 0.043 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 22 mg, 0.043 mmol), and was isolated as an off-white solid (20 mg, 54% yield). MS: 873.3 [M+H].

### Step 2: Synthesis of 1-(5-(4-amino-7-cyclopropyl-7H-pyrrolo[2,3-d]pyrimidin-5-yl)-2-(trifluoromethyl)imidazo[1,2-a]pyridin-8-yl)-3-(4-((4-methylpiperazin-1-yl)methyl)-3-(trifluoromethyl)phenyl)urea

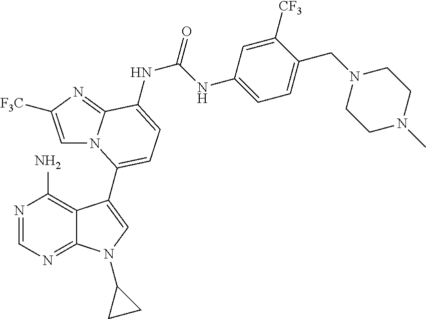 [SMILES: CN1CCN(Cc2ccc(NC(=O)Nc3ccc(-c4cn(C5CC5)c5ncnc(N)c45)n4cc(C(F)(F)F)nc34)cc2C(F)(F)F)CC1]

The title compound was obtained by following a similar procedure described for step 2 of Example 5, starting from tert-butyl (tert-butoxycarbonyl)(7-cyclopropyl-5-(8-(3-(4-((4-methylpiperazin-1-yl)methyl)-3-(trifluoromethyl)phenyl)ureido)-2-(trifluoromethyl)imidazo[1,2-a]pyridin-5-yl)-7H-pyrrolo[2,3-d]pyrimidin-4-yl)carbamate (13 mg, 0.015 mmol), and was isolated as a an off-white solid (2 mg, 20% yield). ¹H NMR (400 MHz, CD₃OD) δ=8.27 (s, 1H), 8.16 (d, J=8.0 Hz, 1H), 7.97-7.99 (m, 2H), 7.71-7.72 (m, 2H), 7.54 (s, 1H), 7.03 (d, J=8.0 Hz, 1H), 3.70 (s, 2H), 3.57-3.61 (m, 1H), 2.83 (bs, 4H), 2.64 (bs, 4H), 2.54 (s, 3H), 1.15-1.20 (m, 4H). MS: 673.2 [M+H].

### Example 48

### 1-(5-(4-Amino-7-Cyclopropyl-7H-Pyrrolo[2,3-D]Pyrimidin-5-Yl)-3-Methylimidazo[1,2-A]Pyridin-8-Yl)-3-(5-(1-(Trifluoromethyl)Cyclopropyl)Isoxazol-3-Yl)Urea

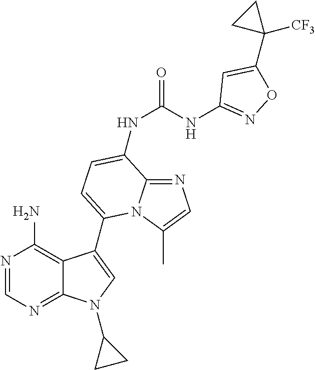 [SMILES: Cc1cnc2c(NC(=O)Nc3cc(C4(C(F)(F)F)CC4)on3)ccc(-c3cn(C4CC4)c4ncnc(N)c34)n12]

The title compound is prepared by following a similar two-step procedure described for Example 5, starting from tert-butyl (5-(8-amino-3-methylimidazo[1,2-a]pyridin-5-yl)-7-cyclopropyl-7H-pyrrolo[2,3-d]pyrimidin-4-yl)(tert-butoxycarbonyl)carbamate (B8, 50 mg, 0.096 mmol) and phenyl (5-(1-(trifluoromethyl)cyclopropyl)isoxazol-3-yl)carbamate (C3, 30 mg, 0.096 mmol) in step 1. MS (expected): 538.2 [M+H].

### Example 49

### 1-(5-(4-Amino-7-Cyclopropyl-7H-Pyrrolo[2,3-D]Pyrimidin-5-Yl)-3-Methylimidazo[1,2-A]Pyridin-8-Yl)-3-(4-((4-Methylpiperazin-1-Yl)Methyl)-3-(Trifluoromethyl)Phenyl)Urea

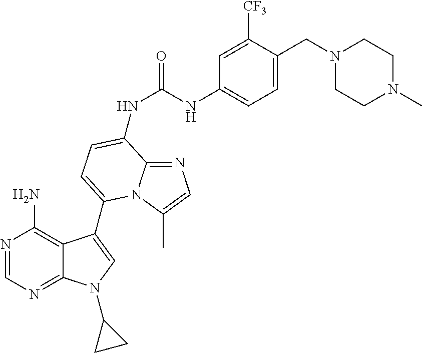 [SMILES: Cc1cnc2c(NC(=O)Nc3ccc(CN4CCN(C)CC4)c(C(F)(F)F)c3)ccc(-c3cn(C4CC4)c4ncnc(N)c34)n12]

The title compound is prepared by following a similar two-step procedure described for Example 5, starting from tert-butyl (5-(8-amino-3-methylimidazo[1,2-a]pyridin-5-yl)-7-cyclopropyl-7H-pyrrolo[2,3-d]pyrimidin-4-yl)(tert-butoxycarbonyl)carbamate (B8, 50 mg, 0.096 mmol) and phenyl (4-((4-methylpiperazin-1-yl)methyl)-3-(trifluoromethyl)phenyl)carbamate (C5, 38 mg, 0.096 mmol) in step 1. MS (expected): 619.3 [M+H].

### Example 50

### 1-(5-(4-Amino-7-Cyclopropyl-7H-Pyrrolo[2,3-D]Pyrimidin-5-Yl)Imidazo[1,2-A]Pyrazin-8-Yl)-3-(5-(1-(Trifluoromethyl)Cyclopropyl)Isoxazol-3-Yl)Urea

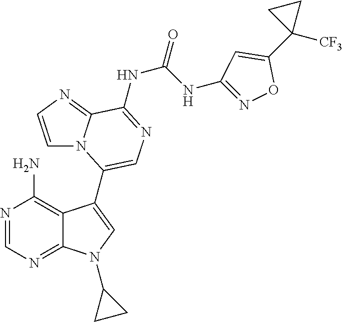 [SMILES: Nc1ncnc2c1c(-c1cnc(NC(=O)Nc3cc(C4(C(F)(F)F)CC4)on3)c3nccn13)cn2C1CC1]

The title compound was obtained by following the general procedure for urea formation (Method B), starting from 5-(8-aminoimidazo[1,2-a]pyrazin-5-yl)-7-cyclopropyl-7H-pyrrolo[2,3-d]pyrimidin-4-amine (B9, 100 mg, 0.326 mmol) and phenyl (5-(1-(trifluoromethyl)cyclopropyl)isoxazol-3-yl)carbamate (C3, 102 mg, 0.326 mmol), and was isolated as an off-white solid (2 mg, 1.2% yield). MS: 525.4 [M+H].

### Example 51

### 1-(5-(4-Amino-7-Cyclopropyl-7H-Pyrrolo[2,3-D]Pyrimidin-5-Yl)Imidazo[1,2-A]Pyrazin-8-Yl)-3-(4-((4-Methylpiperazin-1-Yl)Methyl)-3-(Trifluoromethyl)Phenyl)Urea

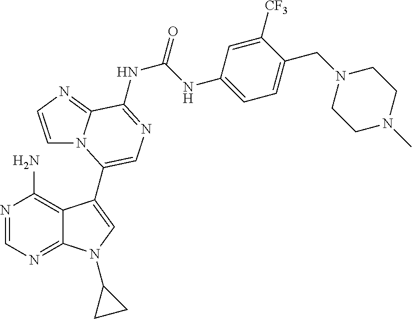 [SMILES: CN1CCN(Cc2ccc(NC(=O)Nc3ncc(-c4cn(C5CC5)c5ncnc(N)c45)n4ccnc34)cc2C(F)(F)F)CC1]

The title compound was obtained by following a similar procedure described for Intermediate B1, starting from 1-(5-bromoimidazo[1,2-a]pyrazin-8-yl)-3-(4-((4-methylpiperazin-1-yl)methyl)-3-(trifluoromethyl)phenyl)urea (D14, 31 mg, 0.060 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 30 mg, 0.060 mmol), and was isolated as an off-white solid (2 mg, 5.6% yield). Cleavage of the di-Boc protecting group was observed during the reaction. LCMS: 606.2 [M+H]

### Example 52

### 1-(4-(4-Amino-7-Cyclopropyl-7H-Pyrrolo[2,3-D]Pyrimidin-5-Yl)-2,3-Dihydrobenzofuran-7-Yl)-3-(5-(1-(Trifluoromethyl)Cyclopropyl)Isoxazol-3-Yl)Urea

### Step 1: Synthesis of tert-butyl (tert-butoxycarbonyl)(7-cyclopropyl-5-(7-(3-(5-(1-(trifluoromethyl)cyclopropyl)isoxazol-3-yl)ureido)-2,3-dihydrobenzofuran-4-yl)-7H-pyrrolo[2,3-d]pyrimidin-4-yl)carbamate

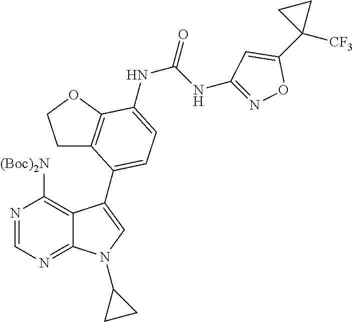

The title compound was obtained by following a similar procedure described for Intermediate B1, starting from 1-(4-bromo-2,3-dihydrobenzofuran-7-yl)-3-(5-(1-(trifluoromethyl)cyclopropyl)isoxazol-3-yl)urea (D15, 59 mg, 0.136 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 68 mg, 0.136 mmol), and was isolated as an off-white solid (20 mg, 20% yield). MS: 726.4 [M+H].

### Step 2: Synthesis of 1-(4-(4-amino-7-cyclopropyl-7H-pyrrolo[2,3-d]pyrimidin-5-yl)-2,3-dihydrobenzofuran-7-yl)-3-(5-(1-(trifluoromethyl)cyclopropyl)isoxazol-3-yl)urea

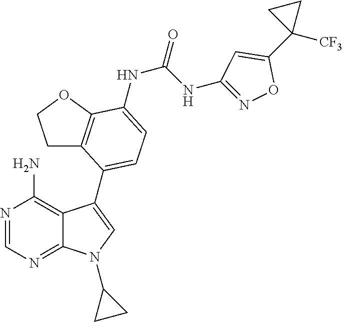 [SMILES: Nc1ncnc2c1c(-c1ccc(NC(=O)Nc3cc(C4(C(F)(F)F)CC4)on3)c3c1CCO3)cn2C1CC1]

The title compound was obtained by following a similar procedure described for step 2 of Example 5, starting from tert-butyl (tert-butoxycarbonyl)(7-cyclopropyl-5-(7-(3-(5-(1-(trifluoromethyl)cyclopropyl)isoxazol-3-yl)ureido)-2,3-dihydrobenzofuran-4-yl)-7H-pyrrolo[2,3-d]pyrimidin-4-yl)carbamate (20 mg, 0.032 mmol), and was isolated as an off-white solid (5 mg, 29% yield). ¹H NMR (400 MHz, DMSO-d₆) δ=9.95 (s, 1H), 8.58 (s, 1H), 8.15 (s, 1H), 7.91 (d, J=8.4 Hz, 1H), 7.22 (s, 1H), 6.86 (s, 1H), 6.78 (d, J=8.4 Hz, 1H), 6.02 (bs, 2H), 4.64 (t, J=8.8 Hz, 2H), 3.54-3.60 (m, 1H), 3.25 (t, J=8.8 Hz, 2H), 1.46-1.56 (m, 4H), 1.01-1.04 (m, 4H). MS: 526.2 [M+H].

### Example 53

### 1-(4-(4-Amino-7-Cyclopropyl-7H-Pyrrolo[2,3-D]Pyrimidin-5-Yl)-2,3-Dihydrobenzofuran-7-Yl)-3-(4-((4-Methylpiperazin-1-Yl)Methyl)-3-(Trifluoromethyl)Phenyl)Urea

### Step 1: Synthesis of tert-butyl (tert-butoxycarbonyl)(7-cyclopropyl-5-(7-(3-(4-((4-methylpiperazin-1-yl)methyl)-3-(trifluoromethyl)phenyl)ureido)-2,3-dihydrobenzofuran-4-yl)-7H-pyrrolo[2,3-d]pyrimidin-4-yl)carbamate

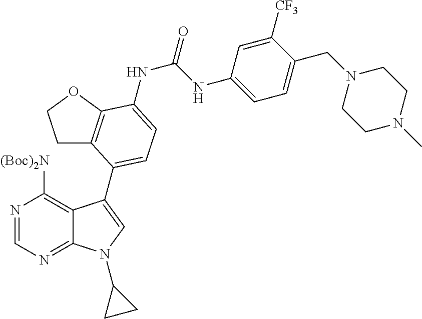

The title compound was obtained by following a similar procedure described for Intermediate B1, starting from 1-(4-bromo-2,3-dihydrobenzofuran-7-yl)-3-(4-((4-methylpiperazin-1-yl)methyl)-3-(trifluoromethyl)phenyl)urea (D16, 215 mg, 0.420 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 210 mg, 0.420 mmol), and was isolated as a pale brown gum (20 mg, 8% yield). MS: 807.3 [M+H].

### Step 2: Synthesis of 1-(4-(4-amino-7-cyclopropyl-7H-pyrrolo[2,3-d]pyrimidin-5-yl)-2,3-dihydrobenzofuran-7-yl)-3-(4-((4-methylpiperazin-1-yl)methyl)-3-(trifluoromethyl)phenyl)urea

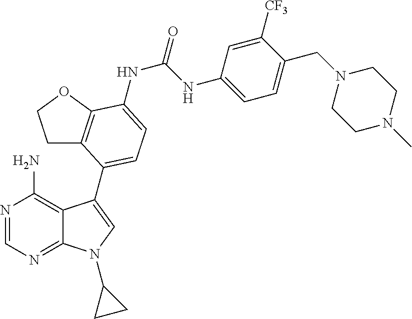 [SMILES: CN1CCN(Cc2ccc(NC(=O)Nc3ccc(-c4cn(C5CC5)c5ncnc(N)c45)c4c3OCC4)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((4-methylpiperazin-1-yl)methyl)-3-(trifluoromethyl)phenyl)ureido)-2,3-dihydrobenzofuran-4-yl)-7H-pyrrolo[2,3-d]pyrimidin-4-yl)carbamate (20 mg, 0.025 mmol), and was isolated as an off-white solid (4 mg, 17% yield). ¹H NMR (400 MHz, CD₃OD) δ=8.19 (s, 1H), 7.90 (d, J=2.4 Hz, 1H), 7.86 (d, J=8.4 Hz, 1H), 7.70 (d, J=8.4 Hz, 1H), 7.62-7.64 (m, 1H), 7.15 (s, 1H), 6.86 (d, J=8.4 Hz, 1H), 4.67-4.71 (m, 2H), 3.65 (s, 2H), 3.49-3.54 (m, 1H), 3.27-3.29 (m, 2H), 2.57 (bs, 8H), 2.37 (s, 3H), 1.13-1.16 (m, 2H), 1.07-1.10 (m, 2H). MS: 607.7 [M+H].

### Example 54

### 1-(4-(4-Amino-7-Cyclopropyl-7H-Pyrrolo[2,3-D]Pyrimidin-5-Yl)-2,3-Dihydrobenzofuran-7-Yl)-3-(4-((4-Ethylpiperazin-1-Yl)Methyl)-3-(Trifluoromethyl)Phenyl)Urea

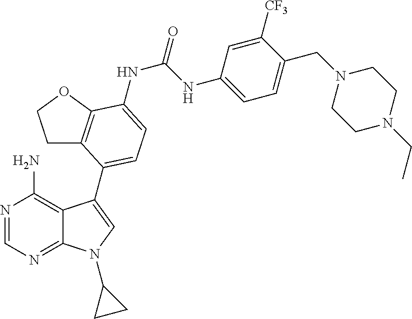 [SMILES: CCN1CCN(Cc2ccc(NC(=O)Nc3ccc(-c4cn(C5CC5)c5ncnc(N)c45)c4c3OCC4)cc2C(F)(F)F)CC1]

The title compound was obtained by following a similar procedure described for Intermediate B1, starting from tert-butyl (5-(7-amino-2,3-dihydrobenzofuran-4-yl)-7-cyclopropyl-7H-pyrrolo[2,3-d]pyrimidin-4-yl)(tert-butoxycarbonyl)carbamate (B10, 25 mg, 0.049 mmol) and phenyl (4-((4-ethylpiperazin-1-yl)methyl)-3-(trifluoromethyl)phenyl)carbamate (C33, 20 mg, 0.049 mmol), and was isolated as an off-white solid (3 mg, 10% yield over 2 steps). ¹H NMR (400 MHz, CD₃OD) δ=8.19 (s, 1H), 7.90 (d, J=2.4 Hz, 1H), 7.86 (d, J=8.4 Hz, 1H), 7.70 (d, J=8.4 Hz, 1H), 7.63-7.65 (m, 1H), 7.15 (s, 1H), 6.86 (d, J=8.0 Hz, 1H), 4.67-4.71 (m, 2H), 3.66 (s, 2H), 3.49-3.54 (m, 1H), 3.24-3.32 (m, 2H), 2.62-2.76 (m, 10H), 1.07-1.21 (m, 7H). LCMS: 621.8 [M+H].

### Step 1: Synthesis of tert-butyl (tert-butoxycarbonyl)(7-cyclopropyl-5-(7-(3-(4-((4-ethylpiperazin-1-yl)methyl)-3-(trifluoromethyl)phenyl)ureido)-2,3-dihydrobenzofuran-4-yl)-7H-pyrrolo[2,3-d]pyrimidin-4-yl)carbamate

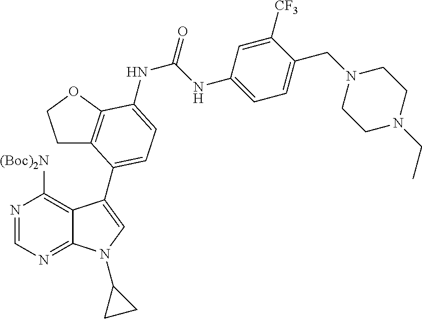

The title compound was obtained by following the general procedure for urea formation (Method B), starting from tert-butyl (5-(7-amino-2,3-dihydrobenzofuran-4-yl)-7-cyclopropyl-7H-pyrrolo[2,3-d]pyrimidin-4-yl)(tert-butoxycarbonyl)carbamate (B10, 25 mg, 0.049 mmol) and phenyl (4-((4-ethylpiperazin-1-yl)methyl)-3-(trifluoromethyl)phenyl)carbamate (C33, 20 mg, 0.049 mmol), and was isolated as a brown gum (20 mg, 50% yield). MS: 821.3 [M+H].

### Step 2: Synthesis of 1-(4-(4-amino-7-cyclopropyl-7H-pyrrolo[2,3-d]pyrimidin-5-yl)-2,3-dihydrobenzofuran-7-yl)-3-(4-((4-ethylpiperazin-1-yl)methyl)-3-(trifluoromethyl)phenyl)urea

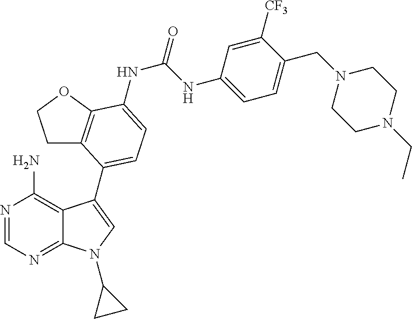 [SMILES: CCN1CCN(Cc2ccc(NC(=O)Nc3ccc(-c4cn(C5CC5)c5ncnc(N)c45)c4c3OCC4)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((4-ethylpiperazin-1-yl)methyl)-3-(trifluoromethyl)phenyl)ureido)-2,3-dihydrobenzofuran-4-yl)-7H-pyrrolo[2,3-d]pyrimidin-4-yl)carbamate (20 mg, 0.024 mmol), and was isolated as an off-white solid (3 mg, 20% yield). ¹H NMR (400 MHz, CD₃OD) δ=8.19 (s, 1H), 7.90 (d, J=2.4 Hz, 1H), 7.86 (d, J=8.4 Hz, 1H), 7.70 (d, J=8.4 Hz, 1H), 7.63-7.65 (m, 1H), 7.15 (s, 1H), 6.86 (d, J=8.0 Hz, 1H), 4.67-4.71 (m, 2H), 3.66 (s, 2H), 3.49-3.54 (m, 1H), 3.24-3.32 (m, 2H), 2.62-2.76 (m, 10H), 1.07-1.21 (m, 7H). MS: 621.8 [M+H].

### Example 55

### 1-(4-(4-Amino-7-Cyclopropyl-7H-Pyrrolo[2,3-D]Pyrimidin-5-Yl)-2,3-Dihydrobenzofuran-7-Yl)-3-(4-((1-Methylpiperidin-4-Yl)Oxy)-3-(Trifluoromethyl)Phenyl)Urea

### Step 1: Synthesis of tert-butyl (tert-butoxycarbonyl)(7-cyclopropyl-5-(7-(3-(4-((1-methylpiperidin-4-yl)oxy)-3-(trifluoromethyl)phenyl)ureido)-2,3-dihydrobenzofuran-4-yl)-7H-pyrrolo[2,3-d]pyrimidin-4-yl)carbamate

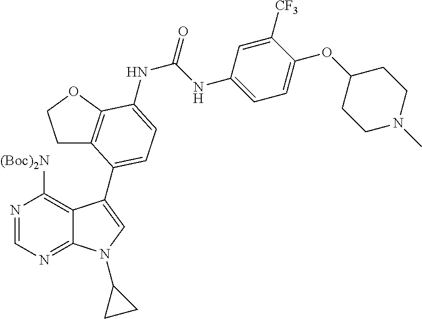

The title compound was obtained by following a similar procedure described for Intermediate B1, starting from 1-(4-bromo-2,3-dihydrobenzofuran-7-yl)-3-(4-((1-methylpiperidin-4-yl)oxy)-3-(trifluoromethyl)phenyl)urea (D17, 51 mg, 0.100 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 50 mg, 0.100 mmol), and was isolated as an off-white solid (10 mg, 12% yield). MS: 808.6 [M+H].

### Step 2: Synthesis of 1-(4-(4-amino-7-cyclopropyl-7H-pyrrolo[2,3-d]pyrimidin-5-yl)-2,3-dihydrobenzofuran-7-yl)-3-(4-((1-methylpiperidin-4-yl)oxy)-3-(trifluoromethyl)phenyl)urea

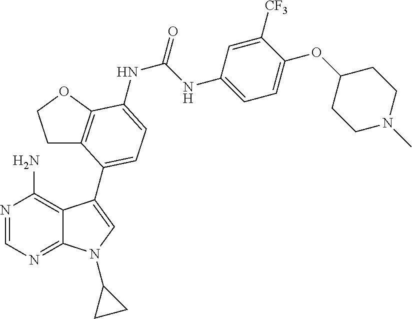 [SMILES: CN1CCC(Oc2ccc(NC(=O)Nc3ccc(-c4cn(C5CC5)c5ncnc(N)c45)c4c3OCC4)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((1-methylpiperidin-4-yl)oxy)-3-(trifluoromethyl)phenyl)ureido)-2,3-dihydrobenzofuran-4-yl)-7H-pyrrolo[2,3-d]pyrimidin-4-yl)carbamate (10 mg, 0.012 mmol), and was isolated as an off-white solid (1 mg, 13% yield). ¹H NMR (400 MHz, CD₃OD) δ=8.18 (s, 1H), 7.83 (d, J=8.4 Hz, 1H), 7.76-7.77 (m, 1H), 7.58-7.61 (m, 1H), 7.19 (d, J=8.8 Hz, 1H), 7.14 (s, 1H), 6.86 (d, J=8.0 Hz, 1H), 4.66-4.71 (m, 1H), 3.01-3.03 (m, 1H), 2.78 (bs, 4H), 2.61 (bs, 4H), 2.41 (s, 3H), 2.03-2.04 (m, 2H), 1.94-1.96 (m, 2H), 1.10-1.18 (m, 4H). MS: 608.2 [M+H].

### Example 56

### 1-(4-(4-Amino-7-Cyclopropyl-7H-Pyrrolo[2,3-D]Pyrimidin-5-Yl)-2-Methyl-2,3-Dihydrobenzofuran-7-Yl)-3-(5-(1-(Trifluoromethyl)Cyclopropyl)Isoxazol-3-Yl)Urea

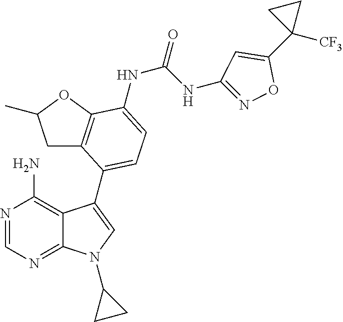 [SMILES: CC1Cc2c(-c3cn(C4CC4)c4ncnc(N)c34)ccc(NC(=O)Nc3cc(C4(C(F)(F)F)CC4)on3)c2O1]

The title compound is prepared by following a similar procedure described for Intermediate B1, starting from 1-(4-bromo-2-methyl-2,3-dihydrobenzofuran-7-yl)-3-(5-(1-(trifluoromethyl)cyclopropyl)isoxazol-3-yl)urea (D18, 50 mg, 0.112 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 56 mg, 0.112 mmol). MS (expected): 540.2 [M+H].

### Step 1: Synthesis of tert-butyl (tert-butoxycarbonyl)(7-cyclopropyl-5-(2-methyl-7-(3-(5-(1-(trifluoromethyl)cyclopropyl)isoxazol-3-yl)ureido)-2,3-dihydrobenzofuran-4-yl)-7H-pyrrolo[2,3-d]pyrimidin-4-yl)carbamate

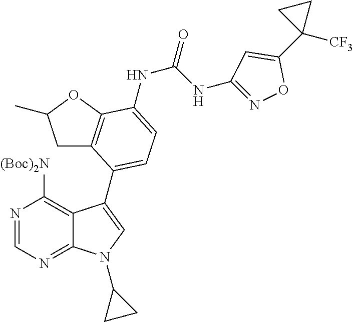

The title compound was obtained by following a similar procedure described for Intermediate B1, starting from 1-(4-bromo-2-methyl-2,3-dihydrobenzofuran-7-yl)-3-(5-(1-(trifluoromethyl)cyclopropyl)isoxazol-3-yl)urea (D18, 50.0 mg, 0.112 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 44.9 mg, 0.112 mmol), and was isolated as an off-white solid (20 mg, 24% yield). MS: 738.2 [M−H].

### Step 2: Synthesis of 1-(4-(4-amino-7-cyclopropyl-7H-pyrrolo[2,3-d]pyrimidin-5-yl)-2-methyl-2,3-dihydrobenzofuran-7-yl)-3-(5-(1-(trifluoromethyl)cyclopropyl)isoxazol-3-yl)urea

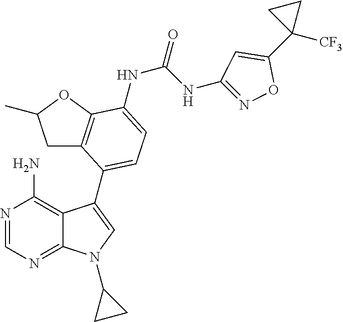 [SMILES: CC1Cc2c(-c3cn(C4CC4)c4ncnc(N)c34)ccc(NC(=O)Nc3cc(C4(C(F)(F)F)CC4)on3)c2O1]

The title compound was obtained by following a similar procedure described for step 2 of Example 5, starting from tert-butyl (tert-butoxycarbonyl)(7-cyclopropyl-5-(2-methyl-7-(3-(5-(1-(trifluoromethyl)cyclopropyl)isoxazol-3-yl)ureido)-2,3-dihydrobenzofuran-4-yl)-7H-pyrrolo[2,3-d]pyrimidin-4-yl)carbamate (20 mg, 0.027 mmol), and was isolated as an off-white solid (10 mg, 69% yield). ¹H NMR (400 MHz, DMSO-d₆) δ=9.95 (bs, 1H), 8.52 (bs, 1H), 8.15 (bs, 1H), 7.91 (d, J=8.4 Hz, 1H), 7.21 (bs, 1H), 6.89 (s, 1H), 6.77 (d, J=8.4 Hz, 1H), 6.02 (bs, 2H), 5.03-5.05 (m, 1H), 3.56-3.59 (m, 1H), 3.30-3.36 (m, 1H), 2.82-2.88 (m, 1H), 1.45-1.54 (m, 7H), 1.04-1.05 (m, 4H). MS: 540.1 [M+H].

### Example 57

### 1-(4-(4-Amino-7-Cyclopropyl-7H-Pyrrolo[2,3-D]Pyrimidin-5-Yl)-2-Methyl-2,3-Dihydrobenzofuran-7-Yl)-3-(4-((4-Methylpiperazin-1-Yl)Methyl)-3-(Trifluoromethyl)Phenyl)Urea

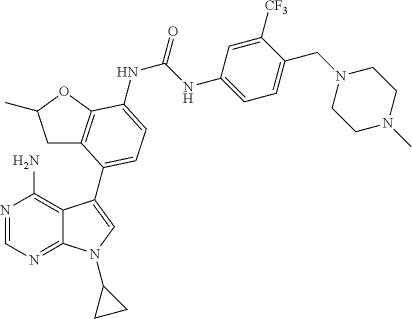

The title compound is prepared by following a similar procedure described for Intermediate B1, starting from 1-(4-bromo-2-methyl-2,3-dihydrobenzofuran-7-yl)-3-(4-((4-methylpiperazin-1-yl)methyl)-3-(trifluoromethyl)phenyl)urea (D19, 50 mg, 0.095 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 47 mg, 0.095 mmol). MS (expected): 621.3 [M+H].

### Step 1: Synthesis of tert-butyl (tert-butoxycarbonyl)(7-cyclopropyl-5-(2-methyl-7-(3-(4-((4-methylpiperazin-1-yl)methyl)-3-(trifluoromethyl)phenyl)ureido)-2,3-dihydrobenzofuran-4-yl)-7H-pyrrolo[2,3-d]pyrimidin-4-yl)carbamate

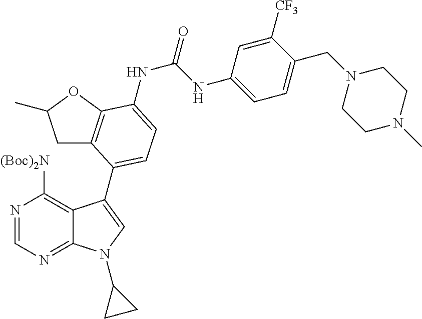

The title compound was prepared by following a similar procedure described for Intermediate B1, starting from 1-(4-bromo-2-methyl-2,3-dihydrobenzofuran-7-yl)-3-(4-((4-methylpiperazin-1-yl)methyl)-3-(trifluoromethyl)phenyl)urea (D19, 150 mg, 0.284 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 142 mg, 0.284 mmol), and was isolated as an off-white solid (50 mg, 21% yield). MS: 821.2 [M+H].

### Step 2: Synthesis of 1-(4-(4-amino-7-cyclopropyl-7H-pyrrolo[2,3-d]pyrimidin-5-yl)-2-methyl-2,3-dihydrobenzofuran-7-yl)-3-(4-((4-methylpiperazin-1-yl)methyl)-3-(trifluoromethyl)phenyl)urea

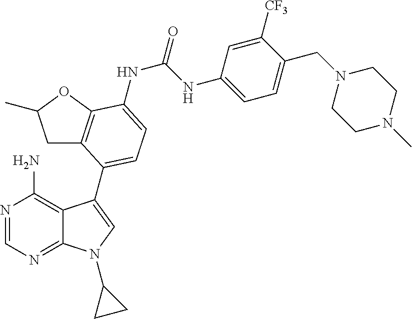

The title compound was obtained by following a similar procedure described for step 2 of Example 5, starting from tert-butyl (tert-butoxycarbonyl)(7-cyclopropyl-5-(2-methyl-7-(3-(4-((4-methylpiperazin-1-yl)methyl)-3-(trifluoromethyl)phenyl)ureido)-2,3-dihydrobenzofuran-4-yl)-7H-pyrrolo[2,3-d]pyrimidin-4-yl)carbamate (50 mg, 0.061 mmol), and was isolated as an off-white solid (25 mg, 66% yield). ¹H NMR (400 MHz, DMSO-d₆) 6=9.48 (bs, 1H), 8.24 (bs, 1H), 8.14-8.16 (m, 1H), 7.94-7.98 (m, 2H), 7.56-7.64 (m, 2H), 7.21 (bs, 1H), 6.77 (d, J=8.40 Hz, 1H), 6.05 (bs, 2H), 5.01-5.07 (m, 1H), 3.56-3.62 (m, 3H), 3.31-3.37 (m, 1H), 3.04-3.15 (m, 8H), 2.84-2.90 (m, 1H), 2.67 (s, 3H), 1.44-1.45 (m, 3H), 1.01-1.04 (m, 4H). MS: 621.4 [M+H].

### Example 58

### 1-(4-(4-Amino-7-Cyclopropyl-7H-Pyrrolo[2,3-D]Pyrimidin-5-Yl)-2-Methyl-2,3-Dihydrobenzofuran-7-Yl)-3-(4-((1-Methylpiperidin-4-Yl)Oxy)-3-(Trifluoromethyl)Phenyl)Urea

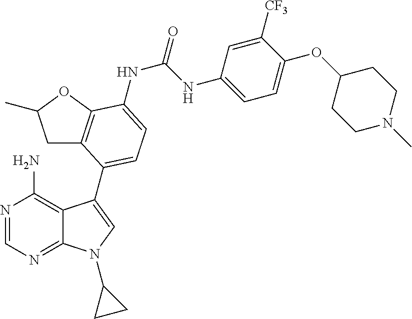

The title compound is prepared by following a similar procedure described for Intermediate B1, starting from 1-(4-bromo-2-methyl-2,3-dihydrobenzofuran-7-yl)-3-(4-((1-methylpiperidin-4-yl)oxy)-3-(trifluoromethyl)phenyl)urea (D20, 50 mg, 0.095 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 47 mg, 0.095 mmol). MS (expected): 622.3 [M+H].

### Step 1: Synthesis of tert-butyl (tert-butoxycarbonyl)(7-cyclopropyl-5-(2-methyl-7-(3-(4-((1-methylpiperidin-4-yl)oxy)-3-(trifluoromethyl)phenyl)ureido)-2,3-dihydrobenzofuran-4-yl)-7H-pyrrolo[2,3-d]pyrimidin-4-yl)carbamate

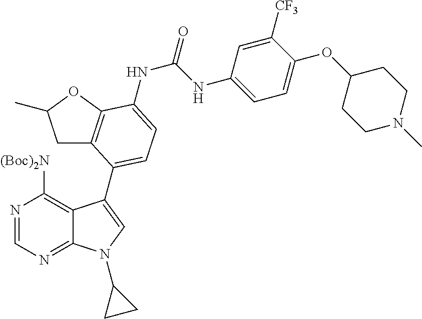

The title compound was obtained by following a similar procedure described for Intermediate B1, starting from 1-(4-bromo-2-methyl-2,3-dihydrobenzofuran-7-yl)-3-(4-((1-methylpiperidin-4-yl)oxy)-3-(trifluoromethyl)phenyl)urea (D20, 60 mg, 0.114 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 57 mg, 0.114 mmol), and was isolated as an off-white solid (10 mg, 14% yield). MS: 822.4 [M+H].

### Step 2: Synthesis of 1-(4-(4-amino-7-cyclopropyl-7H-pyrrolo[2,3-d]pyrimidin-5-yl)-2-methyl-2,3-dihydrobenzofuran-7-yl)-3-(4-((1-methylpiperidin-4-yl)oxy)-3-(trifluoromethyl)phenyl)urea

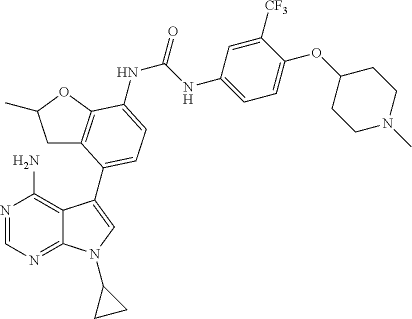

The title compound was obtained by following a similar procedure described for step 2 of Example 5, starting from tert-butyl (tert-butoxycarbonyl)(7-cyclopropyl-5-(2-methyl-7-(3-(4-((1-methylpiperidin-4-yl)oxy)-3-(trifluoromethyl)phenyl)ureido)-2,3-dihydrobenzofuran-4-yl)-7H-pyrrolo[2,3-d]pyrimidin-4-yl)carbamate (10 mg, 0.012 mmol), and was isolated as an off-white solid (4.4 mg, 58% yield). ¹H NMR (400 MHz, CD₃OD) δ=8.18 (s, 1H), 7.84 (d, J=8.4 Hz, 1H), 7.76 (d, J=2.8 Hz, 1H), 7.57-7.60 (m, 1H), 7.17 (d, J=8.8 Hz, 1H), 7.11 (s, 1H), 6.83 (d, J=8.0 Hz, 1H), 5.04-5.06 (m, 1H), 4.60 (bs, 1H), 3.49-3.52 (m, 1H), 3.34-3.39 (m, 1H), 2.83-2.89 (m, 1H), 2.50-2.72 (m, 4H), 2.35 (s, 3H), 1.91-2.04 (m, 4H), 1.50-1.51 (m, 3H), 1.06-1.16 (m, 4H). MS: 622.2 [M+H].

### Example 59

### 1-(4-(4-Amino-7-Cyclopropyl-7H-Pyrrolo[2,3-D]Pyrimidin-5-Yl)-2,2-Dimethyl-2,3-Dihydrobenzofuran-7-Yl)-3-(5-(1-(Trifluoromethyl)Cyclopropyl)Isoxazol-3-Yl)Urea

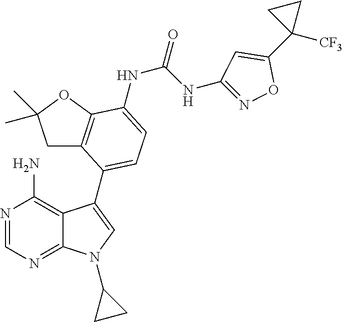 [SMILES: CC1(C)Cc2c(-c3cn(C4CC4)c4ncnc(N)c34)ccc(NC(=O)Nc3cc(C4(C(F)(F)F)CC4)on3)c2O1]

The title compound is prepared by following a similar procedure described for Intermediate B1, starting from 1-(4-bromo-2,2-dimethyl-2,3-dihydrobenzofuran-7-yl)-3-(5-(1-(trifluoromethyl)cyclopropyl)isoxazol-3-yl)urea (D21, 50 mg, 0.109 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 54 mg, 0.109 mmol). MS (expected): 554.2 [M+H].

### Step 1: Synthesis of tert-butyl (tert-butoxycarbonyl)(7-cyclopropyl-5-(2,2-dimethyl-7-(3-(5-(1-(trifluoromethyl)cyclopropyl)isoxazol-3-yl)ureido)-2,3-dihydrobenzofuran-4-yl)-7H-pyrrolo[2,3-d]pyrimidin-4-yl)carbamate

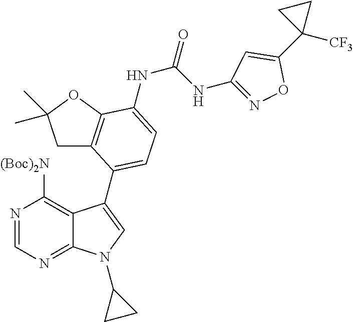

The title compound was obtained by following a similar procedure described for Intermediate B1, starting from 1-(4-bromo-2,2-dimethyl-2,3-dihydrobenzofuran-7-yl)-3-(5-(1-(trifluoromethyl)cyclopropyl)isoxazol-3-yl)urea (D21, 50 mg, 0.109 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 43 mg, 0.109 mmol), and was isolated as an off-white solid (10 mg, 12% yield). MS: 754.2 [M+H].

### Step 2: Synthesis of 1-(4-(4-amino-7-cyclopropyl-7H-pyrrolo[2,3-d]pyrimidin-5-yl)-2,2-dimethyl-2,3-dihydrobenzofuran-7-yl)-3-(5-(1-(trifluoromethyl)cyclopropyl)isoxazol-3-yl)urea

The title compound was obtained by following a similar procedure described for step 2 of Example 5, starting from tert-butyl (tert-butoxycarbonyl)(7-cyclopropyl-5-(2,2-dimethyl-7-(3-(5-(1-(trifluoromethyl)cyclopropyl)isoxazol-3-yl)ureido)-2,3-dihydrobenzofuran-4-yl)-7H-pyrrolo[2,3-d]pyrimidin-4-yl)carbamate (10 mg, 0.013 mmol), and was isolated as an off-white solid (5 mg, 68% yield). ¹H NMR (400 MHz, CD₃OD) δ=8.18 (s, 1H), 7.89 (d, J=8.4 Hz, 1H), 7.12 (s, 1H), 6.82-6.86 (m, 2H), 3.49-3.53 (m, 1H), 3.07 (s, 2H), 1.48-1.58 (m, 10H), 1.06-1.18 (m, 4H). MS: 554.2 [M+H].

### Example 60

### 1-(4-(4-Amino-7-Cyclopropyl-7H-Pyrrolo[2,3-D]Pyrimidin-5-Yl)-2,2-Dimethyl-2,3-Dihydrobenzofuran-7-Yl)-3-(4-((4-Methylpiperazin-1-Yl)Methyl)-3-(Trifluoromethyl)Phenyl)Urea

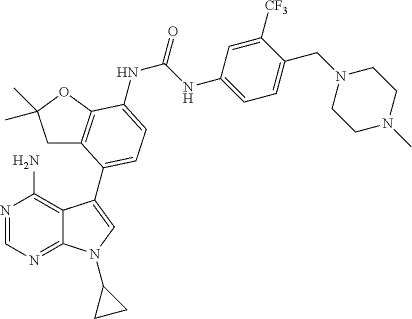

The title compound is prepared by following a similar procedure described for Intermediate B1, starting from 1-(4-bromo-2,2-dimethyl-2,3-dihydrobenzofuran-7-yl)-3-(4-((4-methylpiperazin-1-yl)methyl)-3-(trifluoromethyl)phenyl)urea (D22, 50 mg, 0.092 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 46 mg, 0.092 mmol). MS (expected): 635.3 [M+H].

The title compound was obtained by following a similar procedure described for Intermediate B1, starting from 1-(4-bromo-2,2-dimethyl-2,3-dihydrobenzofuran-7-yl)-3-(4-((4-methylpiperazin-1-yl)methyl)-3-(trifluoromethyl)phenyl)urea (D22, 30 mg, 0.055 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 28 mg, 0.055 mmol), and was isolated as an off-white solid (6 mg, 17% yield). Cleavage of the di-Boc protecting group was observed during the reaction. ¹H NMR (400 MHz, DMSO-d₆) δ=9.48 (bs, 1H), 8.15-8.16 (m, 2H), 7.95-7.99 (m, 2H), 7.47-7.61 (m, 2H), 7.20 (s, 1H), 6.76 (d, J=8.0 Hz, 1H), 6.02 (bs, 2H), 3.53-3.58 (m, 3H), 3.09 (s, 2H), 2.33-2.38 (m, 8H), 2.16 (s, 3H), 1.49 (s, 6H), 1.01-1.04 (m, 4H). MS: 635.2 [M+H].

### Example 61

### 1-(4-(4-Amino-7-Cyclopropyl-7H-Pyrrolo[2,3-D]Pyrimidin-5-Yl)-2,2-Dimethyl-2,3-Dihydrobenzofuran-7-Yl)-3-(4-((1-Methylpiperidin-4-Yl)Oxy)-3-(Trifluoromethyl)Phenyl)Urea

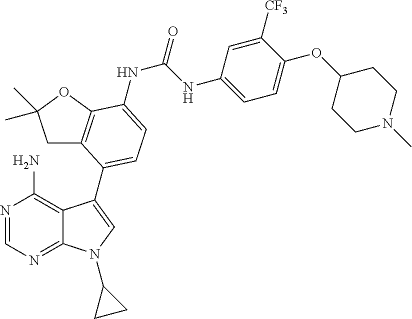

The title compound is prepared by following a similar procedure described for Intermediate B1, starting from 1-(4-bromo-2,2-dimethyl-2,3-dihydrobenzofuran-7-yl)-3-(4-((1-methylpiperidin-4-yl)oxy)-3-(trifluoromethyl)phenyl)urea (D23, 50 mg, 0.092 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 46 mg, 0.092 mmol). MS (expected): 636.3 [M+H].

### Step 1: Synthesis of tert-butyl (tert-butoxycarbonyl)(7-cyclopropyl-5-(2,2-dimethyl-7-(3-(4-((1-methylpiperidin-4-yl)oxy)-3-(trifluoromethyl)phenyl)ureido)-2,3-dihydrobenzofuran-4-yl)-7H-pyrrolo[2,3-d]pyrimidin-4-yl)carbamate

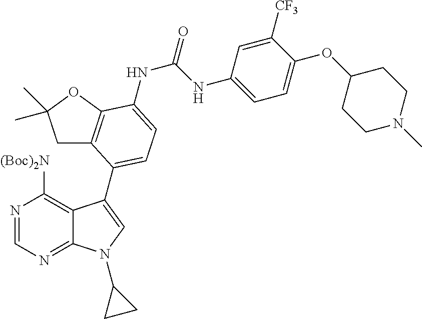

The title compound was obtained by following a similar procedure described for Intermediate B1, starting from starting from 1-(4-bromo-2,2-dimethyl-2,3-dihydrobenzofuran-7-yl)-3-(4-((1-methylpiperidin-4-yl)oxy)-3-(trifluoromethyl)phenyl)urea (D23, 50 mg, 0.092 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 46 mg, 0.092 mmol), and was isolated as an off-white solid (30 mg, 38% yield). MS: 836.3 [M+H].

### Step 2: Synthesis of 1-(4-(4-amino-7-cyclopropyl-7H-pyrrolo[2,3-d]pyrimidin-5-yl)-2,2-dimethyl-2,3-dihydrobenzofuran-7-yl)-3-(4-((1-methylpiperidin-4-yl)oxy)-3-(trifluoromethyl)phenyl)urea

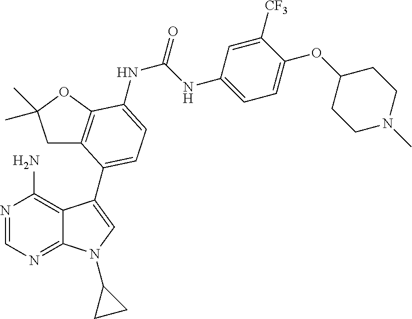

The title compound was obtained by following a similar procedure described for step 2 of Example 5, starting from tert-butyl (tert-butoxycarbonyl)(7-cyclopropyl-5-(2,2-dimethyl-7-(3-(4-((1-methylpiperidin-4-yl)oxy)-3-(trifluoromethyl)phenyl)ureido)-2,3-dihydrobenzofuran-4-yl)-7H-pyrrolo[2,3-d]pyrimidin-4-yl)carbamate (30 mg, 0.036 mmol), and was isolated as an off-white solid (14.3 mg, 63% yield). ¹H NMR (400 MHz, DMSO-d₆) δ=9.25 (bs, 1H), 8.15 (bs, 1H), 8.05 (bs, 1H), 7.95 (d, J=8.4 Hz, 1H), 7.84 (d, J=2.8 Hz, 1H), 7.48-7.51 (m, 1H), 7.25 (d, J=9.2 Hz, 1H), 7.19 (s, 1H), 6.75 (d, J=8.4 Hz, 1H), 6.02 (bs, 2H), 4.52 (bs, 1H), 3.54-3.59 (m, 1H), 3.08 (s, 2H), 2.20-2.25 (m, 2H), 2.17 (s, 3H), 1.88-1.93 (m, 2H), 1.67-1.71 (m, 2H), 1.49 (s, 6H), 1.01-1.05 (m, 4H). MS: 636.3 [M+H].

### Example 62

### 1-(4-(4-Amino-7-Cyclopropyl-7H-Pyrrolo[2,3-D]Pyrimidin-5-Yl)Benzofuran-7-Yl)-3-(3-(Tert-Butyl)-1-Phenyl-1H-Pyrazol-5-Yl)Urea

### Step 1: Synthesis of tert-butyl (tert-butoxycarbonyl)(5-(7-(3-(3-(tert-butyl)-1-phenyl-1H-pyrazol-5-yl)ureido)benzofuran-4-yl)-7-cyclopropyl-7H-pyrrolo[2,3-d]pyrimidin-4-yl)carbamate

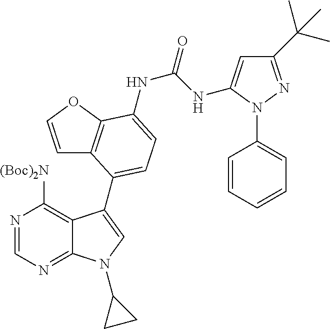

The title compound was obtained by following a similar procedure described for Intermediate B1, starting from 1-(3-(tert-butyl)-1-phenyl-1H-pyrazol-5-yl)-3-(4-iodobenzofuran-7-yl)urea (D24, 150 mg, 0.300 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 150 mg, 0.300 mmol), and was isolated as a white solid (50 mg, 22% yield). MS: 747.3 [M+H].

### Step 2: Synthesis of 1-(4-(4-amino-7-cyclopropyl-7H-pyrrolo[2,3-d]pyrimidin-5-yl)benzofuran-7-yl)-3-(3-(tert-butyl)-1-phenyl-1H-pyrazol-5-yl)urea

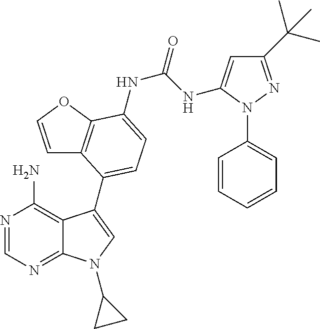 [SMILES: CC(C)(C)c1cc(NC(=O)Nc2ccc(-c3cn(C4CC4)c4ncnc(N)c34)c3ccoc23)n(-c2ccccc2)n1]

The title compound was obtained by following a similar procedure described for step 2 of Example 5, starting from tert-butyl (tert-butoxycarbonyl)(5-(7-(3-(3-(tert-butyl)-1-phenyl-1H-pyrazol-5-yl)ureido)benzofuran-4-yl)-7-cyclopropyl-7H-pyrrolo[2,3-d]pyrimidin-4-yl)carbamate (50 mg, 0.067 mmol), and was isolated as an off-white solid (30 mg, 81% yield). ¹H NMR (400 MHz, DMSO-d₆) δ=9.42 (s, 1H), 8.99 (s, 1H), 8.46 (s, 1H), 8.09-8.12 (m, 2H), 7.54-7.60 (m, 5H), 7.44-7.47 (m, 1H), 7.20 (d, J=8.4 Hz, 1H), 6.97 (d, J=2.0 Hz, 1H), 6.45 (s, 1H), 3.71-3.75 (m, 1H), 1.30 (s, 9H), 1.10-1.18 (m, 4H). MS: 547.3 [M+H].

### Example 63

### 1-(4-(4-Amino-7-Cyclopropyl-7H-Pyrrolo[2,3-D]Pyrimidin-5-Yl)Benzofuran-7-Yl)-3-(3-(Tert-Butyl)Isoxazol-5-Yl)Urea

### Step 1: Synthesis of tert-butyl (tert-butoxycarbonyl)(5-(7-(3-(3-(tert-butyl)isoxazol-5-yl)ureido)benzofuran-4-yl)-7-cyclopropyl-7H-pyrrolo[2,3-d]pyrimidin-4-yl)carbamate

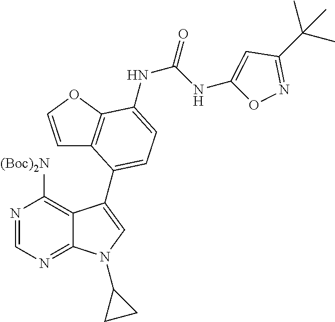

The title compound was obtained by following a similar procedure described for Intermediate C1, starting from 1-(3-(tert-butyl)isoxazol-5-yl)-3-(4-iodobenzofuran-7-yl)urea (D25, 130 mg, 0.306 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 150 mg, 0.300 mmol), and was isolated as a pale brown solid (50 mg, 25% yield). MS: 672.3 [M+H].

### Step 2: Synthesis of 1-(4-(4-amino-7-cyclopropyl-7H-pyrrolo[2,3-d]pyrimidin-5-yl)benzofuran-7-yl)-3-(3-(tert-butyl)isoxazol-5-yl)urea

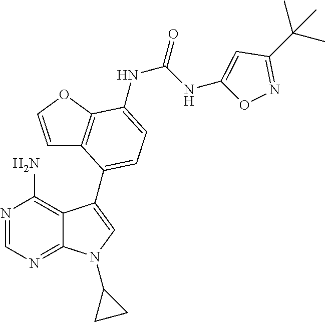 [SMILES: CC(C)(C)c1cc(NC(=O)Nc2ccc(-c3cn(C4CC4)c4ncnc(N)c34)c3ccoc23)on1]

The title compound was obtained by following a similar procedure described for step 2 of Example 5, starting from tert-butyl (tert-butoxycarbonyl)(5-(7-(3-(3-(tert-butyl)isoxazol-5-yl)ureido)benzofuran-4-yl)-7-cyclopropyl-7H-pyrrolo[2,3-d]pyrimidin-4-yl)carbamate (20 mg, 0.030 mmol), and was isolated as an off-white solid (12 mg, 86% yield). ¹H NMR (400 MHz, DMSO-d₆) δ=10.44 (s, 1H), 9.21 (s, 1H), 8.43 (s, 1H), 8.16 (d, J=2.0 Hz, 1H), 8.06 (d, J=8.4 Hz, 1H), 7.56 (s, 1H), 7.23 (d, J=8.0 Hz, 1H), 7.00 (d, J=2.0 Hz, 1H), 6.12 (s, 1H), 3.72-3.73 (m, 1H), 1.28 (s, 9H), 1.10-1.17 (m, 4H). MS: 472.3 [M+H].

### Example 64

### 1-(4-(4-Amino-7-Cyclopropyl-7H-Pyrrolo[2,3-D]Pyrimidin-5-Yl)Benzofuran-7-Yl)-3-(3-(1-(Trifluoromethyl)Cyclopropyl)Isoxazol-5-Yl)Urea

### Step 1: Synthesis of tert-butyl (tert-butoxycarbonyl)(7-cyclopropyl-5-(7-(3-(3-(1-(trifluoromethyl)cyclopropyl)isoxazol-5-yl)ureido)benzofuran-4-yl)-7H-pyrrolo[2,3-d]pyrimidin-4-yl)carbamate

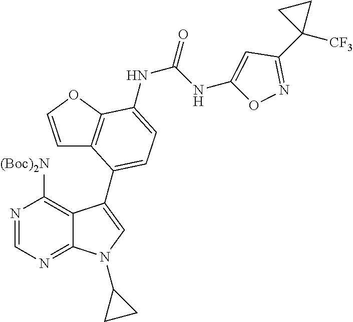

The title compound was obtained by following a similar procedure described for Intermediate B1, starting from 1-(4-iodobenzofuran-7-yl)-3-(3-(1-(trifluoromethyl)cyclopropyl)isoxazol-5-yl)urea (D26, 57 mg, 0.120 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 60 mg, 0.120 mmol), and was isolated as a pale brown solid (48 mg, 55% yield). MS: 724.2 [M+H].

### Step 2: Synthesis of 1-(4-(4-amino-7-cyclopropyl-7H-pyrrolo[2,3-d]pyrimidin-5-yl)benzofuran-7-yl)-3-(3-(1-(trifluoromethyl)cyclopropyl)isoxazol-5-yl)urea

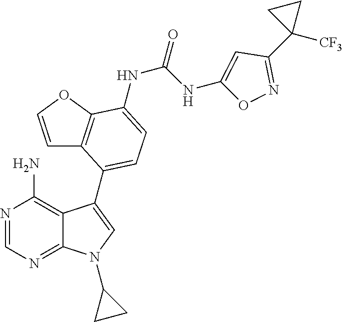 [SMILES: Nc1ncnc2c1c(-c1ccc(NC(=O)Nc3cc(C4(C(F)(F)F)CC4)no3)c3occc13)cn2C1CC1]

The title compound was obtained by following a similar procedure described for step 2 of Example 5, starting from tert-butyl (tert-butoxycarbonyl)(5-(7-(3-(3-(tert-butyl)isoxazol-5-yl)ureido)benzofuran-4-yl)-7-cyclopropyl-7H-pyrrolo[2,3-d]pyrimidin-4-yl)carbamate (48 mg, 0.066 mmol), and was isolated as an off-white solid (6 mg, 17% yield). ¹H NMR (400 MHz, CD₃OD) δ=8.21 (s, 1H), 8.00 (d, J=8.0 Hz, 1H), 7.88 (d, J=2.0 Hz, 1H), 7.27 (d, J=8.0 Hz, 1H), 7.23 (s, 1H), 6.86 (d, J=2.4 Hz, 1H), 6.34 (s, 1H), 3.53-3.57 (m, 1H), 1.39-1.49 (m, 4H), 1.11-1.18 (m, 4H). MS: 524.3 [M+H].

### Example 65

### 1-(4-(4-Amino-7-Cyclopropyl-7H-Pyrrolo[2,3-D]Pyrimidin-5-Yl)Benzofuran-7-Yl)-3-(5-(1-(Trifluoromethyl)Cyclopropyl)Isoxazol-3-Yl)Urea

### Step 1: Synthesis of tert-butyl (tert-butoxycarbonyl)(7-cyclopropyl-5-(7-(3-(5-(1-(trifluoromethyl)cyclopropyl)isoxazol-3-yl)ureido)benzofuran-4-yl)-7H-pyrrolo[2,3-d]pyrimidin-4-yl)carbamate

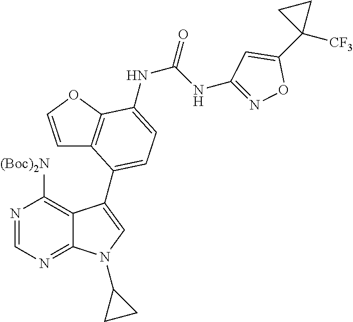

The title compound was obtained by following a similar procedure described for Intermediate B1, starting from 1-(4-iodobenzofuran-7-yl)-3-(5-(1-(trifluoromethyl)cyclopropyl)isoxazol-3-yl)urea (D27, 67 mg, 0.140 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 70 mg, 0.140 mmol), and was isolated as a pale yellow solid (38 mg, 38% yield). MS: 724.3 [M+H].

### Step 2: Synthesis of 1-(4-(4-amino-7-cyclopropyl-7H-pyrrolo[2,3-d]pyrimidin-5-yl)benzofuran-7-yl)-3-(5-(1-(trifluoromethyl)cyclopropyl)isoxazol-3-yl)urea

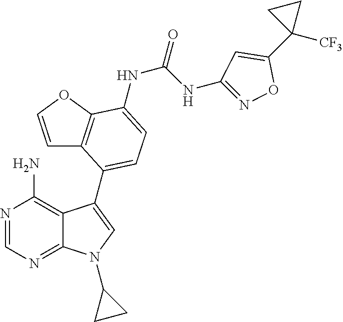 [SMILES: Nc1ncnc2c1c(-c1ccc(NC(=O)Nc3cc(C4(C(F)(F)F)CC4)on3)c3occc13)cn2C1CC1]

The title compound was obtained by following a similar procedure described for step 2 of Example 5, starting from tert-butyl (tert-butoxycarbonyl)(7-cyclopropyl-5-(7-(3-(5-(1-(trifluoromethyl)cyclopropyl)isoxazol-3-yl)ureido)benzofuran-4-yl)-7H-pyrrolo[2,3-d]pyrimidin-4-yl)carbamate (35 mg, 0.048 mmol), and was isolated as an off-white solid (8 mg, 32% yield). ¹H NMR (400 MHz, DMSO-d₆) δ=10.07 (s, 1H), 9.26 (s, 1H), 8.42 (s, 1H), 8.16 (d, J=2.4 Hz, 1H), 8.07 (d, J=8.0 Hz, 1H), 7.54 (s, 1H), 7.23 (d, J=8.0 Hz, 1H), 6.99 (d, J=2.4 Hz, 1H), 6.92 (s, 1H), 3.72-3.73 (m, 1H), 1.49-1.58 (m, 4H), 1.08-1.24 (m, 4H). MS: 524.2 [M+H].

### Example 66

### 1-(4-(4-Amino-7-Cyclopropyl-7H-Pyrrolo[2,3-D]Pyrimidin-5-Yl)Benzofuran-7-Yl)-3-(4-((4-Methylpiperazin-1-Yl)Methyl)-3-(Trifluoromethyl)Phenyl)Urea

### Step 1: Synthesis of tert-butyl (tert-butoxycarbonyl)(7-cyclopropyl-5-(7-(3-(4-((4-methylpiperazin-1-yl)methyl)-3-(trifluoromethyl)phenyl)ureido)benzofuran-4-yl)-7H-pyrrolo[2,3-d]pyrimidin-4-yl)carbamate

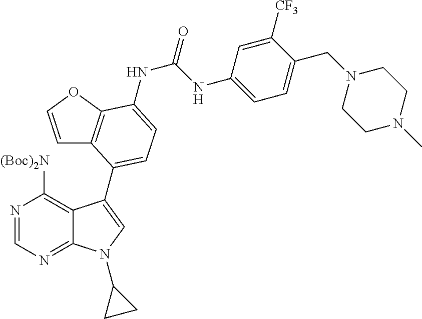

The title compound was obtained by following a similar procedure described for Intermediate B1, starting from 1-(4-iodobenzofuran-7-yl)-3-(4-((4-methylpiperazin-1-yl)methyl)-3-(trifluoromethyl)phenyl)urea (D28, 100 mg, 0.180 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 90 mg, 0.180 mmol), and was isolated as a pale yellow gum (40 mg, 28% yield). MS: 805.3 [M+H].

### Step 2: Synthesis of 1-(4-(4-amino-7-cyclopropyl-7H-pyrrolo[2,3-d]pyrimidin-5-yl)benzofuran-7-yl)-3-(4-((4-methylpiperazin-1-yl)methyl)-3-(trifluoromethyl)phenyl)urea

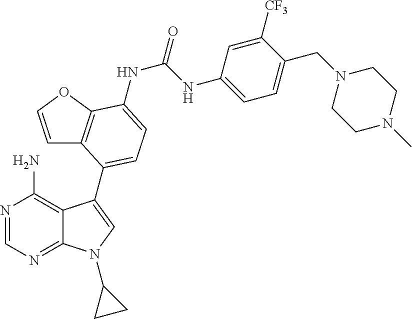 [SMILES: CN1CCN(Cc2ccc(NC(=O)Nc3ccc(-c4cn(C5CC5)c5ncnc(N)c45)c4ccoc34)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((4-methylpiperazin-1-yl)methyl)-3-(trifluoromethyl)phenyl)ureido)benzofuran-4-yl)-7H-pyrrolo[2,3-d]pyrimidin-4-yl)carbamate (40 mg, 0.050 mmol), and was isolated as an off-white solid (11 mg, 37% yield). ¹H NMR (400 MHz, DMSO-d₆) δ=9.57 (s, 1H), 9.10 (s, 1H), 8.41 (s, 1H), 8.16 (d, J=2.0 Hz, 1H), 8.08 (d, J=8.0 Hz, 1H), 8.04 (s, 1H), 7.62-7.67 (m, 2H), 7.51 (s, 1H), 7.22 (d, J=8.0 Hz, 1H), 7.00 (d, J=2.0 Hz, 1H), 3.69-3.73 (m, 1H), 3.65 (s, 2H), 3.40-3.49 (m, 2H), 2.90-3.03 (m, 4H), 2.82 (s, 3H), 2.33-2.35 (m, 2H), 1.09-1.16 (m, 4H). MS: 605.3 [M+H].

### Example 67

### 1-(4-(4-Amino-7-Cyclopropyl-7H-Pyrrolo[2,3-D]Pyrimidin-5-Yl)Benzofuran-7-Yl)-3-(4-((1-Methylpiperidin-4-Yl)Oxy)-3-(Trifluoromethyl)Phenyl)Urea

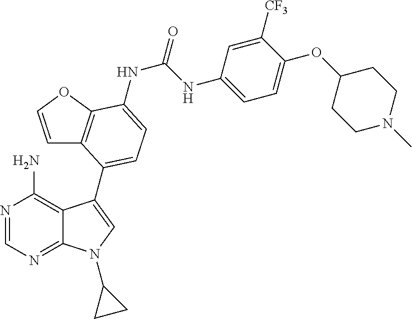 [SMILES: CN1CCC(Oc2ccc(NC(=O)Nc3ccc(-c4cn(C5CC5)c5ncnc(N)c45)c4ccoc34)cc2C(F)(F)F)CC1]

The title compound is prepared by following a similar procedure described for Intermediate B1, starting from 1-(4-iodobenzofuran-7-yl)-3-(4-((1-methylpiperidin-4-yl)oxy)-3-(trifluoromethyl)phenyl)urea (D29, 50 mg, 0.089 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 45 mg, 0.089 mmol). MS (expected): 606.2 [M+H].

The title compound was obtained by following a similar procedure described for Intermediate B1, starting from 1-(4-iodobenzofuran-7-yl)-3-(4-((1-methylpiperidin-4-yl)oxy)-3-(trifluoromethyl)phenyl)urea (D29, 15 mg, 0.027 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 13 mg, 0.027 mmol), and was isolated as an off-white solid (1.5 mg, 9% yield). Cleavage of the di-Boc protecting group was observed during the reaction. MS: 606.3 [M+H].

### Example 68

### 1-(4-(4-Amino-7-Cyclopropyl-7H-Pyrrolo[2,3-D]Pyrimidin-5-Yl)Benzo[D]Isoxazol-7-Yl)-3-(5-(1-(Trifluoromethyl)Cyclopropyl)Isoxazol-3-Yl)Urea

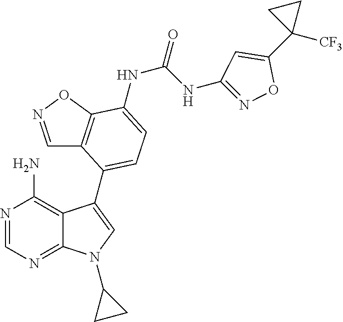 [SMILES: Nc1ncnc2c1c(-c1ccc(NC(=O)Nc3cc(C4(C(F)(F)F)CC4)on3)c3oncc13)cn2C1CC1]

The title compound was obtained by following the general procedure for urea formation (Method B), starting from 4-(4-amino-7-cyclopropyl-7H-pyrrolo[2,3-d]pyrimidin-5-yl)benzo[d]isoxazol-7-amine (B13, 60 mg, 0.196 mmol) and phenyl (5-(1-(trifluoromethyl)cyclopropyl)isoxazol-3-yl)carbamate (C3, 61 mg, 0.196 mmol), and was isolated as an off-white solid (7 mg, 7% yield). ¹H NMR (400 MHz, DMSO-d₆) δ=11.21 (bs, 1H), 10.29 (s, 1H), 8.89 (s, 1H), 8.41 (s, 1H), 8.27 (d, J=8.4 Hz, 1H), 7.60 (s, 1H), 7.00 (d, J=8.0 Hz, 1H), 6.88 (s, 1H), 3.71-3.73 (m, 1H), 1.54-1.55 (m, 2H), 1.48-1.49 (m, 2H), 1.10-1.12 (m, 4H). MS: 525.3 [M−H].

### Example 69

### 1-(4-(4-Amino-7-Cyclopropyl-7H-Pyrrolo[2,3-D]Pyrimidin-5-Yl)Benzo[D]Isoxazol-7-Yl)-3-(4-((4-Methylpiperazin-1-Yl)Methyl)-3-(Trifluoromethyl)Phenyl)Urea

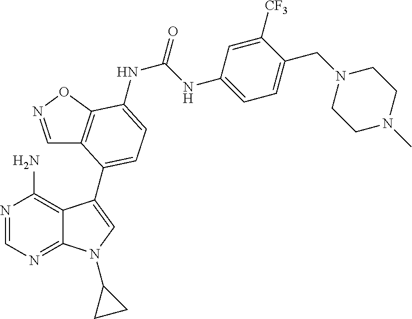 [SMILES: CN1CCN(Cc2ccc(NC(=O)Nc3ccc(-c4cn(C5CC5)c5ncnc(N)c45)c4cnoc34)cc2C(F)(F)F)CC1]

The title compound was obtained by following a similar procedure described for Intermediate B1, starting from 1-(4-bromobenzo[d]isoxazol-7-yl)-3-(4-((4-methylpiperazin-1-yl)methyl)-3-(trifluoromethyl)phenyl)urea (D30, 154 mg, 0.300 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 150 mg, 0.300 mmol), and was isolated as an off-white solid (9 mg, 5% yield). Cleavage of the di-Boc protecting group was observed during the reaction. ¹H NMR (400 MHz, DMSO-d₆) δ=9.97 (bs, 1H), 8.92 (bs, 1H), 8.75 (bs, 1H), 8.51 (s, 1H), 8.05 (d, J=7.6 Hz, 1H), 7.99 (s, 1H), 7.59-7.65 (m, 2H), 7.45 (s, 1H), 7.19 (bs, 1H), 6.33 (bs, 2H), 3.63-3.67 (m, 1H), 3.59 (s, 2H), 2.92-2.94 (bs, 8H), 2.68 (s, 3H), 1.08-1.10 (m, 4H). MS: 606.2 [M+H].

### Example 70

### 1-(4-(4-Amino-7-Cyclopropyl-7H-Pyrrolo[2,3-D]Pyrimidin-5-Yl)Benzo[D]Isoxazol-7-Yl)-3-(4-((1-Methylpiperidin-4-Yl)Oxy)-3-(Trifluoromethyl)Phenyl)Urea

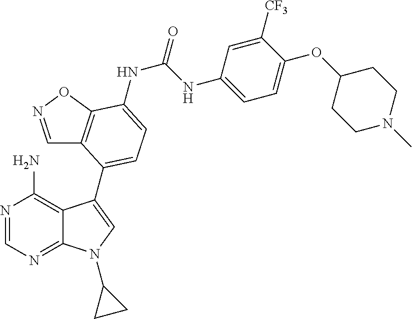 [SMILES: CN1CCC(Oc2ccc(NC(=O)Nc3ccc(-c4cn(C5CC5)c5ncnc(N)c45)c4cnoc34)cc2C(F)(F)F)CC1]

The title compound is prepared by following a similar procedure described for Intermediate B1, starting from 1-(4-bromobenzo[d]isoxazol-7-yl)-3-(4-((1-methylpiperidin-4-yl)oxy)-3-(trifluoromethyl)phenyl)urea (D31, 50 mg, 0.097 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 49 mg, 0.097 mmol). MS (expected): 607.2 [M+H].

### Step 1: Synthesis of tert-butyl (tert-butoxycarbonyl)(7-cyclopropyl-5-(7-(3-(4-((1-methylpiperidin-4-yl)oxy)-3-(trifluoromethyl)phenyl)ureido)benzo[d]isoxazol-4-yl)-7H-pyrrolo[2,3-d]pyrimidin-4-yl)carbamate

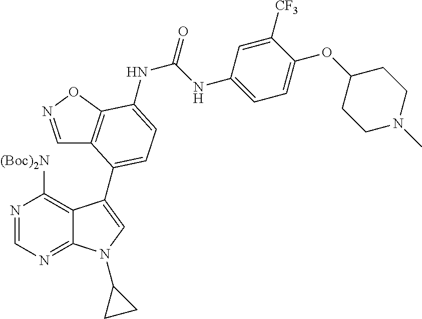

The title compound was obtained by following a similar procedure described for Intermediate B1, starting from 1-(4-bromobenzo[d]isoxazol-7-yl)-3-(4-((1-methylpiperidin-4-yl)oxy)-3-(trifluoromethyl)phenyl)urea (D31, 103 mg, 0.200 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 100 mg, 0.200 mmol), and was isolated as an off-white solid (30 mg, 19% yield). MS: 805.2 [M−H].

### Step 2: Synthesis of 1-(4-(4-amino-7-cyclopropyl-7H-pyrrolo[2,3-d]pyrimidin-5-yl)benzo[d]isoxazol-7-yl)-3-(4-((1-methylpiperidin-4-yl)oxy)-3-(trifluoromethyl)phenyl)urea

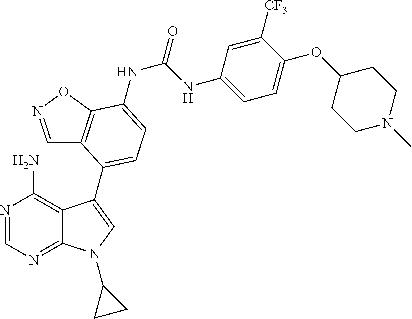 [SMILES: CN1CCC(Oc2ccc(NC(=O)Nc3ccc(-c4cn(C5CC5)c5ncnc(N)c45)c4cnoc34)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((1-methylpiperidin-4-yl)oxy)-3-(trifluoromethyl)phenyl)ureido)benzo[d]isoxazol-4-yl)-7H-pyrrolo[2,3-d]pyrimidin-4-yl)carbamate (30 mg, 0.112 mmol), and was isolated as an off-white solid (8 mg, 25% yield). ¹H NMR (400 MHz, DMSO-d₆) δ=9.66 (bs, 1H), 8.58 (bs, 1H), 8.15 (s, 1H), 7.99 (d, J=7.6 Hz, 1H), 7.87 (d, J=2.8 Hz, 1H), 7.54-7.57 (m, 1H), 7.26 (d, J=9.2 Hz, 1H), 7.17 (s, 1H), 7.0 (d, J=8.8 Hz, 1H), 6.82 (d, J=2.8 Hz, 1H), 6.02 (bs, 2H), 4.61 (bs, 1H), 3.54-3.60 (m, 1H), 2.81 (bs, 2H), 2.60-2.69 (m, 2H), 2.46 (s, 3H), 1.79-1.99 (m, 4H), 1.03-1.05 (m, 4H). MS: 607.3 [M+H].

### Example 71

### 1-(4-(4-Amino-7-Cyclopropyl-7H-Pyrrolo[2,3-D]Pyrimidin-5-Yl)Benzo[D]Oxazol-7-Yl)-3-(5-(1-(Trifluoromethyl)Cyclopropyl)Isoxazol-3-Yl)Urea

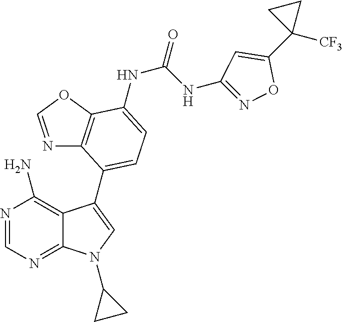 [SMILES: Nc1ncnc2c1c(-c1ccc(NC(=O)Nc3cc(C4(C(F)(F)F)CC4)on3)c3ocnc13)cn2C1CC1]

The title compound was obtained by following a similar procedure described for Intermediate B1, starting from 1-(4-bromobenzo[d]oxazol-7-yl)-3-(5-(1-(trifluoromethyl)cyclopropyl)isoxazol-3-yl)urea (D32, 90 mg, 0.210 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 105 mg, 0.210 mmol), and was isolated as an off-white solid (2 mg, 1.8% yield). MS: 525.1 [M+H].

### Example 72

### 1-(4-(4-Amino-7-Cyclopropyl-7H-Pyrrolo[2,3-D]Pyrimidin-5-Yl)Benzo[D]Oxazol-7-Yl)-3-(4-((4-Methylpiperazin-1-Yl)Methyl)-3-(Trifluoromethyl)Phenyl)Urea

### Step 1: Synthesis of tert-butyl (tert-butoxycarbonyl)(7-cyclopropyl-5-(7-(3-(4-((4-methylpiperazin-1-yl)methyl)-3-(trifluoromethyl)phenyl)ureido)benzo[d]oxazol-4-yl)-7H-pyrrolo[2,3-d]pyrimidin-4-yl)carbamate

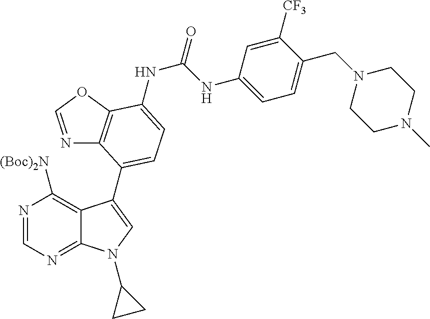

The title compound was obtained by following a similar procedure described for Intermediate B1, starting from 1-(4-bromobenzo[d]oxazol-7-yl)-3-(4-((4-methylpiperazin-1-yl)methyl)-3-(trifluoromethyl)phenyl)urea (D33, 31 mg, 0.060 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 31 mg, 0.060 mmol), and was isolated as pale brown gum (20 mg, 42% yield). MS: 806.3 [M+H].

### Step 2: Synthesis of 1-(4-(4-amino-7-cyclopropyl-7H-pyrrolo[2,3-d]pyrimidin-5-yl)benzo[d]oxazol-7-yl)-3-(4-((4-methylpiperazin-1-yl)methyl)-3-(trifluoromethyl)phenyl)urea

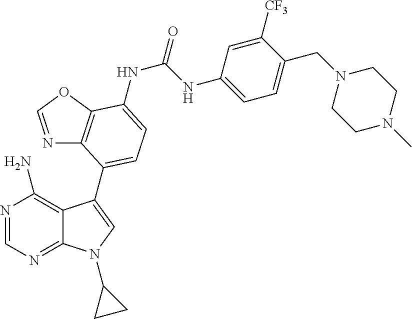 [SMILES: CN1CCN(Cc2ccc(NC(=O)Nc3ccc(-c4cn(C5CC5)c5ncnc(N)c45)c4ncoc34)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((4-methylpiperazin-1-yl)methyl)-3-(trifluoromethyl)phenyl)ureido)benzo[d]oxazol-4-yl)-7H-pyrrolo[2,3-d]pyrimidin-4-yl)carbamate (20 mg, 0.025 mmol), and was isolated as an off-white solid (0.6 mg, 4.3% yield). ¹H NMR (400 MHz, CD₃OD) δ=8.18 (d, J=3.2 Hz, 1H), 8.13 (d, J=8.4 Hz, 1H), 8.08 (s, 1H), 7.92 (d, J=2.0 Hz, 1H), 7.67-7.72 (m, 2H), 7.13 (s, 1H), 6.95 (d, J=8.4 Hz, 1H), 3.68 (s, 2H), 3.50-3.54 (m, 1H), 2.79 (bs, 4H), 2.61 (bs, 4H), 2.49 (s, 3H), 1.09-1.17 (m, 4H). MS: 624.0 [M+H₂O].

### Example 73

### 1-(4-(4-Amino-7-Cyclopropyl-7H-Pyrrolo[2,3-D]Pyrimidin-5-Yl)Benzo[D]Oxazol-7-Yl)-3-(4-((1-Methylpiperidin-4-Yl)Oxy)-3-(Trifluoromethyl)Phenyl)Urea

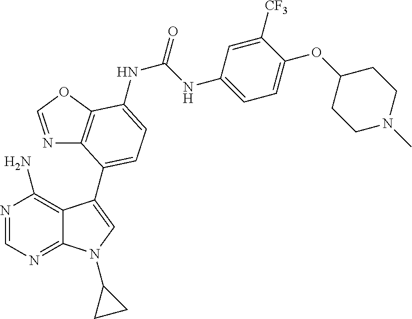 [SMILES: CN1CCC(Oc2ccc(NC(=O)Nc3ccc(-c4cn(C5CC5)c5ncnc(N)c45)c4ncoc34)cc2C(F)(F)F)CC1]

The title compound is prepared by following a similar two step procedure described in Example 7, starting from 1-(4-bromobenzo[d]oxazol-7-yl)-3-(4-((1-methylpiperidin-4-yl)oxy)-3-(trifluoromethyl)phenyl)urea (D34, 50 mg, 0.097 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 49 mg, 0.097 mmol). MS (expected): 607.2 [M+H].

### Example 74

### 1-(7-(4-Amino-7-Cyclopropyl-7H-Pyrrolo[2,3-D]Pyrimidin-5-Yl)Benzo[C][1,2,5]Oxadiazol-4-Yl)-3-(3-(Tert-Butyl)Isoxazol-5-Yl)Urea

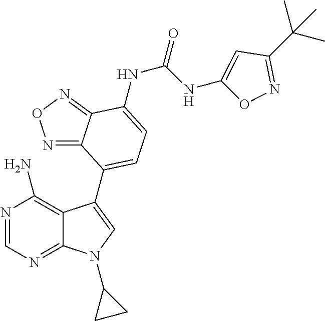 [SMILES: CC(C)(C)c1cc(NC(=O)Nc2ccc(-c3cn(C4CC4)c4ncnc(N)c34)c3nonc23)on1]

The title compound was obtained by following a similar procedure described for Intermediate B1, starting from 1-(7-bromobenzo[c][1,2,5]oxadiazol-4-yl)-3-(3-(tert-butyl)isoxazol-5-yl)urea (D35, 29 mg, 0.076 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 38 mg, 0.076 mmol), and was isolated as a yellow solid (4 mg, 11% yield). ¹H NMR (400 MHz, CD₃OD) δ=8.25 (s, 1H), 8.19 (d, J=7.6 Hz, 1H), 7.55 (s, 1H), 7.48 (d, J=7.2 Hz, 1H), 6.24 (s, 1H), 3.58 (bs, 1H), 1.36 (s, 9H), 1.13-1.26 (m, 4H). MS: 474.2 [M+H].

### Example 75

### 1-(7-(4-Amino-7-Cyclopropyl-7H-Pyrrolo[2,3-D]Pyrimidin-5-Yl)Benzo[C][1,2,5]Oxadiazol-4-Yl)-3-(3-(1-Methylcyclopropyl)Isoxazol-5-Yl)Urea

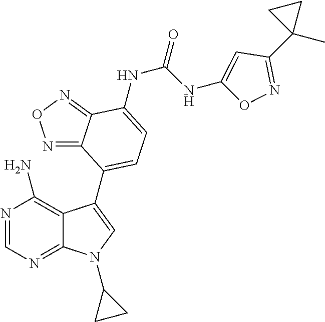 [SMILES: CC1(c2cc(NC(=O)Nc3ccc(-c4cn(C5CC5)c5ncnc(N)c45)c4nonc34)on2)CC1]

The title compound is prepared by following a similar procedure described for Intermediate B1, starting from 1-(7-bromobenzo[c][1,2,5]oxadiazol-4-yl)-3-(3-(1-methylcyclopropyl)isoxazol-5-yl)urea (D36, 50 mg, 0.132 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 66 mg, 0.132 mmol). MS (expected): 472.2 [M+H].

### Example 76

### 1-(7-(4-Amino-7-Cyclopropyl-7H-Pyrrolo[2,3-D]Pyrimidin-5-Yl)Benzo[C][1,2,5]Oxadiazol-4-Yl)-3-(3-(1-(Trifluoromethyl)Cyclopropyl)Isoxazol-5-Yl)Urea

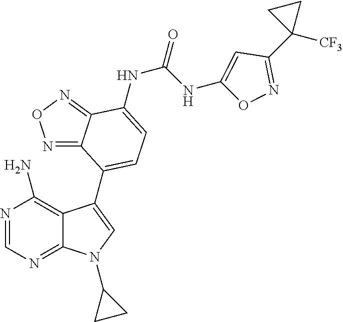 [SMILES: Nc1ncnc2c1c(-c1ccc(NC(=O)Nc3cc(C4(C(F)(F)F)CC4)no3)c3nonc13)cn2C1CC1]

The title compound is prepared by following a similar two-step procedure described for Example 5, starting from tert-butyl (5-(7-aminobenzo[c][1,2,5]oxadiazol-4-yl)-7-cyclopropyl-7H-pyrrolo[2,3-d]pyrimidin-4-yl)(tert-butoxycarbonyl)carbamate (B14, 50 mg, 0.099 mmol) and phenyl (3-(1-(trifluoromethyl)cyclopropyl)isoxazol-5-yl)carbamate (C1, 31 mg, 0.099 mmol) in step 1. MS (expected): 526.2 [M+H].

### Example 77

### 1-(7-(4-Amino-7-Cyclopropyl-7H-Pyrrolo[2,3-D]Pyrimidin-5-Yl)Benzo[C][1,2,5]Oxadiazol-4-Yl)-3-(5-(1-(Trifluoromethyl)Cyclopropyl)Isoxazol-3-Yl)Urea

### Step 1: Synthesis of tert-butyl (tert-butoxycarbonyl)(7-cyclopropyl-5-(7-(3-(5-(1-(trifluoromethyl)cyclopropyl)isoxazol-3-yl)ureido)benzo[c][1,2,5]oxadiazol-4-yl)-7H-pyrrolo[2,3-d]pyrimidin-4-yl)carbamate

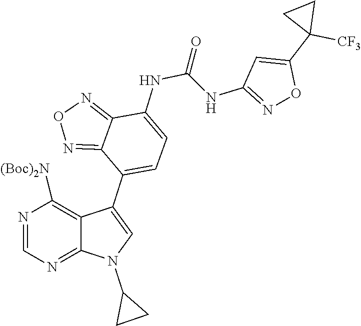

The title compound was obtained by following the general procedure for urea formation (Method B), starting from tert-butyl (5-(7-aminobenzo[c][1,2,5]oxadiazol-4-yl)-7-cyclopropyl-7H-pyrrolo[2,3-d]pyrimidin-4-yl)(tert-butoxycarbonyl)carbamate (B14, 80 mg, 0.158 mmol) and phenyl (5-(1-(trifluoromethyl)cyclopropyl)isoxazol-3-yl)carbamate (C3, 49 mg, 0.158 mmol), and was isolated as yellow solid (20 mg, 17% yield). MS: 726.2 [M+H].

### Step 2: Synthesis of 1-(7-(4-amino-7-cyclopropyl-7H-pyrrolo[2,3-d]pyrimidin-5-yl)benzo[c][1,2,5]oxadiazol-4-yl)-3-(5-(1-(trifluoromethyl)cyclopropyl)isoxazol-3-yl)urea

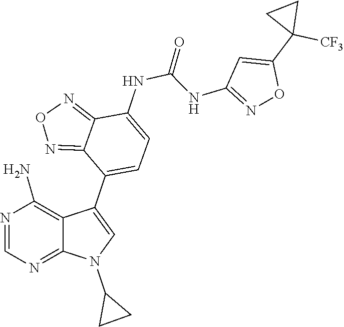 [SMILES: Nc1ncnc2c1c(-c1ccc(NC(=O)Nc3cc(C4(C(F)(F)F)CC4)on3)c3nonc13)cn2C1CC1]

The title compound was obtained by following a similar procedure described for step 2 of Example 5, starting from tert-butyl (tert-butoxycarbonyl)(7-cyclopropyl-5-(7-(3-(5-(1-(trifluoromethyl)cyclopropyl)isoxazol-3-yl)ureido)benzo[c][1,2,5]oxadiazol-4-yl)-7H-pyrrolo[2,3-d]pyrimidin-4-yl)carbamate (20 mg, 0.028 mmol), and was isolated as a yellow solid (1 mg, 7% yield). ¹H NMR (400 MHz, CD₃OD) δ=8.25 (s, 1H), 8.20 (d, J=7.6 Hz, 1H), 7.55 (s, 1H), 7.48 (d, J=7.6 Hz, 1H), 6.90 (s, 1H), 3.58-3.59 (m, 1H), 1.56-1.60 (m, 4H), 1.12-1.20 (m, 4H). MS: 526.2 [M+H].

### Example 78

### 1-(7-(4-Amino-7-Cyclopropyl-7H-Pyrrolo[2,3-D]Pyrimidin-5-Yl)Benzo[C][1,2,5]Oxadiazol-4-Yl)-3-(4-((4-Methylpiperazin-1-Yl)Methyl)-3-(Trifluoromethyl)Phenyl)Urea

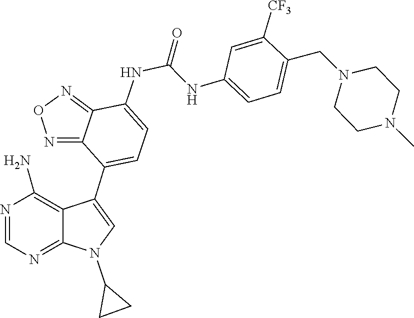 [SMILES: CN1CCN(Cc2ccc(NC(=O)Nc3ccc(-c4cn(C5CC5)c5ncnc(N)c45)c4nonc34)cc2C(F)(F)F)CC1]

The title compound was obtained by following a similar procedure described for Intermediate B1, starting from 1-(7-bromobenzo[c][1,2,5]oxadiazol-4-yl)-3-(4-((4-methylpiperazin-1-yl)methyl)-3-(trifluoromethyl)phenyl)urea (D37, 31 mg, 0.060 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 30 mg, 0.060 mmol), and was isolated as a yellow solid (3 mg, 8% yield). ¹H NMR (400 MHz, CD₃OD) δ=8.39 (s, 1H), 8.19 (d, J=7.6 Hz, 1H), 8.00 (d, J=2.0 Hz, 1H), 7.71-7.76 (m, 3H), 7.54 (d, J=7.2 Hz, 1H), 3.73-3.77 (m, 3H), 3.49-3.51 (m, 2H), 3.28-3.28 (m, 2H), 3.14-3.17 (m, 2H), 2.93 (s, 3H), 2.50 (bs, 2H), 1.20-1.25 (m, 4H). LCMS: 607.2 [M+H].

### Example 79

### 1-(7-(4-Amino-7-Cyclopropyl-7H-Pyrrolo[2,3-D]Pyrimidin-5-Yl)Benzo[C][1,2,5]Oxadiazol-4-Yl)-3-(4-((1-Methylpiperidin-4-Yl)Oxy)-3-(Trifluoromethyl)Phenyl)Urea

### Step 1: Synthesis of tert-butyl (tert-butoxycarbonyl)(7-cyclopropyl-5-(7-(3-(4-((1-methylpiperidin-4-yl)oxy)-3-(trifluoromethyl)phenyl)ureido)benzo[c][1,2,5]oxadiazol-4-yl)-7H-pyrrolo[2,3-d]pyrimidin-4-yl)carbamate

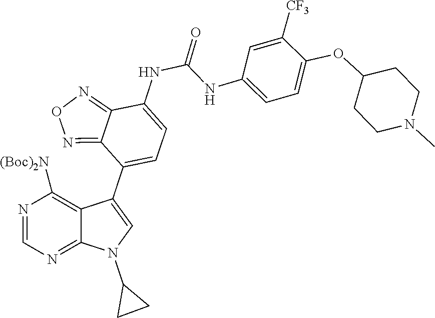

The title compound was obtained by following the general procedure for urea formation (Method B), starting from 4-((1-methylpiperidin-4-yl)oxy)-3-(trifluoromethyl)aniline (synthesized as reported in PCT Publication No. WO 2017/125530, 18 mg, 0.067 mmol) and tert-butyl (tert-butoxycarbonyl)(7-cyclopropyl-5-(7-((phenoxycarbonyl)amino)benzo[c][1,2,5]oxadiazol-4-yl)-7H-pyrrolo[2,3-d]pyrimidin-4-yl)carbamate (C36, 42 mg, 0.299 mmol), and was isolated as brown gum (30 mg, 56% yield). MS: 808.3 [M+H].

### Step 2: synthesis of 1-(7-(4-amino-7-cyclopropyl-7H-pyrrolo[2,3-d]pyrimidin-5-yl)benzo[c][1,2,5]oxadiazol-4-yl)-3-(4-((1-methylpiperidin-4-yl)oxy)-3-(trifluoromethyl)phenyl)urea

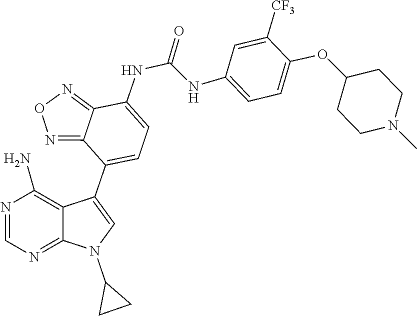 [SMILES: CN1CCC(Oc2ccc(NC(=O)Nc3ccc(-c4cn(C5CC5)c5ncnc(N)c45)c4nonc34)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((1-methylpiperidin-4-yl)oxy)-3-(trifluoromethyl)phenyl)ureido)benzo[c][1,2,5]oxadiazol-4-yl)-7H-pyrrolo[2,3-d]pyrimidin-4-yl)carbamate (25 mg, 0.031 mmol), and was isolated as a yellow solid (5 mg, 16% yield). ¹H NMR (400 MHz, DMSO-d₆) δ=9.55 (s, 1H), 9.45 (s, 1H), 8.36 (s, 1H), 8.11 (d, J=7.6 Hz, 1H), 7.92-7.96 (m, 1H), 7.60-7.63 (m, 2H), 7.32-7.46 (m, 2H), 4.67-4.69 (m, 1H), 3.63-3.67 (m, 1H), 2.93-3.10 (m, 2H), 2.83 (s, 3H), 2.03-2.16 (m, 4H), 1.75-1.78 (m, 2H), 1.10-1.11 (m, 4H). MS: 608.3 [M+H].

### Example 80

### 1-(5-(4-Amino-1-Cyclopropyl-1H-Pyrrolo[3,2-C]Pyridin-3-Yl)Imidazo[1,2-A]Pyridin-8-Yl)-3-(5-(1-(Trifluoromethyl)Cyclopropyl)Isoxazol-3-Yl)Urea

### Step 1: Synthesis of 1-(5-(1-cyclopropyl-4-((2,4-dimethoxybenzyl)amino)-1H-pyrrolo[3,2-c]pyridin-3-yl)imidazo[1,2-a]pyridin-8-yl)-3-(5-(1-(trifluoromethyl)cyclopropyl)isoxazol-3-yl)urea

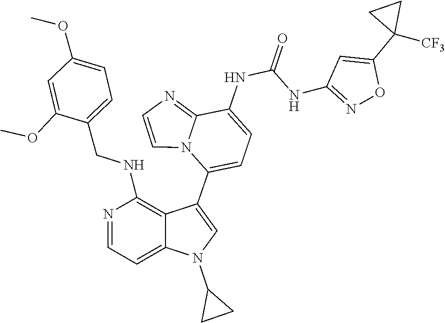 [SMILES: COc1ccc(CNc2nccc3c2c(-c2ccc(NC(=O)Nc4cc(C5(C(F)(F)F)CC5)on4)c4nccn24)cn3C2CC2)c(OC)c1]

The title compound is prepared by following the general procedure for urea formation (Method B), starting from 5-(1-cyclopropyl-4-((2,4-dimethoxybenzyl)amino)-1H-pyrrolo[3,2-c]pyridin-3-yl)imidazo[1,2-a]pyridin-8-amine (B15, 100 mg, 0.22 mmol) and phenyl (5-(1-(trifluoromethyl)cyclopropyl)isoxazol-3-yl)carbamate (C3, 69 mg, 0.22 mmol). MS (expected): 673.2 [M+H].

The title compound was prepared by following the general procedure for urea formation (Method B), starting from 5-(1-cyclopropyl-4-((2,4-dimethoxybenzyl)amino)-1H-pyrrolo[3,2-c]pyridin-3-yl)imidazo[1,2-a]pyridin-8-amine (B15, 20 mg, 0.044 mmol) and phenyl (5-(1-(trifluoromethyl)cyclopropyl)isoxazol-3-yl)carbamate (C3, 14 mg, 0.044 mmol), and was isolated as an off-white solid (12 mg, 39% yield). MS: 673.2 [M+H].

### Step 2: Synthesis of 1-(5-(4-amino-1-cyclopropyl-1H-pyrrolo[3,2-c]pyridin-3-yl)imidazo[1,2-a]pyridin-8-yl)-3-(5-(1-(trifluoromethyl)cyclopropyl)isoxazol-3-yl)urea

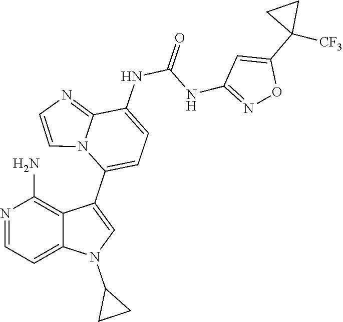 [SMILES: Nc1nccc2c1c(-c1ccc(NC(=O)Nc3cc(C4(C(F)(F)F)CC4)on3)c3nccn13)cn2C1CC1]

Triethylsilane (8.6 mg, 0.074 mmol) and TFA (8.5 mg, 0.074 mmol) are added to a solution of 1-(5-(1-cyclopropyl-4-((2,4-dimethoxybenzyl)amino)-1H-pyrrolo[3,2-c]pyridin-3-yl)imidazo[1,2-a]pyridin-8-yl)-3-(5-(1-(trifluoromethyl)cyclopropyl)isoxazol-3-yl)urea (50 mg, 0.074 mmol) in DCM (3 mL) at 0° C. and the resulting mixture is stirred at 25° C. for 12 h. Following completion of the reaction (as indicated by LCMS), the reaction mixture is concentrated under reduced pressure to give crude material which is purified by preparative HPLC. MS (expected): 523.2 [M+H].

Triethylsilane (2.07 mg, 0.018 mmol) and TFA (2.03 mg, 0.018 mmol) were added to a solution of 1-(5-(1-cyclopropyl-4-((2,4-dimethoxybenzyl)amino)-1H-pyrrolo[3,2-c]pyridin-3-yl)imidazo[1,2-a]pyridin-8-yl)-3-(5-(1-(trifluoromethyl)cyclopropyl)isoxazol-3-yl)urea (12 mg, 0.018 mmol) in DCM (3 mL) at 0° C. and the resulting mixture was stirred at 45° C. for 12 h. Following completion of the reaction (as indicated by LCMS), the reaction mixture was concentrated under reduced pressure to give crude material which was purified by preparative HPLC to afford the title compound as an off-white solid (6.5 mg, 69% yield). ¹H NMR (400 MHz, DMSO-d₆) δ=10.46 (bs, 1H), 9.53 (bs, 1H), 7.98 (d, J=7.6 Hz, 1H), 7.74 (d, J=6.0 Hz, 1H), 7.57-7.62 (m, 3H), 6.93-6.96 (m, 2H), 6.84 (d, J=8.0 Hz, 1H), 5.13 (bs, 2H), 3.50-3.53 (m, 1H), 1.52-1.58 (m, 4H), 1.05-1.09 (m, 4H). MS: 523.2 [M+H].

### Example 81

### 1-(5-(4-Amino-1-Cyclopropyl-1H-Pyrrolo[3,2-C]Pyridin-3-Yl)Imidazo[1,2-A]Pyridin-8-Yl)-3-(4-((4-Methylpiperazin-1-Yl)Methyl)-3-(Trifluoromethyl)Phenyl)Urea

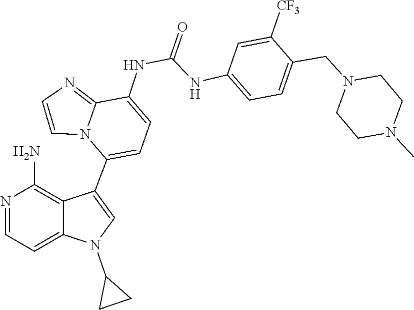 [SMILES: CN1CCN(Cc2ccc(NC(=O)Nc3ccc(-c4cn(C5CC5)c5ccnc(N)c45)n4ccnc34)cc2C(F)(F)F)CC1]

The title compound is prepared by following a similar 2 steps procedure described for Example 80, starting from 5-(1-cyclopropyl-4-((2,4-dimethoxybenzyl)amino)-1H-pyrrolo[3,2-c]pyridin-3-yl)imidazo[1,2-a]pyridin-8-amine (B15, 100 mg, 0.22 mmol) and phenyl (4-((4-methylpiperazin-1-yl)methyl)-3-(trifluoromethyl)phenyl)carbamate (C5, 87 mg, 0.22 mmol) in step 1 and using triethylsilane and TFA in step 2. MS (expected): 604.3 [M+H].

### Step 1: Synthesis of 1-(5-(1-cyclopropyl-4-((2,4-dimethoxybenzyl)amino)-1H-pyrrolo[3,2-c]pyridin-3-yl)imidazo[1,2-a]pyridin-8-yl)-3-(4-((4-methylpiperazin-1-yl)methyl)-3-(trifluoromethyl)phenyl)urea

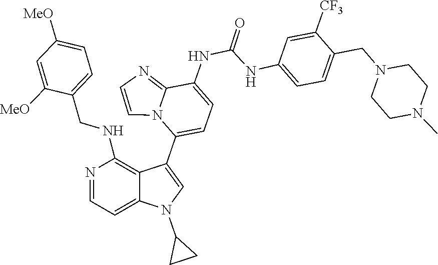 [SMILES: COc1ccc(CNc2nccc3c2c(-c2ccc(NC(=O)Nc4ccc(CN5CCN(C)CC5)c(C(F)(F)F)c4)c4nccn24)cn3C2CC2)c(OC)c1]

The title compound was prepared by following the general procedure for urea formation (Method B), starting from 5-(1-cyclopropyl-4-((2,4-dimethoxybenzyl)amino)-1H-pyrrolo[3,2-c]pyridin-3-yl)imidazo[1,2-a]pyridin-8-amine (B15, 25 mg, 0.055 mmol) and phenyl (4-((4-methylpiperazin-1-yl)methyl)-3-(trifluoromethyl)phenyl)carbamate (C5, 22 mg, 0.055 mmol), and was isolated as an off-white solid (10 mg, 24% yield). MS: 754.3 [M+H].

### Step 2: Synthesis of 1-(5-(4-amino-1-cyclopropyl-1H-pyrrolo[3,2-c]pyridin-3-yl)imidazo[1,2-a]pyridin-8-yl)-3-(4-((4-methylpiperazin-1-yl)methyl)-3-(trifluoromethyl)phenyl)urea

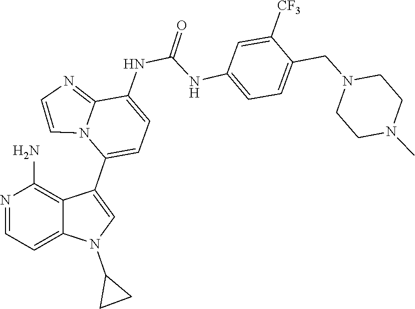 [SMILES: CN1CCN(Cc2ccc(NC(=O)Nc3ccc(-c4cn(C5CC5)c5ccnc(N)c45)n4ccnc34)cc2C(F)(F)F)CC1]

The title compound was prepared by following a similar procedure described for step 2 of Example 80, starting from 1-(5-(1-cyclopropyl-4-((2,4-dimethoxybenzyl)amino)-1H-pyrrolo[3,2-c]pyridin-3-yl)imidazo[1,2-a]pyridin-8-yl)-3-(4-((4-methylpiperazin-1-yl)methyl)-3-(trifluoromethyl)phenyl)urea (10 mg, 0.013 mmol), and was isolated as an off-white solid (2 mg, 25% yield). ¹H NMR (400 MHz, DMSO-d₆) δ=10.01 (bs, 1H), 9.19 (bs, 1H), 7.99-8.04 (m, 2H), 7.74 (d, J=6.0 Hz, 1H), 7.57-7.68 (m, 5H), 6.95 (d, J=6.0 Hz, 1H), 6.84 (d, J=8.0 Hz, 1H), 5.12 (bs, 2H), 3.50-3.55 (m, 3H), 2.40-2.50 (m, 8H), 2.17 (s, 3H), 1.05-1.09 (m, 4H). MS: 604.3 [M+H].

### Example 82

### 1-(5-(4-Amino-1-Cyclopropyl-1H-Pyrrolo[3,2-C]Pyridin-3-Yl)Imidazo[1,2-A]Pyridin-8-Yl)-3-(4-((1-Methylpiperidin-4-Yl)Oxy)-3-(Trifluoromethyl)Phenyl)Urea

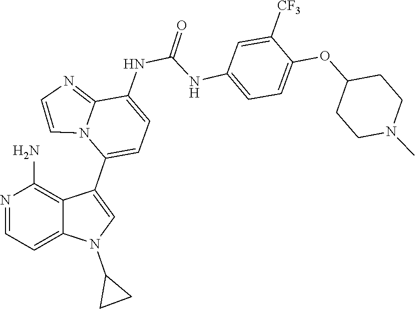 [SMILES: CN1CCC(Oc2ccc(NC(=O)Nc3ccc(-c4cn(C5CC5)c5ccnc(N)c45)n4ccnc34)cc2C(F)(F)F)CC1]

The title compound is prepared by following a similar 2 steps procedure described for Example 80, starting from 5-(1-cyclopropyl-4-((2,4-dimethoxybenzyl)amino)-1H-pyrrolo[3,2-c]pyridin-3-yl)imidazo[1,2-a]pyridin-8-amine (B15, 100 mg, 0.22 mmol) and phenyl (4-((1-methylpiperidin-4-yl)oxy)-3-(trifluoromethyl)phenyl)carbamate (C27, 87 mg, 0.22 mmol) in step 1 and using triethylsilane and TFA in step 2. MS (expected): 605.3 [M+H].

### Example 83

### 1-(5-(4-Amino-7-Isopropyl-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

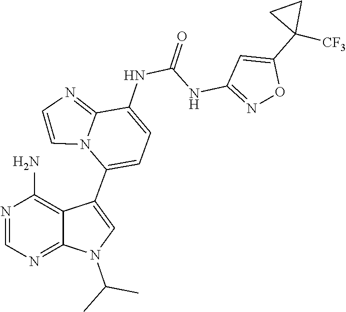 [SMILES: CC(C)n1cc(-c2ccc(NC(=O)Nc3cc(C4(C(F)(F)F)CC4)on3)c3nccn23)c2c(N)ncnc21]

The title compound is prepared by following a similar two-step procedure described for Example 5, starting from tert-butyl (5-(8-aminoimidazo[1,2-a]pyridin-5-yl)-7-isopropyl-7H-pyrrolo[2,3-d]pyrimidin-4-yl)(tert-butoxycarbonyl)carbamate (B16, 100 mg, 0.197 mmol) and phenyl (5-(1-(trifluoromethyl)cyclopropyl)isoxazol-3-yl)carbamate (C3, 62 mg, 0.197 mmol) in step 1. MS (expected): 526.2 [M+H].

### Step 1: Synthesis of 1-(5-(4-((2,4-dimethoxybenzyl)amino)-7-isopropyl-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

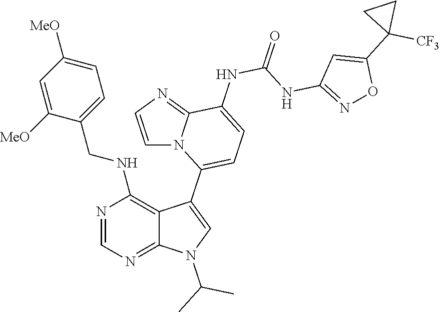 [SMILES: COc1ccc(CNc2ncnc3c2c(-c2ccc(NC(=O)Nc4cc(C5(C(F)(F)F)CC5)on4)c4nccn24)cn3C(C)C)c(OC)c1]

The title compound was prepared by following the general procedure for urea formation (Method B), starting from 5-(8-aminoimidazo[1,2-a]pyridin-5-yl)-N-(2,4-dimethoxybenzyl)-7-isopropyl-7H-pyrrolo[2,3-d]pyrimidin-4-amine (B30, 70 mg, 0.153 mmol) and phenyl (5-(1-(trifluoromethyl)cyclopropyl)isoxazol-3-yl)carbamate (C3, 48 mg, 0.153 mmol), and was isolated as an off-white solid (40 mg, 38% yield). ¹H NMR (400 MHz, DMSO-d₆) δ=10.47 (bs, 1H), 9.56 (bs, 1H), 8.24 (s, 1H), 7.99 (d, J=10.4 Hz, 1H), 7.81 (s, 1H), 7.66 (d, J=1.6 Hz, 1H), 7.57 (d, J=1.2 Hz, 1H), 6.86-6.99 (m, 3H), 6.37-6.46 (m, 2H), 5.80-5.84 (m, 1H), 4.99-5.06 (m, 1H), 4.42-4.44 (m, 2H), 3.70 (s, 3H), 3.59 (s, 3H), 1.49-1.56 (m, 10H). MS: 676.2 [M+H].

### Step 2: Synthesis of 1-(5-(4-amino-7-isopropyl-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

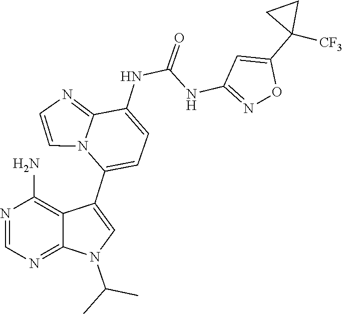 [SMILES: CC(C)n1cc(-c2ccc(NC(=O)Nc3cc(C4(C(F)(F)F)CC4)on3)c3nccn23)c2c(N)ncnc21]

Triethylsilane (6.87 mg, 0.059 mmol) and TFA (6.87 mg, 0.059 mmol) were added to a solution of 1-(5-(4-((2,4-dimethoxybenzyl)amino)-7-isopropyl-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea (40 mg, 0.059 mmol) in DCM (5 mL) at 0° C. and the resulting mixture was stirred at 45° C. for 16 h. Following completion of the reaction (as indicated by LCMS), the reaction mixture was concentrated under reduced pressure to give crude material which was purified by preparative HPLC to afford the title compound as an off-white solid (22 mg, 71% yield). ¹H NMR (400 MHz, DMSO-d₆) δ=10.45 (bs, 1H), 9.53 (bs, 1H), 8.18 (s, 1H), 7.97 (d, J=8.0 Hz, 1H), 7.78 (s, 1H), 7.65 (d, J=1.2 Hz, 1H), 7.58 (d, J=1.2 Hz, 1H), 6.94 (s, 1H), 6.84 (d, J=7.6 Hz, 1H), 6.15 (bs, 2H), 4.99-5.04 (m, 1H), 1.49-1.58 (m, 10H). MS: 526.1 [M+H].

### Example 84

### 1-(5-(4-Amino-7-Cyclobutyl-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

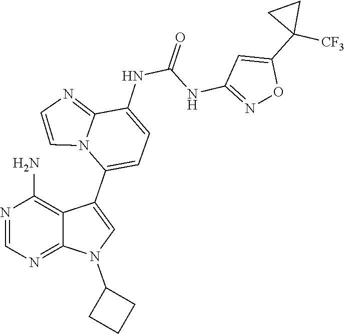 [SMILES: Nc1ncnc2c1c(-c1ccc(NC(=O)Nc3cc(C4(C(F)(F)F)CC4)on3)c3nccn13)cn2C1CCC1]

The title compound is prepared by following a similar two-step procedure described for Example 5, starting from tert-butyl (5-(8-aminoimidazo[1,2-a]pyridin-5-yl)-7-cyclobutyl-7H-pyrrolo[2,3-d]pyrimidin-4-yl)(tert-butoxycarbonyl)carbamate (B17, 100 mg, 0.192 mmol) and phenyl (5-(1-(trifluoromethyl)cyclopropyl)isoxazol-3-yl)carbamate (C3, 60 mg, 0.192 mmol) in step 1. MS (expected): 538.2 [M+H].

The title compound was prepared by following the general procedure for urea formation (Method B), starting from 5-(8-aminoimidazo[1,2-a]pyridin-5-yl)-7-cyclobutyl-7H-pyrrolo[2,3-d]pyrimidin-4-amine (B17, 40 mg, 0.125 mmol) and phenyl (5-(1-(trifluoromethyl)cyclopropyl)isoxazol-3-yl)carbamate (C3, 39 mg, 0.125 mmol), and was isolated as an off-white solid (5 mg, 7.3% yield). ¹H NMR (400 MHz, CD₃OD) δ=8.21 (s, 1H), 8.05 (d, J=8.0 Hz, 1H), 7.81 (s, 1H), 7.59-7.65 (m, 2H), 6.96 (d, J=7.6 Hz, 1H), 6.84 (s, 1H), 5.28-5.32 (m, 1H), 2.57-2.62 (m, 4H), 1.98-2.00 (m, 2H), 1.49-1.59 (m, 4H). MS: 538.1 [M+H].

### Example 85

### 1-(5-(4-Amino-7-(2-Hydroxyethyl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

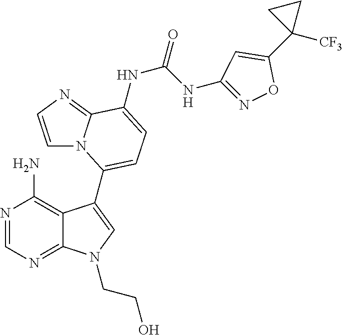 [SMILES: Nc1ncnc2c1c(-c1ccc(NC(=O)Nc3cc(C4(C(F)(F)F)CC4)on3)c3nccn13)cn2CCO]

The title compound is prepared by following a similar two-step procedure described for Example 5, starting from tert-butyl (5-(8-aminoimidazo[1,2-a]pyridin-5-yl)-7-(2-hydroxyethyl)-7H-pyrrolo[2,3-d]pyrimidin-4-yl)(tert-butoxycarbonyl)carbamate (B18, 100 mg, 0.196 mmol) and phenyl (5-(1-(trifluoromethyl)cyclopropyl)isoxazol-3-yl)carbamate (C3, 61 mg, 0.196 mmol) in step 1. MS (expected): 528.2 [M+H].

### Step 1: Synthesis of tert-butyl (tert-butoxycarbonyl)(7-(2-hydroxyethyl)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-4-yl)carbamate

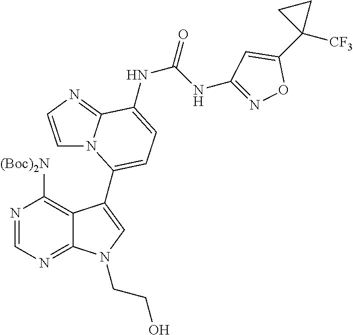

The title compound was prepared by following the general procedure for urea formation (Method B), starting from tert-butyl (5-(8-aminoimidazo[1,2-a]pyridin-5-yl)-7-(2-hydroxyethyl)-7H-pyrrolo[2,3-d]pyrimidin-4-yl)(tert-butoxycarbonyl)carbamate (B18, 30 mg, 0.059 mmol) and phenyl (5-(1-(trifluoromethyl)cyclopropyl)isoxazol-3-yl)carbamate (C3, 18 mg, 0.059 mmol), and was isolated as an off-white solid (10 mg, 23% yield). MS: 728.2 [M+H].

### Step 2: Synthesis of 1-(5-(4-amino-7-(2-hydroxyethyl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

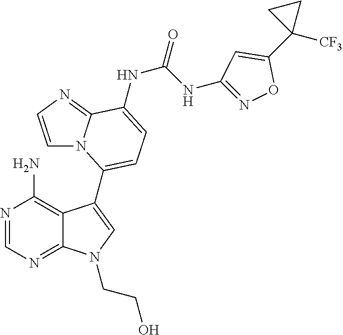 [SMILES: Nc1ncnc2c1c(-c1ccc(NC(=O)Nc3cc(C4(C(F)(F)F)CC4)on3)c3nccn13)cn2CCO]

The title compound was obtained by following a similar procedure described for step 2 of Example 5, starting from tert-butyl (tert-butoxycarbonyl)(7-(2-hydroxyethyl)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-4-yl)carbamate (10 mg, 0.014 mmol), and was isolated as an off-white solid (6.5 mg, 90% yield). ¹H NMR (400 MHz, DMSO-d₆) δ=10.45 (bs, 1H), 9.52 (bs, 1H), 8.18 (s, 1H), 7.98 (d, J=8.0 Hz, 1H), 7.71 (d, J=1.2 Hz, 1H), 7.65 (s, 1H), 7.59 (d, J=1.2 Hz, 1H), 6.93 (s, 1H), 6.82 (d, J=7.6 Hz, 1H), 6.15 (bs, 2H), 4.99 (t, J=5.6 Hz, 1H), 4.27 (t, J=5.6 Hz, 2H), 3.77-3.81 (m, 2H), 1.52-1.56 (m, 4H). MS: 528.3 [M+H].

### Example 86

### 1-(5-(4-Amino-7-(2-Hydroxy-2-Methylpropyl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

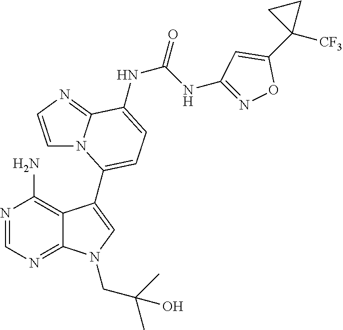 [SMILES: CC(C)(O)Cn1cc(-c2ccc(NC(=O)Nc3cc(C4(C(F)(F)F)CC4)on3)c3nccn23)c2c(N)ncnc21]

The title compound is prepared by following a similar two-step procedure described for Example 5, starting from tert-butyl (5-(8-aminoimidazo[1,2-a]pyridin-5-yl)-7-(2-hydroxy-2-methylpropyl)-7H-pyrrolo[2,3-d]pyrimidin-4-yl)(tert-butoxycarbonyl)carbamate (B19, 100 mg, 0.186 mmol) and phenyl (5-(1-(trifluoromethyl)cyclopropyl)isoxazol-3-yl)carbamate (C3, 58 mg, 0.186 mmol) in step 1. MS (expected): 556.2 [M+H].

### Step 1: Synthesis of tert-butyl (7-(2-hydroxy-2-methylpropyl)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-4-yl)carbamate

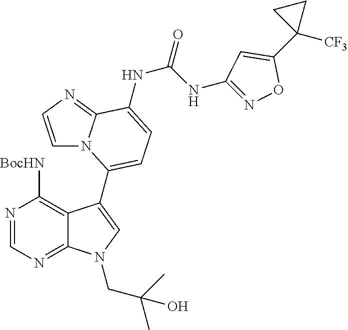 [SMILES: CC(C)(O)Cn1cc(-c2ccc(NC(=O)Nc3cc(C4(C(F)(F)F)CC4)on3)c3nccn23)c2c(NC(=O)OC(C)(C)C)ncnc21]

The title compound was prepared by following the general procedure for urea formation (Method B), starting from tert-butyl (5-(8-aminoimidazo[1,2-a]pyridin-5-yl)-7-(2-hydroxy-2-methylpropyl)-7H-pyrrolo[2,3-d]pyrimidin-4-yl)carbamate (B19, 18 mg, 0.041 mmol) and phenyl (5-(1-(trifluoromethyl)cyclopropyl)isoxazol-3-yl)carbamate (C3, 13 mg, 0.041 mmol), and was isolated as an off-white solid (10 mg, 37% yield). MS: 654.2 [M−H].

### Step 2: Synthesis of 1-(5-(4-amino-7-(2-hydroxy-2-methylpropyl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

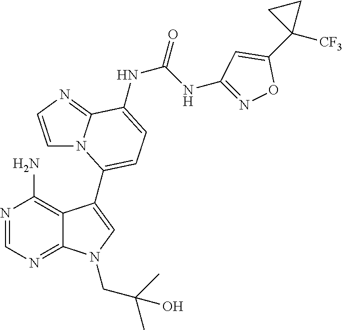 [SMILES: CC(C)(O)Cn1cc(-c2ccc(NC(=O)Nc3cc(C4(C(F)(F)F)CC4)on3)c3nccn23)c2c(N)ncnc21]

The title compound was obtained by following a similar procedure described for step 2 of Example 5, starting from tert-butyl (7-(2-hydroxy-2-methylpropyl)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-4-yl)carbamate (5 mg, 0.008 mmol), and was isolated as an off-white solid (1.1 mg, 26% yield). ¹H NMR (400 MHz, CD₃OD) δ=8.34 (s, 1H), 8.05 (d, J=7.6 Hz, 1H), 7.72-7.82 (m, 3H), 7.14 (d, J=8.0 Hz, 1H), 6.83 (s, 1H), 4.36 (s, 2H), 1.50-1.60 (m, 4H), 1.27 (s, 6H). MS: 556.2 [M+H].

### Example 87

### 1-(5-(4-Amino-7-(3-Hydroxycyclobutyl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

### Step 1: Synthesis of 1-(5-(4-amino-7-(3-(benzyloxy)cyclobutyl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

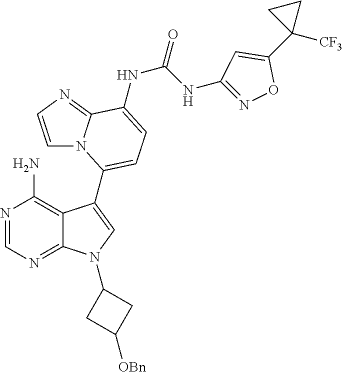 [SMILES: Nc1ncnc2c1c(-c1ccc(NC(=O)Nc3cc(C4(C(F)(F)F)CC4)on3)c3nccn13)cn2C1CC(OCc2ccccc2)C1]

The title compound is prepared by following a similar two-step procedure described for Example 5, starting from tert-butyl (5-(8-aminoimidazo[1,2-a]pyridin-5-yl)-7-(3-(benzyloxy)cyclobutyl)-7H-pyrrolo[2,3-d]pyrimidin-4-yl)(tert-butoxycarbonyl)carbamate (B20, 100 mg, 0.160 mmol) and phenyl (5-(1-(trifluoromethyl)cyclopropyl)isoxazol-3-yl)carbamate (C3, 50 mg, 0.160 mmol) in step 1. MS (expected): 644.2 [M+H].

The title compound was prepared by following the general procedure for urea formation (Method B), starting from 5-(8-aminoimidazo[1,2-a]pyridin-5-yl)-7-(3-(benzyloxy)cyclobutyl)-7H-pyrrolo[2,3-d]pyrimidin-4-amine (B20, 20 mg, 0.047 mmol) and phenyl (5-(1-(trifluoromethyl)cyclopropyl)isoxazol-3-yl)carbamate (C3, 15 mg, 0.047 mmol), and was isolated as an off-white solid (10 mg, 33% yield). MS: 426.2 [M+H].

### Step 2: Synthesis of 1-(5-(4-amino-7-(3-hydroxycyclobutyl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

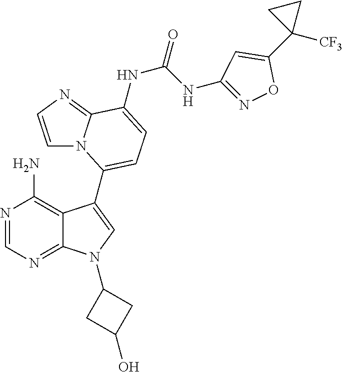 [SMILES: Nc1ncnc2c1c(-c1ccc(NC(=O)Nc3cc(C4(C(F)(F)F)CC4)on3)c3nccn13)cn2C1CC(O)C1]

Boron trichloride (1M in DCM, 621 μL, 0.621 mmol) is added dropwise to a solution of 1-(5-(4-amino-7-(3-(benzyloxy)cyclobutyl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea (50 mg, 0.078 mmol) in DCM (3.5 mL) at −60° C. and the resulting mixture is stirred at 0° C. for 3 h. Following completion of the reaction (as indicated by TLC and LCMS), the reaction mixture is cooled to −70° C., neutralized with NH₄OH (25% in water), and extracted with DCM (2×10 mL). The combined organic extracts are dried over Na₂SO₄, filtered, and concentrated under reduced pressure to yield crude material which is purified by preparative HPLC (eluting with a gradient of 1% TFA in water and ACN), affording the title product. MS (expected): 554.2 [M+H].

Boron trichloride (1M in DCM, 0.023 mL, 0.023 mmol) was added dropwise to a solution of 1-(5-(4-amino-7-(3-(benzyloxy)cyclobutyl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea (15 mg, 0.023 mmol) in DCM (5 mL) at −60° C. and the resulting mixture was stirred at 25° C. for 16 h. Following completion of the reaction (as indicated by LCMS), the reaction mixture was cooled to −70° C., neutralized with NH₄OH (25% in water), and extracted with DCM (2×10 mL). The combined organic extracts were dried over Na₂SO₄, filtered, and concentrated under reduced pressure to yield crude material which was purified by preparative HPLC (eluting with 10 mM NH₄HCO₃ in water and ACN), affording the title product as an off-white solid (7 mg. 54% yield). ¹H NMR (400 MHz, DMSO-d₆) δ=10.46 (bs, 1H), 9.53 (bs, 1H), 8.18 (s, 1H), 7.97 (d, J=8.0 Hz, 1H), 7.88 (s, 1H), 7.68 (d, J=1.2 Hz, 1H), 7.58 (d, J=1.2 Hz, 1H), 6.94 (s, 1H), 6.86 (d, J=7.6 Hz, 1H), 6.23 (bs, 2H), 5.42-5.46 (m, 1H), 5.22-5.23 (m, 1H), 4.47-4.48 (m, 1H), 2.74-2.80 (m, 2H), 2.33-2.44 (m, 2H), 1.24-1.58 (m, 4H). MS: 554.1 [M+H].

### Example 88

### 3-(4-Amino-5-(8-(3-(5-(1-(Trifluoromethyl)Cyclopropyl)Isoxazol-3-Yl)Ureido)Imidazo[1,2-A]Pyridin-5-Yl)-7H-Pyrrolo[2,3-D]Pyrimidin-7-Yl)Propanoic Acid

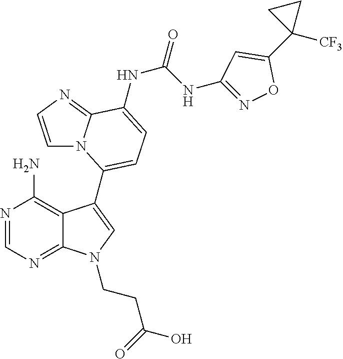 [SMILES: Nc1ncnc2c1c(-c1ccc(NC(=O)Nc3cc(C4(C(F)(F)F)CC4)on3)c3nccn13)cn2CCC(=O)O]

The title compound is prepared by following a similar two-step procedure described for Example 5, starting from tert-butyl 3-(5-(8-aminoimidazo[1,2-a]pyridin-5-yl)-4-(bis(tert-butoxycarbonyl)amino)-7H-pyrrolo[2,3-d]pyrimidin-7-yl)propanoate (B21, 100 mg, 0.168 mmol) and phenyl (5-(1-(trifluoromethyl)cyclopropyl)isoxazol-3-yl)carbamate (C3, 53 mg, 0.168 mmol) in step 1. Cleavage of the t-butyl ester protecting group is expected in step 2 under Boc deprotection conditions (TFA in DCM). MS (expected): 556.2 [M+H].

### Step 1: Synthesis of tert-butyl 3-(4-((2,4-dimethoxybenzyl)amino)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-7-yl)propanoate

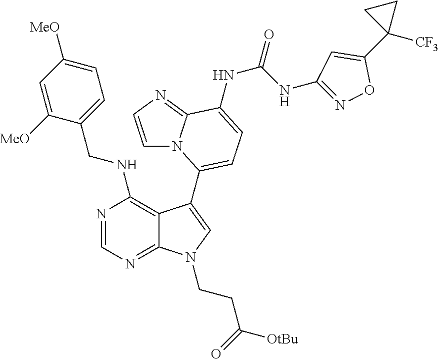 [SMILES: COc1ccc(CNc2ncnc3c2c(-c2ccc(NC(=O)Nc4cc(C5(C(F)(F)F)CC5)on4)c4nccn24)cn3CCC(=O)OC(C)(C)C)c(OC)c1]

The title compound was obtained by following a similar procedure described for Intermediate B1, starting from starting from 1-(5-bromoimidazo[1,2-a]pyridin-8-yl)-3-(5-(1-(trifluoromethyl)cyclopropyl)isoxazol-3-yl)urea (D38, 125 mg, 0.232 mmol) and tert-butyl 3-(4-((2,4-dimethoxybenzyl)amino)-5-(4,4,5,5-tetramethyl-1,3,2-dioxaborolan-2-yl)-7H-pyrrolo[2,3-d]pyrimidin-7-yl)propanoate (B21, 125 mg, 0.232 mmol), and was isolated as an off-white solid (30 mg, 17% yield). MS: 762.3 [M+H].

### Step 2: Synthesis of 3-(4-amino-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-7-yl)propanoic acid

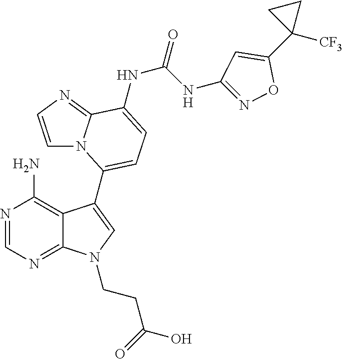 [SMILES: Nc1ncnc2c1c(-c1ccc(NC(=O)Nc3cc(C4(C(F)(F)F)CC4)on3)c3nccn13)cn2CCC(=O)O]

The title compound was prepared by following a similar procedure described for step 2 of Example 80, starting from tert-butyl 3-(4-((2,4-dimethoxybenzyl)amino)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-7-yl)propanoate (30 mg, 0.039 mmol). MS: 556.2 [M+H].

### Example 89

### 1-(5-(4-Amino-7-(2-Methoxyethyl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

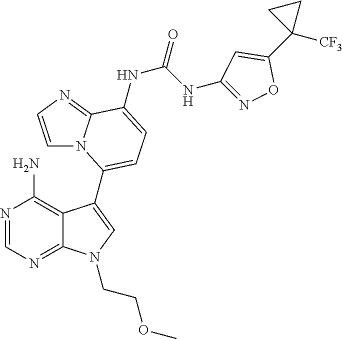 [SMILES: COCCn1cc(-c2ccc(NC(=O)Nc3cc(C4(C(F)(F)F)CC4)on3)c3nccn23)c2c(N)ncnc21]

The title compound is prepared by following a similar two-step procedure described for Example 5, starting from tert-butyl (5-(8-aminoimidazo[1,2-a]pyridin-5-yl)-7-(2-methoxyethyl)-7H-pyrrolo[2,3-d]pyrimidin-4-yl)(tert-butoxycarbonyl)carbamate (B22, 100 mg, 0.191 mmol) and phenyl (5-(1-(trifluoromethyl)cyclopropyl)isoxazol-3-yl)carbamate (C3, 60 mg, 0.191 mmol) in step 1. MS (expected): 542.2 [M+H].

### Step 1: Synthesis of tert-butyl (tert-butoxycarbonyl)(7-(2-methoxyethyl)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-4-yl)carbamate

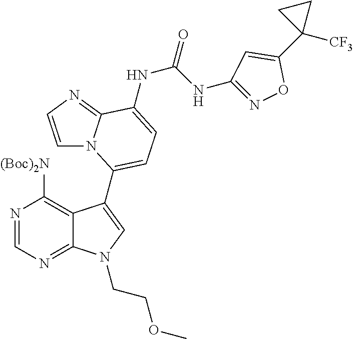

The title compound was prepared by following the general procedure for urea formation (Method B), starting from tert-butyl (5-(8-aminoimidazo[1,2-a]pyridin-5-yl)-7-(2-methoxyethyl)-7H-pyrrolo[2,3-d]pyrimidin-4-yl)(tert-butoxycarbonyl)carbamate (B22, 50 mg, 0.095 mmol) and phenyl (5-(1-(trifluoromethyl)cyclopropyl)isoxazol-3-yl)carbamate (C3, 30 mg, 0.095 mmol), and was isolated as an off-white solid (10 mg, 14% yield). MS: 742.2 [M+H].

### Step 2: Synthesis of 1-(5-(4-amino-7-(2-methoxyethyl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

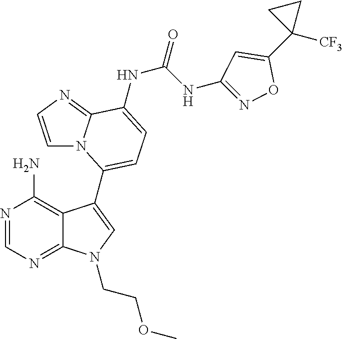 [SMILES: COCCn1cc(-c2ccc(NC(=O)Nc3cc(C4(C(F)(F)F)CC4)on3)c3nccn23)c2c(N)ncnc21]

The title compound was obtained by following a similar procedure described for step 2 of Example 5, starting from tert-butyl (tert-butoxycarbonyl)(7-(2-methoxyethyl)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-4-yl)carbamate (6 mg, 0.008 mmol), and was isolated as an off-white solid (1.8 mg, 39% yield). ¹H NMR (400 MHz, DMSO-d₆) δ=10.46 (bs, 1H), 9.53 (bs, 1H), 8.20 (s, 1H), 7.97 (d, J=7.6 Hz, 1H), 7.59-7.67 (m, 3H), 6.94 (s, 1H), 6.84 (d, J=8.0 Hz, 1H), 6.27 (bs, 2H), 4.39 (t, J=5.6 Hz, 2H), 3.75 (t, J=5.6 Hz, 2H), 3.24 (s, 3H), 1.52-1.58 (m, 4H). MS: 542.1 [M+H].

### Example 90

### 1-(5-(4-Amino-7-(Oxetan-3-Yl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

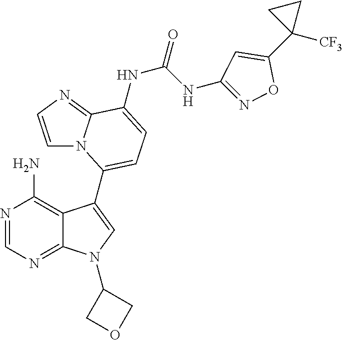 [SMILES: Nc1ncnc2c1c(-c1ccc(NC(=O)Nc3cc(C4(C(F)(F)F)CC4)on3)c3nccn13)cn2C1COC1]

The title compound is prepared by following a similar two-step procedure described for Example 5, starting from tert-butyl (5-(8-aminoimidazo[1,2-a]pyridin-5-yl)-7-(oxetan-3-yl)-7H-pyrrolo[2,3-d]pyrimidin-4-yl)(tert-butoxycarbonyl)carbamate (B23, 100 mg, 0.192 mmol) and phenyl (5-(1-(trifluoromethyl)cyclopropyl)isoxazol-3-yl)carbamate (C3, 60 mg, 0.191 mmol) in step 1. MS (expected): 540.2 [M+H].

### Example 91

### 1-(5-(4-Amino-7-(Pyridin-3-Yl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

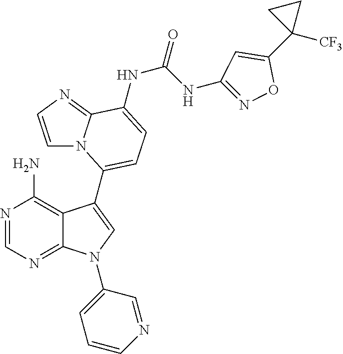 [SMILES: Nc1ncnc2c1c(-c1ccc(NC(=O)Nc3cc(C4(C(F)(F)F)CC4)on3)c3nccn13)cn2-c1cccnc1]

The title compound is prepared by following a similar two-step procedure described for Example 5, starting from tert-butyl (5-(8-aminoimidazo[1,2-a]pyridin-5-yl)-7-(pyridin-3-yl)-7H-pyrrolo[2,3-d]pyrimidin-4-yl)(tert-butoxycarbonyl)carbamate (B24, 100 mg, 0.184 mmol) and phenyl (5-(1-(trifluoromethyl)cyclopropyl)isoxazol-3-yl)carbamate (C3, 58 mg, 0.184 mmol) in step 1. MS (expected): 561.2 [M+H].

The title compound was prepared by following the general procedure for urea formation (Method B), starting from 5-(8-aminoimidazo[1,2-a]pyridin-5-yl)-7-(pyridin-3-yl)-7H-pyrrolo[2,3-d]pyrimidin-4-amine (B24, 15 mg, 0.044 mmol) and phenyl (5-(1-(trifluoromethyl)cyclopropyl)isoxazol-3-yl)carbamate (C3, 14 mg, 0.044 mmol), and was isolated as an off-white solid (0.6 mg, 2.4% yield). ¹H NMR (400 MHz, CD₃OD) δ=9.13 (bs, 1H), 8.63 (bs, 1H), 8.36-8.39 (m, 1H), 8.29 (s, 1H), 8.10 (d, J=7.6 Hz, 1H), 7.96 (s, 1H), 7.78 (s, 1H), 7.67-7.70 (m, 1H), 7.62 (s, 1H), 7.08 (d, J=7.6 Hz, 1H), 6.85 (s, 1H), 1.51-1.57 (m, 4H). MS: 561.2 [M+H].

### Example 92

### 1-(5-(4-Amino-7-(Pyridin-4-Yl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

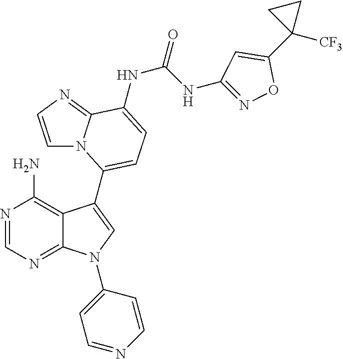 [SMILES: Nc1ncnc2c1c(-c1ccc(NC(=O)Nc3cc(C4(C(F)(F)F)CC4)on3)c3nccn13)cn2-c1ccncc1]

The title compound is prepared by following a similar two-step procedure described for Example 5, starting from tert-butyl (5-(8-aminoimidazo[1,2-a]pyridin-5-yl)-7-(pyridin-4-yl)-7H-pyrrolo[2,3-d]pyrimidin-4-yl)(tert-butoxycarbonyl)carbamate (B25, 100 mg, 0.184 mmol) and phenyl (5-(1-(trifluoromethyl)cyclopropyl)isoxazol-3-yl)carbamate (C3, 58 mg, 0.184 mmol) in step 1. MS (expected): 561.2 [M+H].

### Example 93

### 1-(5-(4-Amino-7-(1-Methylpyrrolidin-3-Yl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

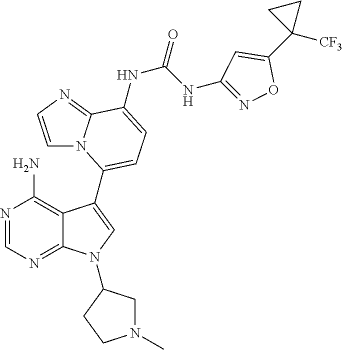 [SMILES: CN1CCC(n2cc(-c3ccc(NC(=O)Nc4cc(C5(C(F)(F)F)CC5)on4)c4nccn34)c3c(N)ncnc32)C1]

The title compound is prepared by following a similar two-step procedure described for Example 5, starting from tert-butyl (5-(8-aminoimidazo[1,2-a]pyridin-5-yl)-7-(1-methylpyrrolidin-3-yl)-7H-pyrrolo[2,3-d]pyrimidin-4-yl)(tert-butoxycarbonyl)carbamate (B26, 100 mg, 0.182 mmol) and phenyl (5-(1-(trifluoromethyl)cyclopropyl)isoxazol-3-yl)carbamate (C3, 57 mg, 0.182 mmol) in step 1. MS (expected): 567.2 [M+H].

### Example 94

### 1-(5-(4-Amino-7-(Piperidin-4-Yl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

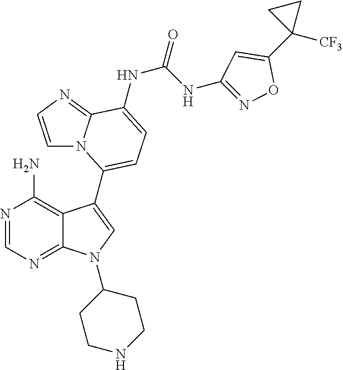 [SMILES: Nc1ncnc2c1c(-c1ccc(NC(=O)Nc3cc(C4(C(F)(F)F)CC4)on3)c3nccn13)cn2C1CCNCC1]

The title compound is prepared by following a similar two-step procedure described for Example 5, starting from tert-butyl 4-(5-(8-aminoimidazo[1,2-a]pyridin-5-yl)-4-(bis(tert-butoxycarbonyl)amino)-7H-pyrrolo[2,3-d]pyrimidin-7-yl)piperidine-1-carboxylate (B27, 100 mg, 0.154 mmol) and phenyl (5-(1-(trifluoromethyl)cyclopropyl)isoxazol-3-yl)carbamate (C3, 48 mg, 0.154 mmol) in step 1. Cleavage of all three Boc protecting groups is expected under TFA conditions in step 2. MS (expected): 556.2 [M+H].

### Step 1: Synthesis of tert-butyl 4-(4-((2,4-dimethoxybenzyl)amino)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-7-yl)piperidine-1-carboxylate

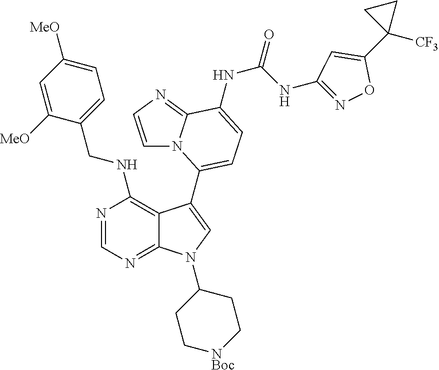 [SMILES: COc1ccc(CNc2ncnc3c2c(-c2ccc(NC(=O)Nc4cc(C5(C(F)(F)F)CC5)on4)c4nccn24)cn3C2CCN(C(=O)OC(C)(C)C)CC2)c(OC)c1]

The title compound was prepared by following the general procedure for urea formation (Method B), starting from tert-butyl 4-(5-(8-aminoimidazo[1,2-a]pyridin-5-yl)-4-((2,4-dimethoxybenzyl)amino)-7H-pyrrolo[2,3-d]pyrimidin-7-yl)piperidine-1-carboxylate (B31, 100 mg, 0.167 mmol) and phenyl (5-(1-(trifluoromethyl)cyclopropyl)isoxazol-3-yl)carbamate (C3, 52.2 mg, 0.167 mmol), and was isolated as an off-white solid (33 mg, 24% yield). MS: 817.3 [M+H].

### Step 2: Synthesis of 1-(5-(4-amino-7-(piperidin-4-yl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

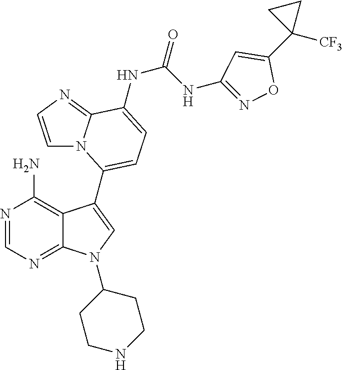 [SMILES: Nc1ncnc2c1c(-c1ccc(NC(=O)Nc3cc(C4(C(F)(F)F)CC4)on3)c3nccn13)cn2C1CCNCC1]

The title compound is prepared by following a similar procedure described for step 2 of Example 83, starting from tert-butyl 4-(4-((2,4-dimethoxybenzyl)amino)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-7-yl)piperidine-1-carboxylate (10 mg, 0.012 mmol), and was isolated as an off-white solid (3 mg, 42% yield). MS: 567.2 [M+H].

### Example 95

### 1-(5-(4-Amino-7-(1-Methylpiperidin-4-Yl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

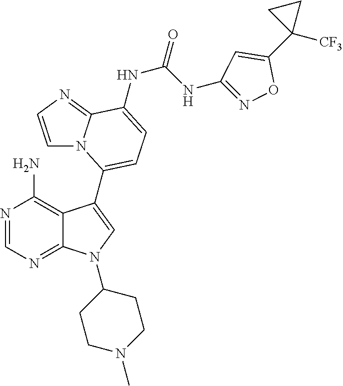 [SMILES: CN1CCC(n2cc(-c3ccc(NC(=O)Nc4cc(C5(C(F)(F)F)CC5)on4)c4nccn34)c3c(N)ncnc32)CC1]

Paraformaldehyde (16 mg, 0.529 mmol) and acetic acid (0.5 μL, 0.009 mmol) are added to a solution of 1-(5-(4-amino-7-(piperidin-4-yl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea (Example 94, 100 mg, 0.176 mmol) in MeOH (5 mL) and the resulting mixture is stirred at 50° C. for 2 h. NaBH₃CN (55 mg, 0.882 mmol) is then added and the reaction mixture is stirred at 50° C. for another 12 h. Following completion of the reaction (as indicated by LCMS), the reaction mixture is concentrated under reduced pressure to yield crude material which is purified by preparative HPLC. MS (expected): 581.2 [M+H].

### Step 1: Synthesis of 1-(5-(4-((2,4-dimethoxybenzyl)amino)-7-(piperidin-4-yl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

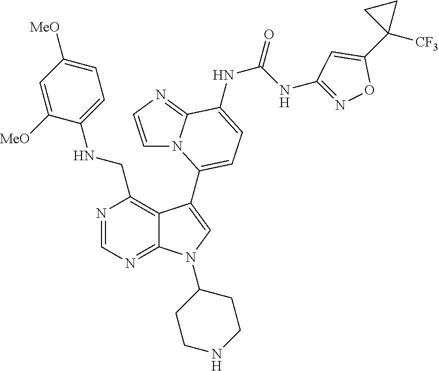 [SMILES: COc1ccc(NCc2ncnc3c2c(-c2ccc(NC(=O)Nc4cc(C5(C(F)(F)F)CC5)on4)c4nccn24)cn3C2CCNCC2)c(OC)c1]

TFA (0.175 mL) was added to a solution of tert-butyl 4-(4-((2,4-dimethoxybenzyl)amino)-5-(8-(3-(5-(1-(trifluoromethyl)cyclopropyl)isoxazol-3-yl)ureido)imidazo[1,2-a]pyridin-5-yl)-7H-pyrrolo[2,3-d]pyrimidin-7-yl)piperidine-1-carboxylate (Step 1 of Example 94, 35 mg, 0.043 mmol) in DCM (2 mL) at 0° C. and the resulting mixture was stirred at 25° C. for 12 h. Following completion of the reaction (as indicated by LCMS), the reaction mixture was concentrated under reduced pressure to afford the title compound (25 mg, crude) which was used without further purification. MS: 717.2 [M+H].

### Step 2: Synthesis of 1-(5-(4-((2,4-dimethoxybenzyl)amino)-7-(1-methylpiperidin-4-yl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

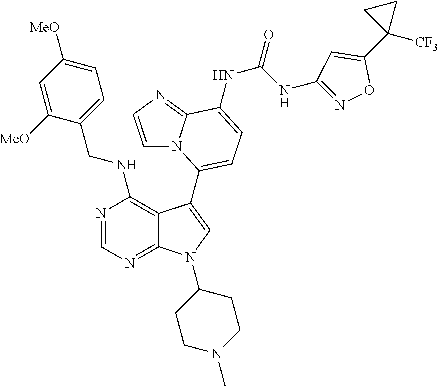 [SMILES: COc1ccc(CNc2ncnc3c2c(-c2ccc(NC(=O)Nc4cc(C5(C(F)(F)F)CC5)on4)c4nccn24)cn3C2CCN(C)CC2)c(OC)c1]

Formaldehyde (37% in water, 2.62 mg, 0.087 mmol) and acetic acid (3.14 mg, 0.052 mmol) were added to a solution of 1-(5-(4-((2,4-dimethoxybenzyl)amino)-7-(piperidin-4-yl)-7H-pyrrolo[2,3-d]pyrimidin-5-yl)imidazo[1,2-a]pyridin-8-yl)-3-(3-(1-(trifluoromethyl)cyclopropyl)isoxazol-5-yl)urea (25 mg, 0.035 mmol) in THF (3 mL) and the resulting mixture was stirred at 25° C. for 2 h. Sodium triacetoxyborohydride (14.72 mg, 0.070 mmol) was then added and the reaction mixture was stirred at 25° C. for another 16 h. Following completion of the reaction (as indicated by LCMS), the reaction mixture was concentrated under reduced pressure to give crude material, which was purified by preparative HPLC, affording the title compound as an off-white solid (20 mg, 77% yield). MS: 731.3 [M+H].

### Step 3: Synthesis of 1-(5-(4-amino-7-(1-methylpiperidin-4-yl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

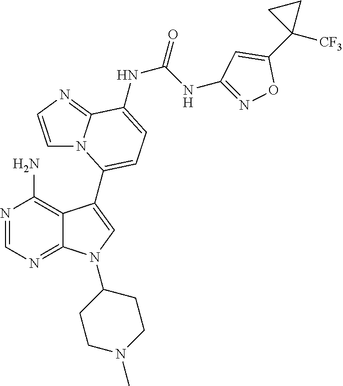 [SMILES: CN1CCC(n2cc(-c3ccc(NC(=O)Nc4cc(C5(C(F)(F)F)CC5)on4)c4nccn34)c3c(N)ncnc32)CC1]

The title compound is prepared by following a similar procedure described for Step 2 of Example 83, starting from 1-(5-(4-((2,4-dimethoxybenzyl)amino)-7-(1-methylpiperidin-4-yl)-7H-pyrrolo[2,3-d]pyrimidin-5-yl)imidazo[1,2-a]pyridin-8-yl)-3-(3-(1-(trifluoromethyl)cyclopropyl)isoxazol-5-yl)urea (20 mg, 0.027 mmol), and was isolated as an off-white solid (7 mg, 44% yield). ¹H NMR (400 MHz, DMSO-d₆) δ=10.45 (bs, 1H), 9.53 (bs, 1H), 8.18 (s, 1H), 7.97 (d, J=7.6 Hz, 1H), 7.78 (s, 1H), 7.64 (s, 1H), 7.57 (s, 1H), 6.94 (s, 1H), 6.83 (d, J=8.0 Hz, 1H), 6.16 (bs, 2H), 4.58-4.60 (m, 1H), 2.91-2.93 (m, 2H), 2.24 (s, 3H), 2.09-2.12 (m, 4H), 1.93-1.95 (m, 2H), 1.52-1.52 (m, 4H). MS: 581.2 [M+H].

### Example 96

### 1-(5-(4-Amino-7-(1-(Methylsulfonyl)Piperidin-4-Yl)-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

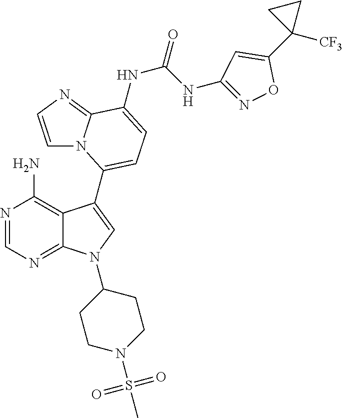 [SMILES: CS(=O)(=O)N1CCC(n2cc(-c3ccc(NC(=O)Nc4cc(C5(C(F)(F)F)CC5)on4)c4nccn34)c3c(N)ncnc32)CC1]

TEA (49 μL, 0.353 mmol) and MsCl (14 μL, 0.176 mmol) are added to a solution of 1-(5-(4-amino-7-(piperidin-4-yl)-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea (Example 94, 100 mg, 0.176 mmol) in DCM (5 mL) at 0° C. and the resulting mixture is stirred at 25° C. for 16 h. Following completion of the reaction (as indicated by TLC), the reaction mixture is diluted with DCM (5 mL) and washed with brine (5 mL). The organic layer is dried over Na₂SO₄, filtered, and concentrated under reduced pressure to give crude material which is purified by preparative HPLC. MS (expected): 645.2 [M+H].

### Example 97

### 1-(5-(4-Amino-1-Cyclopropyl-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

### Step 1: Synthesis of 1-(5-(1-cyclopropyl-4-((2,4-dimethoxybenzyl)amino)-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea

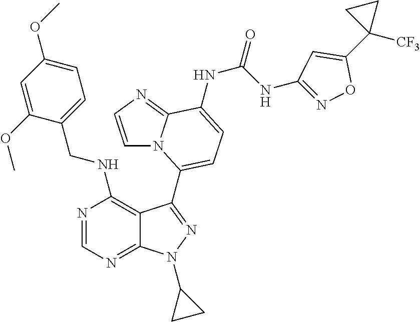 [SMILES: COc1ccc(CNc2ncnc3c2c(-c2ccc(NC(=O)Nc4cc(C5(C(F)(F)F)CC5)on4)c4nccn24)nn3C2CC2)c(OC)c1]

LiCl (17 mg, 0.410 mmol) and CuI (4 mg, 0.020 mmol) are added to a mixture of 1-cyclopropyl-N-(2,4-dimethoxybenzyl)-3-(trimethylstannyl)-1H-pyrazolo[3,4-d]pyrimidin-4-amine (E1, 50 mg, 0.102 mmol) and 1-(5-bromoimidazo[1,2-a]pyridin-8-yl)-3-(5-(1-(trifluoromethyl)cyclopropyl)isoxazol-3-yl)urea (D38, 88 mg, 0.205 mmol) in THF (4 mL) and the resulting mixture is purged with N₂ for 10 minutes. Pd(PPh₃)₄ (12 mg, 0.012 mmol) is then added and the resulting mixture is subjected to microwave irradiation at 100° C. for 1 h. Following completion of the reaction (as indicated by LCMS), the reaction mixture is filtered through a pad of Celite® (i.e., diatomaceous earth) which is then rinsed with THF (2×1 mL). The combined filtrates are concentrated under reduced pressure to give crude material which is purified by preparative HPLC. MS (expected): 675.2 [M+H].

### Step 2: Synthesis of 1-(5-(4-amino-1-cyclopropyl-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea

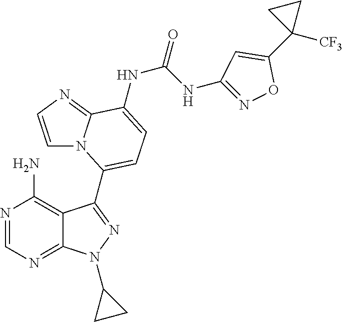 [SMILES: Nc1ncnc2c1c(-c1ccc(NC(=O)Nc3cc(C4(C(F)(F)F)CC4)on3)c3nccn13)nn2C1CC1]

Triethylsilane (0.2 mL) and TFA (0.2 mL) are added to a solution of 1-(5-(1-cyclopropyl-4-((2,4-dimethoxybenzyl)amino)-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea (20 mg, 0.030 mmol) in DCM (2 mL) at 0° C. and the resulting mixture was stirred at 60° C. for 12 h. Following completion of the reaction (as indicated by LCMS), the reaction mixture is concentrated under reduced pressure to give crude material which is purified by preparative HPLC. MS (expected): 525.2 [M+H].

### Example 98

### 1-(5-(4-Amino-1-Cyclopropyl-1H-Pyrazolo[3,4-D]Pyrimidin-3-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of 1-(5-(1-cyclopropyl-4-((2,4-dimethoxybenzyl)amino)-1H-pyrazolo[3,4-d]pyrimidin-3-yl)imidazo[1,2-a]pyridin-8-yl)-3-(4-((4-methylpiperazin-1-yl)methyl)-3-(trifluoromethyl)phenyl)urea

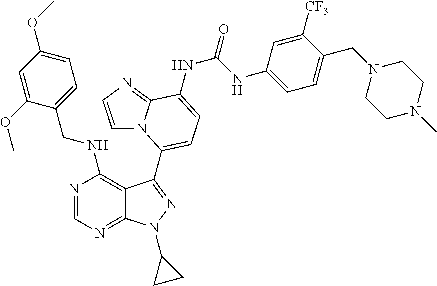 [SMILES: COc1ccc(CNc2ncnc3c2c(-c2ccc(NC(=O)Nc4ccc(CN5CCN(C)CC5)c(C(F)(F)F)c4)c4nccn24)nn3C2CC2)c(OC)c1]

LiCl (21 mg, 0.492 mmol) and CuI (4.68 mg, 0.025 mmol) were added to a mixture of 1-cyclopropyl-N-(2,4-dimethoxybenzyl)-3-(trimethylstannyl)-1H-pyrazolo[3,4-d]pyrimidin-4-amine (E1, 60 mg, 0.123 mmol) and 1-(5-bromoimidazo[1,2-a]pyridin-8-yl)-3-(4-((4-methylpiperazin-1-yl)methyl)-3-(trifluoromethyl)phenyl)urea (D39, 126 mg, 0.246 mmol) in THF (5 mL) and the resulting mixture was purged with N₂ for 10 minutes. Pd(PPh₃)₄ (14 mg, 0.012 mmol) was then added and the resulting mixture was subjected to microwave irradiation at 100° C. for 1 h. Following completion of the reaction (as indicated by LCMS), the reaction mixture was filtered through a pad of Celite® (i.e., diatomaceous earth) which was then rinsed with THF (2×1 mL). The combined filtrates were concentrated under reduced pressure to give crude material which was purified by preparative HPLC (eluting with 0.1% TFA in H₂O and ACN), affording the title product as an off-white solid (20 mg, 21% yield). MS: 756.4 [M+H].

### Step 2: Synthesis of 1-(5-(4-amino-1-cyclopropyl-1H-pyrazolo[3,4-d]pyrimidin-3-yl)imidazo[1,2-a]pyridin-8-yl)-3-(4-((4-methylpiperazin-1-yl)methyl)-3-(trifluoromethyl)phenyl)urea

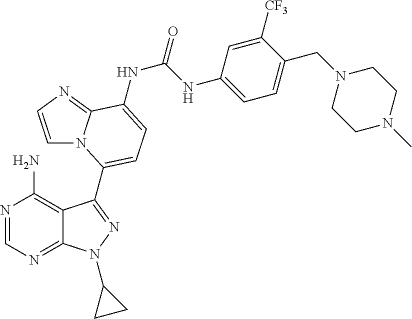 [SMILES: CN1CCN(Cc2ccc(NC(=O)Nc3ccc(-c4nn(C5CC5)c5ncnc(N)c45)n4ccnc34)cc2C(F)(F)F)CC1]

Triethylsilane (0.2 mL) and TFA (0.2 mL) were added to a solution of 1-(5-(1-cyclopropyl-4-((2,4-dimethoxybenzyl)amino)-1H-pyrazolo[3,4-d]pyrimidin-3-yl)imidazo[1,2-a]pyridin-8-yl)-3-(4-((4-methylpiperazin-1-yl)methyl)-3-(trifluoromethyl)phenyl)urea (20 mg, 0.026 mmol) in DCM (2 mL) at 0° C. and the resulting mixture was stirred at 60° C. for 12 h. Following completion of the reaction (as indicated by LCMS), the reaction mixture was concentrated under reduced pressure to give crude material which was purified by preparative HPLC (eluting with 10 mM NH₄HCO₃ in H₂O and ACN), affording the title product as an off-white solid (1 mg, 6.3% yield). ¹H NMR (400 MHz, CD₃OD) δ=8.35 (s, 1H), 8.10-8.12 (m, 2H), 7.98 (d, J=2.0 Hz, 1H), 7.70-7.76 (m, 2H), 7.64 (d, J=1.2 Hz, 1H), 7.17 (d, J=8.0 Hz, 1H), 3.93-3.99 (m, 1H), 3.67 (s, 2H), 2.51 (bs, 8H), 2.34 (s, 3H), 1.34-1.42 (m, 2H), 1.20-1.25 (m, 2H). MS: 606.2 [M+H].

### Example 99

### 1-(5-(4-Amino-1-Cyclopropyl-1H-Pyrazolo[3,4-D]Pyrimidin-3-Yl)Imidazo[1,2-A]Pyridin-8-Yl)-3-(4-((4-Ethylpiperazin-1-Yl)Methyl)-3-(Trifluoromethyl)Phenyl)Urea

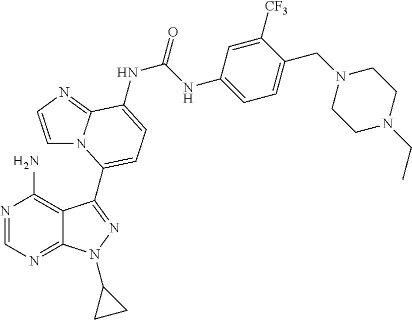 [SMILES: CCN1CCN(Cc2ccc(NC(=O)Nc3ccc(-c4nn(C5CC5)c5ncnc(N)c45)n4ccnc34)cc2C(F)(F)F)CC1]

The title compound is prepared by following a similar 2 steps procedure described for Example 98, starting from 1-cyclopropyl-N-(2,4-dimethoxybenzyl)-3-(trimethylstannyl)-1H-pyrazolo[3,4-d]pyrimidin-4-amine (E1, 50 mg, 0.102 mmol) and 1-(5-bromoimidazo[1,2-a]pyridin-8-yl)-3-(4-((4-ethylpiperazin-1-yl)methyl)-3-(trifluoromethyl)phenyl)urea (D5, 54 mg, 0.102 mmol) in step 1. MS (expected): 620.3 [M+H].

### Example 100

### 1-(5-(4-Amino-1-Cyclopropyl-1H-Pyrazolo[3,4-D]Pyrimidin-3-Yl)Imidazo[1,2-A]Pyridin-8-Yl)-3-(4-((1-Methylpiperidin-4-Yl)Oxy)-3-(Trifluoromethyl)Phenyl)Urea

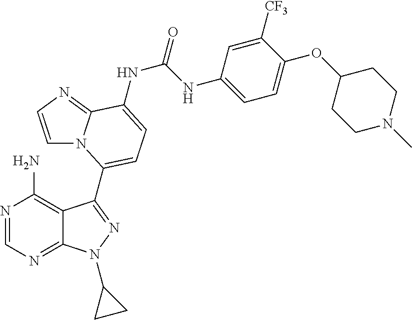 [SMILES: CN1CCC(Oc2ccc(NC(=O)Nc3ccc(-c4nn(C5CC5)c5ncnc(N)c45)n4ccnc34)cc2C(F)(F)F)CC1]

The title compound is prepared by following a similar 2 steps procedure described for Example 98, starting from 1-cyclopropyl-N-(2,4-dimethoxybenzyl)-3-(trimethylstannyl)-1H-pyrazolo[3,4-d]pyrimidin-4-amine (E1, 50 mg, 0.102 mmol) and 1-(5-bromoimidazo[1,2-a]pyridin-8-yl)-3-(4-((1-methylpiperidin-4-yl)oxy)-3-(trifluoromethyl)phenyl)urea (D7, 52 mg, 0.102 mmol) in step 1. MS (expected): 607.2 [M+H].

### Example 101

### 1-(5-(4-Amino-1-Cyclopropyl-1H-Pyrazolo[3,4-D]Pyrimidin-3-Yl)Imidazo[1,2-A]Pyridin-8-Yl)-3-(4-((1-Ethylpiperidin-4-Yl)Oxy)-3-(Trifluoromethyl)Phenyl)Urea

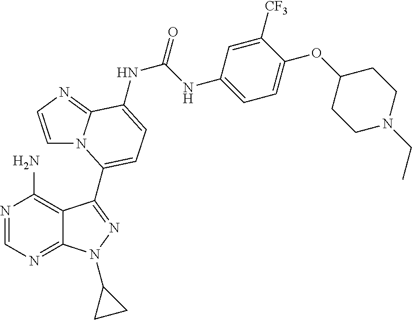 [SMILES: CCN1CCC(Oc2ccc(NC(=O)Nc3ccc(-c4nn(C5CC5)c5ncnc(N)c45)n4ccnc34)cc2C(F)(F)F)CC1]

The title compound is prepared by following a similar 2 steps procedure described for Example 98, starting from 1-cyclopropyl-N-(2,4-dimethoxybenzyl)-3-(trimethylstannyl)-1H-pyrazolo[3,4-d]pyrimidin-4-amine (E1, 50 mg, 0.102 mmol) and 1-(5-bromoimidazo[1,2-a]pyridin-8-yl)-3-(4-((1-ethylpiperidin-4-yl)oxy)-3-(trifluoromethyl)phenyl)urea (D8, 54 mg, 0.102 mmol) in step 1. MS (expected): 621.3 [M+H].

### Example 102

### 1-(5-(4-Amino-1-Cyclopropyl-1H-Pyrazolo[4,3-C]Pyridin-3-Yl)Imidazo[1,2-A]Pyridin-8-Yl)-3-(5-(1-(Trifluoromethyl)Cyclopropyl)Isoxazol-3-Yl)Urea

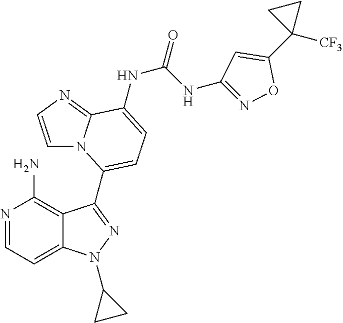 [SMILES: Nc1nccc2c1c(-c1ccc(NC(=O)Nc3cc(C4(C(F)(F)F)CC4)on3)c3nccn13)nn2C1CC1]

The title compound is prepared by following a similar 2 steps procedure described for Example 98, starting from 1-cyclopropyl-N-(2,4-dimethoxybenzyl)-3-(trimethylstannyl)-1H-pyrazolo[4,3-c]pyridin-4-amine (E2, 50 mg, 0.103 mmol) and 1-(5-bromoimidazo[1,2-a]pyridin-8-yl)-3-(5-(1-(trifluoromethyl)cyclopropyl)isoxazol-3-yl)urea (D38, 44 mg, 0.103 mmol) in step 1. MS (expected): 524.2 [M+H].

### Example 103

### 1-(5-(4-Amino-1-Cyclopropyl-1H-Pyrazolo[4,3-C]Pyridin-3-Yl)Imidazo[1,2-A]Pyridin-8-Yl)-3-(4-((4-Methylpiperazin-1-Yl)Methyl)-3-(Trifluoromethyl)Phenyl)Urea

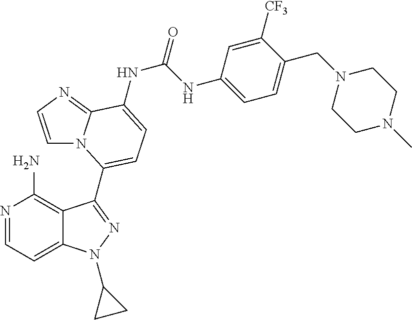 [SMILES: CN1CCN(Cc2ccc(NC(=O)Nc3ccc(-c4nn(C5CC5)c5ccnc(N)c45)n4ccnc34)cc2C(F)(F)F)CC1]

The title compound is prepared by following a similar 2 steps procedure described for Example 98, starting from 1-cyclopropyl-N-(2,4-dimethoxybenzyl)-3-(trimethylstannyl)-1H-pyrazolo[4,3-c]pyridin-4-amine (E2, 50 mg, 0.103 mmol) and 1-(5-bromoimidazo[1,2-a]pyridin-8-yl)-3-(4-((4-methylpiperazin-1-yl)methyl)-3-(trifluoromethyl)phenyl)urea (D39, 52 mg, 0.103 mmol) in step 1. MS (expected): 605.3 [M+H].

### Example 104

### 1-(5-(4-Amino-1-Cyclopropyl-1H-Pyrazolo[4,3-C]Pyridin-3-Yl)Imidazo[1,2-A]Pyridin-8-Yl)-3-(4-((1-Methylpiperidin-4-Yl)Oxy)-3-(Trifluoromethyl)Phenyl)Urea

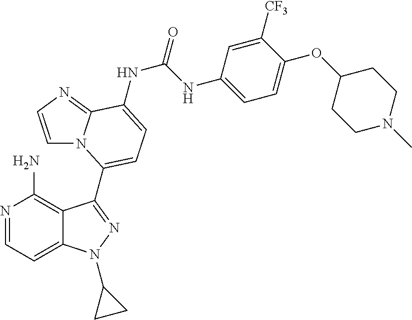 [SMILES: CN1CCC(Oc2ccc(NC(=O)Nc3ccc(-c4nn(C5CC5)c5ccnc(N)c45)n4ccnc34)cc2C(F)(F)F)CC1]

The title compound is prepared by following a similar 2 steps procedure described for Example 98, starting from 1-cyclopropyl-N-(2,4-dimethoxybenzyl)-3-(trimethylstannyl)-1H-pyrazolo[4,3-c]pyridin-4-amine (E2, 50 mg, 0.103 mmol) and 1-(5-bromoimidazo[1,2-a]pyridin-8-yl)-3-(4-((1-methylpiperidin-4-yl)oxy)-3-(trifluoromethyl)phenyl)urea (D7, 53 mg, 0.102 mmol) in step 1. MS (expected): 606.3 [M+H].

### Example 105

### 1-(5-(4-Amino-1-Methyl-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

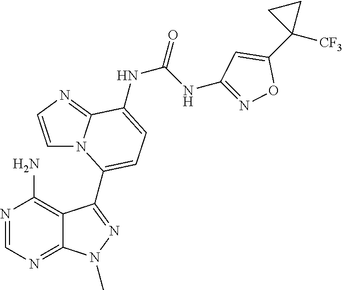 [SMILES: Cn1nc(-c2ccc(NC(=O)Nc3cc(C4(C(F)(F)F)CC4)on3)c3nccn23)c2c(N)ncnc21]

The title compound is prepared by following a similar 2 steps procedure described for Example 98, starting from N-(2,4-dimethoxybenzyl)-1-methyl-3-(trimethylstannyl)-1H-pyrazolo[3,4-d]pyrimidin-4-amine (E2, 50 mg, 0.108 mmol) and 1-(5-bromoimidazo[1,2-a]pyridin-8-yl)-3-(5-(1-(trifluoromethyl)cyclopropyl)isoxazol-3-yl)urea (D38, 47 mg, 0.108 mmol) in step 1. MS (expected): 499.2 [M+H].

### Example 106

### 1-(5-(4-Amino-1-(3,3-Difluorocyclobutyl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

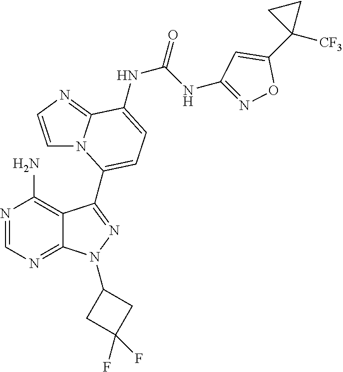 [SMILES: Nc1ncnc2c1c(-c1ccc(NC(=O)Nc3cc(C4(C(F)(F)F)CC4)on3)c3nccn13)nn2C1CC(F)(F)C1]

The title compound is prepared by following a similar 2 steps procedure described for Example 98, starting from 1-(3,3-difluorocyclobutyl)-N-(2,4-dimethoxybenzyl)-3-(trimethylstannyl)-1H-pyrazolo[3,4-d]pyrimidin-4-amine (E4, 50 mg, 0.093 mmol) and 1-(5-bromoimidazo[1,2-a]pyridin-8-yl)-3-(5-(1-(trifluoromethyl)cyclopropyl)isoxazol-3-yl)urea (D38, 40 mg, 0.093 mmol) in step 1. MS (expected): 575.2 [M+H].

### Example 107

### 1-(5-(1-Allyl-4-Amino-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

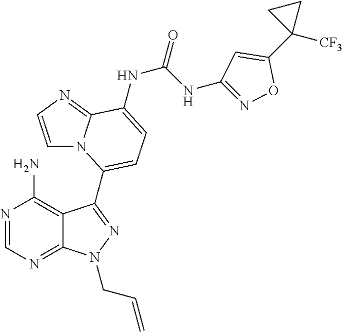 [SMILES: C=CCn1nc(-c2ccc(NC(=O)Nc3cc(C4(C(F)(F)F)CC4)on3)c3nccn23)c2c(N)ncnc21]

The title compound is prepared by following a similar 2 steps procedure described for Example 98, starting from 1-allyl-N-(2,4-dimethoxybenzyl)-3-(trimethylstannyl)-1H-pyrazolo[3,4-d]pyrimidin-4-amine (E5, 50 mg, 0.102 mmol) and 1-(5-bromoimidazo[1,2-a]pyridin-8-yl)-3-(5-(1-(trifluoromethyl)cyclopropyl)isoxazol-3-yl)urea (D38, 44 mg, 0.102 mmol) in step 1. MS (expected): 525.2 [M+H].

### Example 108

### 1-(5-(4-Amino-1-(but-3-Yn-1-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

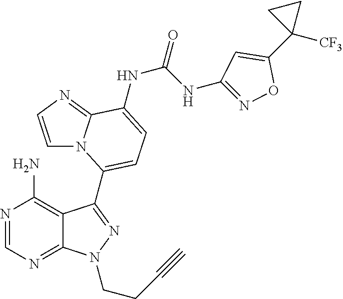 [SMILES: C#CCCn1nc(-c2ccc(NC(=O)Nc3cc(C4(C(F)(F)F)CC4)on3)c3nccn23)c2c(N)ncnc21]

The title compound is prepared by following a similar 2 steps procedure described for Example 98, starting from 1-(but-3-yn-1-yl)-N-(2,4-dimethoxybenzyl)-3-(trimethylstannyl)-1H-pyrazolo[3,4-d]pyrimidin-4-amine (E6, 50 mg, 0.100 mmol) and 1-(5-bromoimidazo[1,2-a]pyridin-8-yl)-3-(5-(1-(trifluoromethyl)cyclopropyl)isoxazol-3-yl)urea (D38, 43 mg, 0.100 mmol) in step 1. MS (expected): 537.2 [M+H].

### Example 109

### 1-(5-(4-Amino-1-(4-Hydroxycyclohexyl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

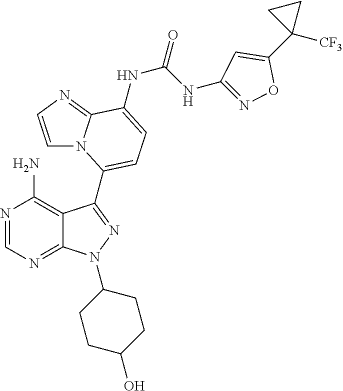 [SMILES: Nc1ncnc2c1c(-c1ccc(NC(=O)Nc3cc(C4(C(F)(F)F)CC4)on3)c3nccn13)nn2C1CCC(O)CC1]

The title compound is prepared by following a similar 2 steps procedure described for Example 98, starting from 4-(4-((2,4-dimethoxybenzyl)amino)-3-(trimethylstannyl)-1H-pyrazolo[3,4-d]pyrimidin-1-yl)cyclohexan-1-ol (E7, 50 mg, 0.092 mmol) and 1-(5-bromoimidazo[1,2-a]pyridin-8-yl)-3-(5-(1-(trifluoromethyl)cyclopropyl)isoxazol-3-yl)urea (D38, 40 mg, 0.092 mmol) in step 1. MS (expected): 583.2 [M+H].

### Example 110

### 1-(5-(4-Amino-1-(Tetrahydrofuran-3-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

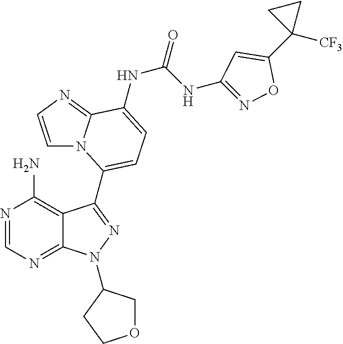 [SMILES: Nc1ncnc2c1c(-c1ccc(NC(=O)Nc3cc(C4(C(F)(F)F)CC4)on3)c3nccn13)nn2C1CCOC1]

The title compound is prepared by following a similar 2 steps procedure described for Example 98, starting from N-(2,4-dimethoxybenzyl)-1-(tetrahydrofuran-3-yl)-3-(trimethylstannyl)-1H-pyrazolo[3,4-d]pyrimidin-4-amine (E8, 50 mg, 0.096 mmol) and 1-(5-bromoimidazo[1,2-a]pyridin-8-yl)-3-(5-(1-(trifluoromethyl)cyclopropyl)isoxazol-3-yl)urea (D38, 42 mg, 0.096 mmol) in step 1. MS (expected): 555.2 [M+H].

### Example 111

### 1-(5-(4-Amino-1-(Tetrahydro-2H-Pyran-4-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

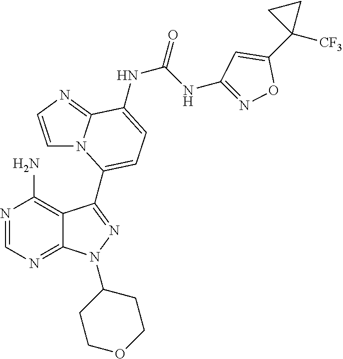 [SMILES: Nc1ncnc2c1c(-c1ccc(NC(=O)Nc3cc(C4(C(F)(F)F)CC4)on3)c3nccn13)nn2C1CCOCC1]

The title compound is prepared by following a similar 2 steps procedure described for Example 98, starting from N-(2,4-dimethoxybenzyl)-1-(tetrahydro-2H-pyran-4-yl)-3-(trimethylstannyl)-1H-pyrazolo[3,4-d]pyrimidin-4-amine (E9, 50 mg, 0.094 mmol) and 1-(5-bromoimidazo[1,2-a]pyridin-8-yl)-3-(5-(1-(trifluoromethyl)cyclopropyl)isoxazol-3-yl)urea (D38, 40 mg, 0.094 mmol) in step 1. MS (expected): 569.2 [M+H].

### Example 112

### 1-(5-(4-Amino-1-(Tetrahydro-2H-Pyran-3-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

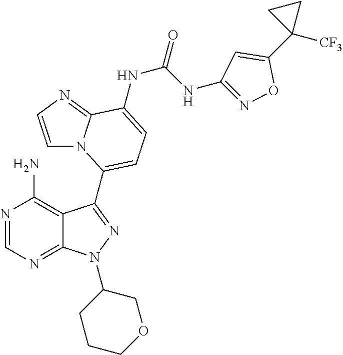 [SMILES: Nc1ncnc2c1c(-c1ccc(NC(=O)Nc3cc(C4(C(F)(F)F)CC4)on3)c3nccn13)nn2C1CCCOC1]

The title compound is prepared by following a similar 2 steps procedure described for Example 98, starting from N-(2,4-dimethoxybenzyl)-1-(tetrahydro-2H-pyran-3-yl)-3-(trimethylstannyl)-1H-pyrazolo[3,4-d]pyrimidin-4-amine (E10, 50 mg, 0.094 mmol) and 1-(5-bromoimidazo[1,2-a]pyridin-8-yl)-3-(5-(1-(trifluoromethyl)cyclopropyl)isoxazol-3-yl)urea (D38, 40 mg, 0.094 mmol) in step 1. MS (expected): 569.2 [M+H].

### Example 113

### 1-(5-(4-Amino-1-(1,1-Dioxidotetrahydrothiophen-3-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

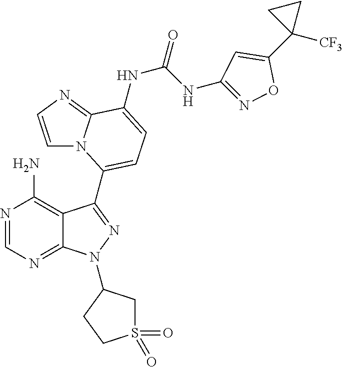 [SMILES: Nc1ncnc2c1c(-c1ccc(NC(=O)Nc3cc(C4(C(F)(F)F)CC4)on3)c3nccn13)nn2C1CCS(=O)(=O)C1]

The title compound is prepared by following a similar 2 steps procedure described for Example 98, starting from 3-(4-((2,4-dimethoxybenzyl)amino)-3-(trimethylstannyl)-1H-pyrazolo[3,4-d]pyrimidin-1-yl)tetrahydrothiophene 1,1-dioxide (E11, 50 mg, 0.088 mmol) and 1-(5-bromoimidazo[1,2-a]pyridin-8-yl)-3-(5-(1-(trifluoromethyl)cyclopropyl)isoxazol-3-yl)urea (D38, 38 mg, 0.088 mmol) in step 1. MS (expected): 603.1 [M+H].

### Example 114

### 1-(5-(4-Amino-1-(Azetidin-3-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

### Step 1: Synthesis of tert-butyl 3-(4-amino-3-(8-(3-(5-(1-(trifluoromethyl)cyclopropyl)isoxazol-3-yl)ureido)imidazo[1,2-a]pyridin-5-yl)-1H-pyrazolo[3,4-d]pyrimidin-1-yl)azetidine-1-carboxylate

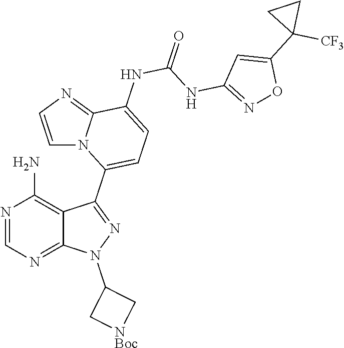 [SMILES: CC(C)(C)OC(=O)N1CC(n2nc(-c3ccc(NC(=O)Nc4cc(C5(C(F)(F)F)CC5)on4)c4nccn34)c3c(N)ncnc32)C1]

The title compound is prepared by following a similar 2 steps procedure described for Example 98, starting from tert-butyl 3-(4-((2,4-dimethoxybenzyl)amino)-3-(trimethylstannyl)-1H-pyrazolo[3,4-d]pyrimidin-1-yl)azetidine-1-carboxylate (E12, 50 mg, 0.083 mmol) and 1-(5-bromoimidazo[1,2-a]pyridin-8-yl)-3-(5-(1-(trifluoromethyl)cyclopropyl)isoxazol-3-yl)urea (D38, 36 mg, 0.083 mmol) in step 1. MS (expected): 640.2 [M+H].

### Step 2: Synthesis of 1-(5-(4-amino-1-(azetidin-3-yl)-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea

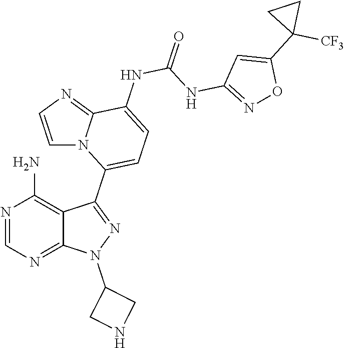 [SMILES: Nc1ncnc2c1c(-c1ccc(NC(=O)Nc3cc(C4(C(F)(F)F)CC4)on3)c3nccn13)nn2C1CNC1]

The title compound was obtained by following a similar procedure described for step 2 of Example 5, starting from tert-butyl 3-(4-amino-3-(8-(3-(5-(1-(trifluoromethyl)cyclopropyl)isoxazol-3-yl)ureido)imidazo[1,2-a]pyridin-5-yl)-1H-pyrazolo[3,4-d]pyrimidin-1-yl)azetidine-1-carboxylate (50 mg, 0.078 mmol). MS (expected): 540.2 [M+H].

### Example 115

### 1-(5-(4-Amino-1-(1-Methylazetidin-3-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

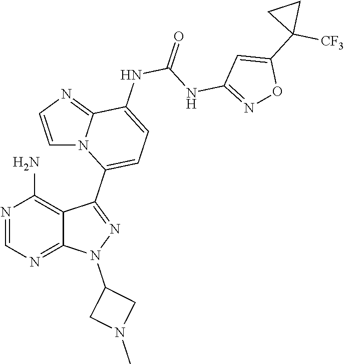 [SMILES: CN1CC(n2nc(-c3ccc(NC(=O)Nc4cc(C5(C(F)(F)F)CC5)on4)c4nccn34)c3c(N)ncnc32)C1]

The title compound was obtained by following a similar procedure described for Example 95, starting from 1-(5-(4-amino-1-(azetidin-3-yl)-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea (50 mg, 0.093 mmol), paraformaldehyde (8.3 mg, 0.278 mmol), acetic acid (0.3 μL, 0.005 mmol), and NaBH₃CN (29 mg, 0.436 mmol). MS (expected): 554.2 [M+H].

### Example 116

### 1-(5-(4-Amino-1-(Piperidin-3-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

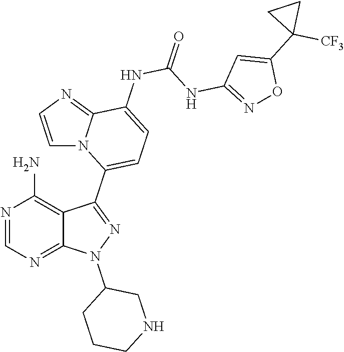 [SMILES: Nc1ncnc2c1c(-c1ccc(NC(=O)Nc3cc(C4(C(F)(F)F)CC4)on3)c3nccn13)nn2C1CCCNC1]

The title compound is prepared by following a similar 2 steps procedure described for Example 114, starting from tert-butyl 3-(4-((2,4-dimethoxybenzyl)amino)-3-(trimethylstannyl)-1H-pyrazolo[3,4-d]pyrimidin-1-yl)piperidine-1-carboxylate (E13, 50 mg, 0.079 mmol) and 1-(5-bromoimidazo[1,2-a]pyridin-8-yl)-3-(5-(1-(trifluoromethyl)cyclopropyl)isoxazol-3-yl)urea (D38, 34 mg, 0.079 mmol) in step 1. MS (expected): 568.2 [M+H].

### Example 117

### 1-(5-(4-Amino-1-(1-Methylpiperidin-3-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

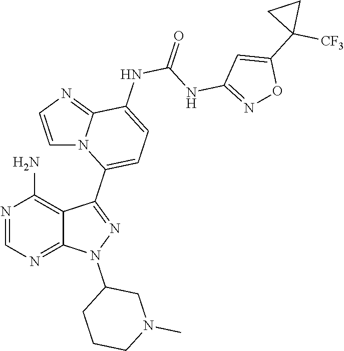 [SMILES: CN1CCCC(n2nc(-c3ccc(NC(=O)Nc4cc(C5(C(F)(F)F)CC5)on4)c4nccn34)c3c(N)ncnc32)C1]

The title compound was obtained by following a similar procedure described for Example 95, starting from 1-(5-(4-amino-1-(piperidin-3-yl)-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea (50 mg, 0.088 mmol), paraformaldehyde (7.9 mg, 0.264 mmol), acetic acid (0.3 μL, 0.004 mmol), and NaBH₃CN (28 mg, 0.440 mmol). MS (expected): 582.2 [M+H].

### Example 118

### 1-(5-(4-Amino-1-(1-(Oxetan-3-Yl)Piperidin-4-Yl)-1H-Pyrazolo[3,4-D]Pyrimidin-3-Yl)Imidazo[1,2-A]Pyridin-8-Yl)-3-(5-(1-(Trifluoromethyl)Cyclopropyl)Isoxazol-3-Yl)Urea

### Steps 1 and 2: Synthesis of 1-(5-(4-amino-1-(piperidin-4-yl)-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea

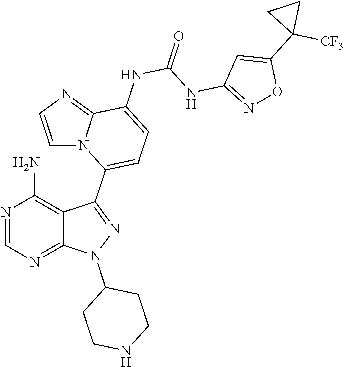 [SMILES: Nc1ncnc2c1c(-c1ccc(NC(=O)Nc3cc(C4(C(F)(F)F)CC4)on3)c3nccn13)nn2C1CCNCC1]

The title compound is prepared by following a similar 2 steps procedure described for Example 114, starting from tert-butyl 4-(4-((2,4-dimethoxybenzyl)amino)-3-(trimethylstannyl)-1H-pyrazolo[3,4-d]pyrimidin-1-yl)piperidine-1-carboxylate (E14, 50 mg, 0.079 mmol) and 1-(5-bromoimidazo[1,2-a]pyridin-8-yl)-3-(5-(1-(trifluoromethyl)cyclopropyl)isoxazol-3-yl)urea (D38, 34 mg, 0.079 mmol) in step 1. MS (expected): 568.2 [M+H].

### Step 2: Synthesis of 1-(5-(4-amino-1-(1-(oxetan-3-yl)piperidin-4-yl)-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea

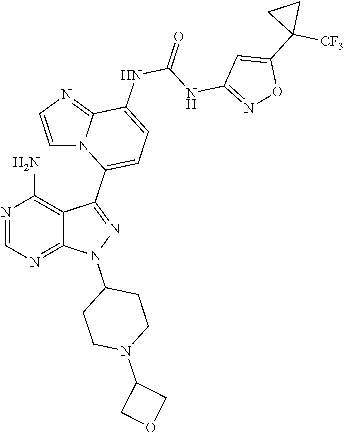 [SMILES: Nc1ncnc2c1c(-c1ccc(NC(=O)Nc3cc(C4(C(F)(F)F)CC4)on3)c3nccn13)nn2C1CCN(C2COC2)CC1]

Oxetan-3-one (5.2 μL, 0.088 mmol) and glacial acetic acid (0.3 μL, 0.004 mmol) are added to a solution of 1-(5-(4-amino-1-(piperidin-4-yl)-1H-pyrazolo[3,4-d]pyrimidin-3-yl)imidazo[1,2-a]pyridin-8-yl)-3-(5-(1-(trifluoromethyl)cyclopropyl)isoxazol-3-yl)urea (50 mg, 0.088 mmol) in DCM (4 mL) and the resulting mixture is stirred at room temperature for 4 h. NaBH₃CN (17 mg, 0.264 mmol) is then added and the resulting mixture is stirred at 25° C. for 12 h. Following completion of the reaction (as indicated by UPLC), the reaction mixture is diluted with DCM (6 mL) and washed with aqueous 10% NaHCO₃ (5 mL) and brine (5 mL). The organic layer is dried over Na₂SO₄, filtered, and concentrated under reduced pressure to give a residue which is purified by reverse phase preparative HPLC. MS (expected): 624.2 [M+H].

### Example 119

### 1-(5-(4-Amino-7-Cyclopropyl-7H-Pyrrolo[2,3-D]Pyrimidin-5-Yl)Imidazo[1,2-A]Pyridin-8-Yl)-3-(3-Cyclopropylisoxazol-5-Yl)Urea

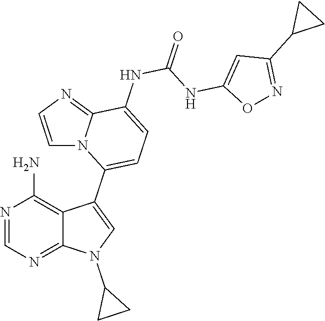 [SMILES: Nc1ncnc2c1c(-c1ccc(NC(=O)Nc3cc(C4CC4)no3)c3nccn13)cn2C1CC1]

The title compound was obtained by following a similar procedure described for Intermediate B1, starting from 1-(5-bromoimidazo[1,2-a]pyridin-8-yl)-3-(3-cyclopropylisoxazol-5-yl)urea (D41, 7.2 mg, 0.020 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 10 mg, 0.020 mmol), and was isolated as an off-white solid (1.3 mg, 13% yield). Cleavage of the di-Boc protecting group was observed during the reaction. ¹H NMR (400 MHz, CD₃OD) δ=8.25 (s, 1H), 8.03 (d, J=8.00 Hz, 1H), 7.65 (d, J=1.60 Hz, 1H), 7.58 (d, J=1.60 Hz, 1H), 7.52 (s, 1H), 6.94 (d, J=8.00 Hz, 1H), 5.96 (s, 1H), 3.59-3.60 (m, 1H), 1.95-1.97 (m, 1H), 1.05-1.31 (m, 8H). MS: 456.2 [M+H].

### Example 120

### 1-(5-(4-Amino-7-Cyclopropyl-7H-Pyrrolo[2,3-D]Pyrimidin-5-Yl)Imidazo[1,2-A]Pyridin-8-Yl)-3-(5-Cyclopropylisoxazol-3-Yl)Urea

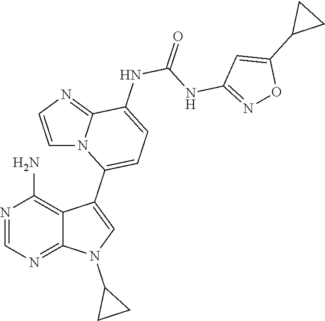 [SMILES: Nc1ncnc2c1c(-c1ccc(NC(=O)Nc3cc(C4CC4)on3)c3nccn13)cn2C1CC1]

The title compound was obtained by following a similar procedure described for Intermediate B1, starting from 1-(5-bromoimidazo[1,2-a]pyridin-8-yl)-3-(5-cyclopropylisoxazol-3-yl)urea (D45, 30.0 mg, 0.083 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 41.4 mg, 0.083 mmol), and was isolated as an off-white solid (5 mg, 13% yield). Cleavage of the di-Boc protecting group was observed during the reaction. ¹H NMR (400 MHz, DMSO-d₆) δ=10.23 (bs, 1H), 9.54 (bs, 1H), 8.20 (s, 1H), 7.96 (d, J=7.6 Hz, 1H), 7.67 (d, J=1.2 Hz, 1H), 7.56-7.57 (m, 2H), 6.81 (d, J=7.6 Hz, 1H), 6.51 (s, 1H), 6.14 (bs, 2H), 3.61-3.67 (m, 1H), 2.09-2.17 (m, 1H), 1.02-1.13 (m, 6H), 0.92-0.93 (m, 2H). MS: 456.1 [M+H].

### Example 121

### 1-(4-(4-Amino-7-Cyclopropyl-7H-Pyrrolo[2,3-D]Pyrimidin-5-Yl)-2-Methyl-2,3-Dihydrobenzofuran-7-Yl)-3-(4-((1-Ethylpiperidin-4-Yl)Oxy)-3-(Trifluoromethyl)Phenyl)Urea

### Step 1: Synthesis of tert-butyl (tert-butoxycarbonyl)(7-cyclopropyl-5-(7-(3-(4-((1-ethylpiperidin-4-yl)oxy)-3-(trifluoromethyl)phenyl)ureido)-2-methyl-2,3-dihydrobenzofuran-4-yl)-7H-pyrrolo[2,3-d]pyrimidin-4-yl)carbamate

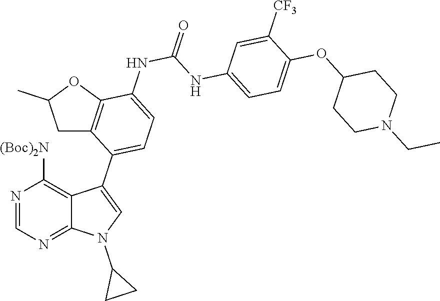

The title compound was obtained by following a similar procedure described for Intermediate B1, starting from 1-(4-bromo-2-methyl-2,3-dihydrobenzofuran-7-yl)-3-(4-((1-ethylpiperidin-4-yl)oxy)-3-(trifluoromethyl)phenyl)urea (D51, 60 mg, 0.111 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 55 mg, 0.111 mmol), and was isolated as an off-white solid (20 mg, 22% yield). MS: 836.2 [M+H].

### Step 2: Synthesis of 1-(4-(4-amino-7-cyclopropyl-7H-pyrrolo[2,3-d]pyrimidin-5-yl)-2-methyl-2,3-dihydrobenzofuran-7-yl)-3-(4-((1-ethylpiperidin-4-yl)oxy)-3-(trifluoromethyl)phenyl)urea

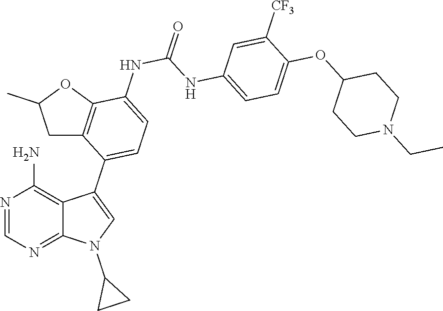 [SMILES: CCN1CCC(Oc2ccc(NC(=O)Nc3ccc(-c4cn(C5CC5)c5ncnc(N)c45)c4c3OC(C)C4)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((1-ethylpiperidin-4-yl)oxy)-3-(trifluoromethyl)phenyl)ureido)-2-methyl-2,3-dihydrobenzofuran-4-yl)-7H-pyrrolo[2,3-d]pyrimidin-4-yl)carbamate (20 mg, 0.024 mmol), and was isolated as an off-white solid (6 mg, 39% yield). ¹H NMR (400 MHz, CD₃OD) δ=8.18 (s, 1H), 7.84 (d, J=8.4 Hz, 1H), 7.76 (d, J=2.8 Hz, 1H), 7.58-7.61 (m, 1H), 7.18 (d, J=9.2 Hz, 1H), 7.13 (s, 1H), 6.84 (d, J=8.4 Hz, 1H), 5.04-5.06 (m, 1H), 4.62 (bs, 1H), 3.49-3.53 (m, 1H), 3.34-3.39 (m, 1H), 2.84-2.90 (m, 3H), 2.50-2.72 (m, 4H), 1.91-2.04 (m, 4H), 1.50-1.51 (m, 3H), 1.06-1.18 (m, 7H). MS: 636.3 [M+H].

### Example 122

### 1-(4-(4-Amino-7-Cyclobutyl-7H-Pyrrolo[2,3-D]Pyrimidin-5-Yl)-2,2-Dimethyl-2,3-Dihydrobenzofuran-7-Yl)-3-(5-(1-(Trifluoromethyl)Cyclopropyl)Isoxazol-3-Yl)Urea

### Step 1: Synthesis of tert-butyl (tert-butoxycarbonyl)(7-cyclobutyl-5-(2,2-dimethyl-7-(3-(5-(1-(trifluoromethyl)cyclopropyl)isoxazol-3-yl)ureido)-2,3-dihydrobenzofuran-4-yl)-7H-pyrrolo[2,3-d]pyrimidin-4-yl)carbamate

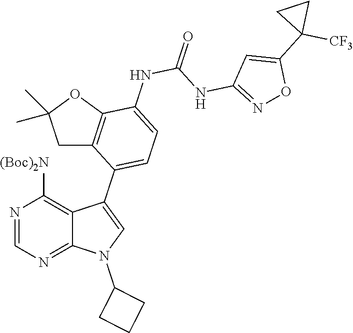

The title compound was obtained by following a similar procedure described for Intermediate B1, starting from 1-(4-bromo-2,2-dimethyl-2,3-dihydrobenzofuran-7-yl)-3-(5-(1-(trifluoromethyl)cyclopropyl)isoxazol-3-yl)urea (D21, 50 mg, 0.109 mmol) and tert-butyl (tert-butoxycarbonyl)(7-cyclobutyl-5-(4,4,5,5-tetramethyl-1,3,2-dioxaborolan-2-yl)-7H-pyrrolo[2,3-d]pyrimidin-4-yl)carbamate (step 2 of Intermediate B17, 56 mg, 0.109 mmol), and was isolated as an off-white solid (50 mg, 69% yield). MS: 768.3 [M+H].

### Step 2: Synthesis of 1-(4-(4-amino-7-cyclobutyl-7H-pyrrolo[2,3-d]pyrimidin-5-yl)-2,2-dimethyl-2,3-dihydrobenzofuran-7-yl)-3-(5-(1-(trifluoromethyl)cyclopropyl)isoxazol-3-yl)urea

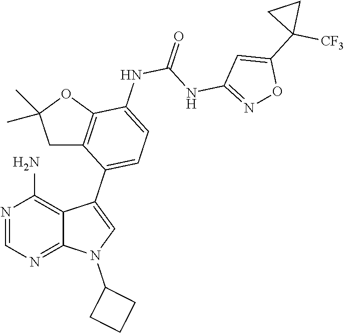 [SMILES: CC1(C)Cc2c(-c3cn(C4CCC4)c4ncnc(N)c34)ccc(NC(=O)Nc3cc(C4(C(F)(F)F)CC4)on3)c2O1]

The title compound was obtained by following a similar procedure described for step 2 of Example 5, starting from tert-butyl (tert-butoxycarbonyl)(7-cyclobutyl-5-(2,2-dimethyl-7-(3-(5-(1-(trifluoromethyl)cyclopropyl)isoxazol-3-yl)ureido)-2,3-dihydrobenzofuran-4-yl)-7H-pyrrolo[2,3-d]pyrimidin-4-yl)carbamate (50 mg, 0.065 mmol), and was isolated as an off-white solid (8 mg, 22% yield). ¹H NMR (400 MHz, DMSO-d₆) δ=9.97 (bs, 1H), 8.47 (bs, 1H), 8.12 (s, 1H), 7.93 (d, J=8.0 Hz, 1H), 7.55 (s, 1H), 6.90 (s, 1H), 6.79 (d, J=8.4 Hz, 1H), 6.02 (bs, 2H), 5.18-5.22 (m, 1H), 3.12 (s, 2H), 2.61-2.69 (m, 2H), 2.40-2.42 (m, 2H), 1.81-1.87 (m, 2H), 1.47-1.57 (m, 10H). MS: 568.3 [M+H].

### Example 123

### 1-(4-(4-Amino-7-Cyclopropyl-7H-Pyrrolo[2,3-D]Pyrimidin-5-Yl)-2,2-Dimethyl-2,3-Dihydrobenzofuran-7-Yl)-3-(4-((1-Ethylpiperidin-4-Yl)Oxy)-3-(Trifluoromethyl)Phenyl)Urea

### Step 1: Synthesis of tert-butyl (tert-butoxycarbonyl)(7-cyclopropyl-5-(7-(3-(4-((1-ethylpiperidin-4-yl)oxy)-3-(trifluoromethyl)phenyl)ureido)-2,2-dimethyl-2,3-dihydrobenzofuran-4-yl)-7H-pyrrolo[2,3-d]pyrimidin-4-yl)carbamate

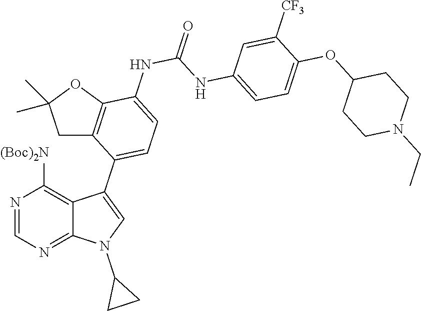

The title compound was obtained by following a similar procedure described for Intermediate B1, starting from 1-(4-bromo-2,2-dimethyl-2,3-dihydrobenzofuran-7-yl)-3-(4-((1-ethylpiperidin-4-yl)oxy)-3-(trifluoromethyl)phenyl)urea (D52, 20 mg, 0.036 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 17.99 mg, 0.036 mmol), and was isolated as an off-white solid (10 mg, 32% yield). MS: 850.5 [M+H].

### Step 2: Synthesis of 1-(4-(4-amino-7-cyclopropyl-7H-pyrrolo[2,3-d]pyrimidin-5-yl)-2,2-dimethyl-2,3-dihydrobenzofuran-7-yl)-3-(4-((1-ethylpiperidin-4-yl)oxy)-3-(trifluoromethyl)phenyl)urea

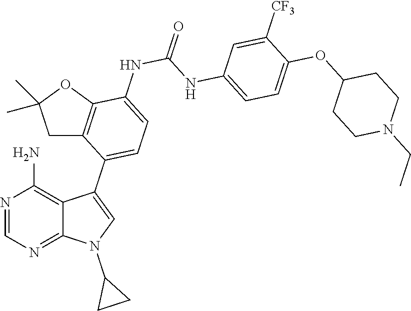 [SMILES: CCN1CCC(Oc2ccc(NC(=O)Nc3ccc(-c4cn(C5CC5)c5ncnc(N)c45)c4c3OC(C)(C)C4)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((1-ethylpiperidin-4-yl)oxy)-3-(trifluoromethyl)phenyl)ureido)-2,2-dimethyl-2,3-dihydrobenzofuran-4-yl)-7H-pyrrolo[2,3-d]pyrimidin-4-yl)carbamate (10 mg, 0.012 mmol), and was isolated as an off-white solid (3 mg, 39% yield). ¹H NMR (400 MHz, CD₃OD) δ=8.18 (s, 1H), 7.86 (d, J=8.4 Hz, 1H), 7.76 (d, J=2.8 Hz, 1H), 7.58-7.61 (m, 1H), 7.19 (d, J=9.2 Hz, 1H), 7.11 (s, 1H), 6.84 (d, J=8.0 Hz, 1H), 4.61-4.68 (m, 1H), 3.49-3.52 (m, 1H), 3.08 (s, 2H), 2.67-2.88 (m, 6H), 1.94-2.11 (m, 4H), 1.53 (s, 6H), 1.14-1.22 (m, 7H). MS: 650.3 [M+H].

### Example 124

### 1-(4-(4-Amino-7-Cyclopropyl-7H-Pyrrolo[2,3-D]Pyrimidin-5-Yl)Benzofuran-7-Yl)-3-(4-((1-Ethylpiperidin-4-Yl)Oxy)-3-(Trifluoromethyl)Phenyl)Urea

### Step 1: Synthesis of tert-butyl (tert-butoxycarbonyl)(7-cyclopropyl-5-(7-(3-(4-((1-ethylpiperidin-4-yl)oxy)-3-(trifluoromethyl)phenyl)ureido)benzofuran-4-yl)-7H-pyrrolo[2,3-d]pyrimidin-4-yl)carbamate

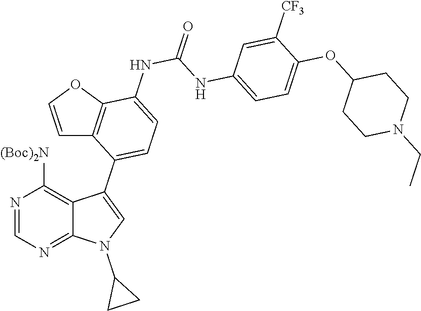

The title compound was obtained by following a similar procedure described for Intermediate B1, starting from 1-(4-((1-ethylpiperidin-4-yl)oxy)-3-(trifluoromethyl)phenyl)-3-(4-iodobenzofuran-7-yl)urea (D53, 115 mg, 0.200 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 100 mg, 0.200 mmol), and was isolated as an off-white solid (50 mg, 30% yield). MS: 820.3 [M+H].

### Step 2: Synthesis of 1-(4-(4-amino-7-cyclopropyl-7H-pyrrolo[2,3-d]pyrimidin-5-yl)benzofuran-7-yl)-3-(4-((1-ethylpiperidin-4-yl)oxy)-3-(trifluoromethyl)phenyl)urea

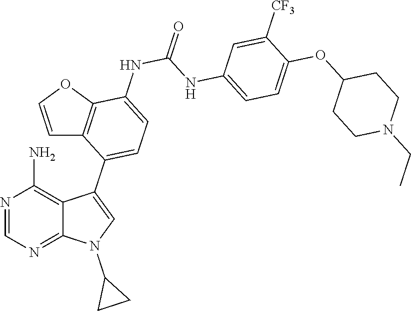 [SMILES: CCN1CCC(Oc2ccc(NC(=O)Nc3ccc(-c4cn(C5CC5)c5ncnc(N)c45)c4ccoc34)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((1-ethylpiperidin-4-yl)oxy)-3-(trifluoromethyl)phenyl)ureido)benzofuran-4-yl)-7H-pyrrolo[2,3-d]pyrimidin-4-yl)carbamate (50 mg, 0.061 mmol), and was isolated as an off-white solid (3.5 mg, 9.2% yield). ¹H NMR (400 MHz, CD₃OD) δ=8.21 (s, 1H), 7.99 (d, J=8.0 Hz, 1H), 7.83-7.88 (m, 2H), 7.67-7.70 (m, 1H), 7.23-7.27 (m, 3H), 6.86 (d, J=2.0 Hz, 1H), 4.61 (bs, 1H), 3.54-3.57 (m, 1H), 3.00-3.16 (m, 6H), 2.15-2.18 (m, 4H), 1.12-1.33 (m, 7H). MS: 620.2 [M+H].

### Example 125

### 1-(4-(4-Amino-7-Cyclopropyl-7H-Pyrrolo[2,3-D]Pyrimidin-5-Yl)Benzo[D]Isoxazol-7-Yl)-3-(4-((1-Ethylpiperidin-4-Yl)Oxy)-3-(Trifluoromethyl)Phenyl)Urea

### Step 1: Synthesis of tert-butyl (tert-butoxycarbonyl)(7-cyclopropyl-5-(7-(3-(4-((1-ethylpiperidin-4-yl)oxy)-3-(trifluoromethyl)phenyl)ureido)benzo[d]isoxazol-4-yl)-7H-pyrrolo[2,3-d]pyrimidin-4-yl)carbamate

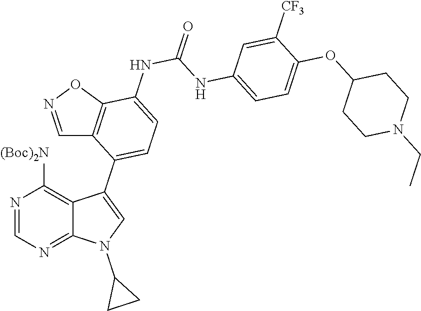

The title compound was obtained by following a similar procedure described for Intermediate B1, starting from 1-(4-bromobenzo[d]isoxazol-7-yl)-3-(4-((1-ethylpiperidin-4-yl)oxy)-3-(trifluoromethyl)phenyl)urea (D54, 53 mg, 0.100 mmol) and tert-butyl (tert-butoxycarbonyl)(7-cyclopropyl-5-(4,4,5,5-tetramethyl-1,3,2-dioxaborolan-2-yl)-7H-pyrrolo[2,3-d]pyrimidin-4-yl)carbamate (step 2 of Intermediate B2, 50 mg, 0.200 mmol), and was isolated as an off-white solid (20 mg, 32% yield). MS: 821.3 [M+H].

### Step 2: Synthesis of 1-(4-(4-amino-7-cyclopropyl-7H-pyrrolo[2,3-d]pyrimidin-5-yl)benzo[d]isoxazol-7-yl)-3-(4-((1-ethylpiperidin-4-yl)oxy)-3-(trifluoromethyl)phenyl)urea

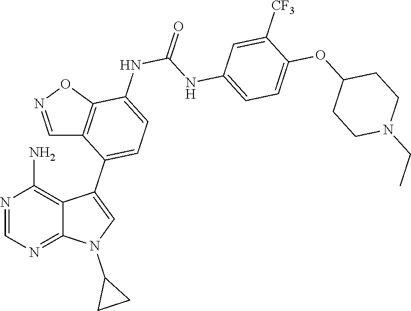 [SMILES: CCN1CCC(Oc2ccc(NC(=O)Nc3ccc(-c4cn(C5CC5)c5ncnc(N)c45)c4cnoc34)cc2C(F)(F)F)CC1]

The title compound was obtained by following a similar procedure described for step 2 of Example 5, starting from tert-butyl (tert-butoxycarbonyl)(7-cyclopropyl-5-(7-(3-(4-((1-ethylpiperidin-4-yl)oxy)-3-(trifluoromethyl)phenyl)ureido)benzo[d]isoxazol-4-yl)-7H-pyrrolo[2,3-i]pyrimidin-4-yl)carbamate (20 mg, 0.024 mmol), and was isolated as an off-white solid (2.5 mg, 16% yield). MS: 621.3 [M+H].

### Example 126

### 1-(5-(4-Amino-7-Methyl-7H-Pyrrolo[2,3-D]Pyrimidin-5-Yl)Imidazo[1,2-A]Pyridin-8-Yl)-3-(5-(1-(Trifluoromethyl)Cyclopropyl)Isoxazol-3-Yl)Urea

### Step 1: Synthesis of 1-(5-(4-((2,4-dimethoxybenzyl)amino)-7-methyl-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

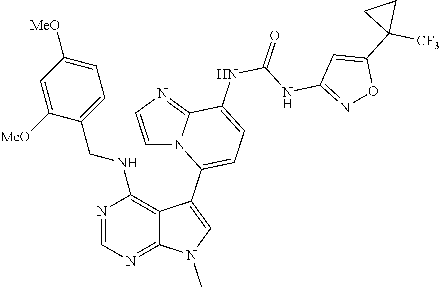 [SMILES: COc1ccc(CNc2ncnc3c2c(-c2ccc(NC(=O)Nc4cc(C5(C(F)(F)F)CC5)on4)c4nccn24)cn3C)c(OC)c1]

The title compound was prepared by following the general procedure for urea formation (Method B), starting from 5-(8-aminoimidazo[1,2-a]pyridin-5-yl)-N-(2,4-dimethoxybenzyl)-7-methyl-7H-pyrrolo[2,3-d]pyrimidin-4-amine (B29, 100 mg, 0.233 mmol) and phenyl (5-(1-(trifluoromethyl)cyclopropyl)isoxazol-3-yl)carbamate (C3, 73 mg, 0.233 mmol), and was isolated as an off-white solid (20 mg, 13% yield). MS: 648.2 [M+H].

### Step 2: Synthesis of 1-(5-(4-amino-7-methyl-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea

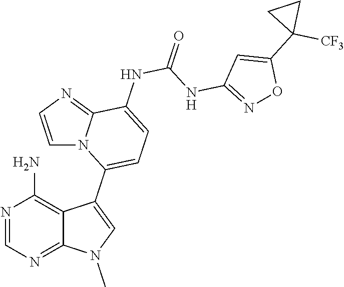 [SMILES: Cn1cc(-c2ccc(NC(=O)Nc3cc(C4(C(F)(F)F)CC4)on3)c3nccn23)c2c(N)ncnc21]

The title compound is prepared by following a similar procedure described for Step 2 of Example 83, starting from 1-(5-(4-((2,4-dimethoxybenzyl)amino)-7-methyl-7H-pyrrolo[2,3-d]pyrimidin-5-yl)imidazo[1,2-a]pyridin-8-yl)-3-(5-(1-(trifluoromethyl)cyclopropyl)isoxazol-3-yl)urea (20 mg, 0.031 mmol), and was isolated as an off-white solid (6.3 mg, 41% yield). ¹H NMR (400 MHz, CD₃OD) δ=8.24 (s, 1H), 8.05 (d, J=7.6 Hz, 1H), 7.69 (d, J=1.2 Hz, 1H), 7.59 (d, J=1.2 Hz, 1H), 7.53 (s, 1H), 6.95 (d, J=7.6 Hz, 1H), 6.84 (s, 1H), 3.90 (s, 3H), 1.50-1.57 (m, 4H). MS: 498.1 [M+H].

### Example 127

### 1-(5-(4-Amino-7-Methyl-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of 1-(5-(4-((2,4-dimethoxybenzyl)amino)-7-methyl-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

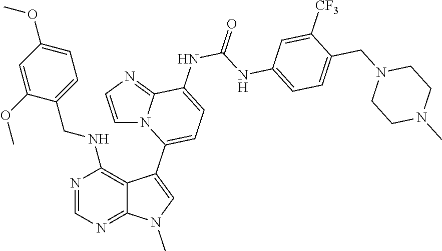

The title compound was prepared by following the general procedure for urea formation (Method B), starting from 5-(8-aminoimidazo[1,2-a]pyridin-5-yl)-N-(2,4-dimethoxybenzyl)-7-methyl-7H-pyrrolo[2,3-d]pyrimidin-4-amine (B29, 50 mg, 0.116 mmol) and phenyl (4-((4-methylpiperazin-1-yl)methyl)-3-(trifluoromethyl)phenyl)carbamate (C5, 46 mg, 0.116 mmol), and was isolated as an off-white solid (22 mg, 26% yield). MS: 729.3 [M+H].

### Step 2: Synthesis of 1-(5-(4-amino-7-methyl-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

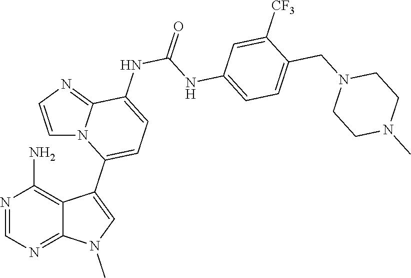 [SMILES: CN1CCN(Cc2ccc(NC(=O)Nc3ccc(-c4cn(C)c5ncnc(N)c45)n4ccnc34)cc2C(F)(F)F)CC1]

The title compound is prepared by following a similar procedure described for Step 2 of Example 83, starting from 1-(5-(4-((2,4-dimethoxybenzyl)amino)-7-methyl-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea (20 mg, 0.027 mmol), and was isolated as an off-white solid (2.1 mg, 13% yield). ¹H NMR (400 MHz, DMSO-d₆) δ=10.00 (bs, 1H), 9.18 (bs, 1H), 8.20 (s, 1H), 7.98-8.04 (m, 2H), 7.57-7.69 (m, 5H), 6.81 (d, J=7.6 Hz, 1H), 6.17 (bs, 2H), 3.79 (s, 3H), 3.55 (s, 2H), 2.56 (bs, 4H), 2.40 (bs, 4H), 2.17 (s, 3H). MS: 579.1 [M+H].

### Example 128

### 1-(5-(4-Amino-7-Isopropyl-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of 1-(5-(4-((2,4-dimethoxybenzyl)amino)-7-isopropyl-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

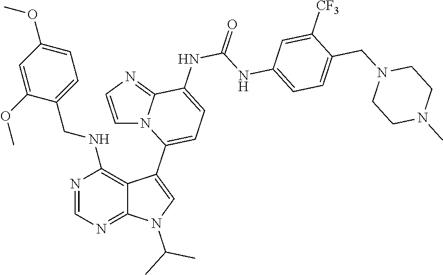

The title compound was obtained by following a similar procedure described for Intermediate B1, starting from 1-(5-bromoimidazo[1,2-a]pyridin-8-yl)-3-(4-((4-methylpiperazin-1-yl)methyl)-3-(trifluoromethyl)phenyl)urea (D39, 68 mg, 0.133 mmol) and N-(2,4-dimethoxybenzyl)-7-isopropyl-5-(4,4,5,5-tetramethyl-1,3,2-dioxaborolan-2-yl)-7H-pyrrolo[2,3-d]pyrimidin-4-amine (step 1 of Intermediate B30, 60 mg, 0.133 mmol), and was isolated as an off-white solid (20 mg, 20% yield). MS: 757.2 [M+H].

### Step 2: Synthesis of 1-(5-(4-amino-7-isopropyl-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

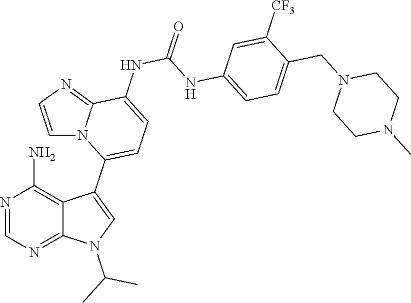 [SMILES: CC(C)n1cc(-c2ccc(NC(=O)Nc3ccc(CN4CCN(C)CC4)c(C(F)(F)F)c3)c3nccn23)c2c(N)ncnc21]

The title compound is prepared by following a similar procedure described for Step 2 of Example 83, starting from 1-(5-(4-((2,4-dimethoxybenzyl)amino)-7-isopropyl-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea (20 mg, 0.026 mmol), and was isolated as an off-white solid (4 mg, 24% yield). ¹H NMR (400 MHz, DMSO-d₆) δ=9.99 (bs, 1H), 9.18 (bs, 1H), 8.18 (s, 1H), 7.98-8.04 (m, 2H), 7.78 (s, 1H), 7.64-7.68 (m, 2H), 7.57-7.60 (m, 2H), 6.83 (d, J=7.6 Hz, 1H), 6.13 (bs, 2H), 4.99-5.03 (m, 1H), 3.55 (s, 2H), 2.33-2.34 (m, 8H), 2.17 (s, 3H), 1.50 (d, J=6.8 Hz, 6H). MS: 607.3 [M+H].

### Example 129

### 1-(5-(4-Amino-7-Cyclobutyl-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

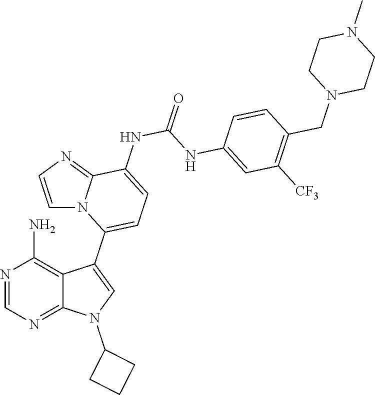 [SMILES: CN1CCN(Cc2ccc(NC(=O)Nc3ccc(-c4cn(C5CCC5)c5ncnc(N)c45)n4ccnc34)cc2C(F)(F)F)CC1]

The title compound was obtained by following a similar procedure described for Intermediate B1, starting from 1-(5-bromoimidazo[1,2-a]pyridin-8-yl)-3-(4-((4-methylpiperazin-1-yl)methyl)-3-(trifluoromethyl)phenyl)urea (D39, 100 mg, 0.196 mmol) and tert-butyl (tert-butoxycarbonyl)(7-cyclobutyl-5-(4,4,5,5-tetramethyl-1,3,2-dioxaborolan-2-yl)-7H-pyrrolo[2,3-d]pyrimidin-4-yl)carbamate (step 2 of Intermediate B17, 151 mg, 0.293 mmol), and was isolated as an off-white solid (10 mg, 8.3% yield). Cleavage of the di-Boc protecting group was observed during the reaction. ¹H NMR (400 MHz, DMSO-d₆) δ=10.00 (bs, 1H), 9.18 (bs, 1H), 8.18 (s, 1H), 7.99-8.04 (m, 2H), 7.91 (s, 1H), 7.58-7.67 (m, 4H), 6.85 (d, J=7.60 Hz, 1H), 6.14 (bs, 2H), 5.20-5.27 (m, 1H), 3.56 (bs, 2H), 2.60-2.68 (m, 4H), 2.32-2.48 (m, 8H), 2.18 (s, 3H), 1.83 (bs, 2H). MS: 619.2 [M+H].

### Example 130

### 1-(5-(4-Amino-7-(2-Hydroxyethyl)-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of tert-butyl (7-(2-hydroxyethyl)-5-(8-(3-(4-((4-methylpiperazin-1-yl)methyl)-3-(trifluoromethyl)phenyl)ureido)imidazo[1,2-a]pyridin-5-yl)-7H-pyrrolo[2,3-d]pyrimidin-4-yl)carbamate

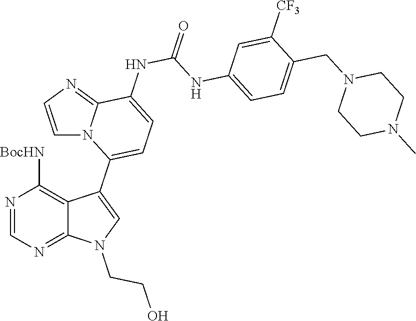 [SMILES: CN1CCN(Cc2ccc(NC(=O)Nc3ccc(-c4cn(CCO)c5ncnc(NC(=O)OC(C)(C)C)c45)n4ccnc34)cc2C(F)(F)F)CC1]

The title compound was obtained by following a similar procedure described for Intermediate B1, starting from 1-(5-bromoimidazo[1,2-a]pyridin-8-yl)-3-(4-((4-methylpiperazin-1-yl)methyl)-3-(trifluoromethyl)phenyl)urea (D39, 100 mg, 0.196 mmol) and tert-butyl (tert-butoxycarbonyl)(7-(2-hydroxyethyl)-5-(4,4,5,5-tetramethyl-1,3,2-dioxaborolan-2-yl)-7H-pyrrolo[2,3-d]pyrimidin-4-yl)carbamate (step 2 of Intermediate B18, 99 mg, 0.196 mmol), and was isolated as an off-white solid (17 mg, 12% yield). Cleavage of one of the two Boc protecting groups was observed during the reaction. MS: 709.4 [M+H].

### Step 2: Synthesis of 1-(5-(4-amino-7-(2-hydroxyethyl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

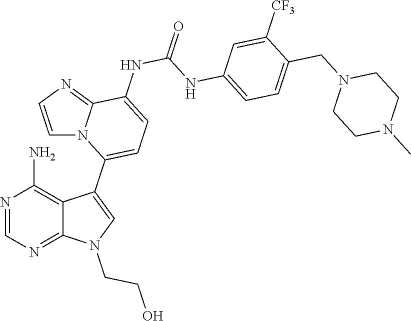 [SMILES: CN1CCN(Cc2ccc(NC(=O)Nc3ccc(-c4cn(CCO)c5ncnc(N)c45)n4ccnc34)cc2C(F)(F)F)CC1]

HCl(g) in dioxane (4M, 0.075 mL) was added to a stirred solution of tert-butyl (7-(2-hydroxyethyl)-5-(8-(3-(4-((4-methylpiperazin-1-yl)methyl)-3-(trifluoromethyl)phenyl)ureido)imidazo[1,2-a]pyridin-5-yl)-7H-pyrrolo[2,3-d]pyrimidin-4-yl)carbamate (15 mg, 0.021 mmol) in DCM (3 mL) at 0° C. and the resulting mixture was stirred at 25° C. for 12 h. Following completion of the reaction (as indicated by LCMS), the reaction mixture was concentrated under reduced pressure, giving crude material which was purified by preparative HPLC (eluting with 10 mM NH₄HCO in H₂O and ACN), affording the title compound as an off-white solid (5 mg, 38% yield). ¹H NMR (400 MHz, CD₃OD) δ=8.23 (s, 1H), 8.03 (d, J=8.0 Hz, 1H), 7.98 (d, J=4.0 Hz, 1H), 7.71-7.73 (m, 3H), 7.59-7.60 (m, 2H), 6.97 (d, J=8.0 Hz, 1H), 4.40-4.43 (m, 2H), 3.96-3.99 (m, 2H), 3.67 (s, 2H), 2.58 (s, 8H), 2.35 (s, 3H). MS: 609.3 [M+H].

### Example 131

### 1-(5-(4-Amino-7-(2-Hydroxy-2-Methylpropyl)-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of tert-butyl (7-(2-hydroxy-2-methylpropyl)-5-(8-(3-(4-((4-methylpiperazin-1-yl)methyl)-3-(trifluoromethyl)phenyl)ureido)imidazo[1,2-a]pyridin-5-yl)-7H-pyrrolo[2,3-d]pyrimidin-4-yl)carbamate

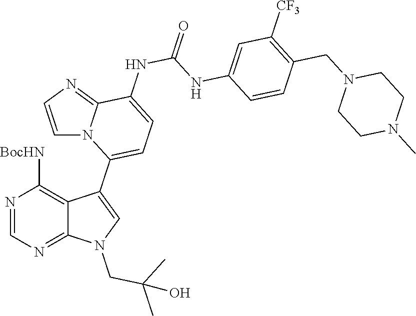 [SMILES: CN1CCN(Cc2ccc(NC(=O)Nc3ccc(-c4cn(CC(C)(C)O)c5ncnc(NC(=O)OC(C)(C)C)c45)n4ccnc34)cc2C(F)(F)F)CC1]

The title compound was obtained by following a similar procedure described for Intermediate B1, starting from 1-(5-bromoimidazo[1,2-a]pyridin-8-yl)-3-(4-((4-methylpiperazin-1-yl)methyl)-3-(trifluoromethyl)phenyl)urea (D39, 100 mg, 0.196 mmol) and tert-butyl (tert-butoxycarbonyl)(7-(2-hydroxy-2-methylpropyl)-5-(4,4,5,5-tetramethyl-1,3,2-dioxaborolan-2-yl)-7H-pyrrolo[2,3-d]pyrimidin-4-yl)carbamate (step 2 of Intermediate B19, 156 mg, 0.293 mmol), and was isolated as an off-white solid (18 mg, 12% yield). Cleavage of one of the two Boc protecting groups was observed during the reaction. MS: 737.4 [M+H].

### Step 2: Synthesis of 1-(5-(4-amino-7-(2-hydroxy-2-methylpropyl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

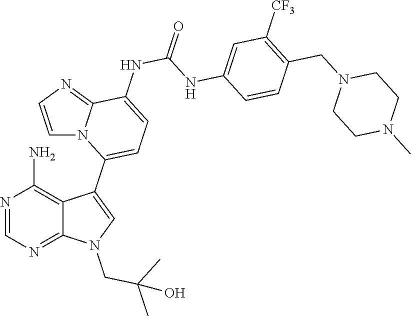 [SMILES: CN1CCN(Cc2ccc(NC(=O)Nc3ccc(-c4cn(CC(C)(C)O)c5ncnc(N)c45)n4ccnc34)cc2C(F)(F)F)CC1]

The title compound was obtained by following a similar procedure described for step 2 of Example 130, starting from tert-butyl (7-(2-hydroxy-2-methylpropyl)-5-(8-(3-(4-((4-methylpiperazin-1-yl)methyl)-3-(trifluoromethyl)phenyl)ureido)imidazo[1,2-a]pyridin-5-yl)-7H-pyrrolo[2,3-d]pyrimidin-4-yl)carbamate (18 mg, 0.024 mmol), and was isolated as an off-white solid (3 mg, 19% yield). ¹H NMR (400 MHz, CD₃OD) δ=8.23 (s, 1H), 8.04 (d, J=8.0 Hz, 1H), 7.98 (d, J=4.0 Hz, 1H), 7.71-7.73 (m, 3H), 7.59-7.60 (m, 2H), 6.98 (d, J=8.0 Hz, 1H), 4.30 (s, 2H), 3.67 (s, 2H), 2.58 (bs, 8H), 2.36 (s, 3H), 1.25 (s, 6H). MS: 637.4 [M+H]

### Example 132

### 1-(5-(4-Amino-7-(3-Hydroxycyclobutyl)-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of 1-(5-(4-amino-7-(3-(benzyloxy)cyclobutyl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

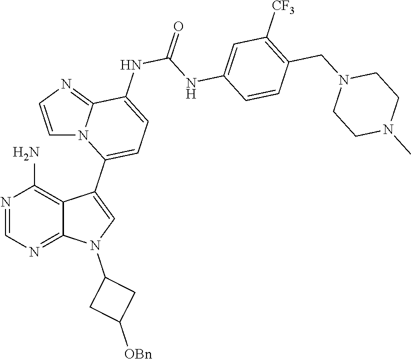 [SMILES: CN1CCN(Cc2ccc(NC(=O)Nc3ccc(-c4cn(C5CC(OCc6ccccc6)C5)c5ncnc(N)c45)n4ccnc34)cc2C(F)(F)F)CC1]

The title compound was obtained by following a similar procedure described for Intermediate B1, starting from 1-(5-bromoimidazo[1,2-a]pyridin-8-yl)-3-(4-((4-methylpiperazin-1-yl)methyl)-3-(trifluoromethyl)phenyl)urea (D39, 82 mg, 0.161 mmol) and tert-butyl (7-(3-(benzyloxy)cyclobutyl)-5-(4,4,5,5-tetramethyl-1,3,2-dioxaborolan-2-yl)-7H-pyrrolo[2,3-d]pyrimidin-4-yl)(tert-butoxycarbonyl)carbamate (step 2 of Intermediate B20, 100 mg, 0.161 mmol), and was isolated as an off-white solid (50 mg, 43% yield). Cleavage of the di-Boc protecting group was observed during the reaction LCMS: 725.3 [M+H].

### Step 2: Synthesis of 1-(5-(4-amino-7-(3-hydroxycyclobutyl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

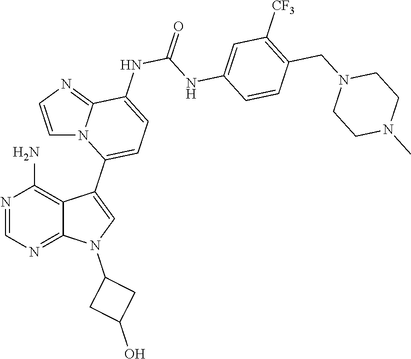 [SMILES: CN1CCN(Cc2ccc(NC(=O)Nc3ccc(-c4cn(C5CC(O)C5)c5ncnc(N)c45)n4ccnc34)cc2C(F)(F)F)CC1]

The title compound was obtained by following a similar procedure described for step 2 of Example 87, starting from 1-(5-(4-amino-7-(3-(benzyloxy)cyclobutyl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea (50 mg, 0.069 mmol), and was isolated as an off-white solid (10 mg. 23% yield). ¹H NMR (400 MHz, CD₃OD) δ=8.22 (bs, 1H), 8.04 (d, J=7.6 Hz, 1H), 7.98 (d, J=2.0 Hz, 1H), 7.78 (s, 1H), 7.65-7.77 (m, 3H), 7.59 (d, J=1.2 Hz, 1H), 6.98 (d, J=8.0 Hz, 1H), 5.49-5.54 (m, 1H), 4.62-4.64 (m, 1H), 3.66 (s, 2H), 2.84-2.91 (m, 2H), 2.48-2.66 (m, 10H), 2.33 (s, 3H). MS: 635.2 [M+H].

### Example 133

### 1-(5-(4-Amino-7-(Piperidin-4-Yl)-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of tert-butyl 4-(4-((2,4-dimethoxybenzyl)amino)-5-(8-(3-(4-((4-methylpiperazin-1-yl)methyl)-3-(trifluoromethyl)phenyl)ureido)imidazo[1,2-a]pyridin-5-yl)-7H-pyrrolo[2,3-d]pyrimidin-7-yl)piperidine-1-carboxylate

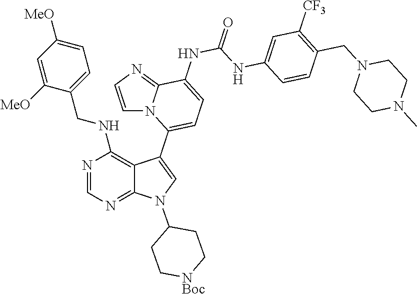 [SMILES: COc1ccc(CNc2ncnc3c2c(-c2ccc(NC(=O)Nc4ccc(CN5CCN(C)CC5)c(C(F)(F)F)c4)c4nccn24)cn3C2CCN(C(=O)OC(C)(C)C)CC2)c(OC)c1]

The title compound was prepared by following the general procedure for urea formation (Method B), starting from tert-butyl 4-(5-(8-aminoimidazo[1,2-a]pyridin-5-yl)-4-((2,4-dimethoxybenzyl)amino)-7H-pyrrolo[2,3-d]pyrimidin-7-yl)piperidine-1-carboxylate (B31, 100 mg, 0.17 mmol) and phenyl (4-((4-methylpiperazin-1-yl)methyl)-3-(trifluoromethyl)phenyl)carbamate (C5, 66 mg, 0.17 mmol), and was isolated as an off-white solid (30 mg, 20% yield). MS: 898.4 [M+H].

### Step 2: Synthesis of 1-(5-(4-amino-7-(piperidin-4-yl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

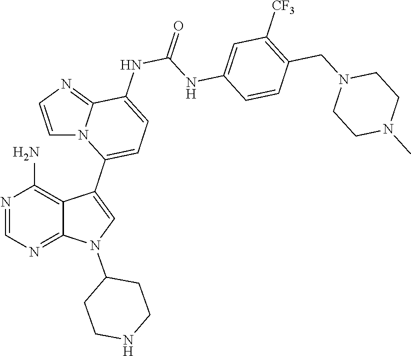 [SMILES: CN1CCN(Cc2ccc(NC(=O)Nc3ccc(-c4cn(C5CCNCC5)c5ncnc(N)c45)n4ccnc34)cc2C(F)(F)F)CC1]

The title compound was obtained by following a similar procedure described for step 2 of Example 87, starting from tert-butyl 4-(4-((2,4-dimethoxybenzyl)amino)-5-(8-(3-(4-((4-methylpiperazin-1-yl)methyl)-3-(trifluoromethyl)phenyl)ureido)imidazo[1,2-a]pyridin-5-yl)-7H-pyrrolo[2,3-d]pyrimidin-7-yl)piperidine-1-carboxylate (20 mg, 0.022 mmol), and was isolated as an off-white solid (2 mg, 14% yield). ¹H NMR (400 MHz, DMSO-d₆) δ=10.00 (bs, 1H), 9.19 (bs, 1H), 8.19 (s, 1H), 8.08 (d, J=8.4 Hz, 1H), 8.00 (d, J=7.6 Hz, 1H), 7.65-7.71 (m, 3H), 7.57-7.60 (m, 2H), 6.84 (d, J=7.6 Hz, 1H), 6.18 (bs, 2H), 4.74-4.79 (m, 1H), 3.55 (s, 2H), 3.18-3.22 (m, 2H), 2.85-2.86 (m, 2H), 2.40-2.61 (m, 10H), 2.17 (s, 3H), 2.04-2.09 (m, 2H). MS: 648.3 [M+H].

### Example 134

### 1-(5-(4-Amino-7-(1-Methylpiperidin-4-Yl)-7H-Pyrrolo[2,3-D]Pyrimidin-5-Yl)Imidazo[1,2-A]Pyridin-8-Yl)-3-(4-((4-Methylpiperazin-1-Yl)Methyl)-3-(Trifluoromethyl)Phenyl)Urea

### Step 1: Synthesis of 1-(5-(4-((2,4-dimethoxybenzyl)amino)-7-(piperidin-4-yl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

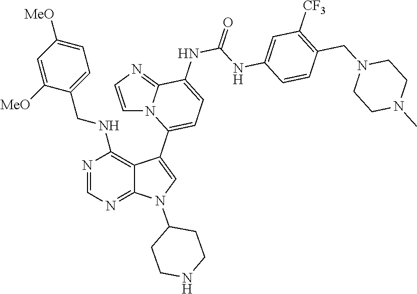 [SMILES: COc1ccc(CNc2ncnc3c2c(-c2ccc(NC(=O)Nc4ccc(CN5CCN(C)CC5)c(C(F)(F)F)c4)c4nccn24)cn3C2CCNCC2)c(OC)c1]

The title compound was obtained by following a similar procedure described for step 2 of Example 5, starting from tert-butyl 4-(4-((2,4-dimethoxybenzyl)amino)-5-(8-(3-(3-(1-(trifluoromethyl)cyclopropyl)isoxazol-5-yl)ureido)imidazo[1,2-a]pyridin-5-yl)-7H-pyrrolo[2,3-d]pyrimidin-7-yl)piperidine-1-carboxylate (Step 1 of Example 133, 30 mg, 0.033 mmol). Crude material (23 mg) was isolated and used without further purification. MS: 798.0 [M+H].

### Step 2: Synthesis of 1-(5-(4-((2,4-dimethoxybenzyl)amino)-7-(1-methylpiperidin-4-yl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

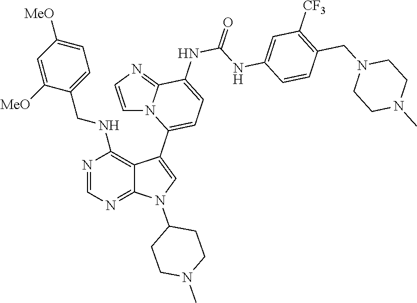 [SMILES: COc1ccc(CNc2ncnc3c2c(-c2ccc(NC(=O)Nc4ccc(CN5CCN(C)CC5)c(C(F)(F)F)c4)c4nccn24)cn3C2CCN(C)CC2)c(OC)c1]

The title compound was obtained by following a similar procedure described for step 2 of Example 95, starting from 1-(5-(4-((2,4-dimethoxybenzyl)amino)-7-(piperidin-4-yl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea (25 mg, 0.031 mmol). Crude material (10 mg) was isolated and used without further purification. MS: 812.3 [M+H].

### Step 3: Synthesis of 1-(5-(4-amino-7-(1-methylpiperidin-4-yl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea

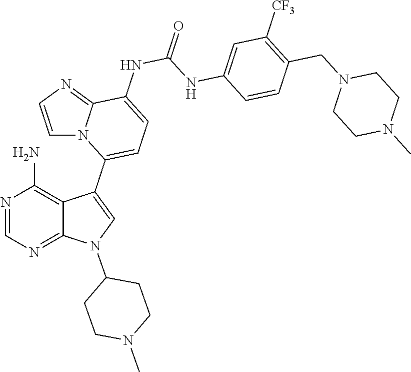 [SMILES: CN1CCC(n2cc(-c3ccc(NC(=O)Nc4ccc(CN5CCN(C)CC5)c(C(F)(F)F)c4)c4nccn34)c3c(N)ncnc32)CC1]

The title compound was prepared by following a similar procedure described for Step 2 of Example 83, starting from 1-(5-(4-((2,4-dimethoxybenzyl)amino)-7-(1-methylpiperidin-4-yl)-7H-pyrrolo[2,3-d]pyrimidin-5-yl)imidazo[1,2-a]pyridin-8-yl)-3-(4-((4-methylpiperazin-1-yl)methyl)-3-(trifluoromethyl)phenyl)urea (10 mg, 0.012 mmol), and was isolated as an off-white solid (2.5 mg, 31% yield). ¹H NMR (400 MHz, CD₃OD) δ=8.23 (s, 1H), 8.04 (d, J=7.6 Hz, 1H), 7.97 (d, J=2.0 Hz, 1H), 7.71-7.73 (m, 2H), 7.69 (s, 1H), 7.64 (d, J=1.2 Hz, 1H), 7.59 (d, J=1.2 Hz, 1H), 6.96 (d, J=7.6 Hz, 1H), 4.74 (s, 2H), 3.66-0.00 (m, 1H), 3.10 (s, 2H), 2.34-2.56 (m, 18H), 2.14-2.17 (m, 2H). MS: 662.3 [M+H].

### Example 135

### 1-(5-(4-Amino-7-Isopropyl-7H-Pyrrolo[2,3-D]Pyrimidin-5-Yl)Imidazo[1,2-A]Pyridin-8-Yl)-3-(4-((1-Methylpiperidin-4-Yl)Oxy)-3-(Trifluoromethyl)Phenyl)Urea

### Step 1: Synthesis of 1-(5-(4-((2,4-dimethoxybenzyl)amino)-7-isopropyl-7H-pyrrolo[2,3-d]pyrimidin-5-yl)imidazo[1,2-a]pyridin-8-yl)-3-(4-((1-methylpiperidin-4-yl)oxy)-3-(trifluoromethyl)phenyl)urea

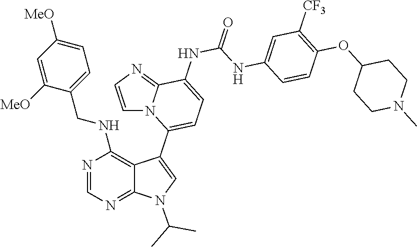 [SMILES: COc1ccc(CNc2ncnc3c2c(-c2ccc(NC(=O)Nc4ccc(OC5CCN(C)CC5)c(C(F)(F)F)c4)c4nccn24)cn3C(C)C)c(OC)c1]

The title compound was prepared by following the general procedure for urea formation (Method B), starting from 5-(8-aminoimidazo[1,2-a]pyridin-5-yl)-N-(2,4-dimethoxybenzyl)-7-isopropyl-7H-pyrrolo[2,3-d]pyrimidin-4-amine (B30, 60 mg, 0.131 mmol) and phenyl (4-((1-methylpiperidin-4-yl)oxy)-3-(trifluoromethyl)phenyl)carbamate (C27, 52 mg, 0.131 mmol), and was obtained as an off-white solid (30 mg, 30% yield). MS: 758.3 [M+H].

### Step 2: Synthesis of 1-(5-(4-amino-7-isopropyl-7H-pyrrolo[2,3-d]pyrimidin-5-yl)imidazo[1,2-a]pyridin-8-yl)-3-(4-((1-methylpiperidin-4-yl)oxy)-3-(trifluoromethyl)phenyl)urea

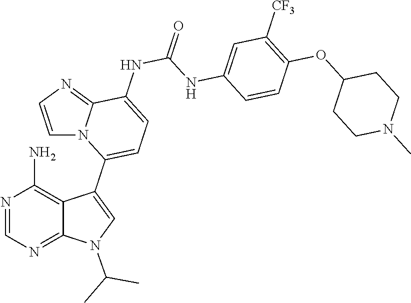 [SMILES: CC(C)n1cc(-c2ccc(NC(=O)Nc3ccc(OC4CCN(C)CC4)c(C(F)(F)F)c3)c3nccn23)c2c(N)ncnc21]

The title compound was prepared by following a similar procedure described for Step 2 of Example 83, starting from 1-(5-(4-((2,4-dimethoxybenzyl)amino)-7-isopropyl-7H-pyrrolo[2,3-d]pyrimidin-5-yl)imidazo[1,2-a]pyridin-8-yl)-3-(4-((1-methylpiperidin-4-yl)oxy)-3-(trifluoromethyl)phenyl)urea (30 mg, 0.028 mmol), and was isolated as an off-white solid (14 mg, 82% yield). ¹H NMR (400 MHz, DMSO-d₆) δ=9.78 (bs, 1H), 9.09 (bs, 1H), 8.18 (s, 1H), 7.98 (d, J=7.6 Hz, 1H), 7.90 (d, J=2.4 Hz, 1H), 7.78 (s, 1H), 7.64 (d, J=1.2 Hz, 1H), 7.54-7.57 (m, 2H), 7.28-7.30 (m, 1H), 6.83 (d, J=7.6 Hz, 1H), 6.13 (bs, 2H), 4.98-5.05 (m, 1H), 4.54-4.55 (m, 1H), 2.58 (bs, 2H), 2.24-2.26 (m, 2H), 2.16 (s, 3H), 1.89-1.92 (m, 2H), 1.69 (bs, 2H), 1.49 (d, J=6.4 Hz, 6H). MS: 608.3 [M+H].

### Example 136

### 1-(5-(4-Amino-7-Isopropyl-7H-Pyrrolo[2,3-D]Pyrimidin-5-Yl)Imidazo[1,2-A]Pyridin-8-Yl)-3-(4-((1-Ethylpiperidin-4-Yl)Oxy)-3-(Trifluoromethyl)Phenyl)Urea

### Step 1: Synthesis of 1-(5-(4-((2,4-dimethoxybenzyl)amino)-7-isopropyl-7H-pyrrolo[2,3-d]pyrimidin-5-yl)imidazo[1,2-a]pyridin-8-yl)-3-(4-((1-ethylpiperidin-4-yl)oxy)-3-(trifluoromethyl)phenyl)urea

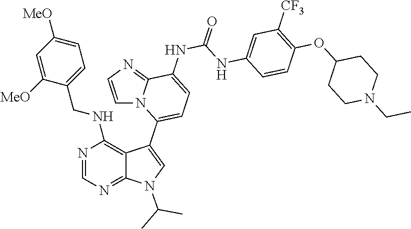 [SMILES: CCN1CCC(Oc2ccc(NC(=O)Nc3ccc(-c4cn(C(C)C)c5ncnc(NCc6ccc(OC)cc6OC)c45)n4ccnc34)cc2C(F)(F)F)CC1]

The title compound was obtained by following a similar procedure described for Intermediate B1, starting from 1-(5-bromoimidazo[1,2-a]pyridin-8-yl)-3-(4-((1-ethylpiperidin-4-yl)oxy)-3-(trifluoromethyl)phenyl)urea (D8, 50 mg, 0.0965 mmol) and N-(2,4-dimethoxybenzyl)-7-isopropyl-5-(4,4,5,5-tetramethyl-1,3,2-dioxaborolan-2-yl)-7H-pyrrolo[2,3-d]pyrimidin-4-amine (step 1 of Intermediate B30, 50 mg, 0.111 mmol), and was obtained as an off-white solid (40 mg, 47% yield). MS: 772.3 [M+H].

### Step 2: Synthesis of 1-(5-(4-amino-7-isopropyl-7H-pyrrolo[2,3-d]pyrimidin-5-yl)imidazo[1,2-a]pyridin-8-yl)-3-(4-((1-ethylpiperidin-4-yl)oxy)-3-(trifluoromethyl)phenyl)urea

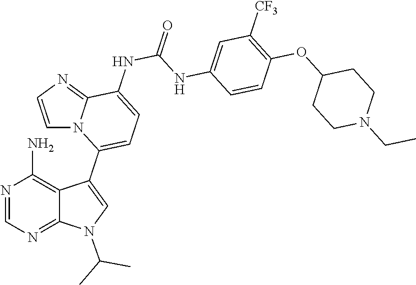 [SMILES: CCN1CCC(Oc2ccc(NC(=O)Nc3ccc(-c4cn(C(C)C)c5ncnc(N)c45)n4ccnc34)cc2C(F)(F)F)CC1]

The title compound was prepared by following a similar procedure described for Step 2 of Example 83, starting from 1-(5-(4-((2,4-dimethoxybenzyl)amino)-7-isopropyl-7H-pyrrolo[2,3-d]pyrimidin-5-yl)imidazo[1,2-a]pyridin-8-yl)-3-(4-((1-ethylpiperidin-4-yl)oxy)-3-(trifluoromethyl)phenyl)urea (50 mg, 0.065 mmol), and was isolated as an off-white solid (8.5 mg, 21% yield). ¹H NMR (400 MHz, DMSO-d₆) δ=9.78 (bs, 1H), 9.09 (bs, 1H), 8.18 (s, 1H), 7.98 (d, J=8.0 Hz, 1H), 7.90 (d, J=2.4 Hz, 1H), 7.78 (s, 1H), 7.64 (d, J=1.2 Hz, 1H), 7.54-7.57 (m, 2H), 7.29 (d, J=8.8 Hz, 1H), 6.83 (d, J=7.6 Hz, 1H), 6.14 (bs, 2H), 4.99-5.03 (m, 1H), 4.55-4.55 (m, 1H), 2.60 (bs, 2H), 2.26-2.36 (m, 4H), 1.91-1.94 (m, 2H), 1.65-1.71 (m, 2H), 1.50 (d, J=6.8 Hz, 6H), 1.00 (t, J=7.2 Hz, 3H). MS: 622.3 [M+H].

### Example 137

### 1-(5-(4-Amino-7-Cyclobutyl-7H-Pyrrolo[2,3-D]Pyrimidin-5-Yl)Imidazo[1,2-A]Pyridin-8-Yl)-3-(4-((1-Ethylpiperidin-4-Yl)Oxy)-3-(Trifluoromethyl)Phenyl)Urea

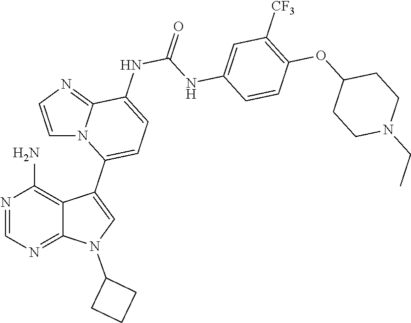 [SMILES: CCN1CCC(Oc2ccc(NC(=O)Nc3ccc(-c4cn(C5CCC5)c5ncnc(N)c45)n4ccnc34)cc2C(F)(F)F)CC1]

The title compound was obtained by following a similar procedure described for Intermediate B1, starting from 1-(5-bromoimidazo[1,2-a]pyridin-8-yl)-3-(4-((1-ethylpiperidin-4-yl)oxy)-3-(trifluoromethyl)phenyl)urea (D8, 50 mg, 0.095 mmol) and tert-butyl (tert-butoxycarbonyl)(7-cyclobutyl-5-(4,4,5,5-tetramethyl-1,3,2-dioxaborolan-2-yl)-7H-pyrrolo[2,3-d]pyrimidin-4-yl)carbamate (step 2 of Intermediate B17, 73 mg, 0.142 mmol), and was obtained as an off-white solid (7 mg, 12% yield). Cleavage of the di-Boc protecting group was observed during the reaction. ¹H NMR (400 MHz, CD₃OD) δ=8.21 (s, 1H), 8.01 (d, J=8.0 Hz, 1H), 7.84 (d, J=2.4 Hz, 1H), 7.80 (s, 1H), 7.64-7.68 (m, 2H), 7.58 (d, J=1.2 Hz, 1H), 7.21 (d, J=8.8 Hz, 1H), 6.96 (d, J=7.6 Hz, 1H), 5.26-5.34 (m, 1H), 4.62 (bs, 1H), 2.84-2.88 (m, 2H), 2.57-2.68 (m, 8H), 1.93-2.12 (m, 6H), 1.20-1.21 (m, 3H). MS: 634.3 [M+H].

### Biological Example 1

### Biochemical Assay of the Compounds

Representative compounds were tested for inhibitory activity against NEK7 and IL-1β release according to the procedures described above. Results are given in the following table.

TABLE 2
Activity of Representative Compounds

| | | IL-1β |
|---|---|---|
| | Nek7 IC₅₀ | release IC₅₀ |
| Compound | (nM) | (nM) |
| I-1 | * | — |
| I-2 | * | — |
| I-3 | * | — |
| I-4 | * | * |
| I-5 | **** | — |
| I-6 | *** | — |
| I-7 | ** | — |
| I-8 | **** | *** |
| I-9 | **** | **** |
| I-10 | **** | — |
| I-11 | **** | **** |
| I-12 | *** | — |
| I-13 | ** | — |
| I-14 | * | — |
| I-15 | *** | — |
| I-16 | **** | — |
| I-17 | **** | **** |
| I-18 | **** | — |
| I-19 | *** | — |
| I-20 | **** | — |
| I-21 | **** | — |
| I-22 | **** | **** |
| I-23 | **** | — |
| I-24 | * | — |
| I-25 | — | — |
| I-26 | *** | — |
| I-27 | **** | — |
| I-28 | — | — |
| I-29 | — | — |
| I-30 | — | — |
| I-31 | **** | — |
| I-32 | **** | * |
| I-33 | *** | — |
| I-34 | **** | **** |
| I-35 | **** | — |
| I-36 | **** | — |
| I-37 | **** | **** |
| I-38 | **** | — |
| I-39 | **** | — |
| I-40 | **** | — |
| I-41 | **** | — |
| I-42 | — | — |
| I-43 | — | — |
| I-44 | — | — |
| I-45 | — | — |
| I-46 | * | — |
| I-47 | * | **** |
| I-48 | — | — |
| I-49 | — | — |
| I-50 | * | — |
| I-51 | * | * |
| I-52 | *** | ** |
| I-53 | **** | **** |
| I-54 | **** | — |
| I-55 | *** | — |
| I-56 | ** | — |
| I-57 | ** | — |
| I-58 | * | — |
| I-59 | *** | — |
| I-60 | * | — |
| I-61 | * | — |
| I-62 | ** | **** |
| I-63 | ** | ** |
| I-64 | * | — |
| I-65 | ** | * |
| I-66 | *** | **** |
| I-67 | ** | — |
| I-68 | **** | *** |
| I-69 | **** | * |
| I-70 | **** | — |
| I-71 | ** | — |
| I-72 | ** | * |
| I-73 | — | — |
| I-74 | **** | **** |
| I-75 | — | — |
| I-76 | — | — |
| I-77 | **** | **** |
| I-78 | **** | — |
| I-79 | **** | **** |
| I-80 | — | — |
| I-81 | — | — |
| I-82 | — | — |
| I-83 | **** | — |
| I-84 | **** | — |
| I-85 | **** | — |
| I-86 | **** | — |
| I-87 | **** | — |
| I-88 | — | — |
| I-89 | **** | — |
| I-90 | — | — |
| I-91 | **** | — |
| I-92 | — | — |
| I-93 | — | — |
| I-94 | **** | — |
| I-95 | *** | — |
| I-96 | — | — |
| I-97 | — | — |
| I-98 | **** | **** |
| I-99 | — | — |
| I-100 | — | — |
| I-101 | — | — |
| I-102 | — | — |
| I-103 | — | — |
| I-104 | — | — |
| I-105 | — | — |
| I-106 | — | — |
| I-107 | — | — |
| I-108 | — | — |
| I-109 | — | — |
| I-110 | — | — |
| I-111 | — | — |
| I-112 | — | — |
| I-113 | — | — |
| I-114 | — | — |
| I-115 | — | — |
| I-116 | — | — |
| I-117 | — | — |
| I-118 | — | — |
| I-119 | *** | |
| I-120 | **** | |
| I-121 | * | |
| I-122 | ** | |
| I-123 | * | |
| I-124 | — | |
| I-125 | *** | |
| I-126 | **** | |
| I-127 | **** | |
| I-128 | **** | |
| I-129 | **** | |
| I-130 | **** | |
| I-131 | **** | |
| I-132 | **** | |
| I-133 | **** | |
| I-134 | *** | |
| I-135 | **** | |
| I-136 | **** | |
| I-137 | **** | |
| — | — | — |

For NEK7 IC₅₀ activity in Table 2:
*represents a value above 2,000 nM
**represents a value between 500 up to 2,000 nM
***represents a value between 200 up to 500 nM
****represents a value below 200 nM
—denotes a value was not determined
For IL-1β release IC₅₀ activity in Table 2:
*represents a value above 1,000 nM
**represents a value between 250 and 1,000 nM
***represents a value between 100 nM and 250 nM
****represents a value below 100 nM
—denotes a value was not determined

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A compound having the following Structure (I): 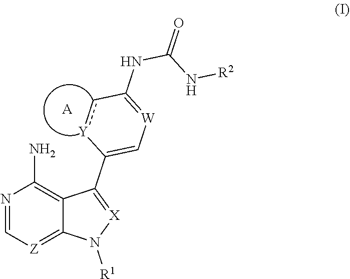
or a stereoisomer, tautomer, or salt thereof, wherein: [symbol]
represents a double or a single bond such that all valences are satisfied;
A is an optionally substituted 5-6-membered heterocyclyl, an optionally substituted 6-membered aryl, or an optionally substituted 5-6-membered heteroaryl;
X is N or CR³;
Y is C or N;
W is CH or N;
Z is CH or N;
R¹ is optionally substituted C₁-C₆ alkyl, optionally substituted C₁-C₆ alkenyl, optionally substituted C₁-C₆ alkynyl, optionally substituted C₁-C₆ hydroxyalkyl, optionally substituted C₁-C₆ alkoxyalkyl, optionally substituted C₁-C₆ carboxyalkyl, optionally substituted C₁-C₆ haloalkyl, optionally substituted C₃-C₈ cycloalkyl, optionally substituted C₆-C₁₀ aryl, optionally substituted 3-10 membered heterocyclyl, or optionally substituted 5-10 membered heteroaryl;
R² is optionally substituted aryl or optionally substituted heteroaryl; and
R³ is hydrogen, halo, cyano, optionally substituted C₁-C₆ alkyl, optionally substituted C₁-C₆ haloalkyl, optionally substituted C₁-C₆ alkoxy, optionally substituted C₁-C₆ haloalkoxy, or optionally substituted C₃-C₈ cycloalkyl.

2. The compound of claim 1, wherein A is a 5-membered heterocyclyl or a 6-membered heterocyclyl.

3. (canceled)

4. The compound of claim 1, wherein A is a 6-membered heteroaryl or a 5-membered heteroaryl.

5. (canceled)

6. The compound of claim 1, wherein A is a 6-membered aryl.

7. The compound of claim 4, wherein Y is N.

8-9. (canceled)

10. The compound of claim 1, wherein A is substituted with one or more substituents selected from the group consisting of halo, cyano, optionally substituted C₁-C₆ alkyl, optionally substituted C₁-C₆ haloalkyl, optionally substituted C₁-C₆ alkoxy, optionally substituted C₁-C₆ haloalkoxy, or optionally substituted C₃-C₈ cycloalkyl.

11-12. (canceled)

13. The compound of claim 1, wherein A is substituted with one or more substituents selected from the group consisting of methyl, fluoro, and trifluoromethyl.

14. The compound of claim 1, wherein 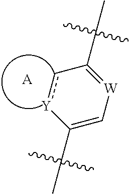
has the following structure:

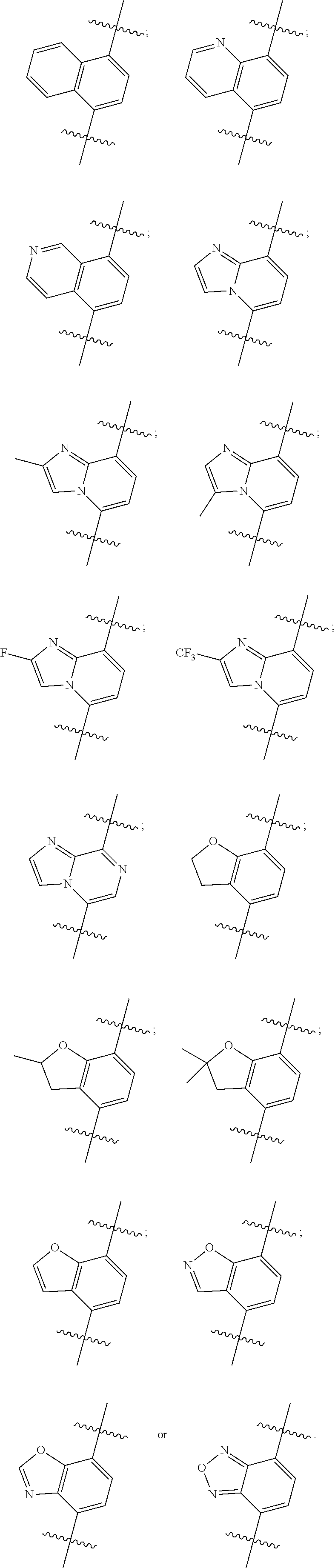

15. The compound of claim 1, wherein Z is CH.

16. The compound of claim 1, wherein Z is N.

17. The compound of claim 1, wherein X is N or CH.

18-20. (canceled)

21. The compound of claim 1, wherein R¹ is optionally substituted C₁-C₆ alkyl, optionally substituted C₁-C₆ alkenyl, optionally substituted C₁-C₆ alkynyl, optionally substituted C₁-C₆ hydroxyalkyl, optionally substituted C₁-C₆ alkoxyalkyl, optionally substituted C₁-C₆ carboxyalkyl, optionally substituted C₃-C₈ cycloalkyl, optionally substituted 3-10 membered heterocyclyl, or optionally substituted 5-10 membered heteroaryl.

22. (canceled)

23. The compound of claim 1, wherein R¹ is methyl, ethyl, or iso-propyl, cyclopropyl, cyclobutyl, cyclopentyl, or cyclohexyl.

24-28. (canceled)

29. The compound of claim 1, wherein R¹ has one of the following structures:

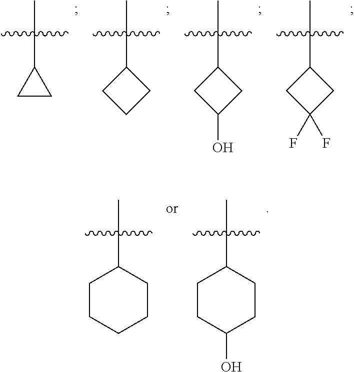

30-33. (canceled)

34. The compound of claim 1, wherein R¹ has one of the following structures:

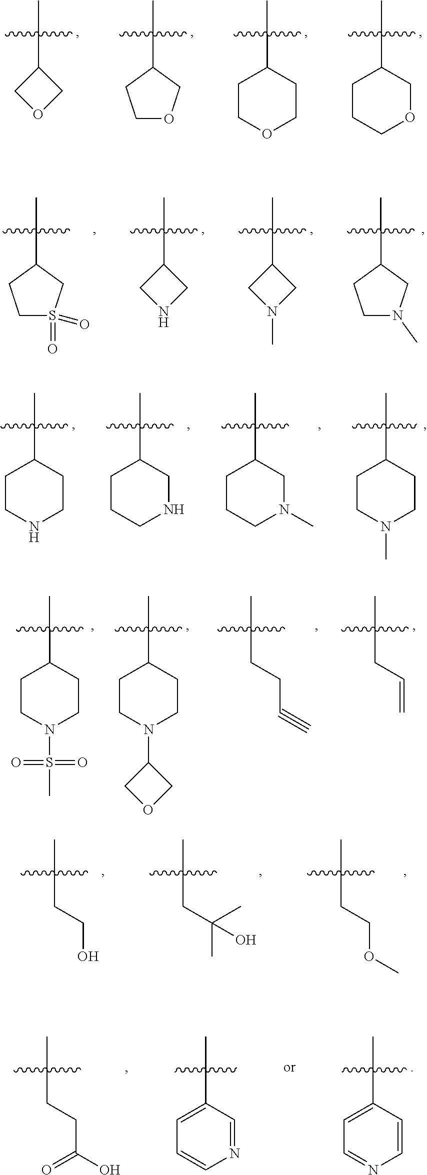

35-38. (canceled)

39. The compound of claim 1, wherein R² is optionally substituted phenyl, optionally substituted isoxazolyl, or optionally substituted pyrazolyl.

40-44. (canceled)

45. The compound of claim 1, wherein R² has one of the following structures:

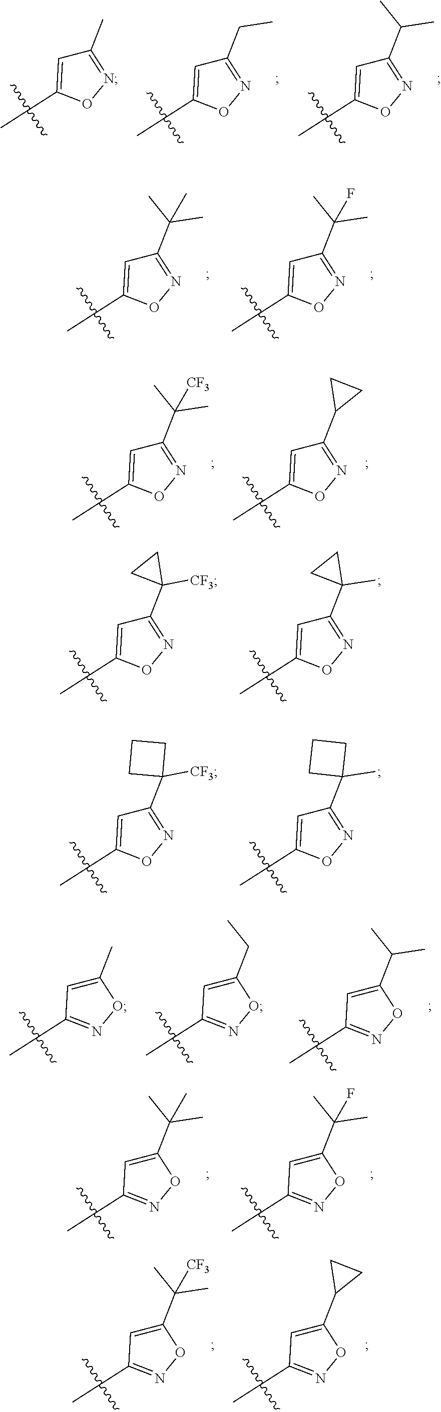

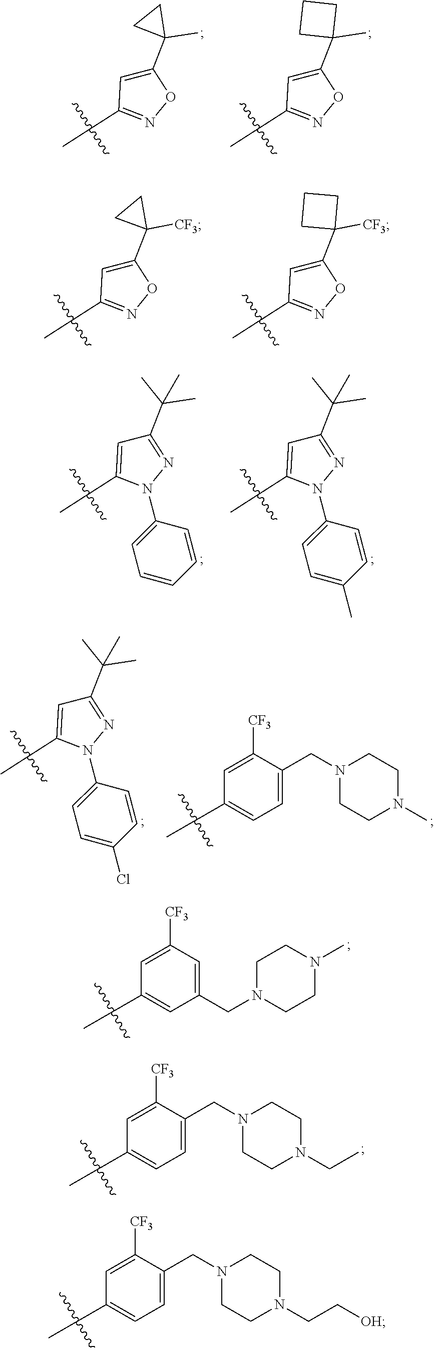

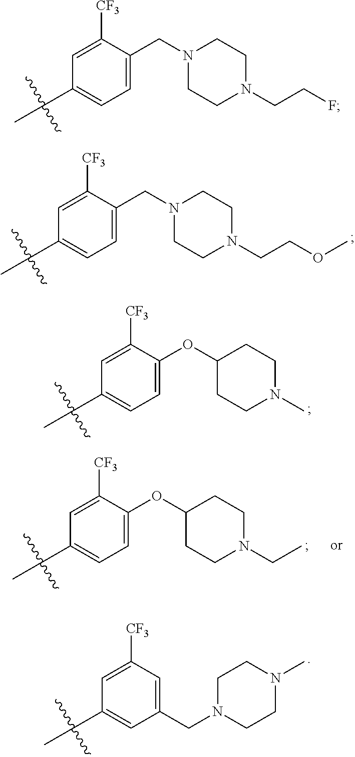 [SMILES: *c1ccc(CN2CCN(CCF)CC2)c(C(F)(F)F)c1 ; *c1ccc(CN2CCN(CCOC)CC2)c(C(F)(F)F)c1 ; *c1ccc(OC2CCN(C)CC2)c(C(F)(F)F)c1 ; *c1ccc(OC2CCN(CC)CC2)c(C(F)(F)F)c1 ; *c1cc(CN2CCN(C)CC2)cc(C(F)(F)F)c1]

46. The compound of claim 1, wherein the compound has one of the following structures 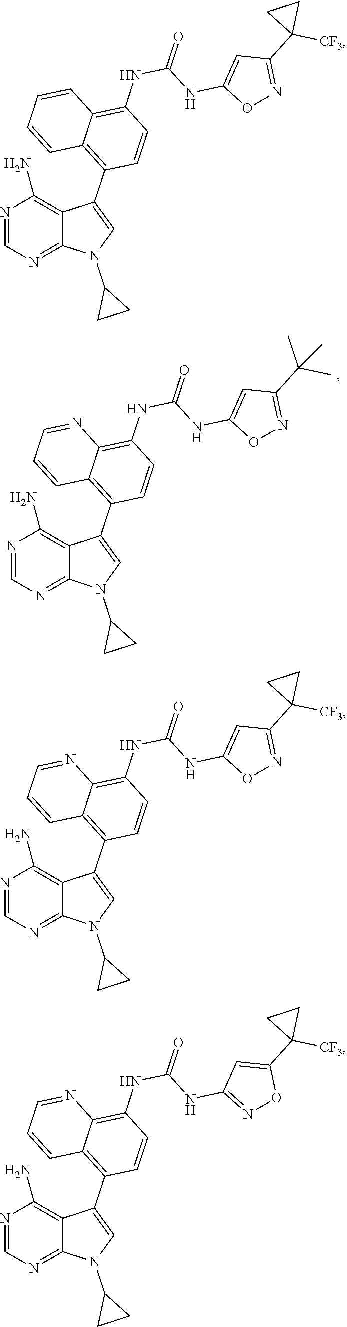 [SMILES: Nc1ncnc2c1c(-c1ccc(NC(=O)Nc3cc(C4(C(F)(F)F)CC4)no3)c3ccccc13)cn2C1CC1 ; CC(C)(C)c1cc(NC(=O)Nc2ccc(-c3cn(C4CC4)c4ncnc(N)c34)c3cccnc23)on1 ; Nc1ncnc2c1c(-c1ccc(NC(=O)Nc3cc(C4(C(F)(F)F)CC4)no3)c3ncccc13)cn2C1CC1 ; Nc1ncnc2c1c(-c1ccc(NC(=O)Nc3cc(C4(C(F)(F)F)CC4)on3)c3ncccc13)cn2C1CC1] 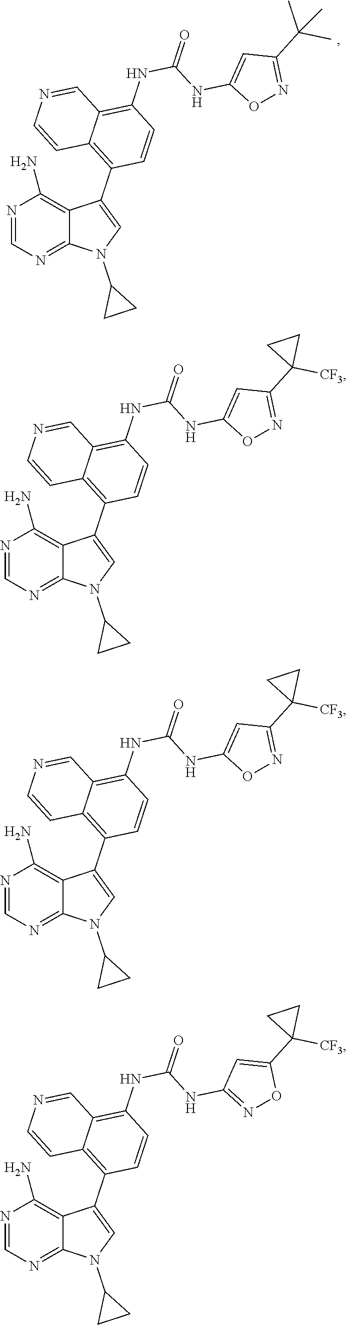 [SMILES: CC(C)(C)c1cc(NC(=O)Nc2ccc(-c3cn(C4CC4)c4ncnc(N)c34)c3ccncc23)on1 ; Nc1ncnc2c1c(-c1ccc(NC(=O)Nc3cc(C4(C(F)(F)F)CC4)no3)c3cnccc13)cn2C1CC1 ; Nc1ncnc2c1c(-c1ccc(NC(=O)Nc3cc(C4(C(F)(F)F)CC4)no3)c3cnccc13)cn2C1CC1 ; Nc1ncnc2c1c(-c1ccc(NC(=O)Nc3cc(C4(C(F)(F)F)CC4)on3)c3cnccc13)cn2C1CC1] 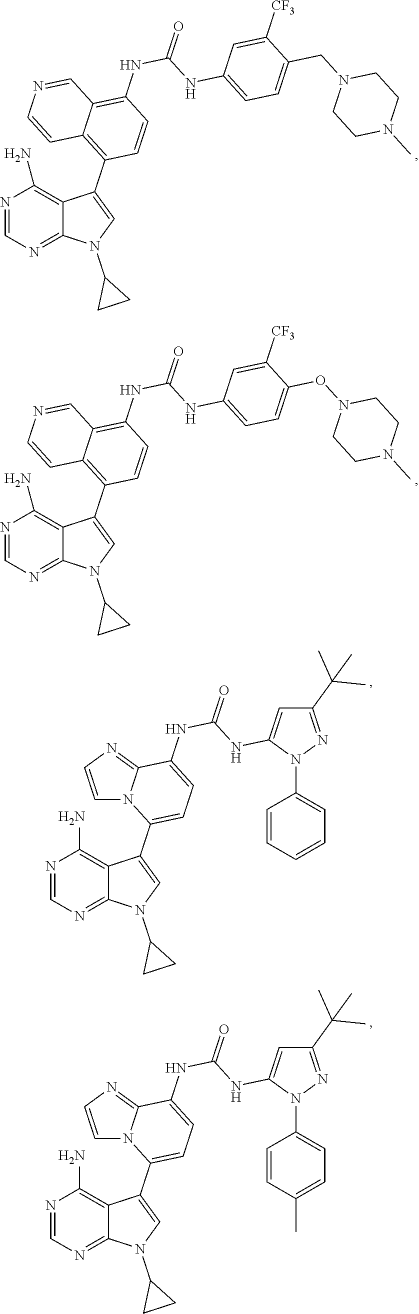 [SMILES: CN1CCN(Cc2ccc(NC(=O)Nc3ccc(-c4cn(C5CC5)c5ncnc(N)c45)c4ccncc34)cc2C(F)(F)F)CC1 ; CN1CCN(Oc2ccc(NC(=O)Nc3ccc(-c4cn(C5CC5)c5ncnc(N)c45)c4ccncc34)cc2C(F)(F)F)CC1 ; CC(C)(C)c1cc(NC(=O)Nc2ccc(-c3cn(C4CC4)c4ncnc(N)c34)n3ccnc23)n(-c2ccccc2)n1 ; Cc1ccc(-n2nc(C(C)(C)C)cc2NC(=O)Nc2ccc(-c3cn(C4CC4)c4ncnc(N)c34)n3ccnc23)cc1] 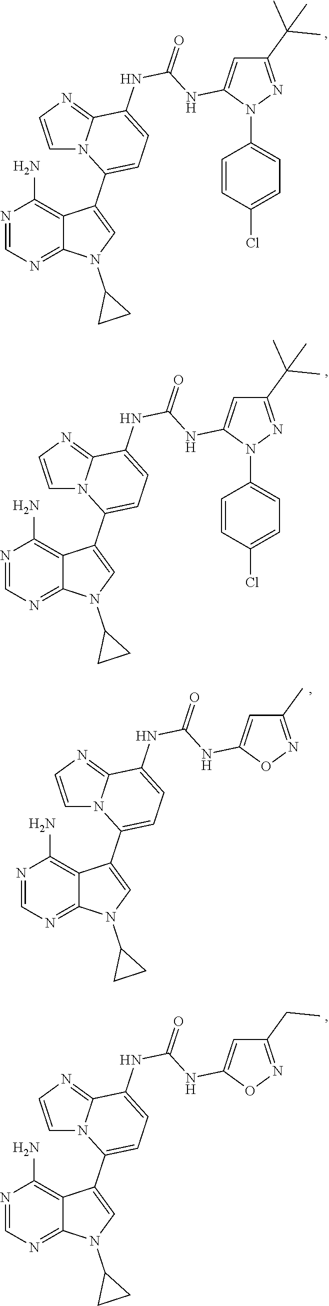 [SMILES: CC(C)(C)c1cc(NC(=O)Nc2ccc(-c3cn(C4CC4)c4ncnc(N)c34)n3ccnc23)n(-c2ccc(Cl)cc2)n1 ; CC(C)(C)c1cc(NC(=O)Nc2ccc(-c3cn(C4CC4)c4ncnc(N)c34)n3ccnc23)n(-c2ccc(Cl)cc2)n1 ; Cc1cc(NC(=O)Nc2ccc(-c3cn(C4CC4)c4ncnc(N)c34)n3ccnc23)on1 ; CCc1cc(NC(=O)Nc2ccc(-c3cn(C4CC4)c4ncnc(N)c34)n3ccnc23)on1] 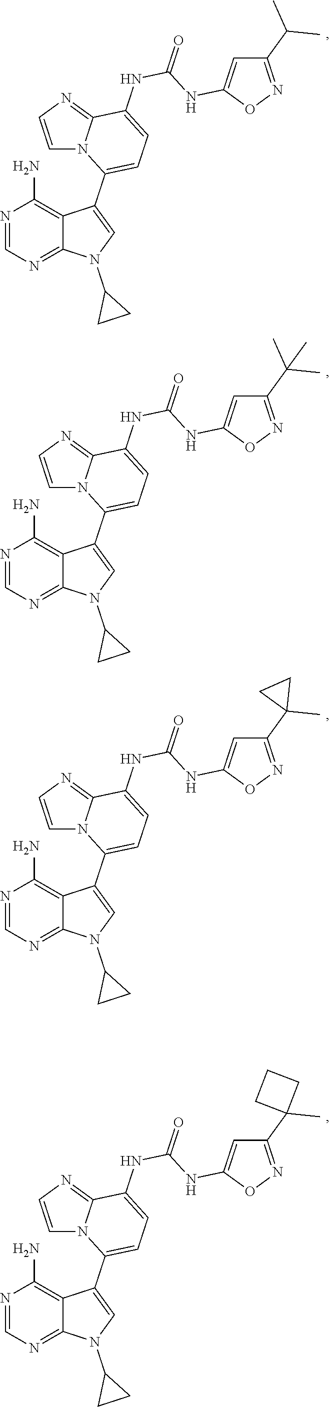 [SMILES: CC(C)c1cc(NC(=O)Nc2ccc(-c3cn(C4CC4)c4ncnc(N)c34)n3ccnc23)on1 ; CC(C)(C)c1cc(NC(=O)Nc2ccc(-c3cn(C4CC4)c4ncnc(N)c34)n3ccnc23)on1 ; CC1(c2cc(NC(=O)Nc3ccc(-c4cn(C5CC5)c5ncnc(N)c45)n4ccnc34)on2)CC1 ; CC1(c2cc(NC(=O)Nc3ccc(-c4cn(C5CC5)c5ncnc(N)c45)n4ccnc34)on2)CCC1] 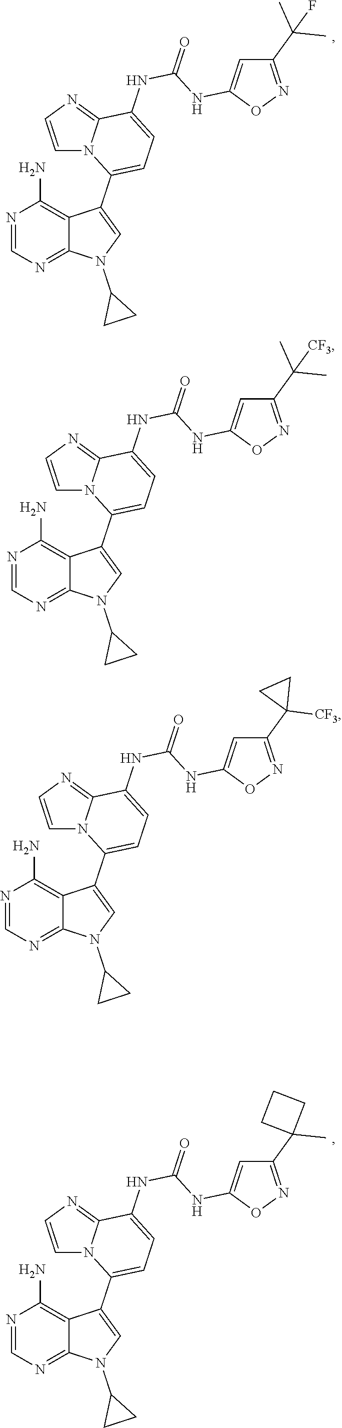 [SMILES: CC(C)(F)c1cc(NC(=O)Nc2ccc(-c3cn(C4CC4)c4ncnc(N)c34)n3ccnc23)on1 ; CC(C)(c1cc(NC(=O)Nc2ccc(-c3cn(C4CC4)c4ncnc(N)c34)n3ccnc23)on1)C(F)(F)F ; Nc1ncnc2c1c(-c1ccc(NC(=O)Nc3cc(C4(C(F)(F)F)CC4)no3)c3nccn13)cn2C1CC1 ; CC1(c2cc(NC(=O)Nc3ccc(-c4cn(C5CC5)c5ncnc(N)c45)n4ccnc34)on2)CCC1] 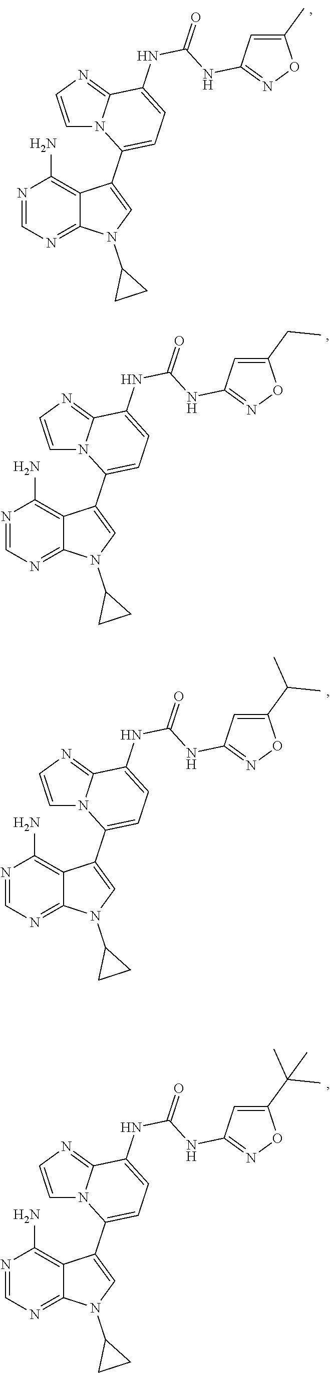 [SMILES: Cc1cc(NC(=O)Nc2ccc(-c3cn(C4CC4)c4ncnc(N)c34)n3ccnc23)no1 ; CCc1cc(NC(=O)Nc2ccc(-c3cn(C4CC4)c4ncnc(N)c34)n3ccnc23)no1 ; CC(C)c1cc(NC(=O)Nc2ccc(-c3cn(C4CC4)c4ncnc(N)c34)n3ccnc23)no1 ; CC(C)(C)c1cc(NC(=O)Nc2ccc(-c3cn(C4CC4)c4ncnc(N)c34)n3ccnc23)no1] 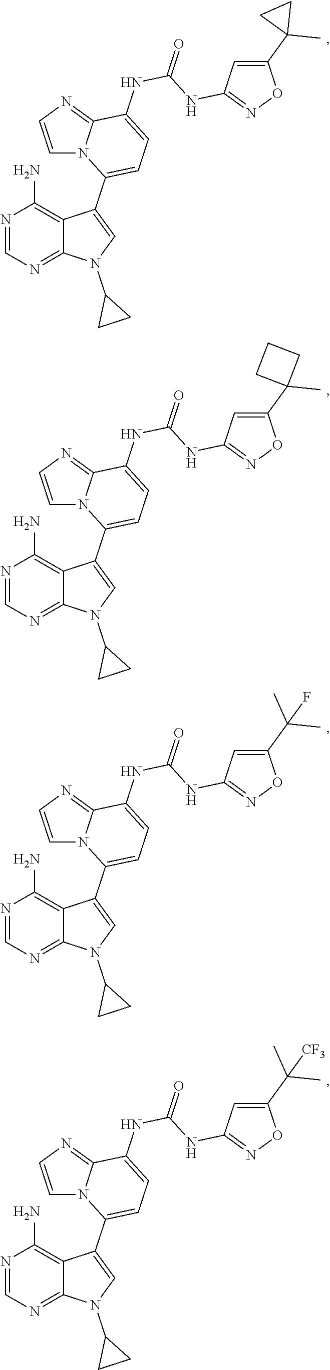 [SMILES: CC1(c2cc(NC(=O)Nc3ccc(-c4cn(C5CC5)c5ncnc(N)c45)n4ccnc34)no2)CC1 ; CC1(c2cc(NC(=O)Nc3ccc(-c4cn(C5CC5)c5ncnc(N)c45)n4ccnc34)no2)CCC1 ; CC(C)(F)c1cc(NC(=O)Nc2ccc(-c3cn(C4CC4)c4ncnc(N)c34)n3ccnc23)no1 ; CC(C)(c1cc(NC(=O)Nc2ccc(-c3cn(C4CC4)c4ncnc(N)c34)n3ccnc23)no1)C(F)(F)F] 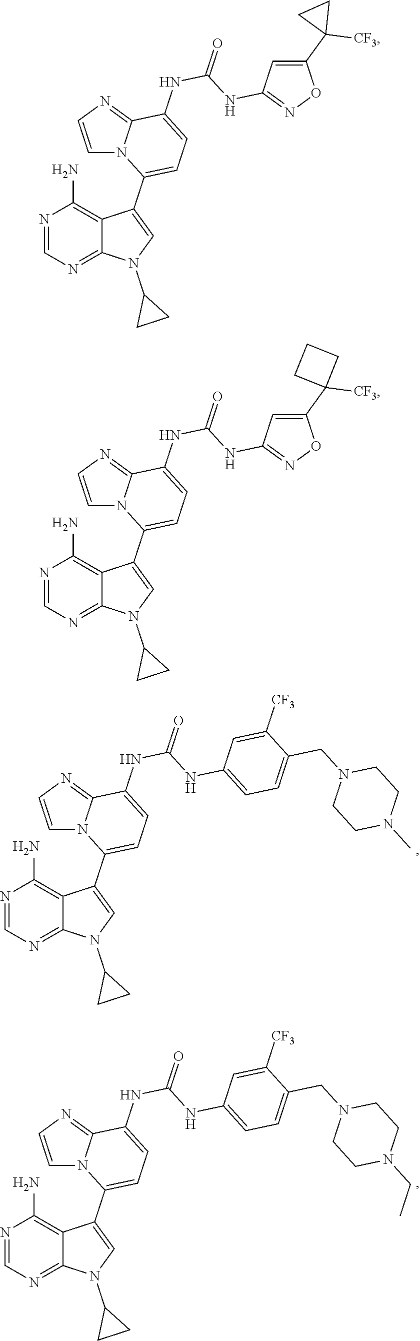 [SMILES: Nc1ncnc2c1c(-c1ccc(NC(=O)Nc3cc(C4(C(F)(F)F)CC4)on3)c3nccn13)cn2C1CC1 ; Nc1ncnc2c1c(-c1ccc(NC(=O)Nc3cc(C4(C(F)(F)F)CCC4)on3)c3nccn13)cn2C1CC1 ; CN1CCN(Cc2ccc(NC(=O)Nc3ccc(-c4cn(C5CC5)c5ncnc(N)c45)n4ccnc34)cc2C(F)(F)F)CC1 ; CCN1CCN(Cc2ccc(NC(=O)Nc3ccc(-c4cn(C5CC5)c5ncnc(N)c45)n4ccnc34)cc2C(F)(F)F)CC1] 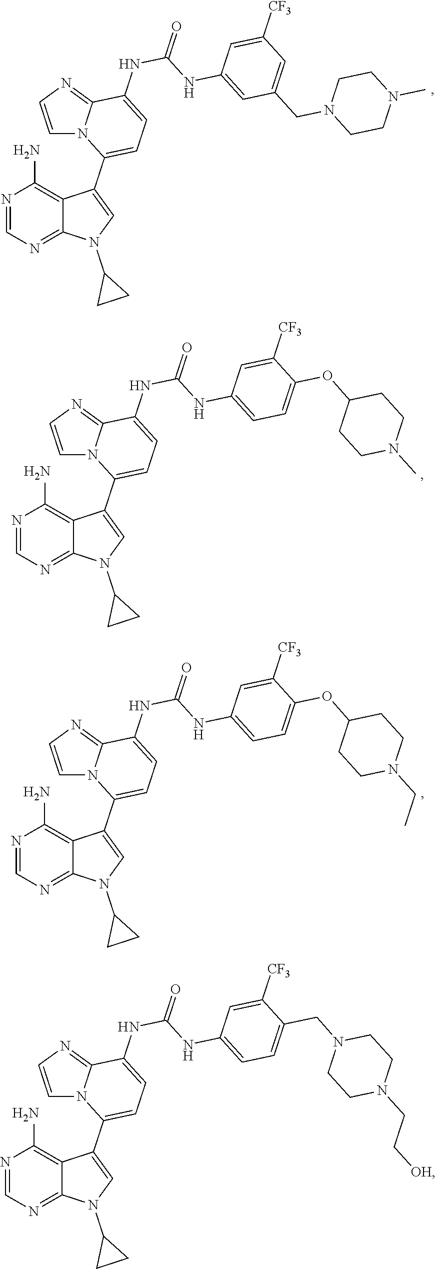 [SMILES: CN1CCN(Cc2cc(NC(=O)Nc3ccc(-c4cn(C5CC5)c5ncnc(N)c45)n4ccnc34)cc(C(F)(F)F)c2)CC1 ; CN1CCC(Oc2ccc(NC(=O)Nc3ccc(-c4cn(C5CC5)c5ncnc(N)c45)n4ccnc34)cc2C(F)(F)F)CC1 ; CCN1CCC(Oc2ccc(NC(=O)Nc3ccc(-c4cn(C5CC5)c5ncnc(N)c45)n4ccnc34)cc2C(F)(F)F)CC1 ; Nc1ncnc2c1c(-c1ccc(NC(=O)Nc3ccc(CN4CCN(CCO)CC4)c(C(F)(F)F)c3)c3nccn13)cn2C1CC1] 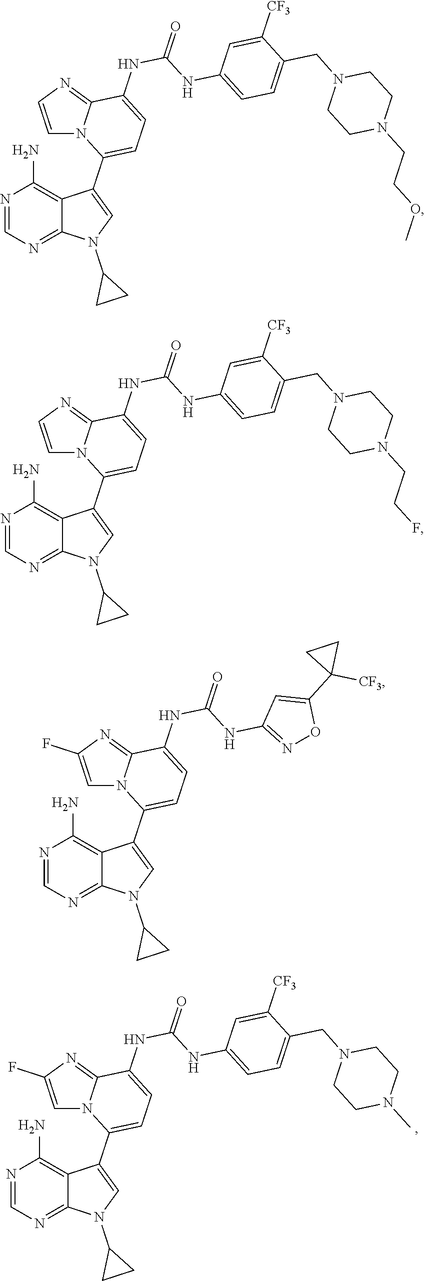 [SMILES: COCCN1CCN(Cc2ccc(NC(=O)Nc3ccc(-c4cn(C5CC5)c5ncnc(N)c45)n4ccnc34)cc2C(F)(F)F)CC1 ; Nc1ncnc2c1c(-c1ccc(NC(=O)Nc3ccc(CN4CCN(CCF)CC4)c(C(F)(F)F)c3)c3nccn13)cn2C1CC1 ; Nc1ncnc2c1c(-c1ccc(NC(=O)Nc3cc(C4(C(F)(F)F)CC4)on3)c3nc(F)cn13)cn2C1CC1 ; CN1CCN(Cc2ccc(NC(=O)Nc3ccc(-c4cn(C5CC5)c5ncnc(N)c45)n4cc(F)nc34)cc2C(F)(F)F)CC1] 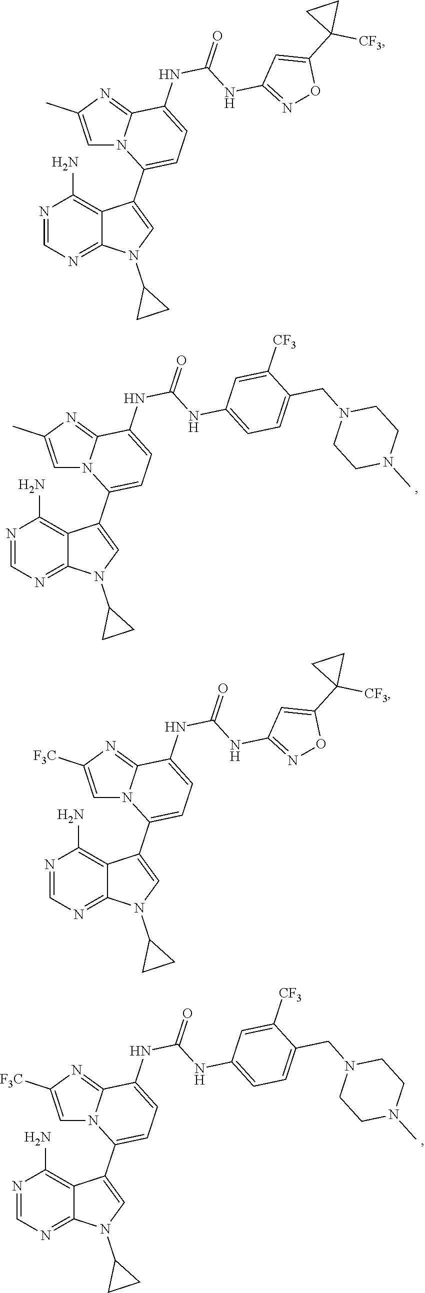 [SMILES: Cc1cn2c(-c3cn(C4CC4)c4ncnc(N)c34)ccc(NC(=O)Nc3cc(C4(C(F)(F)F)CC4)on3)c2n1 ; Cc1cn2c(-c3cn(C4CC4)c4ncnc(N)c34)ccc(NC(=O)Nc3ccc(CN4CCN(C)CC4)c(C(F)(F)F)c3)c2n1 ; Nc1ncnc2c1c(-c1ccc(NC(=O)Nc3cc(C4(C(F)(F)F)CC4)on3)c3nc(C(F)(F)F)cn13)cn2C1CC1 ; CN1CCN(Cc2ccc(NC(=O)Nc3ccc(-c4cn(C5CC5)c5ncnc(N)c45)n4cc(C(F)(F)F)nc34)cc2C(F)(F)F)CC1] 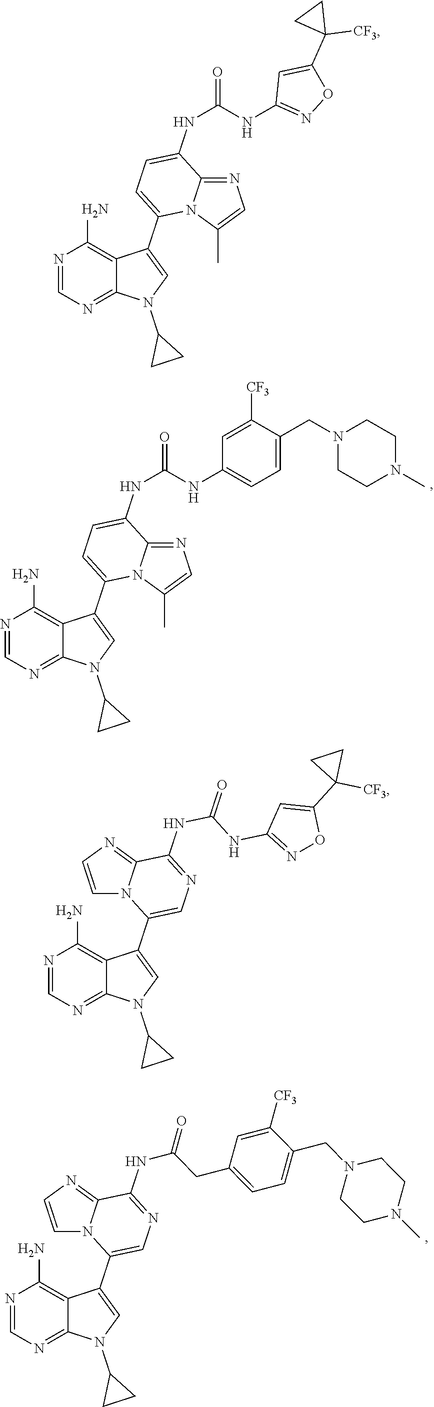 [SMILES: Cc1cnc2c(NC(=O)Nc3cc(C4(C(F)(F)F)CC4)on3)ccc(-c3cn(C4CC4)c4ncnc(N)c34)n12 ; Cc1cnc2c(NC(=O)Nc3ccc(CN4CCN(C)CC4)c(C(F)(F)F)c3)ccc(-c3cn(C4CC4)c4ncnc(N)c34)n12 ; Nc1ncnc2c1c(-c1cnc(NC(=O)Nc3cc(C4(C(F)(F)F)CC4)on3)c3nccn13)cn2C1CC1 ; CN1CCN(Cc2ccc(CC(=O)Nc3ncc(-c4cn(C5CC5)c5ncnc(N)c45)n4ccnc34)cc2C(F)(F)F)CC1] 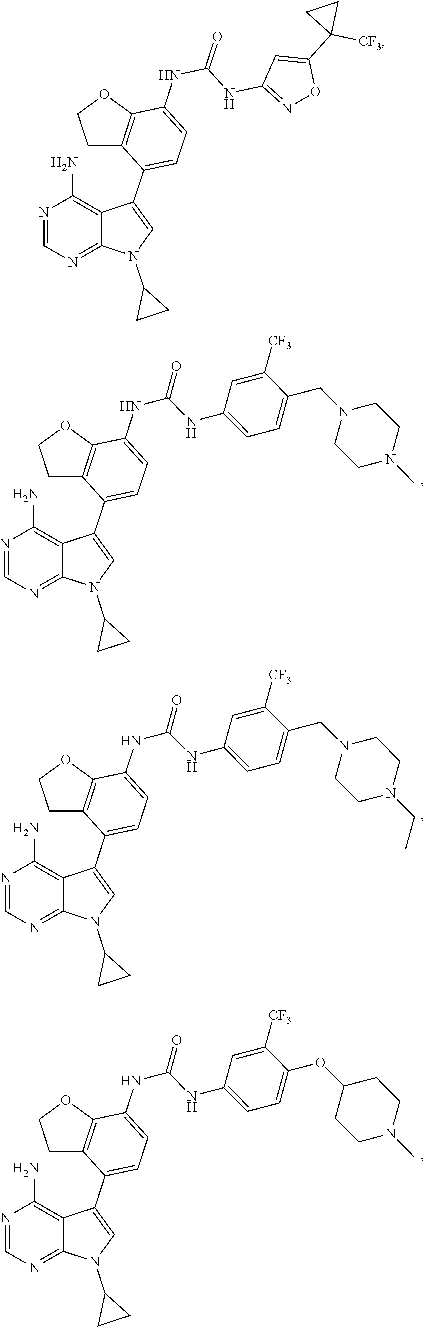 [SMILES: Nc1ncnc2c1c(-c1ccc(NC(=O)Nc3cc(C4(C(F)(F)F)CC4)on3)c3c1CCO3)cn2C1CC1 ; CN1CCN(Cc2ccc(NC(=O)Nc3ccc(-c4cn(C5CC5)c5ncnc(N)c45)c4c3OCC4)cc2C(F)(F)F)CC1 ; CCN1CCN(Cc2ccc(NC(=O)Nc3ccc(-c4cn(C5CC5)c5ncnc(N)c45)c4c3OCC4)cc2C(F)(F)F)CC1 ; CN1CCC(Oc2ccc(NC(=O)Nc3ccc(-c4cn(C5CC5)c5ncnc(N)c45)c4c3OCC4)cc2C(F)(F)F)CC1] 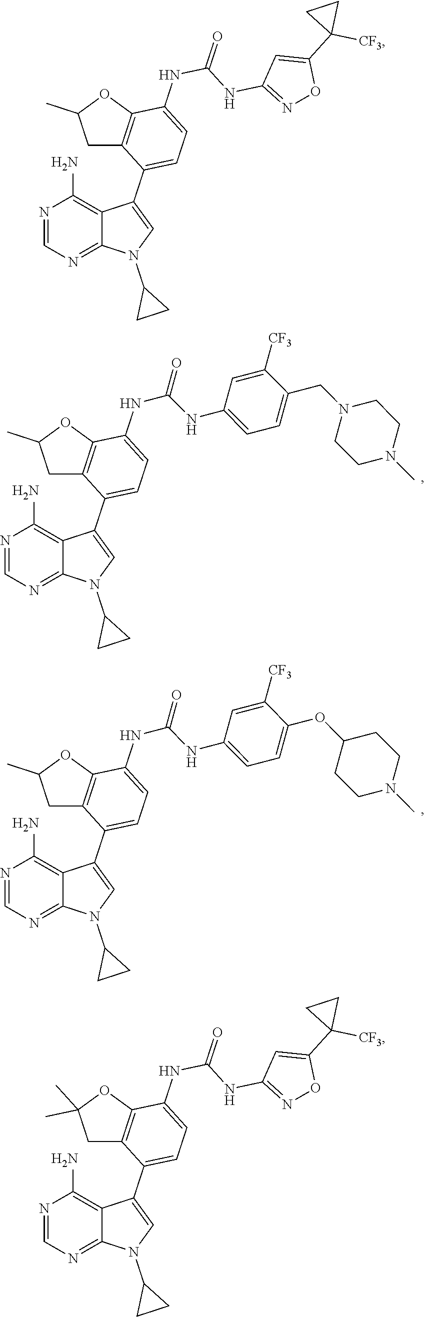 [SMILES: CC1Cc2c(-c3cn(C4CC4)c4ncnc(N)c34)ccc(NC(=O)Nc3cc(C4(C(F)(F)F)CC4)on3)c2O1 ; CC1Cc2c(-c3cn(C4CC4)c4ncnc(N)c34)ccc(NC(=O)Nc3ccc(CN4CCN(C)CC4)c(C(F)(F)F)c3)c2O1 ; CC1Cc2c(-c3cn(C4CC4)c4ncnc(N)c34)ccc(NC(=O)Nc3ccc(OC4CCN(C)CC4)c(C(F)(F)F)c3)c2O1 ; CC1(C)Cc2c(-c3cn(C4CC4)c4ncnc(N)c34)ccc(NC(=O)Nc3cc(C4(C(F)(F)F)CC4)on3)c2O1] 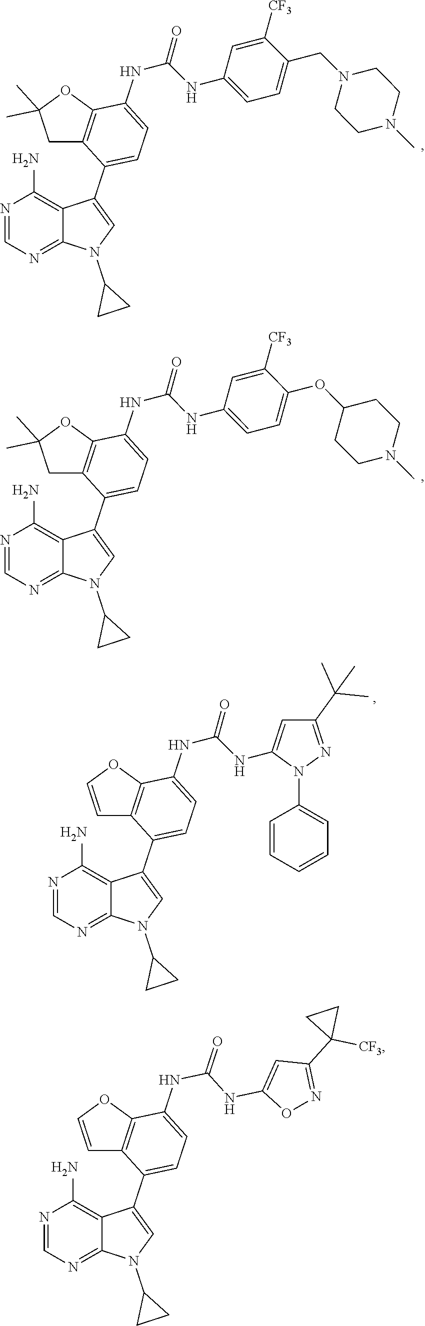 [SMILES: CN1CCN(Cc2ccc(NC(=O)Nc3ccc(-c4cn(C5CC5)c5ncnc(N)c45)c4c3OC(C)(C)C4)cc2C(F)(F)F)CC1 ; CN1CCC(Oc2ccc(NC(=O)Nc3ccc(-c4cn(C5CC5)c5ncnc(N)c45)c4c3OC(C)(C)C4)cc2C(F)(F)F)CC1 ; CC(C)(C)c1cc(NC(=O)Nc2ccc(-c3cn(C4CC4)c4ncnc(N)c34)c3ccoc23)n(-c2ccccc2)n1 ; Nc1ncnc2c1c(-c1ccc(NC(=O)Nc3cc(C4(C(F)(F)F)CC4)no3)c3occc13)cn2C1CC1] 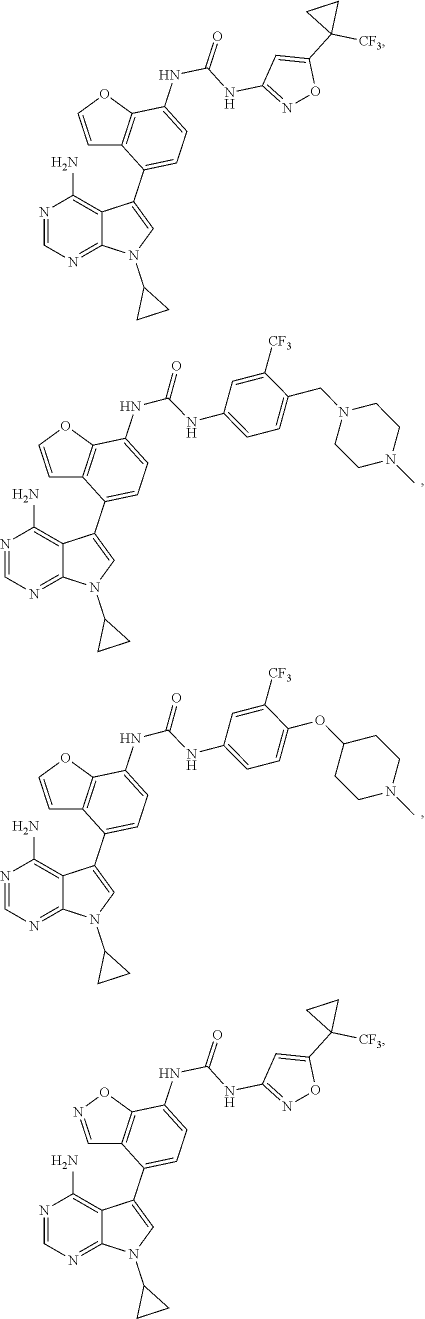 [SMILES: Nc1ncnc2c1c(-c1ccc(NC(=O)Nc3cc(C4(C(F)(F)F)CC4)on3)c3occc13)cn2C1CC1 ; CN1CCN(Cc2ccc(NC(=O)Nc3ccc(-c4cn(C5CC5)c5ncnc(N)c45)c4ccoc34)cc2C(F)(F)F)CC1 ; CN1CCC(Oc2ccc(NC(=O)Nc3ccc(-c4cn(C5CC5)c5ncnc(N)c45)c4ccoc34)cc2C(F)(F)F)CC1 ; Nc1ncnc2c1c(-c1ccc(NC(=O)Nc3cc(C4(C(F)(F)F)CC4)on3)c3oncc13)cn2C1CC1] 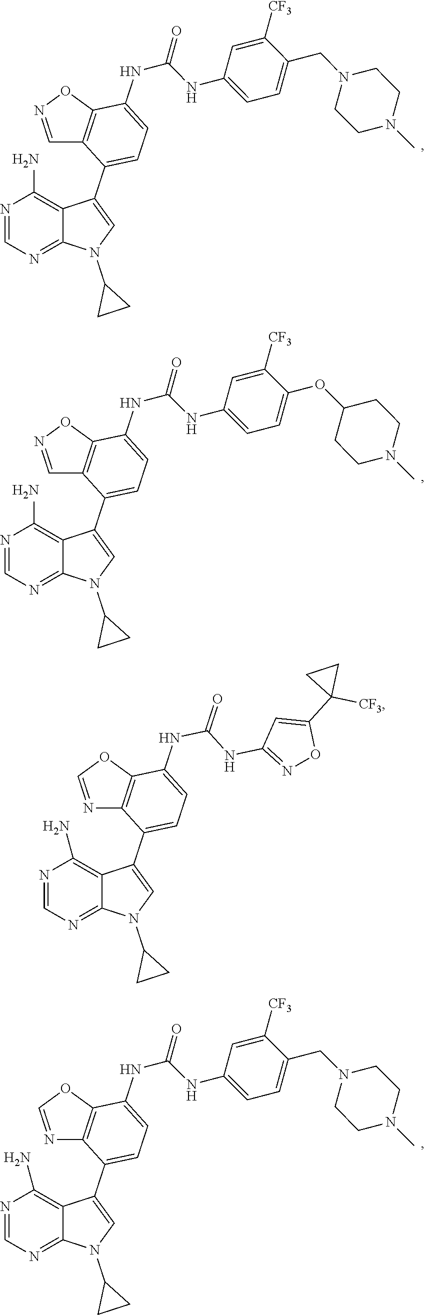 [SMILES: CN1CCN(Cc2ccc(NC(=O)Nc3ccc(-c4cn(C5CC5)c5ncnc(N)c45)c4cnoc34)cc2C(F)(F)F)CC1 ; CN1CCC(Oc2ccc(NC(=O)Nc3ccc(-c4cn(C5CC5)c5ncnc(N)c45)c4cnoc34)cc2C(F)(F)F)CC1 ; Nc1ncnc2c1c(-c1ccc(NC(=O)Nc3cc(C4(C(F)(F)F)CC4)on3)c3ocnc13)cn2C1CC1 ; CN1CCN(Cc2ccc(NC(=O)Nc3ccc(-c4cn(C5CC5)c5ncnc(N)c45)c4ncoc34)cc2C(F)(F)F)CC1] 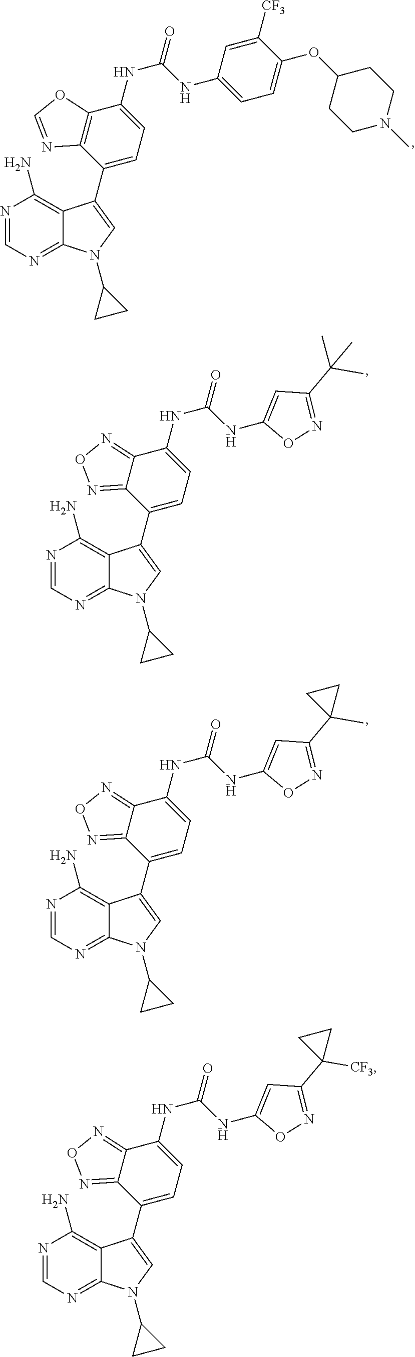 [SMILES: CN1CCC(Oc2ccc(NC(=O)Nc3ccc(-c4cn(C5CC5)c5ncnc(N)c45)c4ncoc34)cc2C(F)(F)F)CC1 ; CC(C)(C)c1cc(NC(=O)Nc2ccc(-c3cn(C4CC4)c4ncnc(N)c34)c3nonc23)on1 ; CC1(c2cc(NC(=O)Nc3ccc(-c4cn(C5CC5)c5ncnc(N)c45)c4nonc34)on2)CC1 ; Nc1ncnc2c1c(-c1ccc(NC(=O)Nc3cc(C4(C(F)(F)F)CC4)no3)c3nonc13)cn2C1CC1] 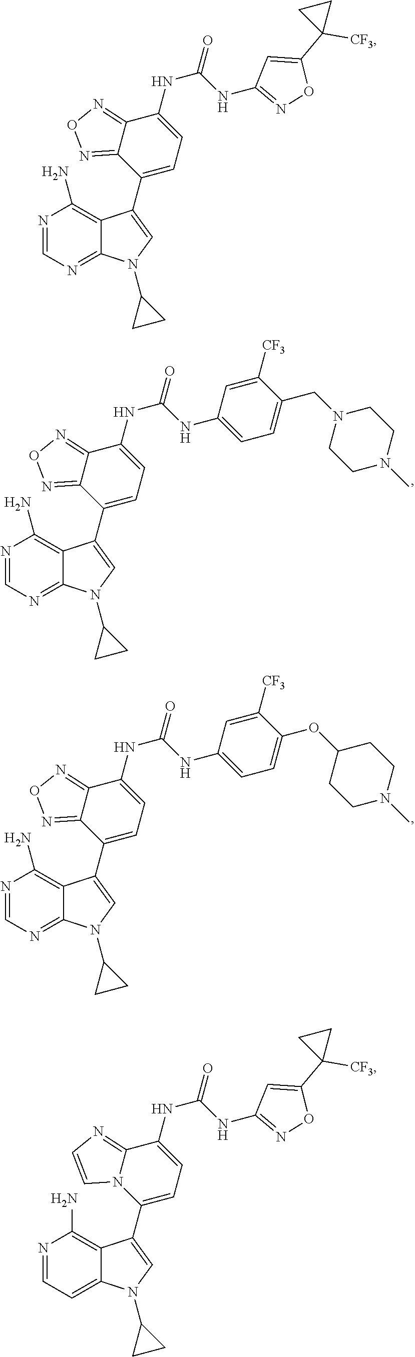 [SMILES: Nc1ncnc2c1c(-c1ccc(NC(=O)Nc3cc(C4(C(F)(F)F)CC4)on3)c3nonc13)cn2C1CC1 ; CN1CCN(Cc2ccc(NC(=O)Nc3ccc(-c4cn(C5CC5)c5ncnc(N)c45)c4nonc34)cc2C(F)(F)F)CC1 ; CN1CCC(Oc2ccc(NC(=O)Nc3ccc(-c4cn(C5CC5)c5ncnc(N)c45)c4nonc34)cc2C(F)(F)F)CC1 ; Nc1nccc2c1c(-c1ccc(NC(=O)Nc3cc(C4(C(F)(F)F)CC4)on3)c3nccn13)cn2C1CC1] 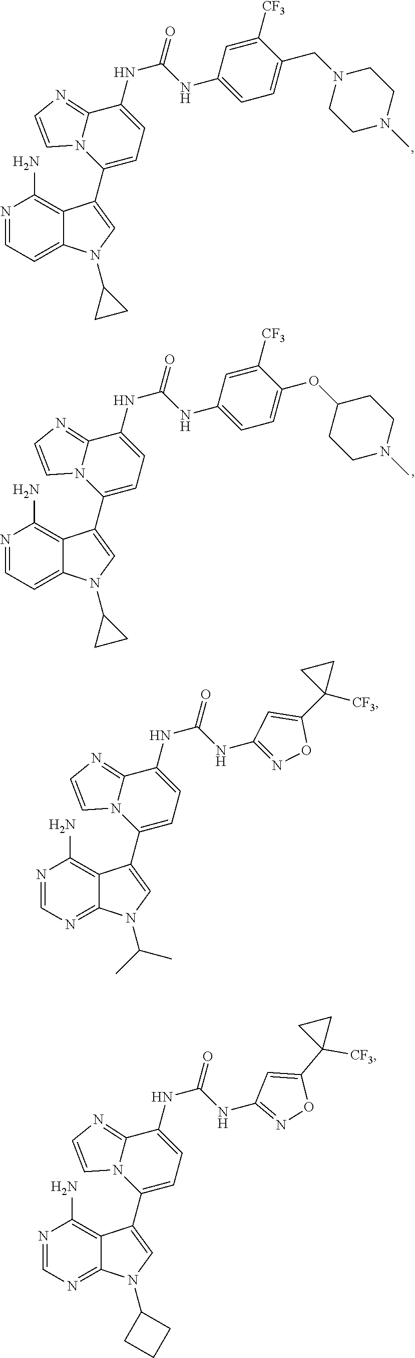 [SMILES: CN1CCN(Cc2ccc(NC(=O)Nc3ccc(-c4cn(C5CC5)c5ccnc(N)c45)n4ccnc34)cc2C(F)(F)F)CC1 ; CN1CCC(Oc2ccc(NC(=O)Nc3ccc(-c4cn(C5CC5)c5ccnc(N)c45)n4ccnc34)cc2C(F)(F)F)CC1 ; CC(C)n1cc(-c2ccc(NC(=O)Nc3cc(C4(C(F)(F)F)CC4)on3)c3nccn23)c2c(N)ncnc21 ; Nc1ncnc2c1c(-c1ccc(NC(=O)Nc3cc(C4(C(F)(F)F)CC4)on3)c3nccn13)cn2C1CCC1] 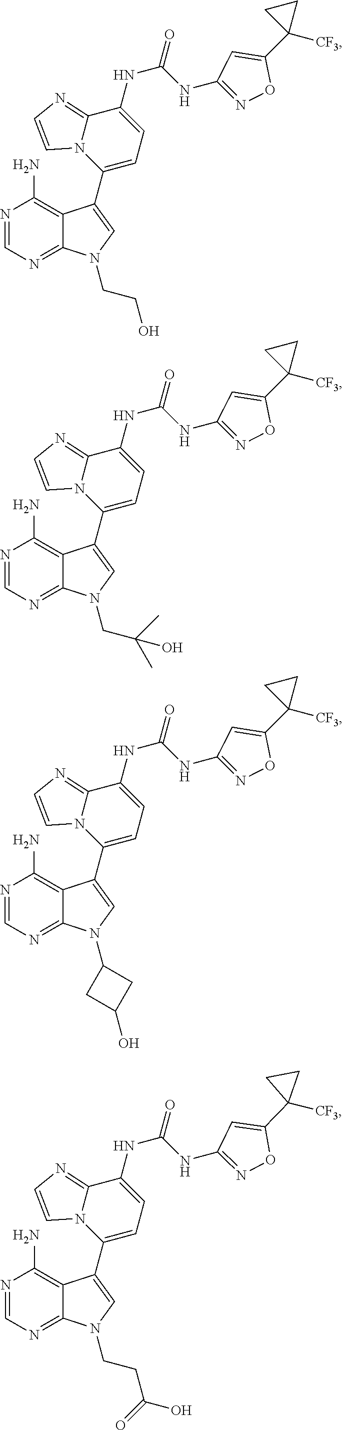 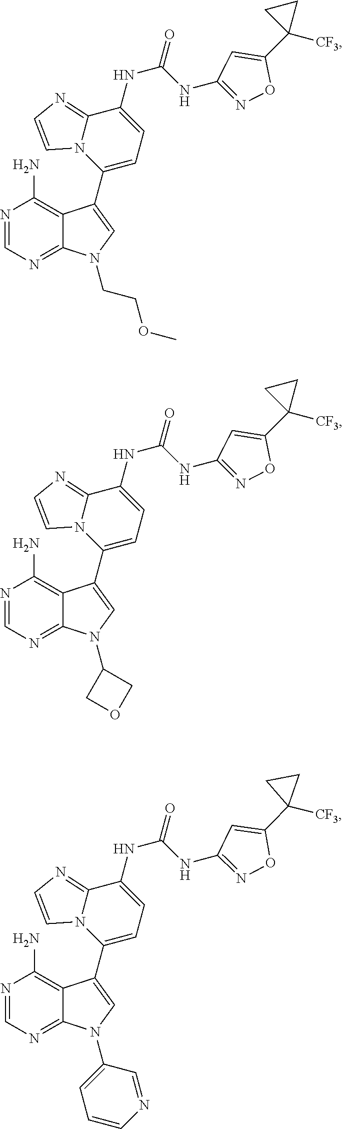 [SMILES: COCCn1cc(-c2ccc(NC(=O)Nc3cc(C4(C(F)(F)F)CC4)on3)c3nccn23)c2c(N)ncnc21 ; Nc1ncnc2c1c(-c1ccc(NC(=O)Nc3cc(C4(C(F)(F)F)CC4)on3)c3nccn13)cn2C1COC1 ; Nc1ncnc2c1c(-c1ccc(NC(=O)Nc3cc(C4(C(F)(F)F)CC4)on3)c3nccn13)cn2-c1cccnc1] 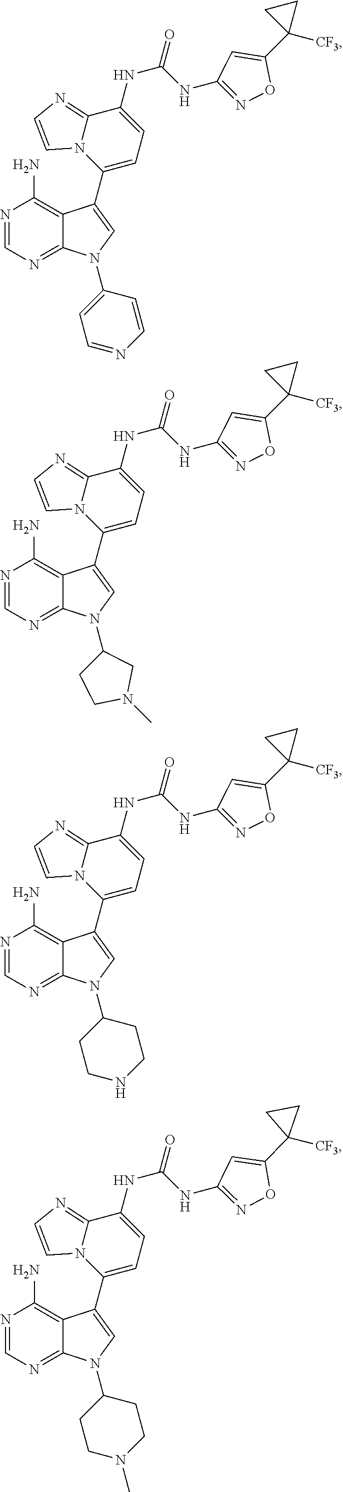 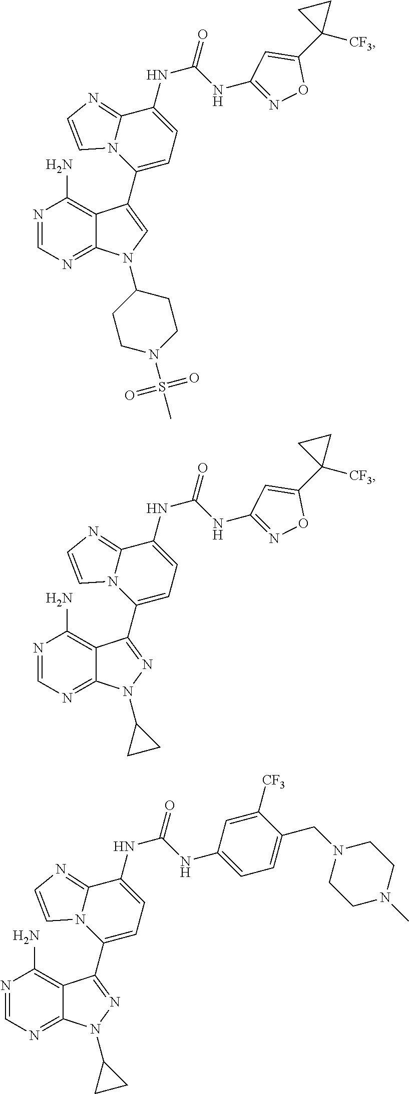 [SMILES: CS(=O)(=O)N1CCC(n2cc(-c3ccc(NC(=O)Nc4cc(C5(C(F)(F)F)CC5)on4)c4nccn34)c3c(N)ncnc32)CC1 ; Nc1ncnc2c1c(-c1ccc(NC(=O)Nc3cc(C4(C(F)(F)F)CC4)on3)c3nccn13)nn2C1CC1 ; CN1CCN(Cc2ccc(NC(=O)Nc3ccc(-c4nn(C5CC5)c5ncnc(N)c45)n4ccnc34)cc2C(F)(F)F)CC1] 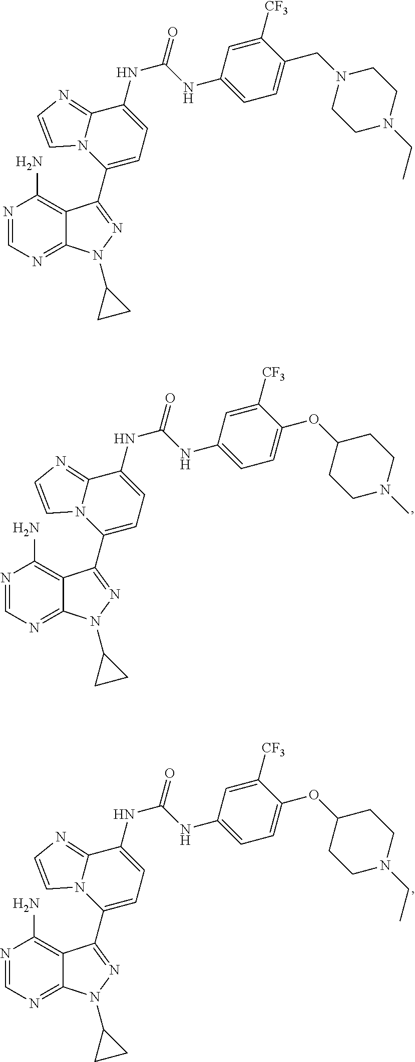 [SMILES: CCN1CCN(Cc2ccc(NC(=O)Nc3ccc(-c4nn(C5CC5)c5ncnc(N)c45)n4ccnc34)cc2C(F)(F)F)CC1 ; CN1CCC(Oc2ccc(NC(=O)Nc3ccc(-c4nn(C5CC5)c5ncnc(N)c45)n4ccnc34)cc2C(F)(F)F)CC1 ; CCN1CCC(Oc2ccc(NC(=O)Nc3ccc(-c4nn(C5CC5)c5ncnc(N)c45)n4ccnc34)cc2C(F)(F)F)CC1] 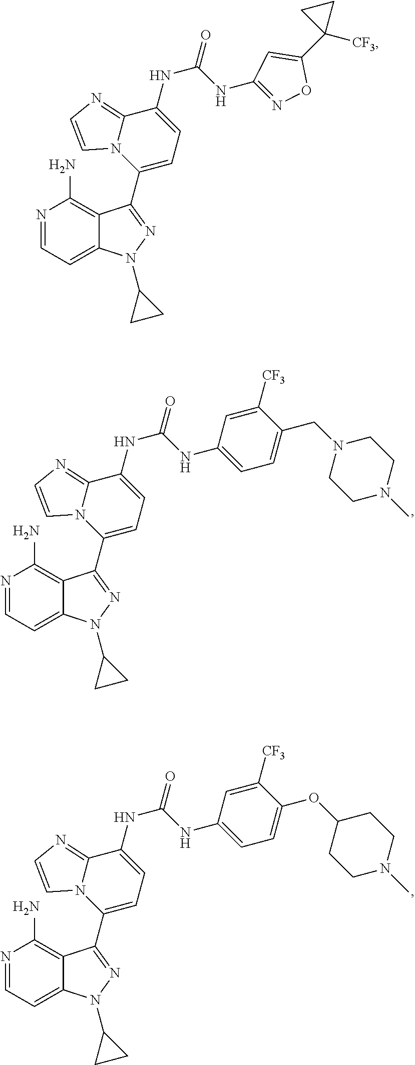 [SMILES: Nc1nccc2c1c(-c1ccc(NC(=O)Nc3cc(C4(C(F)(F)F)CC4)on3)c3nccn13)nn2C1CC1 ; CN1CCN(Cc2ccc(NC(=O)Nc3ccc(-c4nn(C5CC5)c5ccnc(N)c45)n4ccnc34)cc2C(F)(F)F)CC1 ; CN1CCC(Oc2ccc(NC(=O)Nc3ccc(-c4nn(C5CC5)c5ccnc(N)c45)n4ccnc34)cc2C(F)(F)F)CC1] 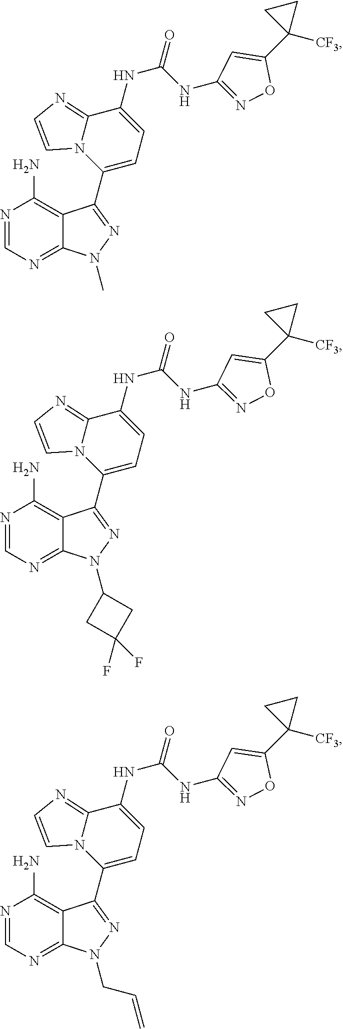 [SMILES: Cn1nc(-c2ccc(NC(=O)Nc3cc(C4(C(F)(F)F)CC4)on3)c3nccn23)c2c(N)ncnc21 ; Nc1ncnc2c1c(-c1ccc(NC(=O)Nc3cc(C4(C(F)(F)F)CC4)on3)c3nccn13)nn2C1CC(F)(F)C1 ; C=CCn1nc(-c2ccc(NC(=O)Nc3cc(C4(C(F)(F)F)CC4)on3)c3nccn23)c2c(N)ncnc21] 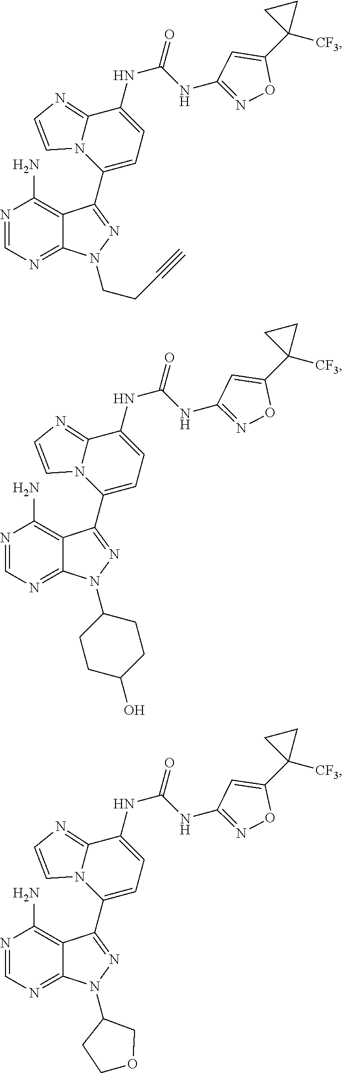 [SMILES: C#CCCn1nc(-c2ccc(NC(=O)Nc3cc(C4(C(F)(F)F)CC4)on3)c3nccn23)c2c(N)ncnc21 ; Nc1ncnc2c1c(-c1ccc(NC(=O)Nc3cc(C4(C(F)(F)F)CC4)on3)c3nccn13)nn2C1CCC(O)CC1 ; Nc1ncnc2c1c(-c1ccc(NC(=O)Nc3cc(C4(C(F)(F)F)CC4)on3)c3nccn13)nn2C1CCOC1] 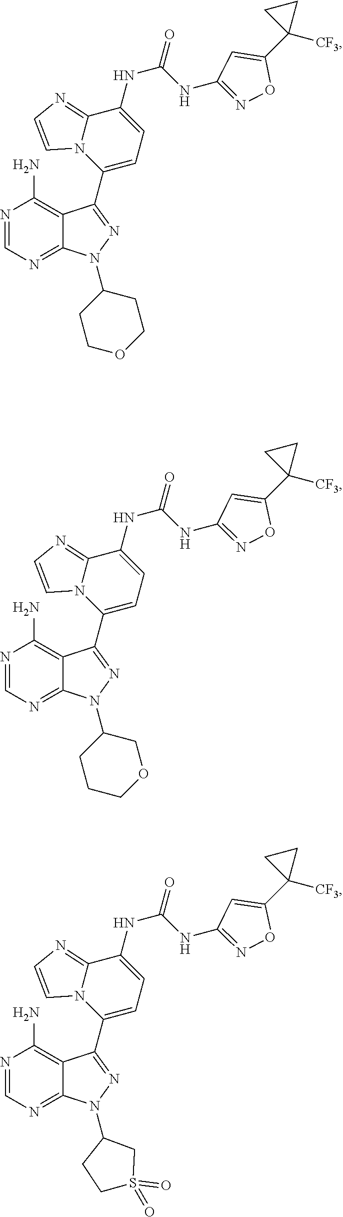 [SMILES: Nc1ncnc2c1c(-c1ccc(NC(=O)Nc3cc(C4(C(F)(F)F)CC4)on3)c3nccn13)nn2C1CCOCC1 ; Nc1ncnc2c1c(-c1ccc(NC(=O)Nc3cc(C4(C(F)(F)F)CC4)on3)c3nccn13)nn2C1CCCOC1 ; Nc1ncnc2c1c(-c1ccc(NC(=O)Nc3cc(C4(C(F)(F)F)CC4)on3)c3nccn13)nn2C1CCS(=O)(=O)C1] 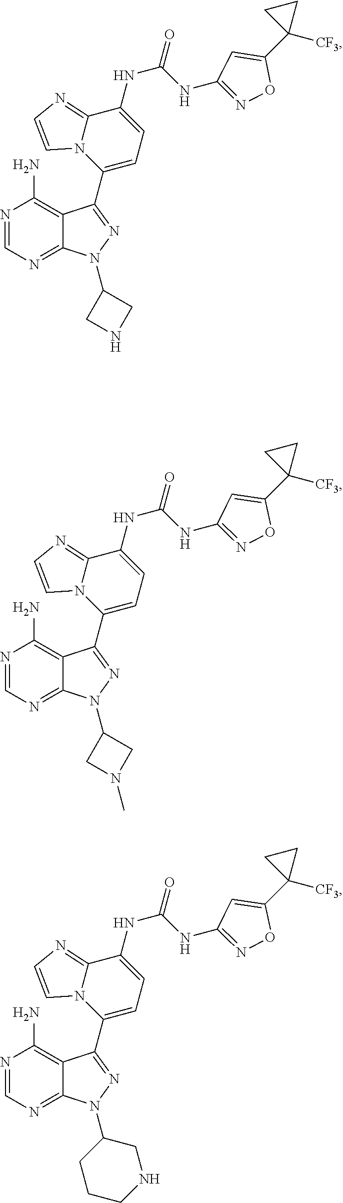 [SMILES: Nc1ncnc2c1c(-c1ccc(NC(=O)Nc3cc(C4(C(F)(F)F)CC4)on3)c3nccn13)nn2C1CNC1 ; CN1CC(n2nc(-c3ccc(NC(=O)Nc4cc(C5(C(F)(F)F)CC5)on4)c4nccn34)c3c(N)ncnc32)C1 ; Nc1ncnc2c1c(-c1ccc(NC(=O)Nc3cc(C4(C(F)(F)F)CC4)on3)c3nccn13)nn2C1CCCNC1] 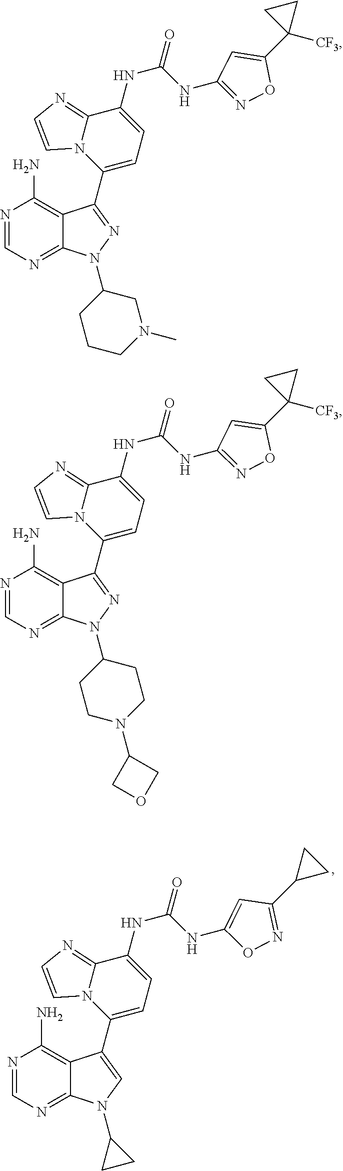 [SMILES: CN1CCCC(n2nc(-c3ccc(NC(=O)Nc4cc(C5(C(F)(F)F)CC5)on4)c4nccn34)c3c(N)ncnc32)C1 ; Nc1ncnc2c1c(-c1ccc(NC(=O)Nc3cc(C4(C(F)(F)F)CC4)on3)c3nccn13)nn2C1CCN(C2COC2)CC1 ; Nc1ncnc2c1c(-c1ccc(NC(=O)Nc3cc(C4CC4)no3)c3nccn13)cn2C1CC1] 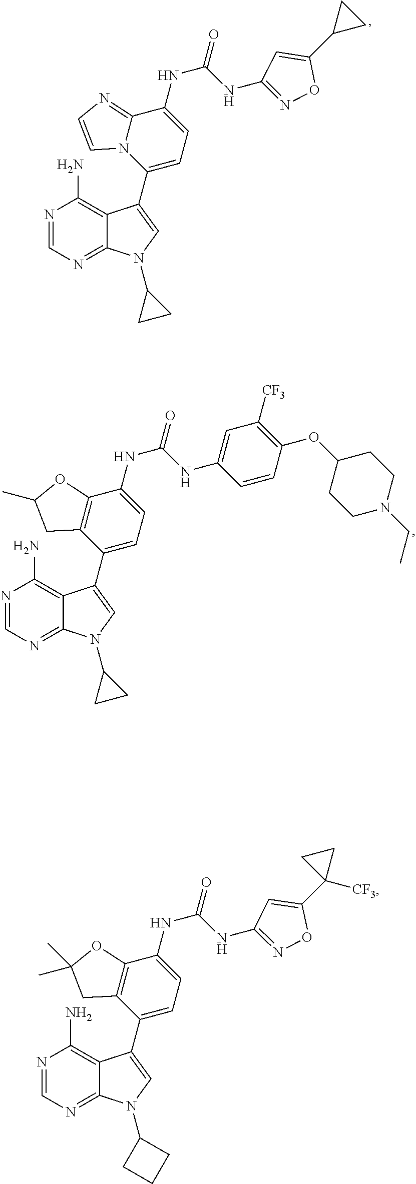 [SMILES: Nc1ncnc2c1c(-c1ccc(NC(=O)Nc3cc(C4CC4)on3)c3nccn13)cn2C1CC1 ; CCN1CCC(Oc2ccc(NC(=O)Nc3ccc(-c4cn(C5CC5)c5ncnc(N)c45)c4c3OC(C)C4)cc2C(F)(F)F)CC1 ; CC1(C)Cc2c(-c3cn(C4CCC4)c4ncnc(N)c34)ccc(NC(=O)Nc3cc(C4(C(F)(F)F)CC4)on3)c2O1] 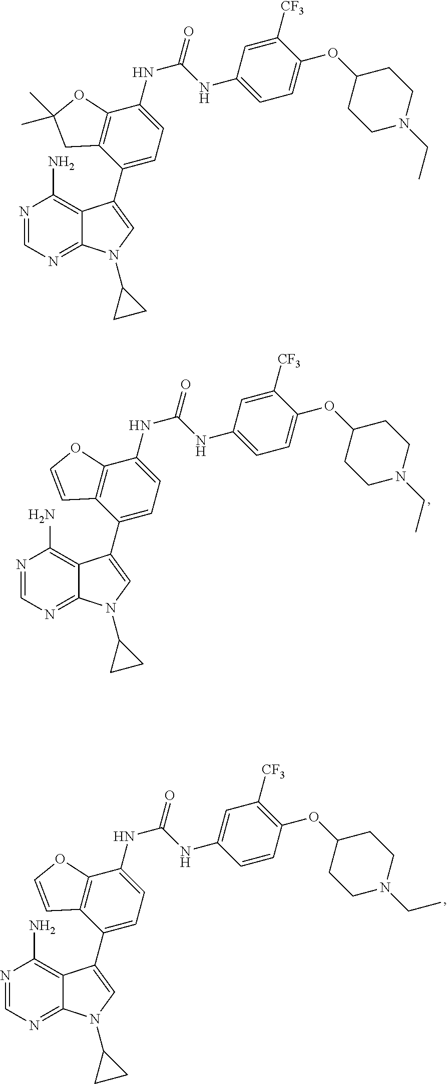 [SMILES: CCN1CCC(Oc2ccc(NC(=O)Nc3ccc(-c4cn(C5CC5)c5ncnc(N)c45)c4c3OC(C)(C)C4)cc2C(F)(F)F)CC1 ; CCN1CCC(Oc2ccc(NC(=O)Nc3ccc(-c4cn(C5CC5)c5ncnc(N)c45)c4ccoc34)cc2C(F)(F)F)CC1 ; CCN1CCC(Oc2ccc(NC(=O)Nc3ccc(-c4cn(C5CC5)c5ncnc(N)c45)c4ccoc34)cc2C(F)(F)F)CC1] 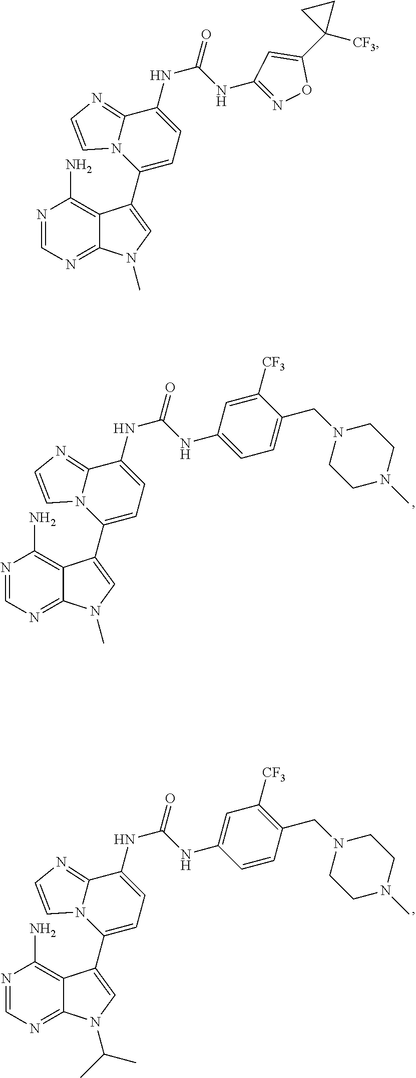 [SMILES: Cn1cc(-c2ccc(NC(=O)Nc3cc(C4(C(F)(F)F)CC4)on3)c3nccn23)c2c(N)ncnc21 ; CN1CCN(Cc2ccc(NC(=O)Nc3ccc(-c4cn(C)c5ncnc(N)c45)n4ccnc34)cc2C(F)(F)F)CC1 ; CC(C)n1cc(-c2ccc(NC(=O)Nc3ccc(CN4CCN(C)CC4)c(C(F)(F)F)c3)c3nccn23)c2c(N)ncnc21] 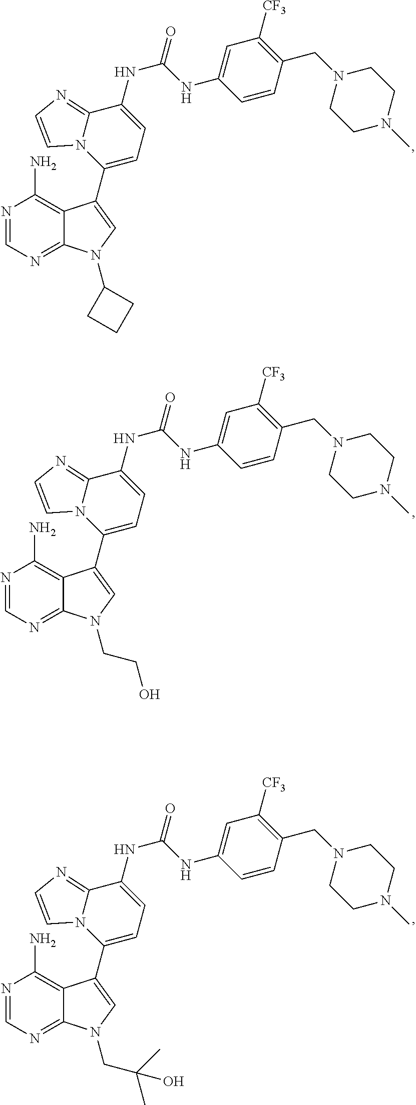 [SMILES: CN1CCN(Cc2ccc(NC(=O)Nc3ccc(-c4cn(C5CCC5)c5ncnc(N)c45)n4ccnc34)cc2C(F)(F)F)CC1 ; CN1CCN(Cc2ccc(NC(=O)Nc3ccc(-c4cn(CCO)c5ncnc(N)c45)n4ccnc34)cc2C(F)(F)F)CC1 ; CN1CCN(Cc2ccc(NC(=O)Nc3ccc(-c4cn(CC(C)(C)O)c5ncnc(N)c45)n4ccnc34)cc2C(F)(F)F)CC1] 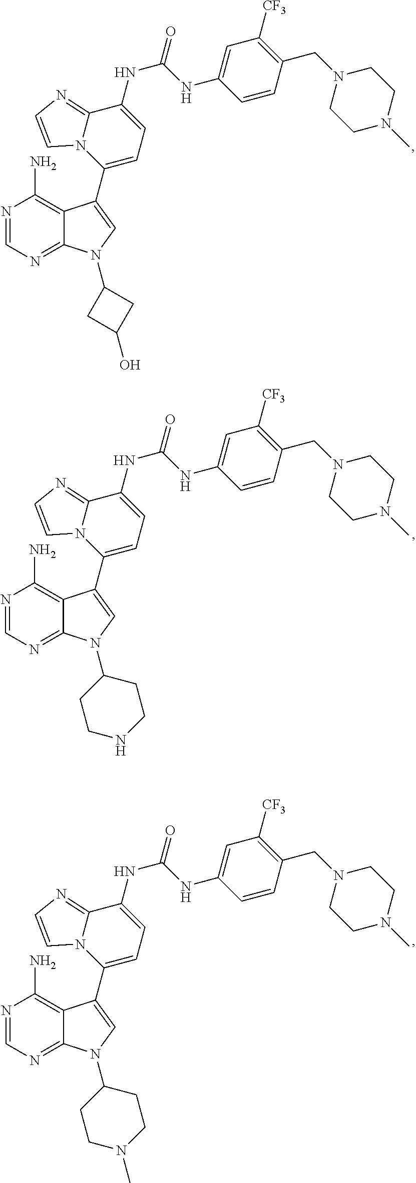 [SMILES: CN1CCN(Cc2ccc(NC(=O)Nc3ccc(-c4cn(C5CC(O)C5)c5ncnc(N)c45)n4ccnc34)cc2C(F)(F)F)CC1 ; CN1CCN(Cc2ccc(NC(=O)Nc3ccc(-c4cn(C5CCNCC5)c5ncnc(N)c45)n4ccnc34)cc2C(F)(F)F)CC1 ; CN1CCC(n2cc(-c3ccc(NC(=O)Nc4ccc(CN5CCN(C)CC5)c(C(F)(F)F)c4)c4nccn34)c3c(N)ncnc32)CC1] 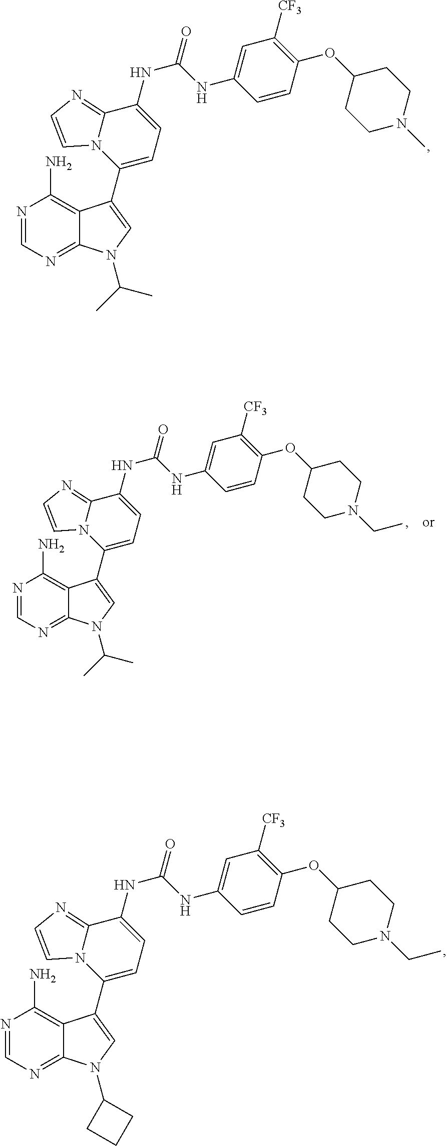 [SMILES: CC(C)n1cc(-c2ccc(NC(=O)Nc3ccc(OC4CCN(C)CC4)c(C(F)(F)F)c3)c3nccn23)c2c(N)ncnc21 ; CCN1CCC(Oc2ccc(NC(=O)Nc3ccc(-c4cn(C(C)C)c5ncnc(N)c45)n4ccnc34)cc2C(F)(F)F)CC1 ; CCN1CCC(Oc2ccc(NC(=O)Nc3ccc(-c4cn(C5CCC5)c5ncnc(N)c45)n4ccnc34)cc2C(F)(F)F)CC1]
or a stereoisomer, tautomer, or salt thereof.

47. The compound of claim 1, wherein the compound is a modulator of the NLRP3 inflammasome, an inhibitor of NEK7, or both.

48. (canceled)

49. A pharmaceutical composition comprising the compound of claim 1, and a pharmaceutically acceptable carrier, diluent, or excipient.

50. A method of treating a NLRP3-mediated disorder, the method comprising administering a therapeutically effective amount of a compound of claim 1, to a subject in need thereof wherein the disorder is selected from type II diabetes, atherosclerosis, Alzheimer's disease, fatty liver, asthma, psoriasis, obesity, acute and chronic tissue damage caused by infection, gout, arthritis, macular degeneration, enteritis, hepatitis, peritonitis, silicosis, UV-induced skin sunburn, contact hypersensitivity, sepsis, cancer, neurodegenerative disease, multiple sclerosis, inflammatory bowel disease, and Muckle-Wells syndrome.

51. A method for treating a NLRP3-mediated disorder, the method comprising administering a therapeutically effective amount of a compound of claim 1 wherein the disorder is an inflammatory disorder.

52. (canceled)
